(19) United States
(12) Patent Application Publication
(10) Pub. No.: US 2015/0214491 A1
(43) Pub. Date: Jul. 30, 2015

(54) NOVEL AROMATIC HETEROCYCLIC DERIVATIVE, ORGANIC ELECTROLUMINESCENT ELEMENT MATERIAL, ORGANIC ELECTROLUMINESCENT ELEMENT MATERIAL SOLUTION, AND ORGANIC ELECTROLUMINESCENT ELEMENT

(71) Applicant: IDEMITSU KOSAN CO., LTD., Chiyoda-ku, Tokyo (JP)

(72) Inventors: Kiyoshi Ikeda, Sodegaura-shi (JP); Hironori Kawakami, Katsushika-ku (JP); Akinori Yomogita, Ichihara-shi (JP); Mitsunori Ito, Sodegaura-shi (JP)

(73) Assignee: IDEMITSU KOSAN CO., LTD., Chiyoda-ku, Tokyo (JP)

(21) Appl. No.: 14/426,614

(22) PCT Filed: Sep. 6, 2013
(86) PCT No.: PCT/JP2013/074154

§ 371 Date: Mar. 6, 2015

(30) Foreign Application Priority Data
Sep. 7, 2012 (JP) 2012-197944

(51) Int. Cl. H01L 51/00; C09K 11/06; C07D 209/86; C09K 11/02; C07D 403/10; C07D 403/04

(52) U.S. Cl. CPC H01L 51/0072; C07D 403/10; C07D 403/04; C07D 209/86; C09K 11/025; C09K 11/06; H01L 51/0067; H01L 51/0085; H01L 51/0058; C09K 2211/185; H01L 51/5016

(57) ABSTRACT

A novel aromatic heterocyclic derivative has a specific structure in its molecule which combines a hole transporting ability and an electron transporting ability. The aromatic heterocyclic derivative is used in a material for an organic electroluminescence device, a solution of a material for an organic electroluminescence device, and an organic electroluminescence device.

### TECHNICAL FIELD

The present invention relates to novel aromatic heterocyclic derivatives, materials for organic electroluminescence devices, solutions of the materials for organic electroluminescence devices, and organic electroluminescence devices.

### BACKGROUND ART

Organic electroluminescence devices (hereinafter also referred to as “organic EL device”) have been known, in which an organic thin film layer including a light emitting layer is disposed between an anode and a cathode, and the energy of exciton generated by the recombination of hole and electron which are injected into a light emitting layer is converted into light.

Utilizing its advantages as the spontaneous emitting device, the organic EL device has been expected to provide a light emitting device excellent in the emission efficiency, the image quality, the power consumption, and the freedom of design. It has been known to form a light emitting layer by a doping method in which a host is doped with an emission material as a dopant.

In a light emitting layer formed by a doping method, excitons can be efficiently generated from charges injected into a host. The energy of generated excitons is transferred to the dopant, and the light emission from the dopant with high efficiency can be obtained.

To improve the performance of organic EL devices, the recent study is directed also to a doping method, and the search for a suitable host material has been continued.

Patent Document 1 describes a compound including a structure in which two carbazole structures are linked to each other (i.e. biscarbazole structure). A carbazole structure, as represented by a polyvinylcarbazole known for a long time, has been known as a structure with a high hole transporting ability (also referred to as “hole transporting structure”). Therefore, the compound described in Patent Document 1 is preferred as a material for a hole transporting layer. However, since the proposed compound does not include in its molecule a structure with a high electron transporting ability (also referred to as “electron transporting structure”), for example, a nitrogen-containing aromatic ring structure, the carrier balance between holes and electrons are difficult to control. The inventors have found that a good emission performance cannot be obtained by using the compound described in Patent Document 1 as a host material.

Patent Document 2 describes a compound having a partial structure including a carbazolyl group and further describes a compound having a combined structure of a partial structure including a carbazolyl group and an electron transporting structure, such as a nitrogen-containing aromatic ring structure. However, the inventors have found that an organic EL device employing the compound described in Patent Document 2 is insufficient in the performance, for example, in the lifetime.

Patent Document 3 describes a compound including in its molecule a hole transporting structure, such as a biscarbazole structure, and an electron transporting structure, such as a nitrogen-containing aromatic ring structure. This compound is designed so as to balance the charge transport by combining the hole transporting structure and the electron transporting structure.

Patent Document 4 describes a compound including, between two carbazole structures, a structure in which a cyano group is bonded via a phenylene group. A cyano group is known as an electron withdrawing group, and the inventors have found that the hole transporting ability of the carbazole structure is reduced when a cyano group is located between and closely to two carbazole structures as in the compound of Patent Document 4.

The method for forming each layer of an organic EL device is classified roughly into a vapor deposition method, such a vacuum vapor deposition method and a molecular beam evaporation method, and a coating method, such as a dipping method, a spin coating method, a casting method, a bar coating method, and a roll coating method. Unlike the vapor deposition method, a material for an organic EL device for use in the coating method is required to be soluble in a solvent. Therefore, a material useful in the vapor deposition method is not necessarily useful in the coating method.

In the working examples of Patent Documents 1 and 4, the organic EL devices are produced by vapor-depositing the compounds described therein into layers, and the compounds are not made into layers by the coating method. Therefore, it is unclear whether the compounds described in these Patent Documents are soluble in a solvent and usable in the coating method.

### CITATION LIST

### Patent Documents

### Patent Document 1: JP 3139321B

### Patent Document 2: JP 2006-188493A

### Patent Document 3: WO 2012/086170

### Patent Document 4: JP 2009-94486A

### SUMMARY OF THE INVENTION

### Problems to be Solved by the Invention

An object of the invention is to provide a novel aromatic heterocyclic derivative. Another object of the invention is to provide a material for an organic electroluminescence device, a solution of a material for an organic electroluminescence device, and an organic electroluminescence device, each employing the aromatic heterocyclic derivative.

### Means for Solving the Problems

As a result of extensive research in view of achieving the above objects, the inventors have found that a novel aromatic heterocyclic derivative having a specific structure in its molecule which combines the hole transporting ability and the electron transporting ability is soluble and provides a material for an organic EL device suitable for use in a coating process, and further found that a long lifetime organic EL device is realized by the coating process. The present invention is based on these findings.

Thus, the embodiments provided by the present invention include:

1. An aromatic heterocyclic derivative represented by formula (1):

AL¹-B)ₘ (1)

wherein:

A represents a substituted or unsubstituted aromatic hydrocarbon ring group, a substituted or unsubstituted aromatic heterocyclic group, a residue of a ring assembly which comprises at least two substituted or unsubstituted aromatic hydrocarbon rings, a residue of a ring assembly which comprises at least two substituted or unsubstituted aromatic heterocyclic rings, or a residue of a ring assembly which comprises at least one substituted or unsubstituted aromatic hydrocarbon ring and at least one substituted or unsubstituted aromatic heterocyclic ring;

L¹ represents a single bond, a substituted or unsubstituted aromatic hydrocarbon ring group, or a substituted or unsubstituted aromatic heterocyclic group;

B represents a residue of a structure represented by formula (2-b); and

m represents an integer of 2 or more, groups L¹ may be the same or different, and groups B may be the same or different,

provided that a group represented by formula (3) is bonded to at least one of A, L¹ and B;

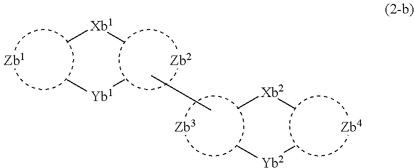

wherein:

one of Xb¹ and Yb¹ represents a single bond, —CR₂—, —NR—, —O—, —S—, —SiR₂—, a group represented by formula (i), or a group represented by formula (ii), and the other represents —NR—, —O—, —S—, —SiR₂—, a group represented by formula (i), or a group represented by formula (ii);

one of Xb² and Yb² represents a single bond, —CR₂—, —NR—, —O—, —S—, —SiR₂—, a group represented by formula (i), or a group represented by formula (ii), and the other represents —NR—, —O—, —S—, —SiR₂—, a group represented by formula (i), or a group represented by formula (ii);

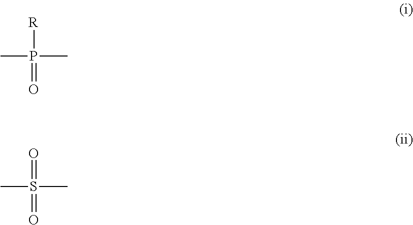

R represent a hydrogen atom, a substituted or unsubstituted alkyl group, a substituted or unsubstituted cycloalkyl group, a substituted or unsubstituted aromatic hydrocarbon ring group, or a substituted or unsubstituted aromatic heterocyclic group;

each of Zb¹, Zb², Zb³, and Zb⁴ independently represents a substituted or unsubstituted aliphatic hydrocarbon ring group, a substituted or unsubstituted aliphatic heterocyclic group, a substituted or unsubstituted aromatic hydrocarbon ring group, or a substituted or unsubstituted aromatic heterocyclic group;

-L³-F (3)

wherein

L³ represents a single bond, a substituted or unsubstituted aromatic hydrocarbon ring group, or a substituted or unsubstituted aromatic heterocyclic group;

when the group represented by formula (3) is bonded to A, F represents a group selected from the group consisting of a cyano group, a fluorine atom, a haloalkyl group, a substituted or unsubstituted triphenylenyl group, a substituted or unsubstituted azafluorenyl group, a substituted or unsubstituted spirofluorenyl group, a substituted or unsubstituted dibenzothiophenyl group, a substituted or unsubstituted bipyridinyl group, a substituted or unsubstituted bipyrimidinyl group, a substituted or unsubstituted quinazolinyl group, a substituted or unsubstituted imidazolyl group, a substituted or unsubstituted benzimidazolyl group, a phosphorus-containing group, a silicon-containing group, and benzene-fused or aza-substituted analogues of the preceding groups; and

when the group represented by formula (3) is bonded to L¹ or B, F represents a group selected from the group consisting of a cyano group, a fluorine atom, a haloalkyl group, a substituted or unsubstituted triphenylenyl group, a substituted or unsubstituted fluorenyl group, a substituted or unsubstituted spirofluorenyl group, a substituted or unsubstituted dibenzothiophenyl group, a substituted or unsubstituted dibenzofuranyl group, a substituted or unsubstituted pyridinyl group, a substituted or unsubstituted pyrimidinyl group, a substituted or unsubstituted triazinyl group, a substituted or unsubstituted bipyridinyl group, a substituted or unsubstituted bipyrimidinyl group, a substituted or unsubstituted quinazolinyl group, a substituted or unsubstituted imidazolyl group, a substituted or unsubstituted benzimidazolyl group, a phosphorus-containing group, a silicon-containing group, and benzene-fused or aza-substituted analogues of the preceding groups;

2. The aromatic heterocyclic derivative of Item 1, wherein the structure represented by formula (2-b) is represented by formula (2-b-1);

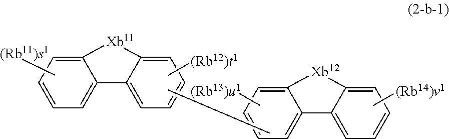

wherein:

each of Xb¹¹ and Xb¹² independently represents —NR—, —O—, —S—, —SiR₂—, the group represented by formula (i), or the group represented by formula (ii);

R is as defined above with respect to R in Xb¹, Xb², Yb¹, and Yb² of formula (2-b);

each of Rb¹¹, Rb¹², Rb¹³ and Rb¹⁴ independently represents a substituted or unsubstituted alkyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted aralkyl group having 7 to 24 carbon atoms, a substituted or unsubstituted silyl group, a substituted or unsubstituted aromatic hydrocarbon ring group having 6 to 24 ring carbon atoms, or a substituted or unsubstituted aromatic heterocyclic group having 2 to 24 ring carbon atoms;

s¹ represents an integer of 0 to 4, and when s¹ is 2 or more, groups Rb¹¹ may be the same or different;

t¹ represents an integer of 0 to 3, and when t¹ is 2 or more, groups Rb¹² may be the same or different;

u¹ represents an integer of 0 to 3, and when u¹ is 2 or more, groups Rb¹³ may be the same or different, and

v¹ represents an integer of 0 to 4, and when v¹ is 2 or more, groups Rb¹⁴ may be the same or different;

3. The aromatic heterocyclic derivative of Item 2, wherein B in formula (1) is a group represented by formula (2-A) or a group represented by formula (2-B):

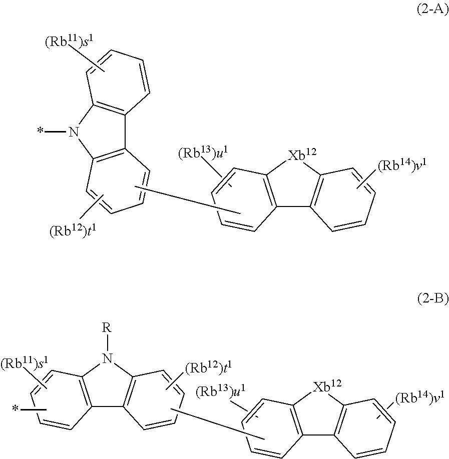

in formula (2-A):

Xb¹², Rb¹¹, Rb¹², Rb¹³, Rb¹⁴, s¹, t¹, u¹, and v¹ are as defined in formula (2-b-1);

* is bonded to L¹ of formula (1);

in formula (2-B):

s¹ is an integer of 0 to 3;

Xb¹², R, Rb¹¹, Rb¹², Rb¹³, Rb¹⁴, t¹, u¹, and v¹ are as defined in formula (2-b-1); and

* is bonded to L¹ of formula (1);

4. The aromatic heterocyclic derivative of any one of Items 1 to 3, wherein A of formula (1) is a residue of a ring assembly which comprises at least one substituted or unsubstituted aromatic hydrocarbon ring and at least one substituted or unsubstituted aromatic heterocyclic ring;
5. The aromatic heterocyclic derivative of Item 4, wherein A of formula (1) is a residue of a ring assembly represented by formula (4-a) or a residue of a ring assembly represented by formula (4-b):

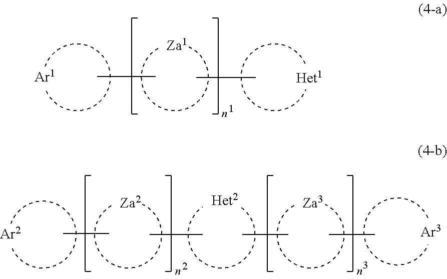

in formula (4-a):

Het¹ represents a substituted or unsubstituted aromatic heterocyclic group;

Ar¹ represents a substituted or unsubstituted aromatic hydrocarbon ring group;

Za¹ represents a substituted or unsubstituted aromatic hydrocarbon ring group or a substituted or unsubstituted aromatic heterocyclic group;

n¹ represents an integer of 0 to 2, and when n¹ is 2, groups Za¹ may be the same or different;

in formula (4-b);

Het² represents a substituted or unsubstituted aromatic heterocyclic group;

each of Ar² and Ar³ independently represents a substituted or unsubstituted aromatic hydrocarbon ring group;

each of Za² and Za³ independently represents a substituted or unsubstituted aromatic hydrocarbon ring group or a substituted or unsubstituted aromatic heterocyclic group;

n² represents an integer of 0 to 2, and when n² is 2, groups Za² may be the same or different; and

n³ represents an integer of 0 to 2, and when n³ is 2, groups Za³ may be the same or different;

6. The aromatic heterocyclic derivative Item 5, wherein each of Het¹ in formula (4-a) and Het² in formula (4-b) is a substituted or unsubstituted nitrogen-containing aromatic heterocyclic group;
7. The aromatic heterocyclic derivative of any one of Items 1 to 6, wherein when the group represented by formula (3) is bonded to A, F is a group selected from the group consisting of a cyano group, a fluorine atom, a haloalkyl group, a substituted or unsubstituted triphenylenyl group, a substituted or unsubstituted azafluorenyl group, and a substituted or unsubstituted bipyridinyl group;
8. The aromatic heterocyclic derivative of Item 7, wherein when the group represented by formula (3) is bonded to A, F is a group selected from the group consisting of a cyano group, a fluorine atom, and a haloalkyl group;
9. The aromatic heterocyclic derivative of any one of Items 1 to 6, wherein when the group represented by formula (3) is bonded to L¹ or B, F is a group selected from the group consisting of a cyano group, a fluorine atom, a haloalkyl group, a substituted or unsubstituted triphenylenyl group, a substituted or unsubstituted azafluorenyl group, a substituted or unsubstituted pyrimidinyl group, and a substituted or unsubstituted bipyridinyl group;
10. The aromatic heterocyclic derivative of Item 9, wherein when the group represented by formula (3) is bonded to L¹ or B, F is a group selected from the group consisting of a cyano group, a fluorine atom, and a haloalkyl group;
11. A material for an organic electroluminescence device comprising the aromatic heterocyclic derivative of any one of Items 1 to 10;
12. A solution of a material for an organic electroluminescence device comprising a solvent and the aromatic heterocyclic derivative of any one of Items 1 to 10 which is dissolved in the solvent;
13. An organic electroluminescence device comprising a cathode, an anode, and one or more organic thin film layers which are disposed between the cathode and the anode and comprise a light emitting layer, wherein at least one layer of the one or more organic thin film layers comprises the aromatic heterocyclic derivative of any one of Items 1 to 10;
14. The organic electroluminescence device of Item 13, wherein the light emitting layer comprises the aromatic heterocyclic derivative of any one of Items 1 to 10 as a host;
15. The organic electroluminescence device of Item 10 or 11, wherein the light emitting layer comprises a phosphorescent material;
16. The organic electroluminescence device of Item 15, wherein the phosphorescent material is an ortho-metallated complex of a metal atom selected from the group consisting of iridium (Ir), osmium (Os), and platinum (Pt);
17. The organic electroluminescence device of any one of Items 13 to 16, wherein the organic electroluminescence device comprises an electron injecting layer between the cathode and the light emitting layer, and the electron injecting layer comprises a nitrogen-containing ring derivative;
18. The organic electroluminescence device of any one of Items 13 to 17, wherein the organic electroluminescence device comprises an electron transporting layer between the cathode and the light emitting layer, and the electron transporting layer comprises the aromatic heterocyclic derivative of any one of Items 1 to 10;
19. The organic electroluminescence device of any one of Items 13 to 17, wherein the organic electroluminescence device comprises a hole transporting layer between the anode and the light emitting layer, and the hole transporting layer comprises the aromatic heterocyclic derivative of any one of Items 1 to 10; and
20. The organic electroluminescence device of any one of Items 13 to 19, wherein a reducing dopant is added to an interfacial region between the cathode and the organic thin film layer.

### Effects of the Invention

The present invention provides a novel aromatic heterocyclic derivative. By using the aromatic heterocyclic derivative, a material for an organic EL device which is soluble and suitable for a coating process is provided. A long lifetime organic EL device is produced by the coating process using a solution obtained by dissolving the aromatic heterocyclic derivative in a solvent.

### BRIEF DESCRIPTION OF THE DRAWINGS

FIG. 1 is a ¹H-NMR chart of the compound H-1 synthesized in Example 1.

FIG. 2 is a ¹H-NMR chart of the compound H-2 synthesized in Example 2.

FIG. 3 is a ¹H-NMR chart of the compound H-3 synthesized in Example 3.

FIG. 4 is a ¹H-NMR chart of the compound H-4 synthesized in Example 4.

FIG. 5 is a ¹H-NMR chart of the compound H-5 synthesized in Example 5.

### MODE FOR CARRYING OUT THE INVENTION

### Aromatic Heterocyclic Derivative

The aromatic heterocyclic derivative of the invention is represented by formula (1):

AL¹-B)ₘ (1)

A represents a substituted or unsubstituted aromatic hydrocarbon ring group, a substituted or unsubstituted aromatic heterocyclic group, a residue of a ring assembly which comprises at least two substituted or unsubstituted aromatic hydrocarbon rings, a residue of a ring assembly which comprises at least two substituted or unsubstituted aromatic heterocyclic rings, or a residue of a ring assembly which comprises at least one substituted or unsubstituted aromatic hydrocarbon ring and at least one substituted or unsubstituted aromatic heterocyclic ring. Preferred embodiments of A will be described later.

L¹ represents a single bond, a substituted or unsubstituted aromatic hydrocarbon ring group, or a substituted or unsubstituted aromatic heterocyclic group.

B represents a residue of a structure represented by formula (2-b). The details of formula (2-b) will be described later.

Subscript m represents an integer of 2 or more. The upper limit of m is determined according to the structure of A and not particularly limited, and m is preferably selected from 2 to 10.

Since m is 2 or more, there are more than one group L¹ and group B, and the groups L¹ may be the same or different and the groups B may be the same or different.

In formula (1), a group represented by formula (3) is bonded to at least one of A, L¹ and B. The details of formula (3) will be described later.

The words “a group represented by formula (3) is bonded to at least one of A, L¹ and B” mean that:

when only one group of formula (3) is included, the group of formula (3) is bonded to any one of A, L¹, and B, for example, the group of formula (3) is bonded to A; and

when two or more groups of formula (3) are included, these groups may be bonded to two or more of A, L¹, and B or may be bonded to one of A, L¹, and B, for example, when two groups of formula (3) are included, two groups may be bonded to A and B, respectively, or may be bonded to only A.

Since m is 2 or more, two or more L¹ and B are present, respectively. When the group represented by formula (3) is bonded to L¹, the group represented by formula (3) is not necessarily needed to be bonded to all of two or more groups L¹, and may be bonded to at least one of two or more groups L¹. For example, when m is 2, the group represented by formula (3) may be bonded to both of two groups L¹ or may be bonded to one of two groups L¹.

The same applies when the group represented by formula (3) is bonded to B.

When the group represented by formula (3) is bonded to L¹, L¹ does not represent a single bond, but represents a substituted or unsubstituted aromatic hydrocarbon ring group or a substituted or unsubstituted aromatic heterocyclic group.

Preferred embodiment of A will be described below.

As described above, A represents a substituted or unsubstituted aromatic hydrocarbon ring group (also referred to as “group A1”), a substituted or unsubstituted aromatic heterocyclic group (also referred to as “group A2”), a residue of a ring assembly which comprises at least two substituted or unsubstituted aromatic hydrocarbon rings (also referred to as “group A3”), a residue of a ring assembly which comprises at least two substituted or unsubstituted aromatic heterocyclic rings (also referred to as “group A4”), or a residue of a ring assembly which comprises at least one substituted or unsubstituted aromatic hydrocarbon ring and at least one substituted or unsubstituted aromatic heterocyclic ring (also referred to as “group A5”).

The group A1 is preferably a residue of a substituted or unsubstituted aromatic hydrocarbon ring having 6 to 30 ring carbon atoms.

Examples of the aromatic hydrocarbon ring having 6 to 30 ring carbon atoms include benzene, naphthalene, fluorene, phenanthrene, triphenylene, perylene, chrysene, fluoranthene, benzofluorene, benzotriphenylene, benzochrysene, anthracene, and benzene-fused or crosslinked analogues of the preceding rings, with benzene, naphthalene, fluorene, and phenanthrene being preferred.

The group A2 is preferably a residue of a substituted or unsubstituted aromatic heterocyclic ring having 2 to 30 ring carbon atoms.

Examples of the aromatic heterocyclic ring having 2 to 30 ring carbon atoms include pyrrole, pyridine, pyrazine, pyrimidine, pyridazine, triazine, indole, isoindole, quinoline, isoquinoline, quinoxaline, acridine, pyrrolizine, dioxane, piperidine, morpholine, piperazine, carbazole, phenanthridine, phenanthroline, furan, benzofuran, isobenzofuran, thiophene, oxazole, oxadiazole, benzoxazole, thiazole, thiadiazole, benzothiazole, triazole, imidazole, benzimidazole, pyran, dibenzofuran, dibenzothiophene, azafluorene, azacarbazole, and benzene-fused or crosslinked analogues of the preceding rings, with pyridine, pyrazine, pyrimidine, pyridazine, and triazine being preferred.

Each of the substituted or unsubstituted aromatic hydrocarbon rings for constituting the group A3 is preferably a substituted or unsubstituted aromatic hydrocarbon ring having 6 to 30 ring carbon atoms.

Example of the aromatic hydrocarbon ring having 6 to 30 ring carbon atoms and its preferred examples are as described above with respect to the group A1.

Each of the substituted or unsubstituted aromatic heterocyclic rings for constituting the group A4 is preferably a substituted or unsubstituted aromatic heterocyclic ring having 2 to 30 ring carbon atoms.

Examples of the aromatic heterocyclic ring having 2 to 30 ring carbon atoms and its preferred examples are as described above with respect to the group A2.

The substituted or unsubstituted aromatic hydrocarbon ring for constituting the group A5 is independently preferably a substituted or unsubstituted aromatic hydrocarbon ring having 6 to 30 ring carbon atoms. The substituted or unsubstituted aromatic heterocyclic ring for constituting the group A5 is independently preferably a substituted or unsubstituted aromatic heterocyclic ring having 2 to 30 ring carbon atoms.

Example of the aromatic hydrocarbon ring having 6 to 30 ring carbon atoms and its preferred examples are as described above with respect to the group A1.

Examples of the aromatic heterocyclic ring having 2 to 30 ring carbon atoms and its preferred examples are as described above with respect to the group A2.

Of the groups A1 to A5, preferred as the group A are the groups A3 and A5, and more preferred is the group A5.

The group A3 is particularly preferably a residue of biphenyl or terphenyl.

The group A5 is particularly preferably a residue of a ring assembly represented by formula (4-a) or a ring assembly represented by formula (4-b):

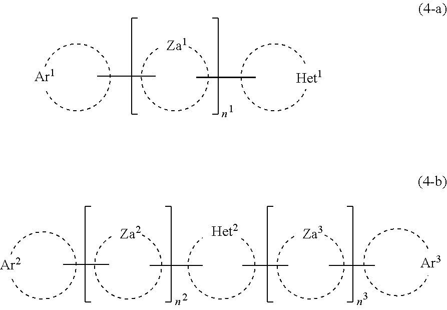

In formula (4-a);

Het¹ represents a substituted or unsubstituted aromatic heterocyclic group;

Ar¹ represents a substituted or unsubstituted aromatic hydrocarbon ring group;

Za¹ represents a substituted or unsubstituted aromatic hydrocarbon ring group or a substituted or unsubstituted aromatic heterocyclic group; and

n¹ represents an integer of 0 to 2, and when n¹ represents 2, groups Za¹ may be the same or different.

Het¹ preferably represents a residue of a substituted or unsubstituted aromatic heterocyclic ring having 2 to 30 ring carbon atoms. Het¹ preferably represents a substituted or unsubstituted nitrogen-containing aromatic heterocyclic group and more preferably a residue of a substituted or unsubstituted pyridine, pyrazine, pyrimidine, pyridazine or triazine.

Ar¹ preferably represents a residue of a substituted or unsubstituted aromatic hydrocarbon ring having 6 to 30 ring carbon atoms and more preferably a residue of a substituted or unsubstituted benzene, naphthalene, fluorene, or phenanthrene.

Za¹ preferably represents a residue of a substituted or unsubstituted aromatic heterocyclic ring having 2 to 30 ring carbon atoms or a residue of a substituted or unsubstituted aromatic hydrocarbon ring having 6 to 30 ring carbon atoms and more preferably a residue of a substituted or unsubstituted benzene, naphthalene, fluorene, phenanthrene, pyridine, pyrazine, pyrimidine, pyridazine, or triazine.

In formula (4-b);

Het² represents a substituted or unsubstituted aromatic heterocyclic group;

each of Ar² and Ar³ independently represents a substituted or unsubstituted aromatic hydrocarbon ring group;

each of Za² and Za³ independently represents a substituted or unsubstituted aromatic hydrocarbon ring group or a substituted or unsubstituted aromatic heterocyclic group;

n² represents an integer of 0 to 2, and when n² is 2, groups Za² may be the same or different; and

n³ represents an integer of 0 to 2, and when n³ is 2, groups Za³ may be the same or different.

Het² is preferably a residue of a substituted or unsubstituted aromatic heterocyclic ring having 2 to 30 ring carbon atoms. Het² is preferably a substituted or unsubstituted nitrogen-containing aromatic heterocyclic group and more preferably a residue of a substituted or unsubstituted pyridine, pyrazine, pyrimidine, pyridazine or triazine.

Each of Ar² and Ar³ is independently preferably a residue of a substituted or unsubstituted aromatic hydrocarbon ring having 6 to 30 ring carbon atoms and more preferably a residue of a substituted or unsubstituted benzene, naphthalene, fluorene, or phenanthrene.

Each of Za² and Za³ is independently preferably a residue of a substituted or unsubstituted aromatic heterocyclic ring having 2 to 30 ring carbon atoms or a residue of a substituted or unsubstituted aromatic hydrocarbon ring having 6 to 30 ring carbon atoms and more preferably a residue of a substituted or unsubstituted benzene, naphthalene, fluorene, phenanthrene, pyridine, pyrazine, pyrimidine, pyridazine, or triazine.

In formula (2-b):

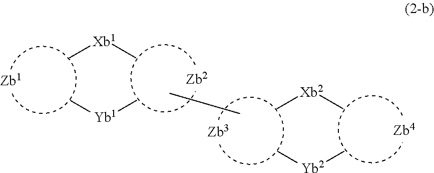

one of Xb¹ and Yb¹ represents a single bond, —CR₂—, —NR—, —O—, —S—, —SiR₂—, a group represented by formula (i), or a group represented by formula (ii), and the other represents —NR—, —O—, —S—, —SiR₂—, a group represented by formula (i), or a group represented by formula (ii);

one of Xb² and Yb² represents a single bond, —CR₂—, —NR—, —O—, —S—, —SiR₂—, a group represented by formula (i), or a group represented by formula (ii), and the other represents —NR—, —O—, —S—, —SiR₂—, a group represented by formula (i), or a group represented by formula (ii);

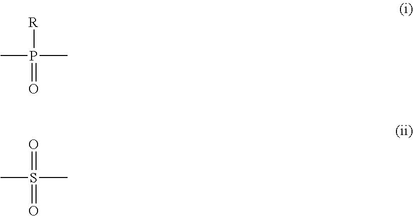

R represents a hydrogen atom, a substituted or unsubstituted alkyl group, a substituted or unsubstituted cycloalkyl group, a substituted or unsubstituted aromatic hydrocarbon ring group, or a substituted or unsubstituted aromatic heterocyclic group; and

each of Zb¹, Zb², Zb³ and Zb⁴ independently represents a substituted or unsubstituted aliphatic hydrocarbon ring group, a substituted or unsubstituted aliphatic heterocyclic ring group, a substituted or unsubstituted aromatic hydrocarbon ring group, or a substituted or unsubstituted aromatic heterocyclic group.

The structure represented by formula (2-b) is preferably represented by formula (2-b-1):

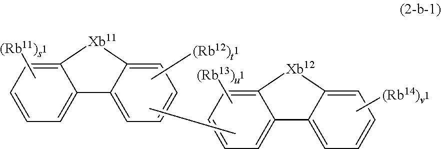

wherein:

each of Xb¹¹ and Xb¹² independently represents —NR—, —O—, —S—, —SiR₂—, the group represented by formula (i), or the group represented by formula (ii);

R is as defined above with respect to R of Xb¹, Xb², Yb¹ and Yb² in formula (2-b);

each of Rb¹¹, Rb¹², Rb¹³, and Rb¹⁴ independently represents a substituted or unsubstituted alkyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted aralkyl group having 7 to 24 carbon atoms, a substituted or unsubstituted silyl group, a substituted or unsubstituted aromatic hydrocarbon ring group having 6 to 24 ring carbon atoms, or a substituted or unsubstituted aromatic heterocyclic group having 2 to 24 ring carbon atoms;

s¹ represents an integer of 0 to 4, and when s¹ is 2 or more, groups Rb¹¹ may be the same or different;

t¹ represents an integer of 0 to 3, and when t¹ is 2 or more, groups Rb¹² may be the same or different;

u¹ represents an integer of 0 to 3, and when u¹ is 2 or more, groups Rb¹³ may be the same or different; and

v¹ represents an integer of 0 to 4, and when v¹ is 2 or more, groups Rb¹⁴ may be the same or different.

B of formula (1) preferably represents a group represented by formula (2-A) or a group represented by formula (2-B);

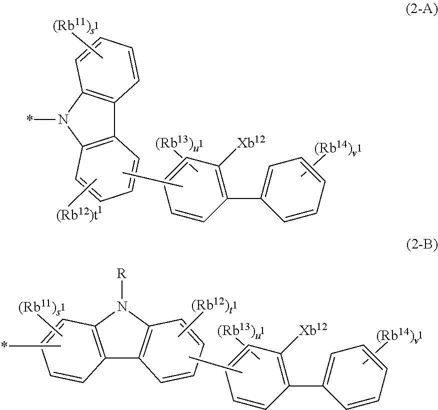

in formula (2-A);

Xb¹², Rb¹¹, Rb¹², Rb¹³, Rb¹⁴, s¹, t¹, u¹, and v¹ are as defined in formula (2-b-1); and

* is bonded to L¹ of formula (1);

in formula (2-B);

s¹ represents an integer of 0 to 3;

Xb¹², R, Rb¹¹, Rb¹², Rb¹³, Rb¹⁴, t¹, u¹, and v¹ are as defined in formula (2-b-1); and

* is bonded to L¹ of formula (1).

R in formula (2-B) is preferably a substituted or unsubstituted alkyl group, a substituted or unsubstituted cycloalkyl group, a substituted or unsubstituted aromatic hydrocarbon ring group, or a substituted or unsubstituted aromatic heterocyclic group.

The group represented by formula (2-A) is more preferably a group represented by any of formulae (2-A-1) to (2-A-3):

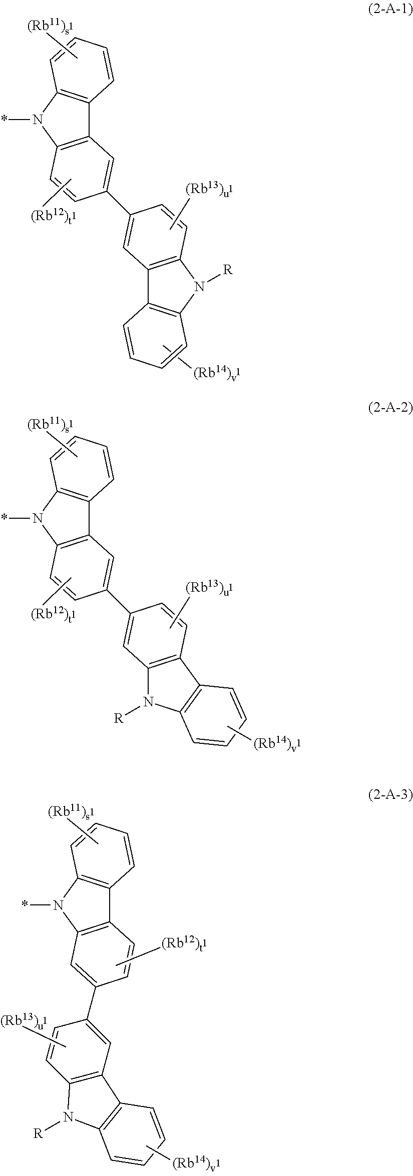

wherein:

R, Rb¹¹, Rb¹², Rb¹³, Rb¹⁴, s¹, t¹, u¹, and v¹ are as defined in formula (2-b-1);

and

* is bonded to L¹ formula (1).

R in formulae (2-A-1) to (2-A-3) is preferably a substituted or unsubstituted alkyl group, a substituted or unsubstituted cycloalkyl group, a substituted or unsubstituted aromatic hydrocarbon ring group, or a substituted or unsubstituted aromatic heterocyclic group.

Formula (3) will be described below.

-L³-F (3)

L³ represents a single bond, a substituted or unsubstituted aromatic hydrocarbon ring group, or a substituted or unsubstituted aromatic heterocyclic group. L³ preferably represents a single bond, a substituted or unsubstituted phenylene group, or a substituted or unsubstituted biphenylyene group.

When the group represented by formula (3) is bonded to A, F represents a group selected from the group consisting of a cyano group, a fluorine atom, a haloalkyl group, a substituted or unsubstituted triphenylenyl group, a substituted or unsubstituted azafluorenyl group, a substituted or unsubstituted spirofluorenyl group, a substituted or unsubstituted dibenzothiophenyl group, a substituted or unsubstituted bipyridinyl group, a substituted or unsubstituted bipyrimidinyl group, a substituted or unsubstituted quinazolinyl group, a substituted or unsubstituted imidazolyl group, a substituted or unsubstituted benzimidazolyl group, a phosphorus-containing group, a silicon-containing group, and benzene-fused or aza-substituted analogues of the preceding groups. The groups referred to by “benzene-fused or aza-substituted analogues of the preceding groups” are those structurally capable of forming benzene-fused or aza-substituted analogues and do not include those, for example, a cyano group, which are structurally incapable of forming benzene-fused or aza-substituted analogues. The same applies to the similar expressions herein.

When the group represented by formula (3) is bonded to L¹ or B, F represents a group selected from the group consisting of a cyano group, a fluorine atom, a substituted or unsubstituted triphenylenyl group, a substituted or unsubstituted fluorenyl group, a substituted or unsubstituted spirofluorenyl group, a substituted or unsubstituted dibenzothiophenyl group, a substituted or unsubstituted dibenzofuranyl group, a substituted or unsubstituted pyridinyl group, a substituted or unsubstituted pyrimidinyl group, a substituted or unsubstituted triazinyl group, a substituted or unsubstituted bipyridinyl group, a substituted or unsubstituted bipyrimidinyl group, a substituted or unsubstituted quinazolinyl group, a substituted or unsubstituted imidazolyl group, a substituted or unsubstituted benzimidazolyl group, a phosphorus-containing group, a silicon-containing group, and benzene-fused or aza-substituted analogues of the preceding groups.

When the group represented by formula (3) is bonded to A, F preferably represents a group selected from the group consisting of a cyano group, a fluorine atom, a haloalkyl group, a substituted or unsubstituted triphenylenyl group, a substituted or unsubstituted azafluorenyl group, and a substituted or unsubstituted bipyridinyl group, with a group selected from the group consisting of a cyano group, a fluorine atom, and a haloalkyl group being more preferred. The haloalkyl group is preferably a fluoroalkyl group having 1 to 3 carbon atoms and particularly preferably a trifluoromethyl group.

When the group represented by formula (3) is bonded to L¹ or B, F preferably represents a group selected from the group consisting of a cyano group, a fluorine atom, a haloalkyl group, a substituted or unsubstituted triphenylenyl group, a substituted or unsubstituted azafluorenyl group, a substituted or unsubstituted pyrimidinyl group, and a substituted or unsubstituted bipyridinyl group, with a group selected from the group consisting of a cyano group, a fluorine atom, and a haloalkyl group being more preferred. The haloalkyl group is preferably a fluoroalkyl group having 1 to 3 carbon atoms and particularly preferably a trifluoromethyl group.

Since the group represented by F is an electron withdrawing group, the electron transporting ability of an electron transporting structure can be further improve when F is bonded thereto. For example, when A is an electron transporting structure and the group represented by formula (3) is bonded to A, LUMO distributes over the portion of A and HOMO distributes over the portion of B, thus HOMO and LUMO are localized separately. The elongated lifetime of EL device employing the aromatic heterocyclic derivative of the invention is attributable to this HOMO-LUMO structure.

In an embodiment of the invention, the aromatic heterocyclic derivative is represented by formula (1) wherein the variables are defined as follows:

AL¹-B)ₘ (1)

A represents a substituted or unsubstituted aromatic hydrocarbon ring group or a substituted or unsubstituted aromatic heterocyclic group;

L¹ represents a single bond, a substituted or unsubstituted aromatic hydrocarbon ring group or a substituted or unsubstituted aromatic heterocyclic group;

B represents a residue of a structure represented by formula (2-b);

m represents an integer of 2 or more, groups L¹ may be the same or different, and groups B may be the same or different;

provided that a group represented by formula (3) is bonded to at least one of A, L¹ and B;

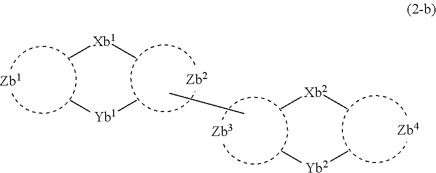

In formula (2-b):

one of Xb¹ and Yb¹ represents a single bond, —CR₂—, —NR—, —O—, —S—, —SiR₂—, a group represented by formula (i), or a group represented by formula (ii), and the other represents —NR—, —O—, —S—, —SiR₂—, a group represented by formula (i), or a group represented by formula (ii); and one of Xb² and Yb² represents a single bond, —CR₂—, —NR—, —O—, —S—, —SiR₂—, a group represented by formula (i), or a group represented by formula (ii), and the other represents —NR—, —O—, —S—, —SiR₂—, a group represented by formula (i), or a group represented by formula (ii);

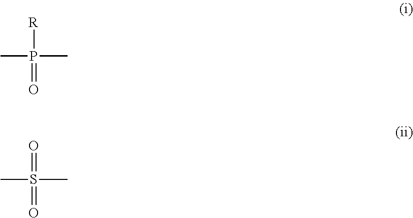

R represents a hydrogen atom, a substituted or unsubstituted alkyl group, a substituted or unsubstituted cycloalkyl group, a substituted or unsubstituted aromatic hydrocarbon ring group, or a substituted or unsubstituted aromatic heterocyclic group; and

each of Zb¹, Zb², Zb³ and Zb⁴ independently represents a substituted or unsubstituted aliphatic hydrocarbon ring group, a substituted or unsubstituted aliphatic heterocyclic ring group, a substituted or unsubstituted aromatic hydrocarbon ring group, or a substituted or unsubstituted aromatic heterocyclic group;

-L³-F (3)

in formula (3):

L³ represents a single bond, a substituted or unsubstituted aromatic hydrocarbon ring group, or a substituted or unsubstituted aromatic heterocyclic group;

when the group represented by formula (3) is bonded to A, F represents a group selected from the group consisting of a cyano group, a fluorine atom, a substituted or unsubstituted triphenylenyl group, a substituted or unsubstituted spirofluorenyl group, a substituted or unsubstituted dibenzothiophenyl group, a substituted or unsubstituted bipyridinyl group, a substituted or unsubstituted bipyrimidinyl group, a substituted or unsubstituted quinazolinyl group, a substituted or unsubstituted imidazolyl group, a substituted or unsubstituted benzimidazolyl group, a phosphorus-containing group, a silicon-containing group, and benzene-fused or aza-substituted analogues of the preceding groups;

when the group represented by formula (3) is bonded to L¹, F represents a group selected from the group consisting of a cyano group, a fluorine atom, a substituted or unsubstituted triphenylenyl group, a substituted or unsubstituted fluorenyl group, a substituted or unsubstituted spirofluorenyl group, a substituted or unsubstituted dibenzothiophenyl group, a substituted or unsubstituted dibenzofuranyl group, a substituted or unsubstituted pyridinyl group, a substituted or unsubstituted pyrimidinyl group, a substituted or unsubstituted triazinyl group, a substituted or unsubstituted bipyridinyl group, a substituted or unsubstituted bipyrimidinyl group, a substituted or unsubstituted quinazolinyl group, a substituted or unsubstituted imidazolyl group, a substituted or unsubstituted benzimidazolyl group, a phosphorus-containing group, a silicon-containing group, and benzene-fused or aza-substituted analogues of the preceding groups;

when the group represented by formula (3) is bonded to B, F represents a group selected from the group consisting of a cyano group, a fluorine atom, a substituted or unsubstituted triphenylenyl group, a substituted or unsubstituted fluorenyl group, a substituted or unsubstituted spirofluorenyl group, a substituted or unsubstituted pyridinyl group, a substituted or unsubstituted pyrimidinyl group, a substituted or unsubstituted triazinyl group, a substituted or unsubstituted bipyridinyl group, a substituted or unsubstituted bipyrimidinyl group, a substituted or unsubstituted quinazolinyl group, a substituted or unsubstituted imidazolyl group, a substituted or unsubstituted benzimidazolyl group, a phosphorus-containing group, a silicon-containing group, and benzene-fused or aza-substituted analogues of the preceding groups; and

provided that when the group represented by formula (3) is bonded to A or L¹ and F is a cyano group, L³ represents an unsubstituted aromatic hydrocarbon ring group or a substituted or unsubstituted aromatic heterocyclic group.

Details of the variables in the above formulae are described below.

Each of the substituted or unsubstituted aromatic hydrocarbon ring groups represented by L¹ of formula (1), R and Zb¹ to Zb⁴ of formula (2-b), R of formula (2-b-1), R of formula (2-A), R of formula (2-B), R of formulae (2-A-1) to (2-A-3), and L³ of formula (3) is preferably a residue of a substituted or unsubstituted aromatic hydrocarbon ring having 6 to 30 ring carbon atoms.

Examples of the aromatic hydrocarbon ring having 6 to 30 ring carbon atoms include benzene, naphthalene, biphenyl, terphenyl, fluorene, phenanthrene, triphenylene, perylene, chrysene, fluoranthene, benzofluorene, benzotriphenylene, benzochrysene, anthracene, and benzene-fused or crosslinked analogues of the preceding groups, with benzene, naphthalene, biphenyl, terphenyl, fluorene, and phenanthrene being preferred.

Each of the substituted or unsubstituted aromatic heterocyclic groups represented by L¹ of formula (1), R and Zb¹ to Zb⁴ of formula (2-b), R of formula (2-b-1), R of formula (2-A), R of formula (2-B), R of formulae (2-A-1) to (2-A-3), and L³ of formula (3) is preferably a residue of a substituted or unsubstituted aromatic heterocyclic ring having 2 to 30 ring carbon atoms.

Examples of the aromatic heterocyclic ring having 2 to 30 ring carbon atoms include pyrrole, pyridine, pyrazine, pyrimidine, pyridazine, triazine, indole, isoindole, quinoline, isoquinoline, quinoxaline, acridine, pyrrolizine, dioxane, piperidine, morpholine, piperazine, carbazole, phenanthridine, phenanthroline, furan, benzofuran, isobenzofuran, thiophene, oxazole, oxadiazole, benzoxazole, thiazole, thiadiazole, benzothiazole, triazole, imidazole, benzimidazole, pyran, dibenzofuran, dibenzothiophene, azafluorene, azacarbazole, and benzene-fused or crosslinked analogues of the preceding rings, with pyridine, pyrazine, pyrimidine, pyridazine, and triazine being preferred.

Each of the substituted or unsubstituted alkyl groups represented by R of formula (2-b), R of formula (2-b-1), R of formula (2-A), R of formula (2-B), and R of formulae (2-A-1) to (2-A-3) is preferably a substituted or unsubstituted alkyl group having 1 to 30 carbon atoms.

Examples of the alkyl group having 1 to 30 carbon atoms include a methyl group, an ethyl group, a propyl group, an isopropyl group, a n-butyl group, a s-butyl group, an isobutyl group, a t-butyl group, a n-pentyl group, a n-hexyl group, a n-heptyl group, a n-octyl group, a n-nonyl group, a n-decyl group, a n-undecyl group, a n-dodecyl group, a n-tridecyl group, a n-tetradecyl group, a n-pentadecyl group, a n-hexadecyl group, a n-heptadecyl group, a n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group, with a methyl group, an ethyl group, a propyl group, an isopropyl group, a n-butyl group, a s-butyl group, an isobutyl group, and a t-butyl group being preferred.

Each of the substituted or unsubstituted cycloalkyl groups represented by R of formula (2-b), R of formula (2-b-1), R of formula (2-A), R of formula (2-B), and R of formulae (2-A-1) to (2-A-3) is preferably a substituted or unsubstituted cycloalkyl group having 3 to 30 ring carbon atoms.

Examples of the cycloalkyl group having 3 to 30 ring carbon atoms include a cyclopropyl group, a cyclobutyl group, a cyclopentyl group, a cyclohexyl group, a cyclooctyl group, and an adamantyl group, with a cyclopentyl group and a cyclohexyl group being preferred.

Each of the substituted or unsubstituted aliphatic hydrocarbon ring groups represented by Zb¹ to Zb⁴ of formula (2-b) is preferably a residue of a substituted or unsubstituted cycloalkane having 3 to 30 ring carbon atoms or a residue of a substituted or unsubstituted cycloalkene having 3 to 30 ring carbon atoms.

Examples of the cycloalkane having 3 to 30 ring carbon atoms include cyclopropane, cyclobutane, cyclopentane, cyclohexane, cyclooctane, and adamantane, with cyclopentane and cyclohexane being preferred.

Examples of the cycloalkene having 3 to 30 ring carbon atoms include cyclopropane, cyclobutene, cyclopentene, cyclohexene, and cyclooctene, with cyclopentene and cyclohexene being preferred.

Each of the substituted or unsubstituted aliphatic heterocyclic ring groups represented by Zb¹ to Zb⁴ of formula (2-b) is preferably a group obtained by replacing one or more ring carbon atoms of the above substituted or unsubstituted aliphatic hydrocarbon ring group with a hetero atom, such as an oxygen atom, a nitrogen atom and a sulfur atom.

Examples of the substituted or unsubstituted alkyl group having 1 to 20 carbon atoms represented by Rb¹¹ to Rb¹⁴ of formula (2-b-1), Rb¹¹ to Rb¹⁴ of formula (2-A), Rb¹¹ to Rb¹⁴ of formula (2-B), Rb¹¹ to Rb¹⁴ of formula (2-A-1), Rb¹¹ to Rb¹⁴ of formula (2-A-2), and Rb¹¹ to Rb¹⁴ of formula (2-A-3) include a methyl group, an ethyl group, a propyl group, an isopropyl group, a n-butyl group, a s-butyl group, a t-butyl group, an isobutyl group, a n-pentyl group, a n-hexyl group, a n-heptyl group, a n-octyl group, a n-nonyl group, a n-decyl group, a n-undecyl group, a n-dodecyl group, a n-tridecyl group, a n-tetradecyl group, a n-pentadecyl group, a n-hexadecyl group, a n-heptadecyl group, a n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Preferred are a methyl group, an ethyl group, a propyl group, an isopropyl group, a n-butyl group, a s-butyl group, an isobutyl group, a t-butyl group, a n-pentyl group, a n-hexyl group, a n-heptyl group, a n-octyl group, a n-nonyl group, a n-decyl group, a n-undecyl group, a n-dodecyl group, a n-tridecyl group, a n-tetradecyl group, a n-pentadecyl group, a n-hexadecyl group, a n-heptadecyl group, a n-octadecyl group, a neopentyl group, a 1-methylpentyl group, a 1-pentylhexyl group, a 1-butylpentyl group, and a 1-heptyloctyl group.

Examples of the substituted or unsubstituted cycloalkyl group having 3 to 20 ring carbon atoms represented by Rb¹¹ to Rb¹⁴ of formula (2-b-1), Rb¹¹ to Rb¹⁴ of formula (2-A), Rb¹¹ to Rb¹⁴ of formula (2-B), Rb¹¹ to Rb¹⁴ of formula (2-A-1), Rb¹¹ to Rb¹⁴ of formula (2-A-2), and Rb¹¹ to Rb¹⁴ of formula (2-A-3) include a cyclopropyl group, a cyclobutyl group, a cyclopentyl group, and a cyclohexyl group, with a cyclobutyl group, a cyclopentyl group and a cyclohexyl group being preferred.

Examples of the substituted or unsubstituted alkoxy group having 1 to 20 carbon atoms represented by Rb¹¹ to Rb¹⁴ of formula (2-b-1), Rb¹¹ to Rb¹⁴ of formula (2-A), Rb¹¹ to Rb¹⁴ of formula (2-B), Rb¹¹ to Rb¹⁴ of formula (2-A-1), Rb¹¹ to Rb¹⁴ of formula (2-A-2), and Rb¹¹ to Rb¹⁴ of formula (2-A-3) include a methoxy group, an ethoxy group, an isopropoxy group, a n-propoxy group, n-butoxy group, a s-butoxy group, and a t-butoxy group, with a methoxy group, an ethoxy group, an isopropoxy group, and a n-propoxy group being preferred.

Examples of the aralkyl group having 7 to 24 carbon atoms in the substituted or unsubstituted aralkyl group having 7 to 24 carbon atoms represented by Rb¹¹ to Rb¹⁴ of formula (2-b-1), Rb¹¹ to Rb¹⁴ of formula (2-A), Rb¹¹ to Rb¹⁴ of formula (2-B), Rb¹¹ to Rb¹⁴ of formula (2-A-1), Rb¹¹ to Rb¹⁴ of formula (2-A-2), and Rb¹¹ to Rb¹⁴ of formula (2-A-3) include a benzyl group, a phenethyl group, and a phenylpropyl group, with a benzyl group being preferred.

Examples of the aromatic hydrocarbon group having 6 to 24 ring carbon atoms represented by Rb¹¹ to Rb¹⁴ of formula (2-b-1), Rb¹¹ to Rb¹⁴ of formula (2-A), Rb¹¹ to Rb¹⁴ of formula (2-B), Rb¹¹ to Rb¹⁴ of formula (2-A-1), Rb¹¹ to Rb¹⁴ of formula (2-A-2), and Rb¹¹ to Rb¹⁴ of formula (2-A-3) include residues of aromatic hydrocarbon rings, such as benzene, naphthalene, biphenyl, terphenyl, fluorene, phenanthrene, triphenylene, perylene, chrysene, fluoranthene, benzofluorene, benzotriphenylene, benzochrysene, and anthracene, with residues of benzene, naphthalene, biphenyl, terphenyl, fluorene, and phenanthrene being preferred.

Examples of the aromatic heterocyclic group having 2 to 24 ring carbon atoms represented by Rb¹¹ to Rb¹⁴ of formula (2-b-1), Rb¹¹ to Rb¹⁴ of formula (2-A), Rb¹¹ to Rb¹⁴ of formula (2-B), Rb¹¹ to Rb¹⁴ of formula (2-A-1), Rb¹¹ to Rb¹⁴ of formula (2-A-2), and Rb¹¹ to Rb¹⁴ of formula (2-A-3) include residues of aromatic heterocyclic rings, such as pyridine, pyridazine, pyrimidine, pyrazine, 1,3,5-triazine, carbazole, dibenzofuran, dibenzothiophene, phenoxazine, phenothiazine, and dihydroacridine, with residues of pyridine, pyridazine, pyrimidine, pyrazine, carbazole, dibenzofuran, dibenzothiophene, phenoxazine, and dihydroacridine being preferred.

Examples of the optional substituent referred to by “substituted or unsubstituted” described above include a halogen atom (fluorine, chlorine, bromine, and iodine), a cyano group, an alkyl group having 1 to 20, preferably 1 to 6 carbon atoms, a cycloalkyl group having 3 to 20, preferably 5 to 12 carbon atoms, an alkoxyl group having 1 to 20, preferably 1 to 5 carbon atoms, a haloalkyl group having 1 to 20, preferably 1 to 5 carbon atoms, a haloalkoxyl group having 1 to 20, preferably 1 to 5 carbon atoms, an alkylsilyl group having 1 to 10, preferably 1 to 5 carbon atoms, an aryl group having 6 to 30, preferably 6 to 18 ring carbon atoms, an aryloxy group having 6 to 30, preferably 6 to 18 ring carbon atoms, an arylsilyl group having 6 to 30, preferably 6 to 18 ring carbon atoms, an aralkyl group having 7 to 30, preferably 7 to 20 carbon atoms, and a heteroaryl group having 2 to 30, preferably 2 to 18 ring carbon atoms.

The carbon number “a to b” in the expression of “a substituted or unsubstituted XX group having a to b carbon atoms” used herein is the carbon number of the unsubstituted XX group and does not include the carbon atom of the optional substituent.

The aromatic hydrocarbon ring group and the aromatic heterocyclic group in this specification include a fused aromatic hydrocarbon ring group and a fused aromatic heterocyclic group.

The “hydrogen atom” referred to herein includes isotopes different from neutron numbers, i.e., light hydrogen (protium), heavy hydrogen (deuterium) and tritium.

Examples of the aromatic heterocyclic derivative of the invention are shown below, although not limited thereto.

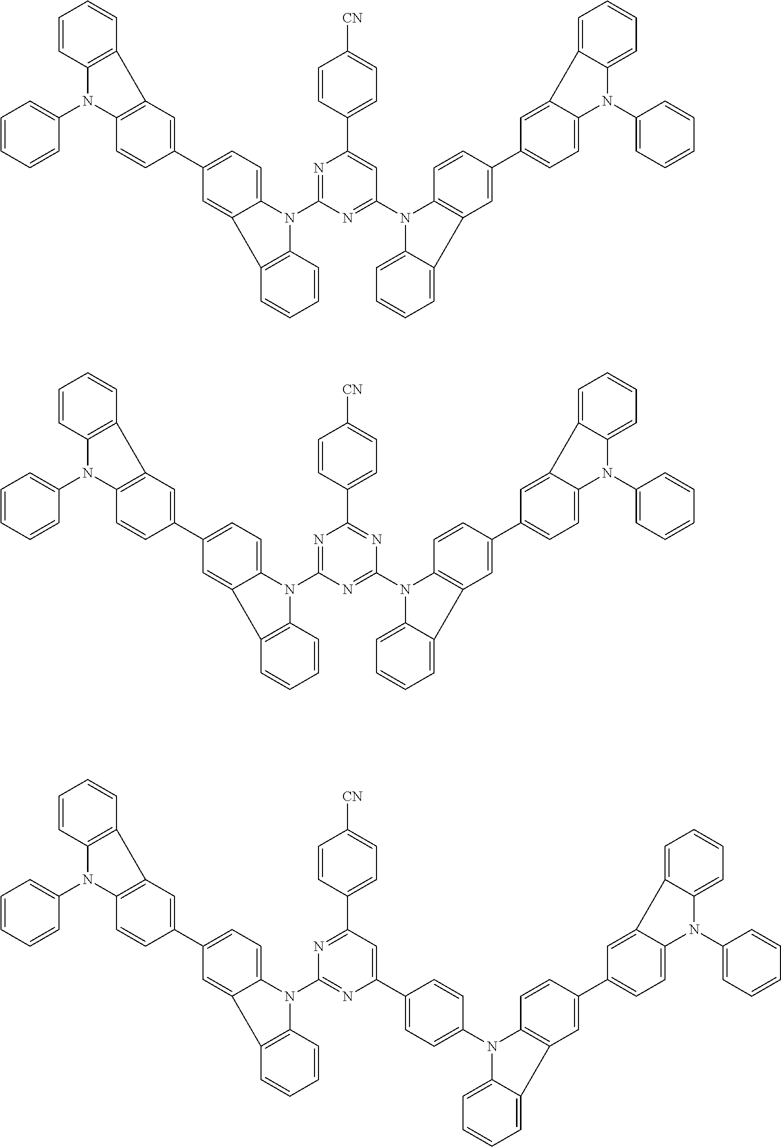 [SMILES: N#Cc1ccc(-c2cc(-n3c4ccccc4c4cc(-c5ccc6c(c5)c5ccccc5n6-c5ccccc5)ccc43)nc(-n3c4ccccc4c4cc(-c5ccc6c(c5)c5ccccc5n6-c5ccccc5)ccc43)n2)cc1 ; N#Cc1ccc(-c2nc(-n3c4ccccc4c4cc(-c5ccc6c(c5)c5ccccc5n6-c5ccccc5)ccc43)nc(-n3c4ccccc4c4cc(-c5ccc6c(c5)c5ccccc5n6-c5ccccc5)ccc43)n2)cc1 ; N#Cc1ccc(-c2cc(-c3ccc(-n4c5ccccc5c5cc(-c6ccc7c(c6)c6ccccc6n7-c6ccccc6)ccc54)cc3)nc(-n3c4ccccc4c4cc(-c5ccc6c(c5)c5ccccc5n6-c5ccccc5)ccc43)n2)cc1]

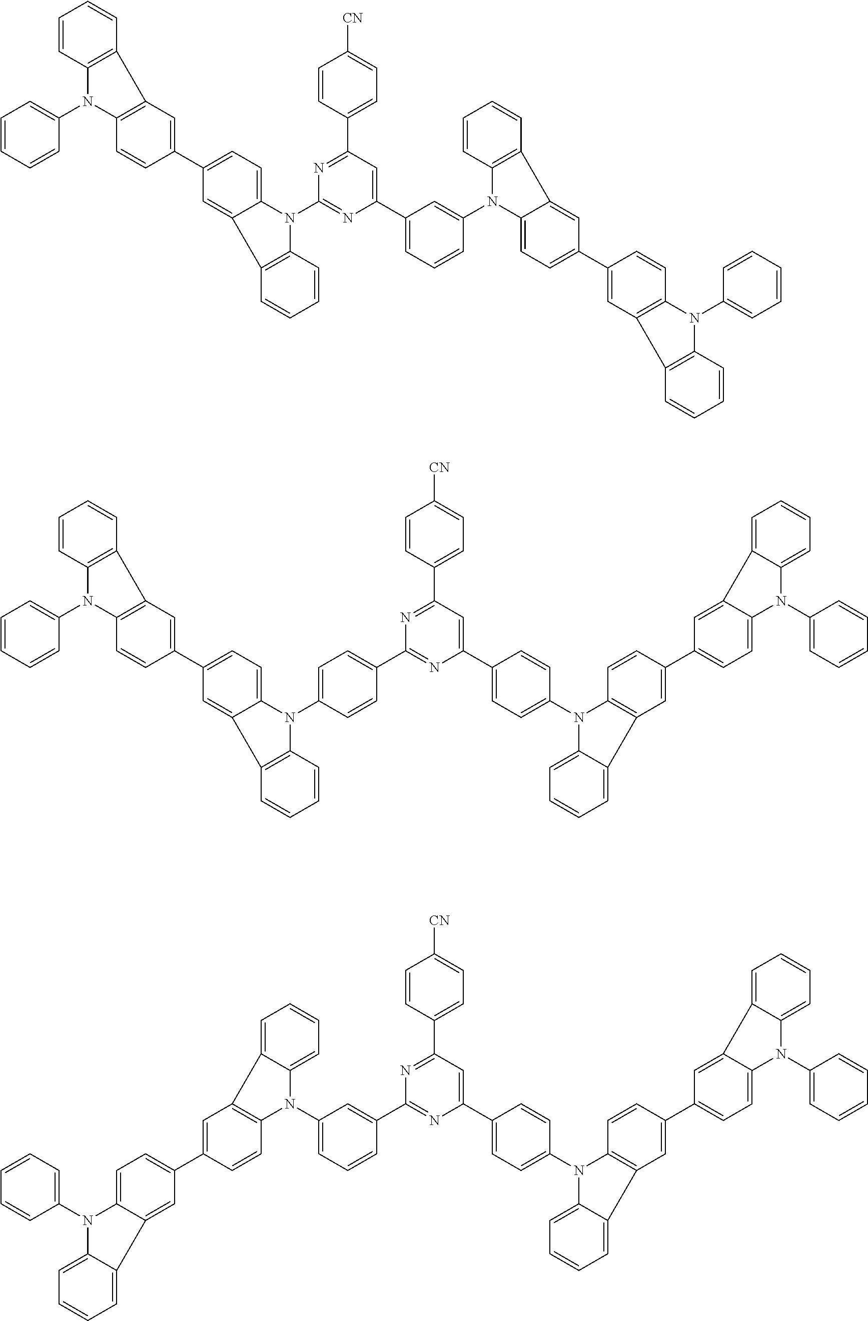 [SMILES: N#Cc1ccc(-c2cc(-c3cccc(-n4c5ccccc5c5cc(-c6ccc7c(c6)c6ccccc6n7-c6ccccc6)ccc54)c3)nc(-n3c4ccccc4c4cc(-c5ccc6c(c5)c5ccccc5n6-c5ccccc5)ccc43)n2)cc1 ; N#Cc1ccc(-c2cc(-c3ccc(-n4c5ccccc5c5cc(-c6ccc7c(c6)c6ccccc6n7-c6ccccc6)ccc54)cc3)nc(-c3ccc(-n4c5ccccc5c5cc(-c6ccc7c(c6)c6ccccc6n7-c6ccccc6)ccc54)cc3)n2)cc1 ; N#Cc1ccc(-c2cc(-c3ccc(-n4c5ccccc5c5cc(-c6ccc7c(c6)c6ccccc6n7-c6ccccc6)ccc54)cc3)nc(-c3cccc(-n4c5ccccc5c5cc(-c6ccc7c(c6)c6ccccc6n7-c6ccccc6)ccc54)c3)n2)cc1]

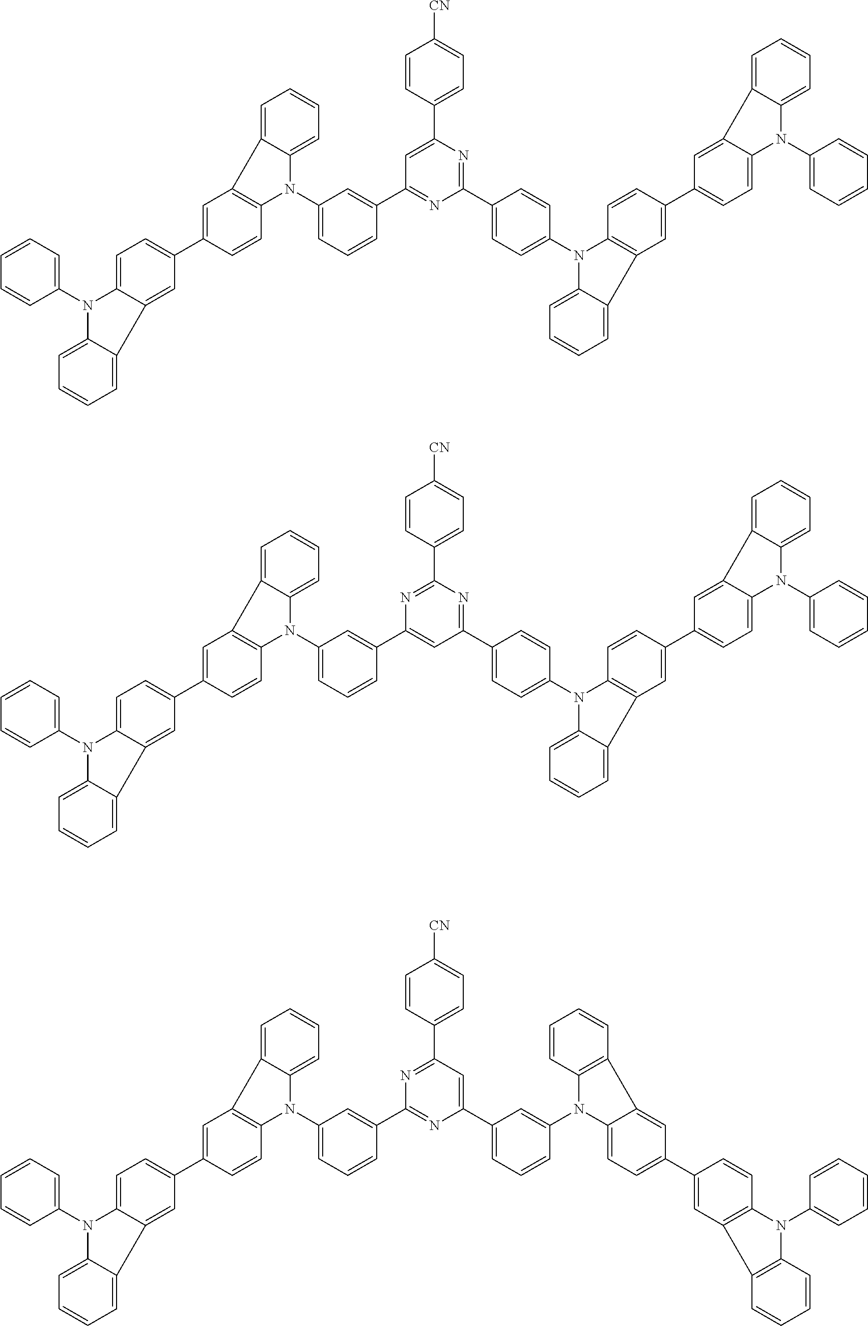 [SMILES: N#Cc1ccc(-c2cc(-c3cccc(-n4c5ccccc5c5cc(-c6ccc7c(c6)c6ccccc6n7-c6ccccc6)ccc54)c3)nc(-c3ccc(-n4c5ccccc5c5cc(-c6ccc7c(c6)c6ccccc6n7-c6ccccc6)ccc54)cc3)n2)cc1 ; N#Cc1ccc(-c2nc(-c3ccc(-n4c5ccccc5c5cc(-c6ccc7c(c6)c6ccccc6n7-c6ccccc6)ccc54)cc3)cc(-c3cccc(-n4c5ccccc5c5cc(-c6ccc7c(c6)c6ccccc6n7-c6ccccc6)ccc54)c3)n2)cc1 ; N#Cc1ccc(-c2cc(-c3cccc(-n4c5ccccc5c5cc(-c6ccc7c(c6)c6ccccc6n7-c6ccccc6)ccc54)c3)nc(-c3cccc(-n4c5ccccc5c5cc(-c6ccc7c(c6)c6ccccc6n7-c6ccccc6)ccc54)c3)n2)cc1]

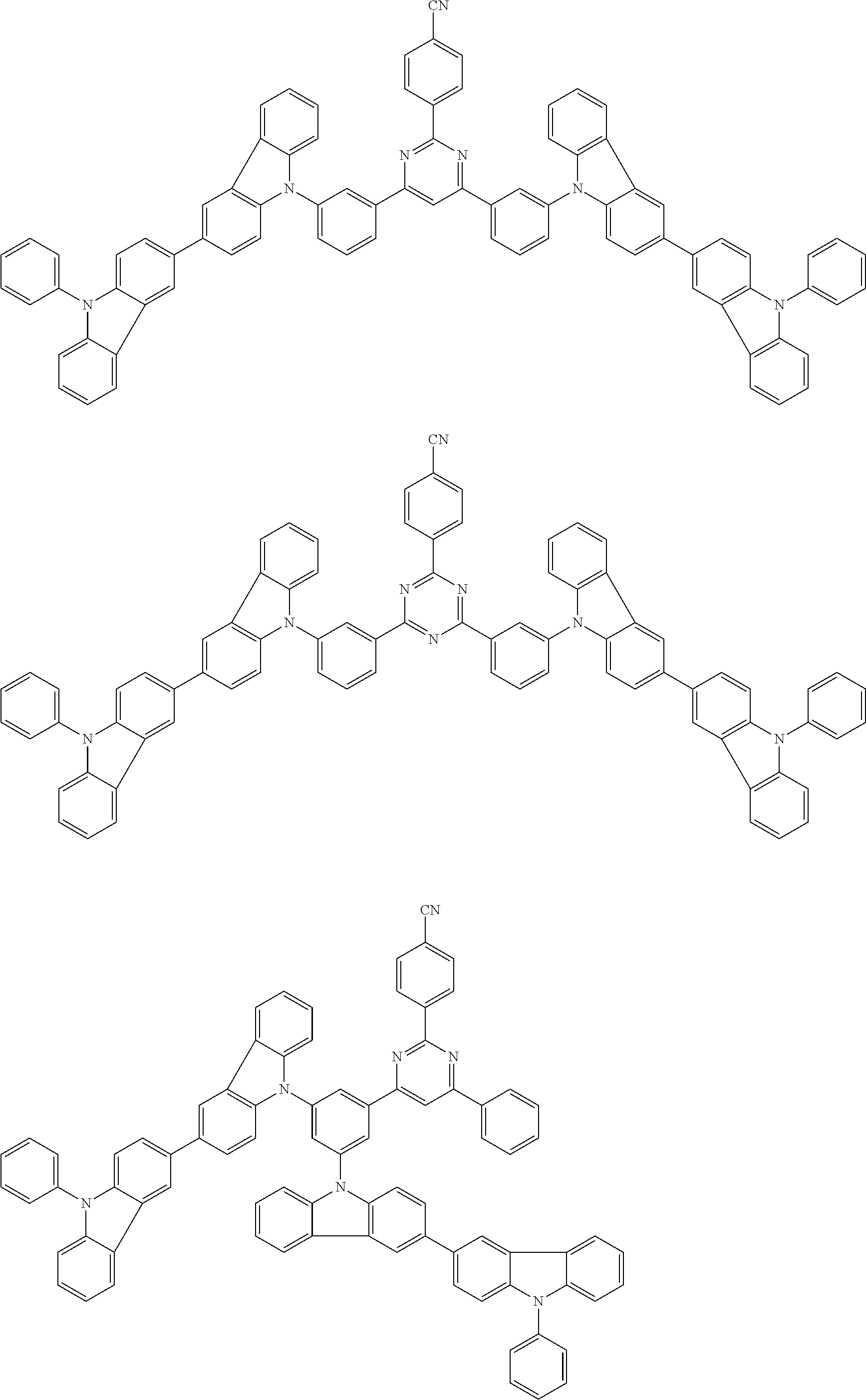 [SMILES: N#Cc1ccc(-c2nc(-c3cccc(-n4c5ccccc5c5cc(-c6ccc7c(c6)c6ccccc6n7-c6ccccc6)ccc54)c3)cc(-c3cccc(-n4c5ccccc5c5cc(-c6ccc7c(c6)c6ccccc6n7-c6ccccc6)ccc54)c3)n2)cc1 ; N#Cc1ccc(-c2nc(-c3cccc(-n4c5ccccc5c5cc(-c6ccc7c(c6)c6ccccc6n7-c6ccccc6)ccc54)c3)nc(-c3cccc(-n4c5ccccc5c5cc(-c6ccc7c(c6)c6ccccc6n7-c6ccccc6)ccc54)c3)n2)cc1 ; N#Cc1ccc(-c2nc(-c3ccccc3)cc(-c3cc(-n4c5ccccc5c5cc(-c6ccc7c(c6)c6ccccc6n7-c6ccccc6)ccc54)cc(-n4c5ccccc5c5cc(-c6ccc7c(c6)c6ccccc6n7-c6ccccc6)ccc54)c3)n2)cc1]

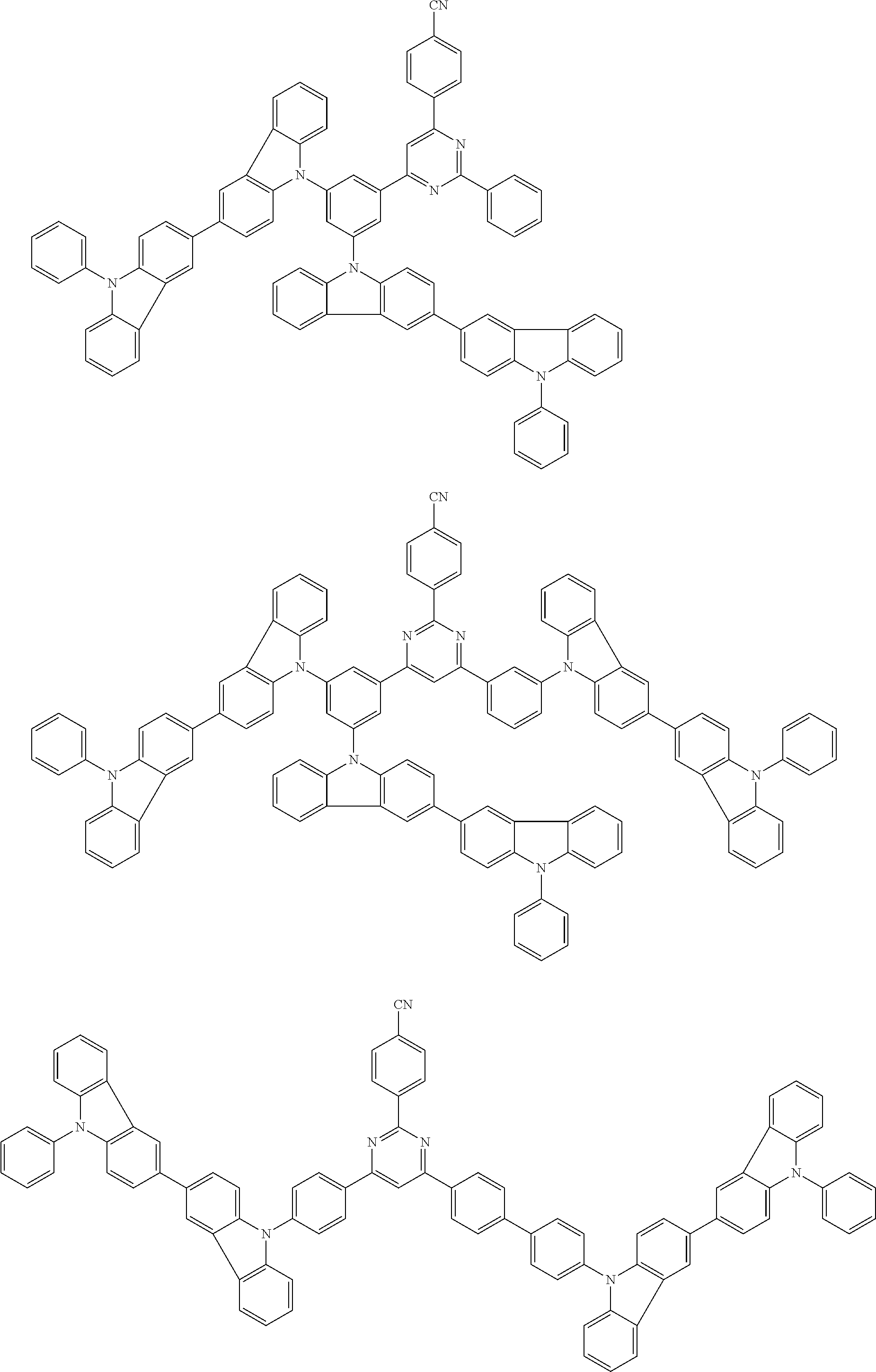

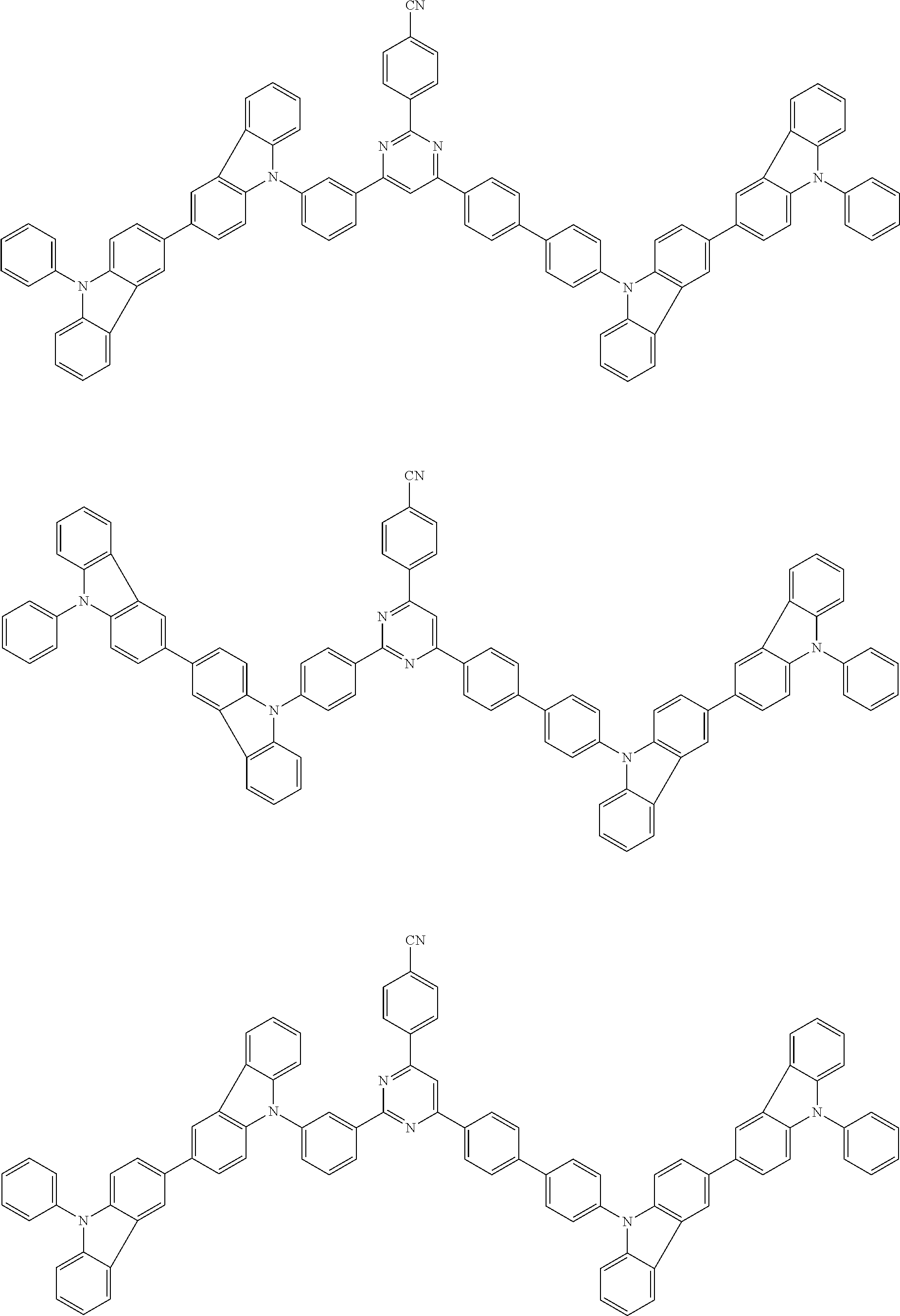

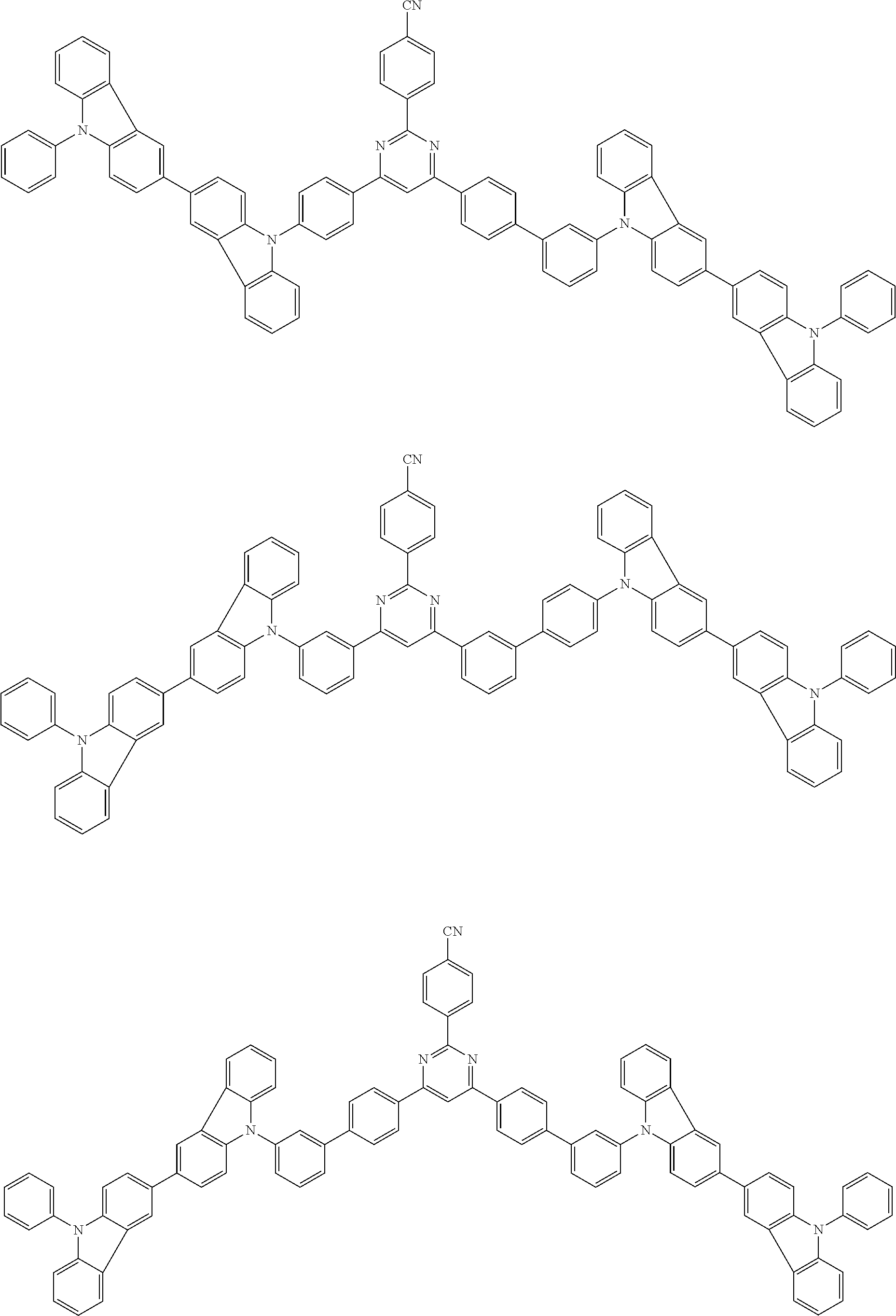

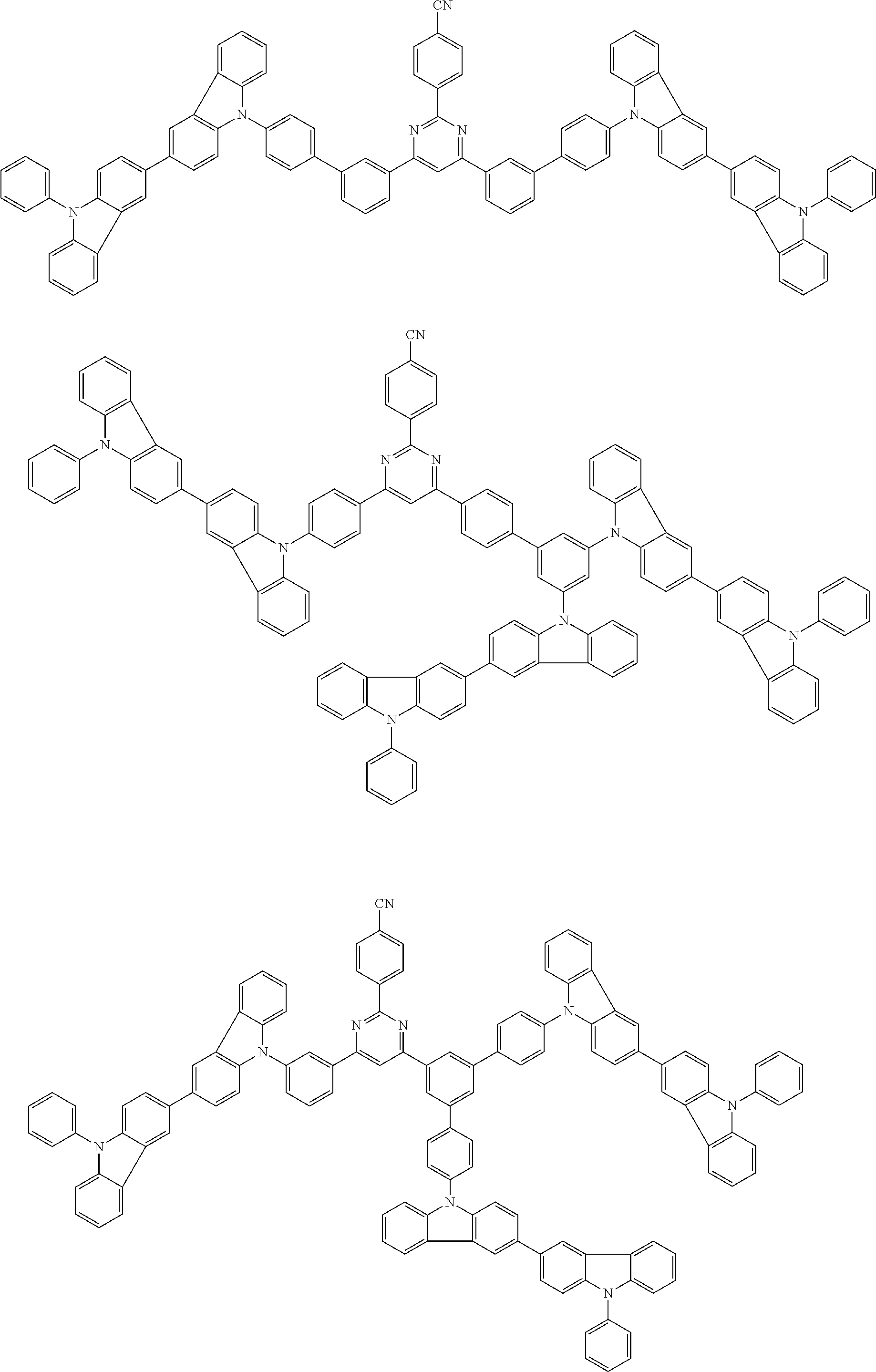

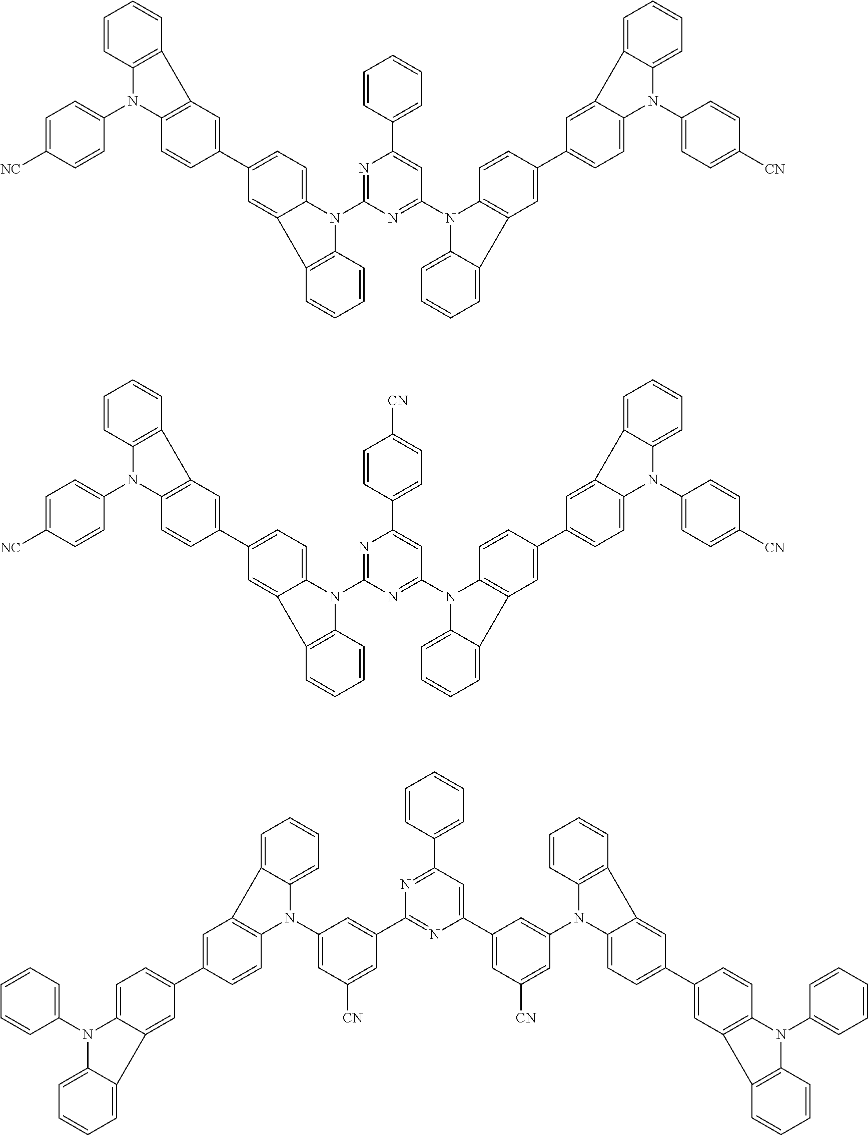 [SMILES: N#Cc1ccc(-n2c3ccccc3c3cc(-c4ccc5c(c4)c4ccccc4n5-c4cc(-c5ccccc5)nc(-n5c6ccccc6c6cc(-c7ccc8c(c7)c7ccccc7n8-c7ccc(C#N)cc7)ccc65)n4)ccc32)cc1 ; N#Cc1ccc(-c2cc(-n3c4ccccc4c4cc(-c5ccc6c(c5)c5ccccc5n6-c5ccc(C#N)cc5)ccc43)nc(-n3c4ccccc4c4cc(-c5ccc6c(c5)c5ccccc5n6-c5ccc(C#N)cc5)ccc43)n2)cc1 ; N#Cc1cc(-c2cc(-c3ccccc3)nc(-c3cc(C#N)cc(-n4c5ccccc5c5cc(-c6ccc7c(c6)c6ccccc6n7-c6ccccc6)ccc54)c3)n2)cc(-n2c3ccccc3c3cc(-c4ccc5c(c4)c4ccccc4n5-c4ccccc4)ccc32)c1]

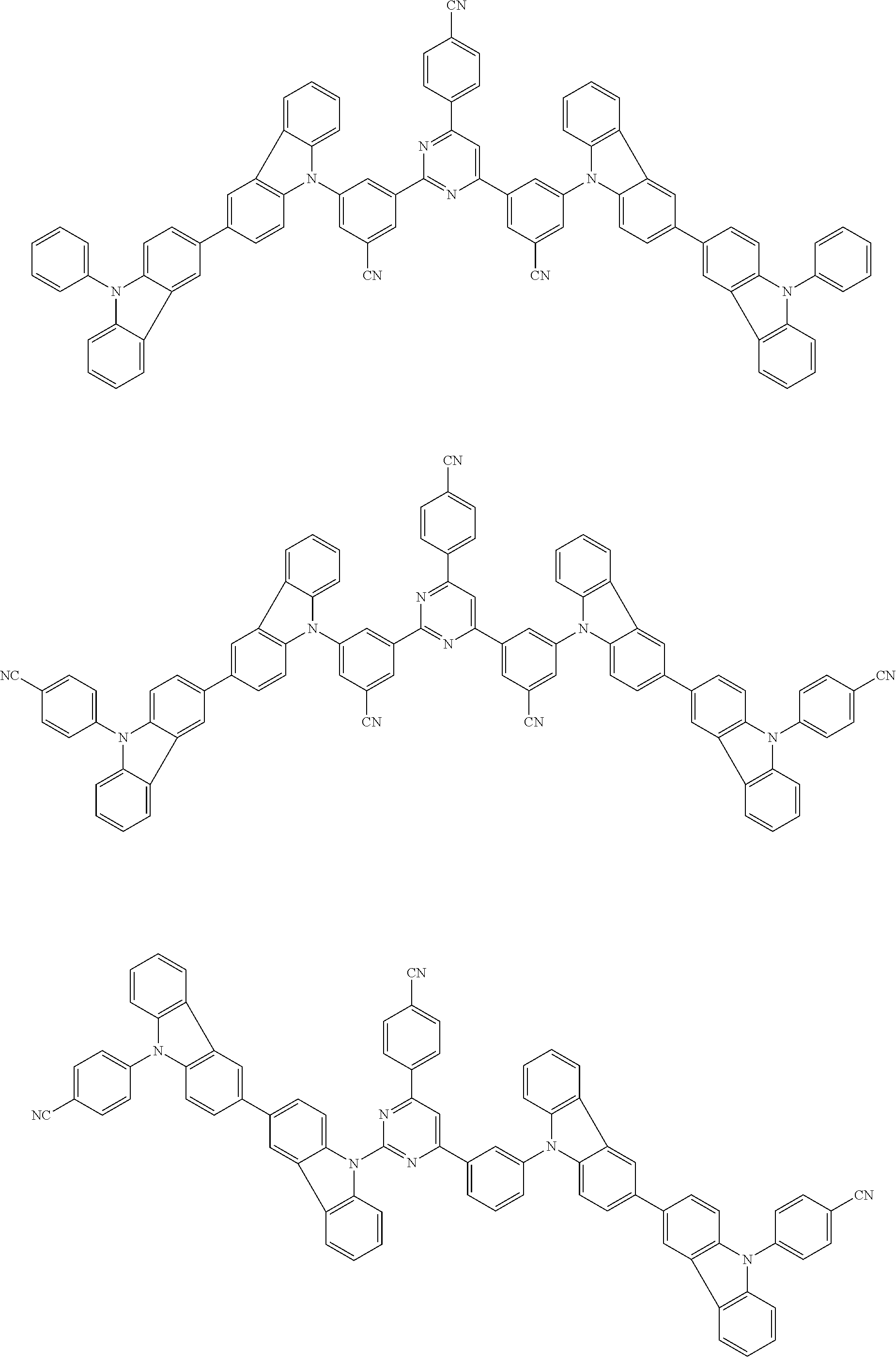 [SMILES: N#Cc1ccc(-c2cc(-c3cc(C#N)cc(-n4c5ccccc5c5cc(-c6ccc7c(c6)c6ccccc6n7-c6ccccc6)ccc54)c3)nc(-c3cc(C#N)cc(-n4c5ccccc5c5cc(-c6ccc7c(c6)c6ccccc6n7-c6ccccc6)ccc54)c3)n2)cc1 ; N#Cc1ccc(-c2cc(-c3cc(C#N)cc(-n4c5ccccc5c5cc(-c6ccc7c(c6)c6ccccc6n7-c6ccc(C#N)cc6)ccc54)c3)nc(-c3cc(C#N)cc(-n4c5ccccc5c5cc(-c6ccc7c(c6)c6ccccc6n7-c6ccc(C#N)cc6)ccc54)c3)n2)cc1 ; N#Cc1ccc(-c2cc(-c3cccc(-n4c5ccccc5c5cc(-c6ccc7c(c6)c6ccccc6n7-c6ccc(C#N)cc6)ccc54)c3)nc(-n3c4ccccc4c4cc(-c5ccc6c(c5)c5ccccc5n6-c5ccc(C#N)cc5)ccc43)n2)cc1]

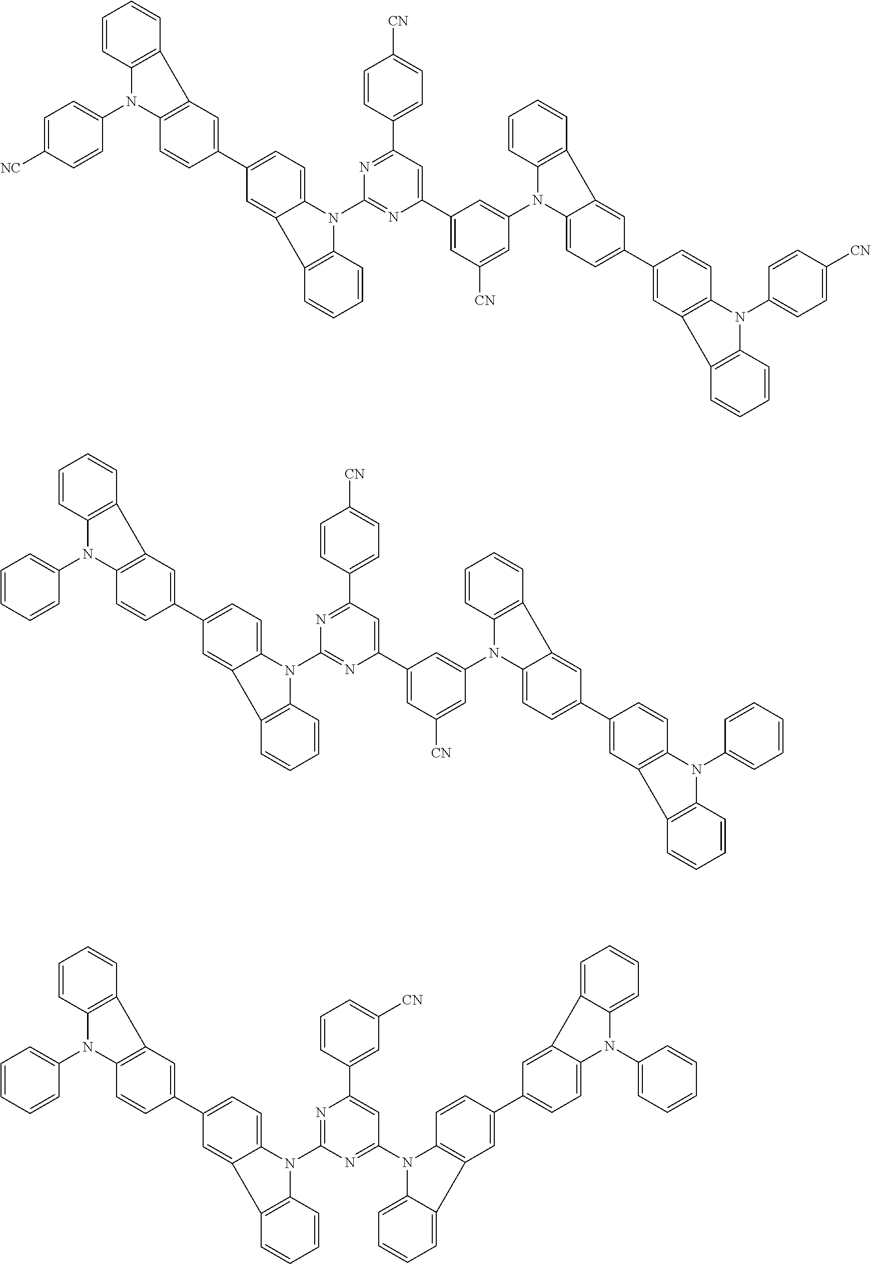 [SMILES: N#Cc1ccc(-c2cc(-c3cc(C#N)cc(-n4c5ccccc5c5cc(-c6ccc7c(c6)c6ccccc6n7-c6ccc(C#N)cc6)ccc54)c3)nc(-n3c4ccccc4c4cc(-c5ccc6c(c5)c5ccccc5n6-c5ccc(C#N)cc5)ccc43)n2)cc1 ; N#Cc1ccc(-c2cc(-c3cc(C#N)cc(-n4c5ccccc5c5cc(-c6ccc7c(c6)c6ccccc6n7-c6ccccc6)ccc54)c3)nc(-n3c4ccccc4c4cc(-c5ccc6c(c5)c5ccccc5n6-c5ccccc5)ccc43)n2)cc1 ; N#Cc1cccc(-c2cc(-n3c4ccccc4c4cc(-c5ccc6c(c5)c5ccccc5n6-c5ccccc5)ccc43)nc(-n3c4ccccc4c4cc(-c5ccc6c(c5)c5ccccc5n6-c5ccccc5)ccc43)n2)c1]

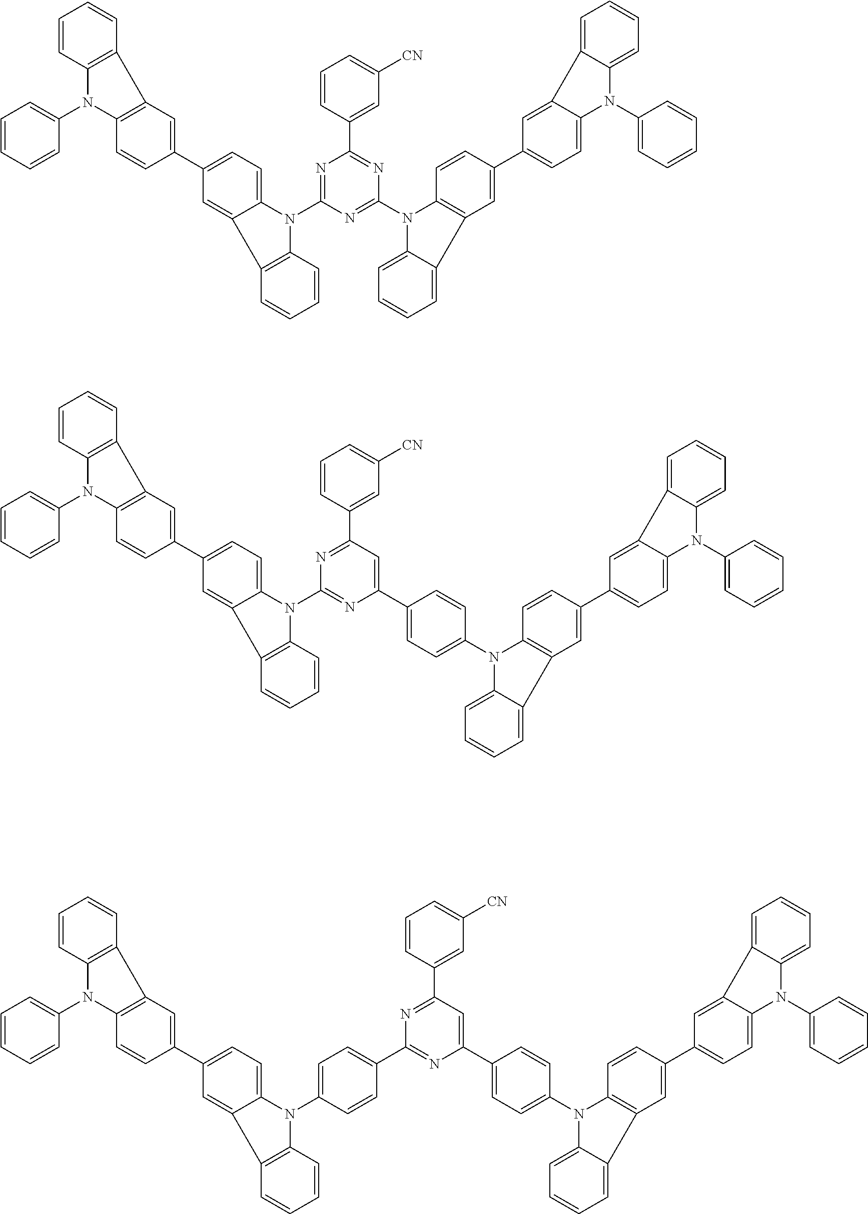 [SMILES: N#Cc1cccc(-c2nc(-n3c4ccccc4c4cc(-c5ccc6c(c5)c5ccccc5n6-c5ccccc5)ccc43)nc(-n3c4ccccc4c4cc(-c5ccc6c(c5)c5ccccc5n6-c5ccccc5)ccc43)n2)c1 ; N#Cc1cccc(-c2cc(-c3ccc(-n4c5ccccc5c5cc(-c6ccc7c(c6)c6ccccc6n7-c6ccccc6)ccc54)cc3)nc(-n3c4ccccc4c4cc(-c5ccc6c(c5)c5ccccc5n6-c5ccccc5)ccc43)n2)c1 ; N#Cc1cccc(-c2cc(-c3ccc(-n4c5ccccc5c5cc(-c6ccc7c(c6)c6ccccc6n7-c6ccccc6)ccc54)cc3)nc(-c3ccc(-n4c5ccccc5c5cc(-c6ccc7c(c6)c6ccccc6n7-c6ccccc6)ccc54)cc3)n2)c1]

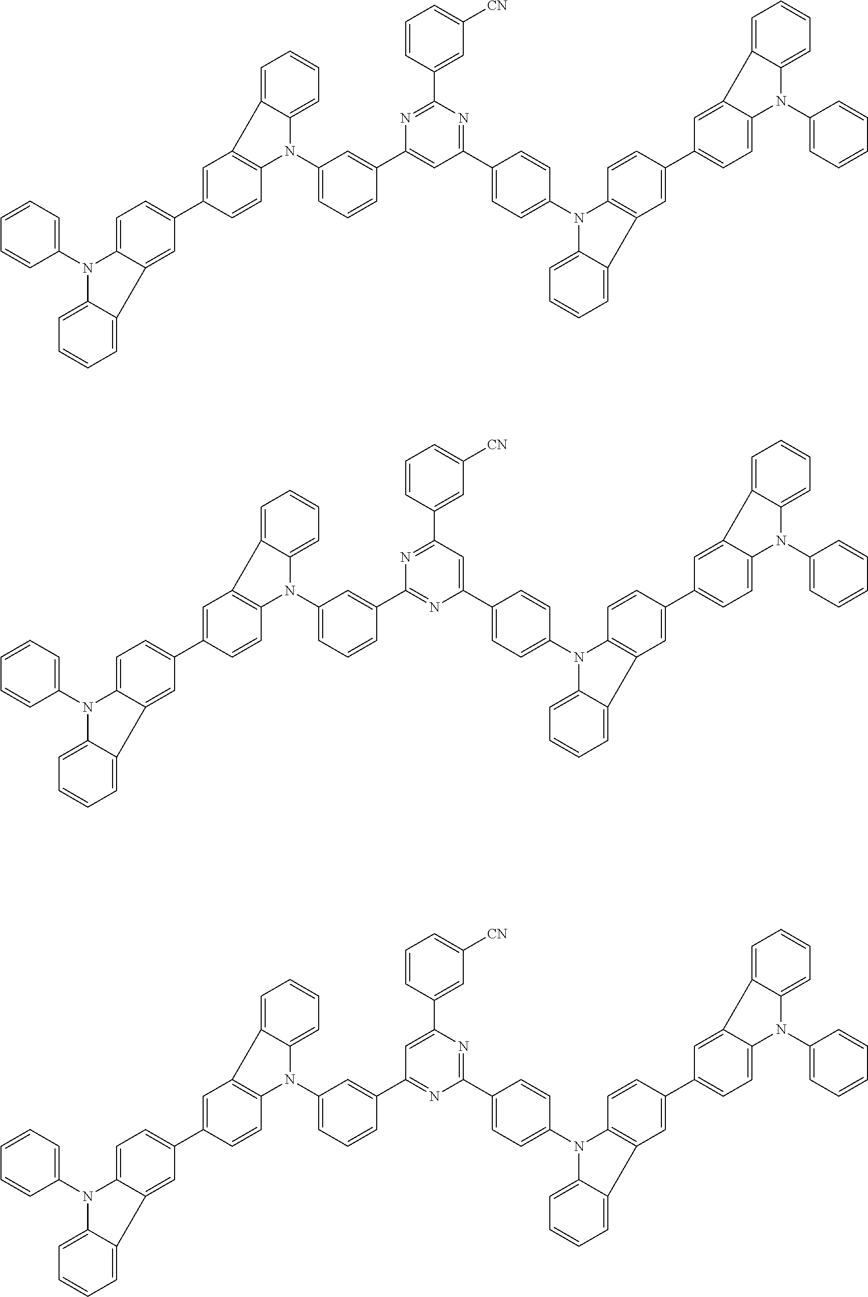 [SMILES: N#Cc1cccc(-c2nc(-c3ccc(-n4c5ccccc5c5cc(-c6ccc7c(c6)c6ccccc6n7-c6ccccc6)ccc54)cc3)cc(-c3cccc(-n4c5ccccc5c5cc(-c6ccc7c(c6)c6ccccc6n7-c6ccccc6)ccc54)c3)n2)c1 ; N#Cc1cccc(-c2cc(-c3ccc(-n4c5ccccc5c5cc(-c6ccc7c(c6)c6ccccc6n7-c6ccccc6)ccc54)cc3)nc(-c3cccc(-n4c5ccccc5c5cc(-c6ccc7c(c6)c6ccccc6n7-c6ccccc6)ccc54)c3)n2)c1 ; N#Cc1cccc(-c2cc(-c3cccc(-n4c5ccccc5c5cc(-c6ccc7c(c6)c6ccccc6n7-c6ccccc6)ccc54)c3)nc(-c3ccc(-n4c5ccccc5c5cc(-c6ccc7c(c6)c6ccccc6n7-c6ccccc6)ccc54)cc3)n2)c1]

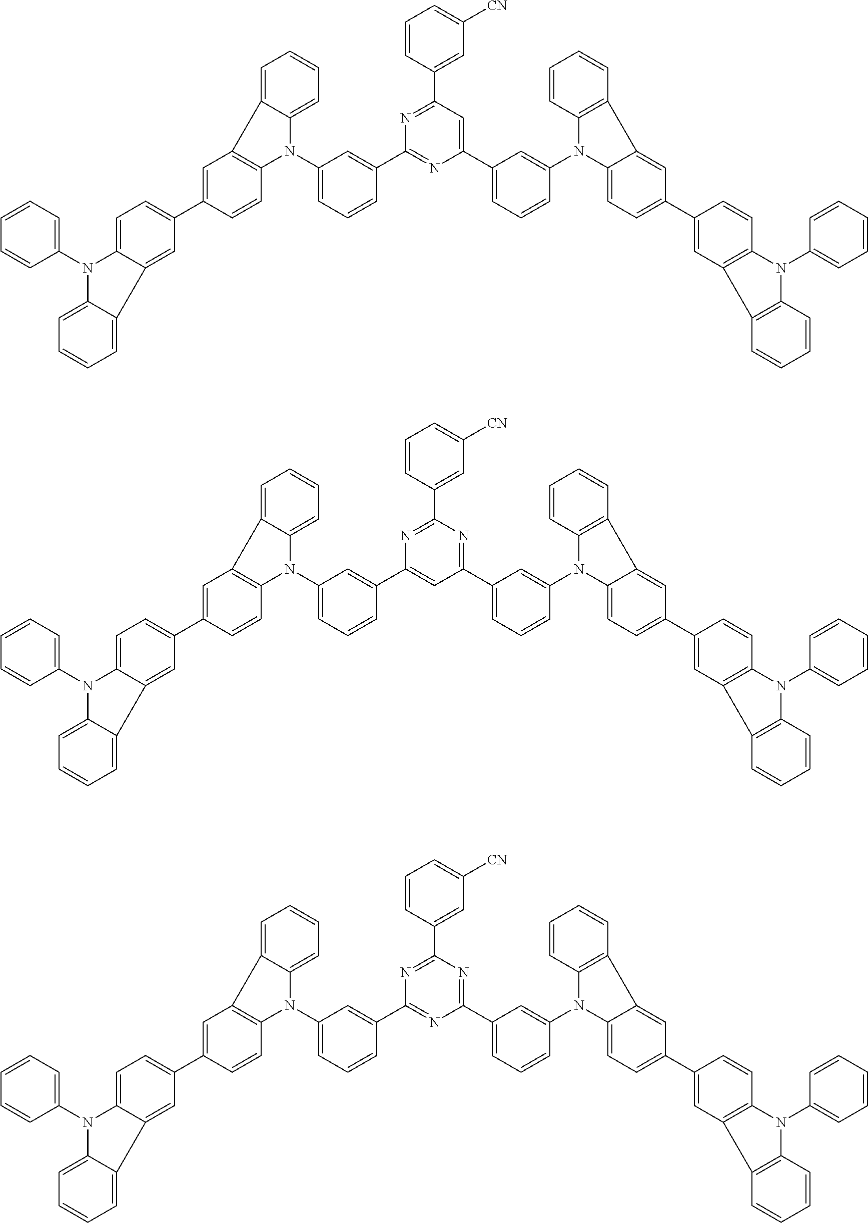 [SMILES: N#Cc1cccc(-c2cc(-c3cccc(-n4c5ccccc5c5cc(-c6ccc7c(c6)c6ccccc6n7-c6ccccc6)ccc54)c3)nc(-c3cccc(-n4c5ccccc5c5cc(-c6ccc7c(c6)c6ccccc6n7-c6ccccc6)ccc54)c3)n2)c1 ; N#Cc1cccc(-c2nc(-c3cccc(-n4c5ccccc5c5cc(-c6ccc7c(c6)c6ccccc6n7-c6ccccc6)ccc54)c3)cc(-c3cccc(-n4c5ccccc5c5cc(-c6ccc7c(c6)c6ccccc6n7-c6ccccc6)ccc54)c3)n2)c1 ; N#Cc1cccc(-c2nc(-c3cccc(-n4c5ccccc5c5cc(-c6ccc7c(c6)c6ccccc6n7-c6ccccc6)ccc54)c3)nc(-c3cccc(-n4c5ccccc5c5cc(-c6ccc7c(c6)c6ccccc6n7-c6ccccc6)ccc54)c3)n2)c1]

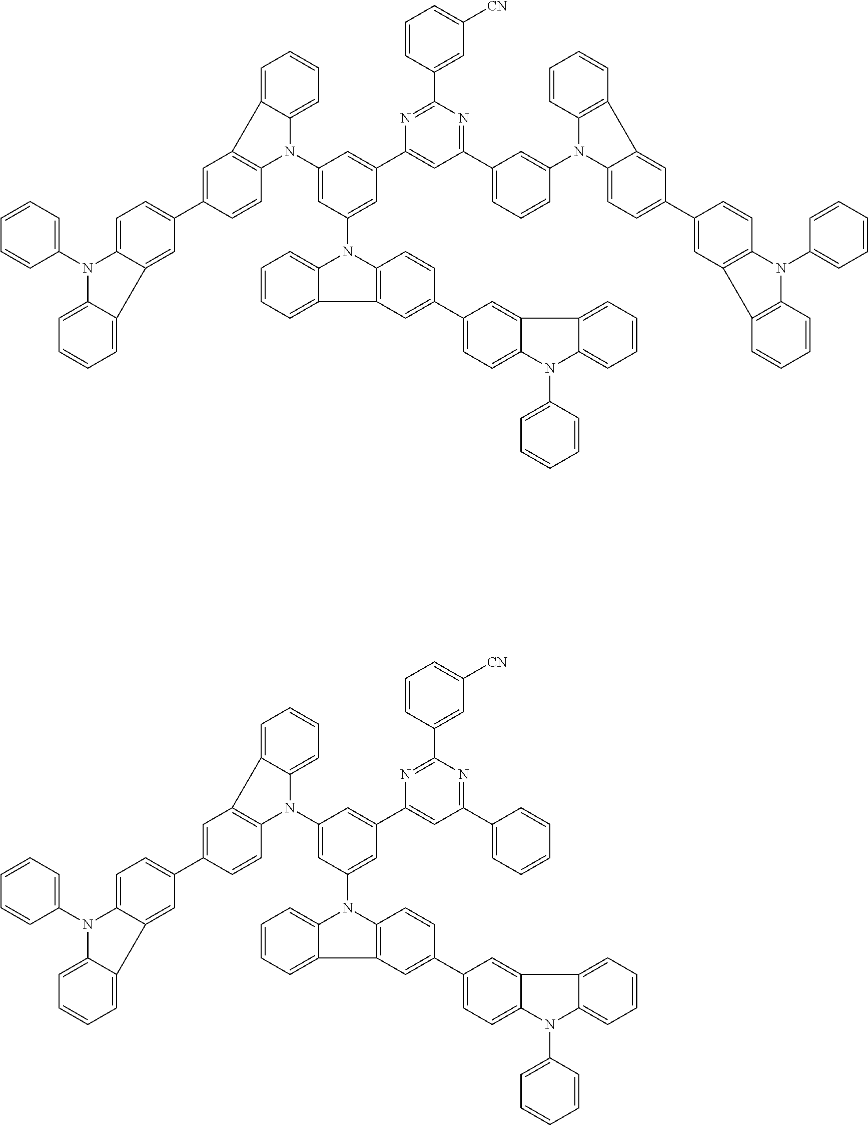 [SMILES: N#Cc1cccc(-c2nc(-c3cccc(-n4c5ccccc5c5cc(-c6ccc7c(c6)c6ccccc6n7-c6ccccc6)ccc54)c3)cc(-c3cc(-n4c5ccccc5c5cc(-c6ccc7c(c6)c6ccccc6n7-c6ccccc6)ccc54)cc(-n4c5ccccc5c5cc(-c6ccc7c(c6)c6ccccc6n7-c6ccccc6)ccc54)c3)n2)c1 ; N#Cc1cccc(-c2nc(-c3ccccc3)cc(-c3cc(-n4c5ccccc5c5cc(-c6ccc7c(c6)c6ccccc6n7-c6ccccc6)ccc54)cc(-n4c5ccccc5c5cc(-c6ccc7c(c6)c6ccccc6n7-c6ccccc6)ccc54)c3)n2)c1]

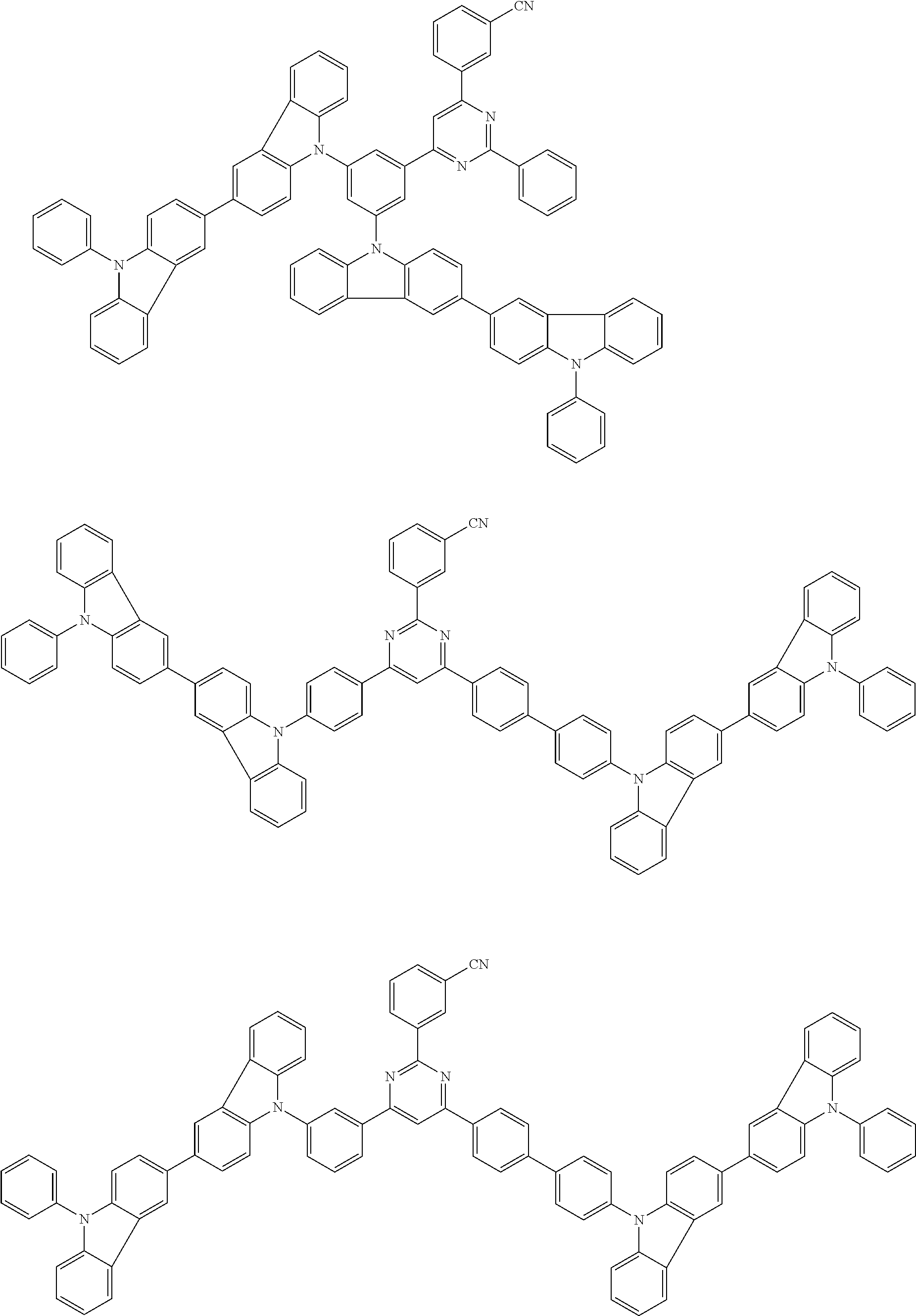 [SMILES: N#Cc1cccc(-c2cc(-c3cc(-n4c5ccccc5c5cc(-c6ccc7c(c6)c6ccccc6n7-c6ccccc6)ccc54)cc(-n4c5ccccc5c5cc(-c6ccc7c(c6)c6ccccc6n7-c6ccccc6)ccc54)c3)nc(-c3ccccc3)n2)c1 ; N#Cc1cccc(-c2nc(-c3ccc(-c4ccc(-n5c6ccccc6c6cc(-c7ccc8c(c7)c7ccccc7n8-c7ccccc7)ccc65)cc4)cc3)cc(-c3ccc(-n4c5ccccc5c5cc(-c6ccc7c(c6)c6ccccc6n7-c6ccccc6)ccc54)cc3)n2)c1 ; N#Cc1cccc(-c2nc(-c3ccc(-c4ccc(-n5c6ccccc6c6cc(-c7ccc8c(c7)c7ccccc7n8-c7ccccc7)ccc65)cc4)cc3)cc(-c3cccc(-n4c5ccccc5c5cc(-c6ccc7c(c6)c6ccccc6n7-c6ccccc6)ccc54)c3)n2)c1]

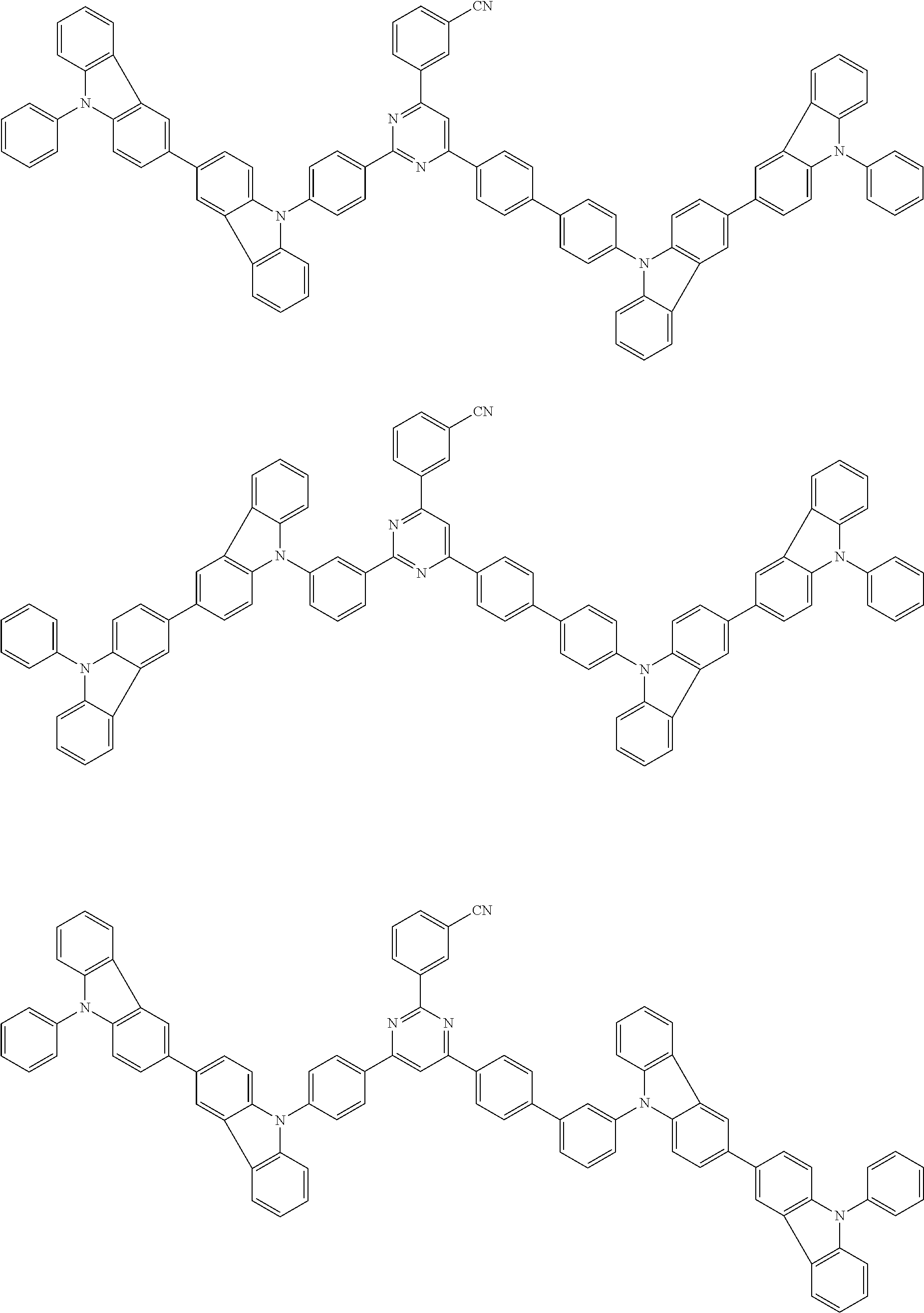

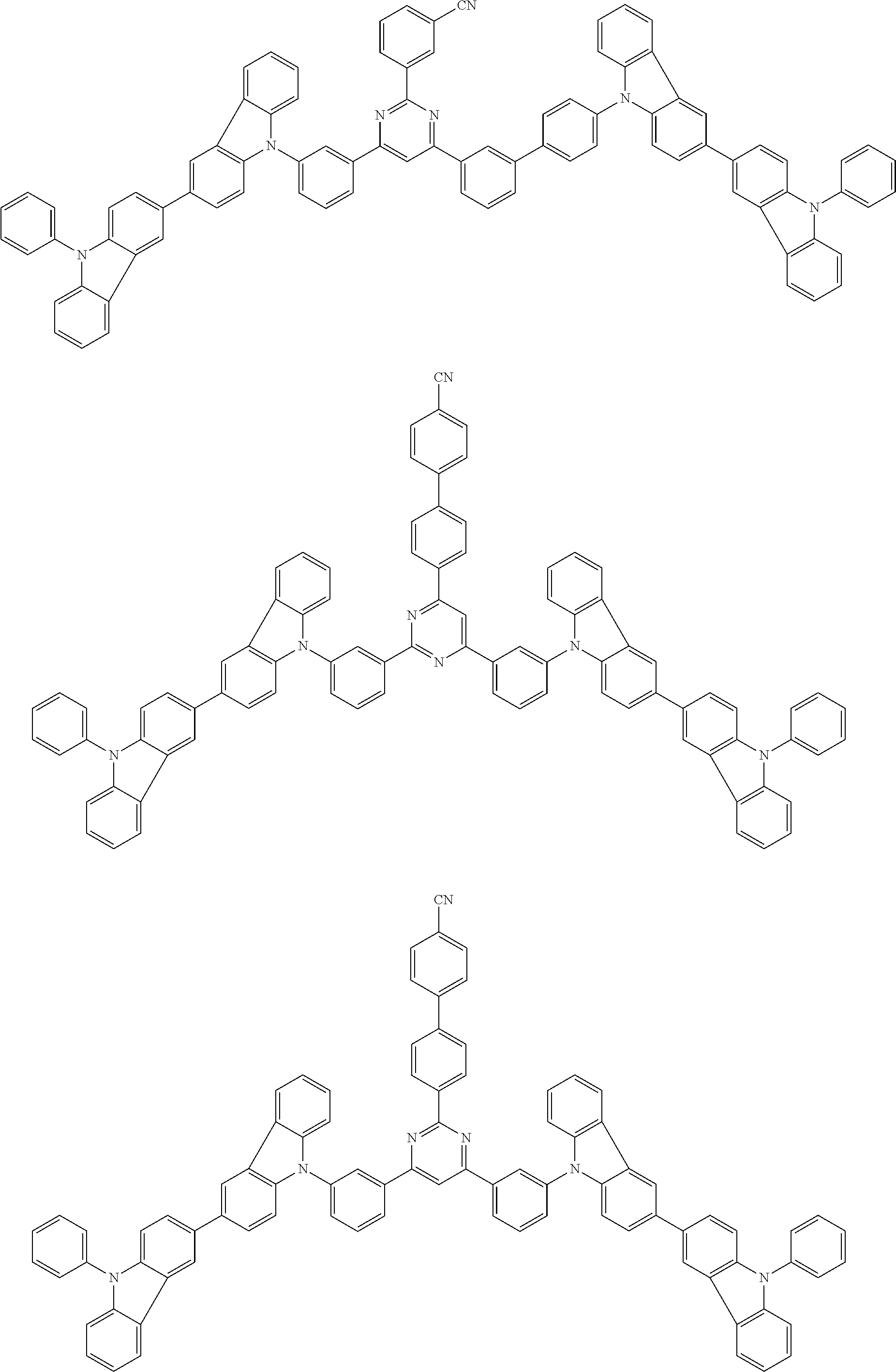

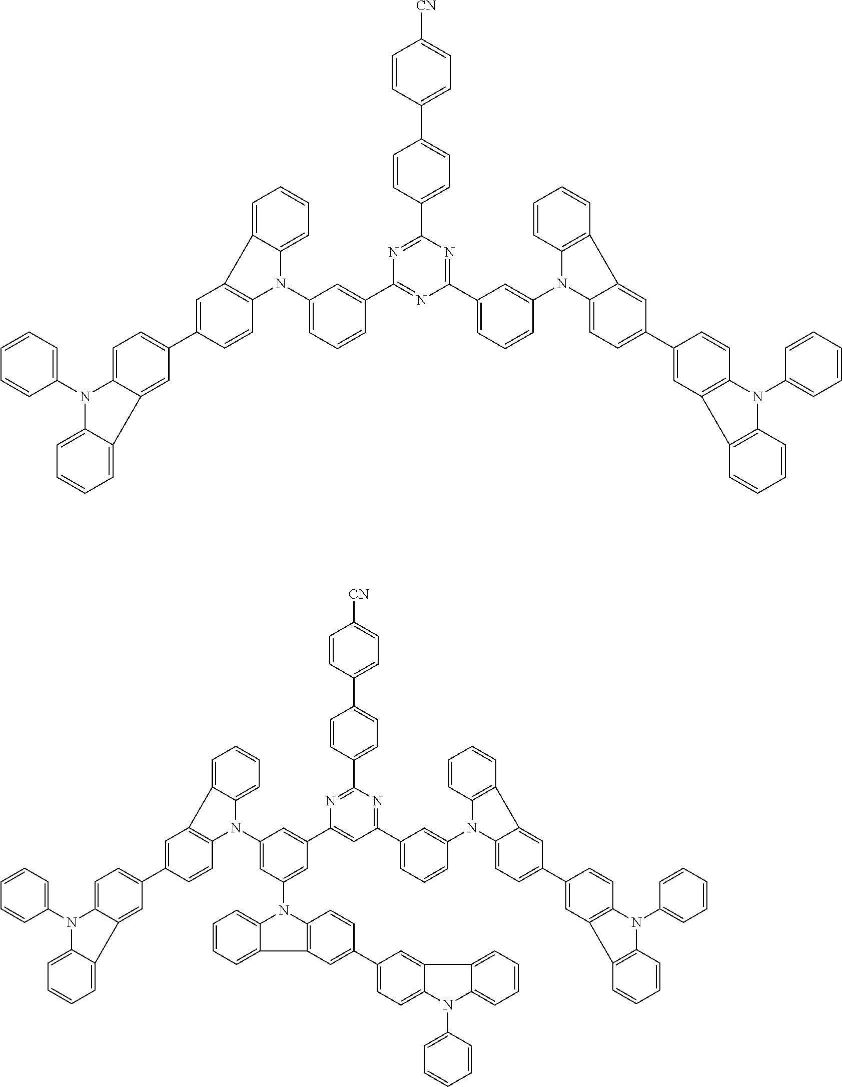 [SMILES: N#Cc1ccc(-c2ccc(-c3nc(-c4cccc(-n5c6ccccc6c6cc(-c7ccc8c(c7)c7ccccc7n8-c7ccccc7)ccc65)c4)nc(-c4cccc(-n5c6ccccc6c6cc(-c7ccc8c(c7)c7ccccc7n8-c7ccccc7)ccc65)c4)n3)cc2)cc1 ; N#Cc1ccc(-c2ccc(-c3nc(-c4cccc(-n5c6ccccc6c6cc(-c7ccc8c(c7)c7ccccc7n8-c7ccccc7)ccc65)c4)cc(-c4cc(-n5c6ccccc6c6cc(-c7ccc8c(c7)c7ccccc7n8-c7ccccc7)ccc65)cc(-n5c6ccccc6c6cc(-c7ccc8c(c7)c7ccccc7n8-c7ccccc7)ccc65)c4)n3)cc2)cc1]

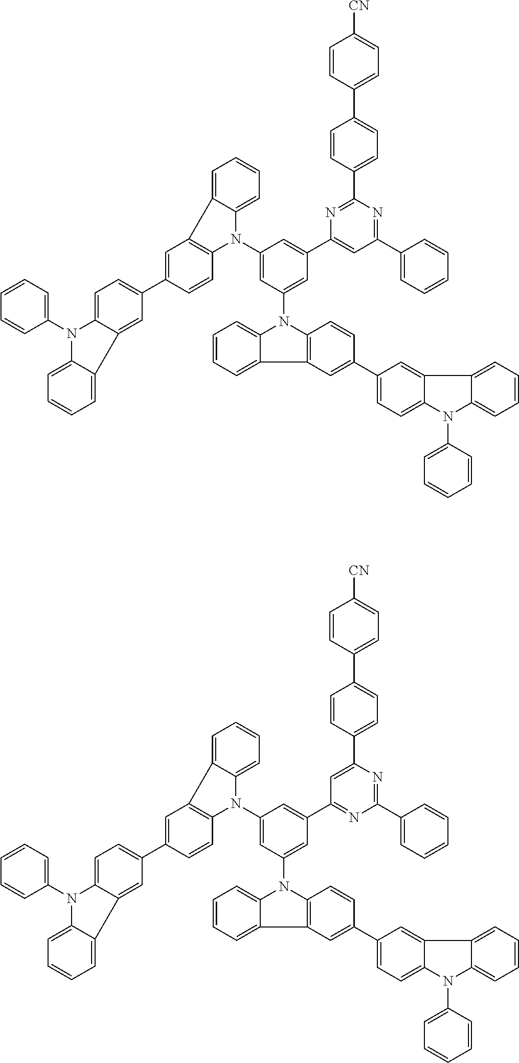 [SMILES: N#Cc1ccc(-c2ccc(-c3nc(-c4ccccc4)cc(-c4cc(-n5c6ccccc6c6cc(-c7ccc8c(c7)c7ccccc7n8-c7ccccc7)ccc65)cc(-n5c6ccccc6c6cc(-c7ccc8c(c7)c7ccccc7n8-c7ccccc7)ccc65)c4)n3)cc2)cc1 ; N#Cc1ccc(-c2ccc(-c3cc(-c4cc(-n5c6ccccc6c6cc(-c7ccc8c(c7)c7ccccc7n8-c7ccccc7)ccc65)cc(-n5c6ccccc6c6cc(-c7ccc8c(c7)c7ccccc7n8-c7ccccc7)ccc65)c4)nc(-c4ccccc4)n3)cc2)cc1]

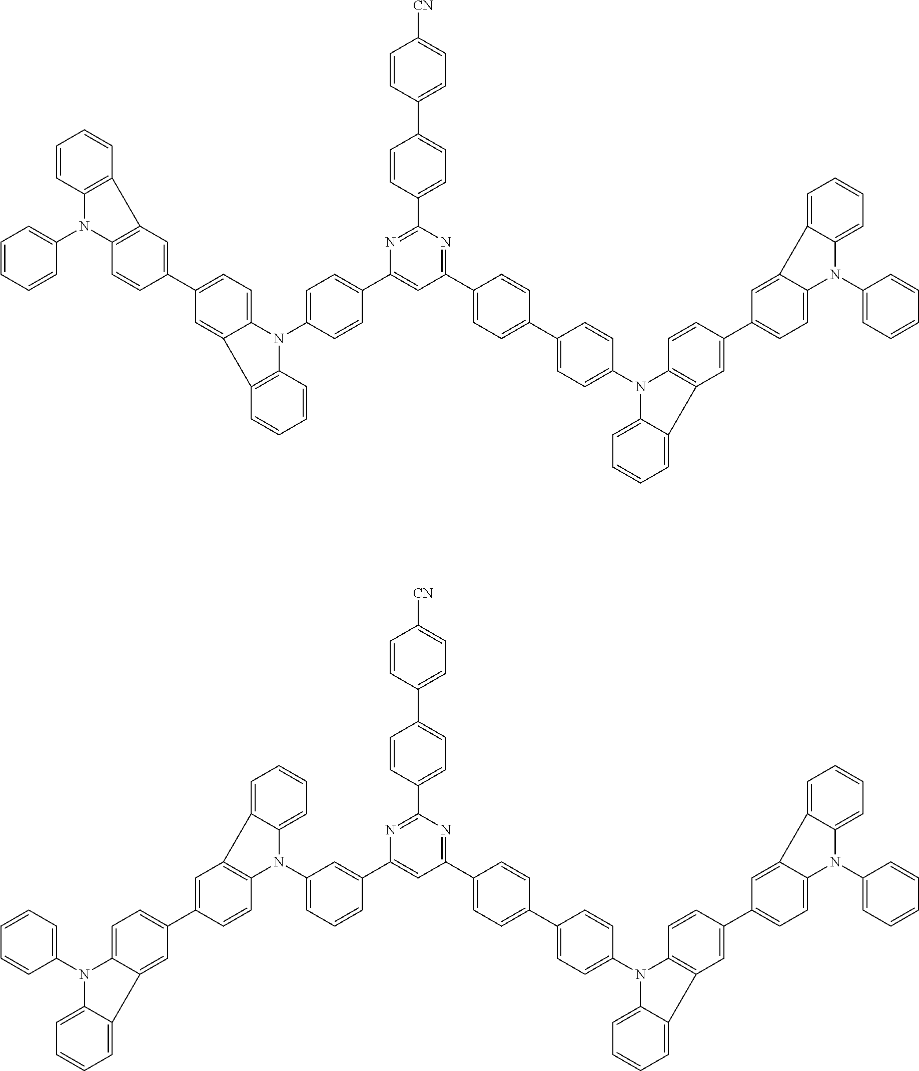 [SMILES: N#Cc1ccc(-c2ccc(-c3nc(-c4ccc(-c5ccc(-n6c7ccccc7c7cc(-c8ccc9c(c8)c8ccccc8n9-c8ccccc8)ccc76)cc5)cc4)cc(-c4ccc(-n5c6ccccc6c6cc(-c7ccc8c(c7)c7ccccc7n8-c7ccccc7)ccc65)cc4)n3)cc2)cc1 ; N#Cc1ccc(-c2ccc(-c3nc(-c4ccc(-c5ccc(-n6c7ccccc7c7cc(-c8ccc9c(c8)c8ccccc8n9-c8ccccc8)ccc76)cc5)cc4)cc(-c4cccc(-n5c6ccccc6c6cc(-c7ccc8c(c7)c7ccccc7n8-c7ccccc7)ccc65)c4)n3)cc2)cc1]

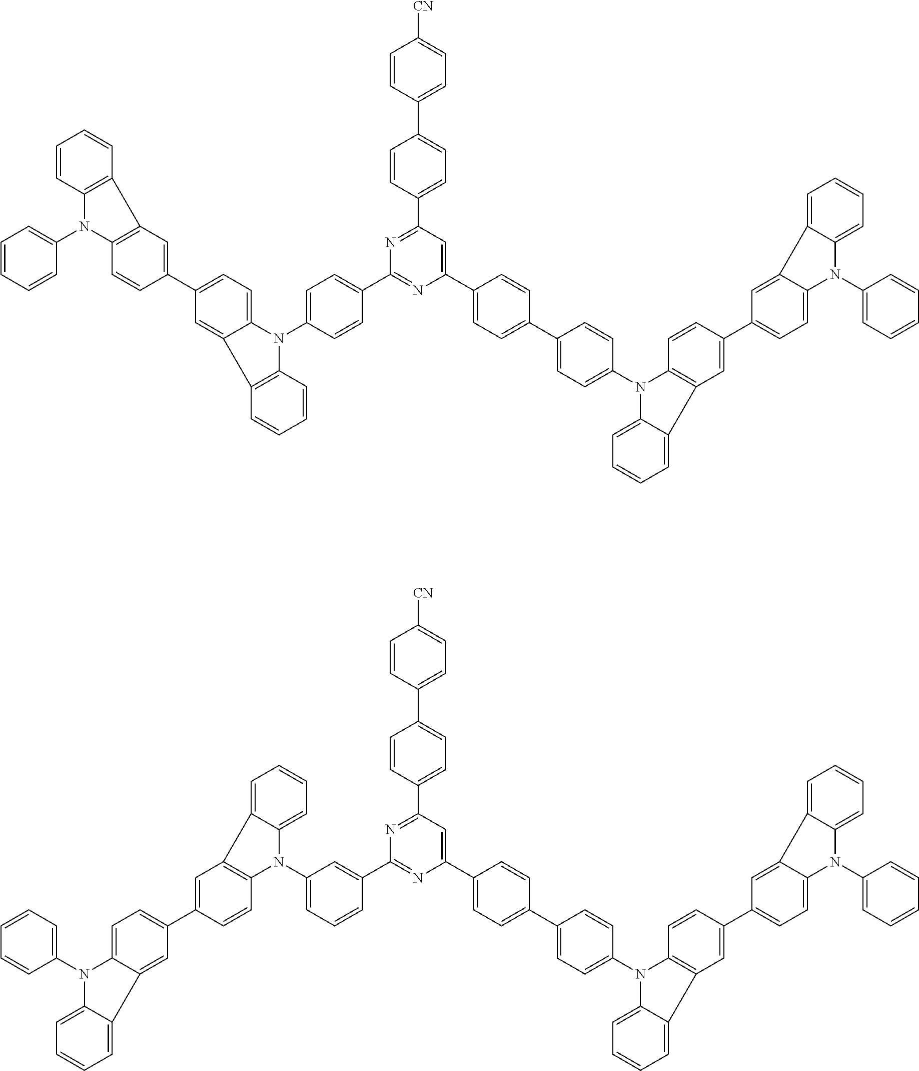 [SMILES: N#Cc1ccc(-c2ccc(-c3cc(-c4ccc(-c5ccc(-n6c7ccccc7c7cc(-c8ccc9c(c8)c8ccccc8n9-c8ccccc8)ccc76)cc5)cc4)nc(-c4ccc(-n5c6ccccc6c6cc(-c7ccc8c(c7)c7ccccc7n8-c7ccccc7)ccc65)cc4)n3)cc2)cc1 ; N#Cc1ccc(-c2ccc(-c3cc(-c4ccc(-c5ccc(-n6c7ccccc7c7cc(-c8ccc9c(c8)c8ccccc8n9-c8ccccc8)ccc76)cc5)cc4)nc(-c4cccc(-n5c6ccccc6c6cc(-c7ccc8c(c7)c7ccccc7n8-c7ccccc7)ccc65)c4)n3)cc2)cc1]

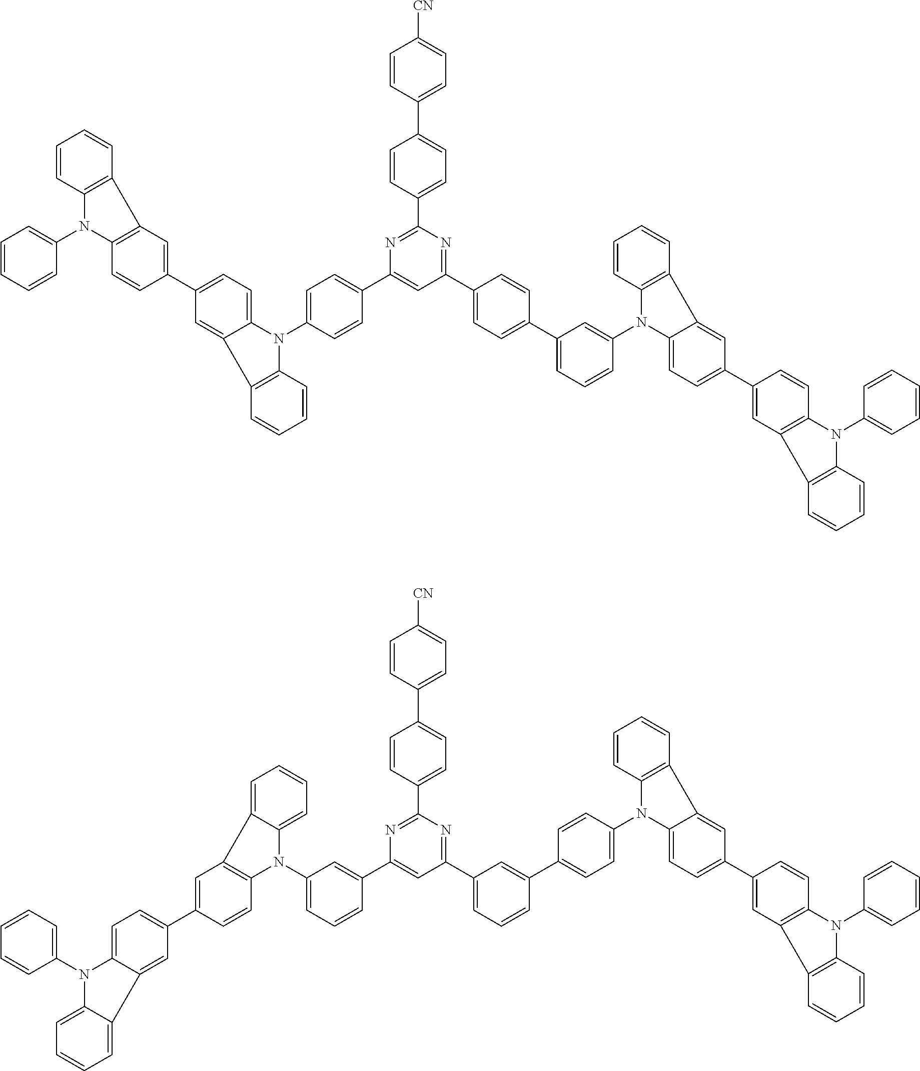 [SMILES: N#Cc1ccc(-c2ccc(-c3nc(-c4ccc(-c5cccc(-n6c7ccccc7c7cc(-c8ccc9c(c8)c8ccccc8n9-c8ccccc8)ccc76)c5)cc4)cc(-c4ccc(-n5c6ccccc6c6cc(-c7ccc8c(c7)c7ccccc7n8-c7ccccc7)ccc65)cc4)n3)cc2)cc1 ; N#Cc1ccc(-c2ccc(-c3nc(-c4cccc(-c5ccc(-n6c7ccccc7c7cc(-c8ccc9c(c8)c8ccccc8n9-c8ccccc8)ccc76)cc5)c4)cc(-c4cccc(-n5c6ccccc6c6cc(-c7ccc8c(c7)c7ccccc7n8-c7ccccc7)ccc65)c4)n3)cc2)cc1]

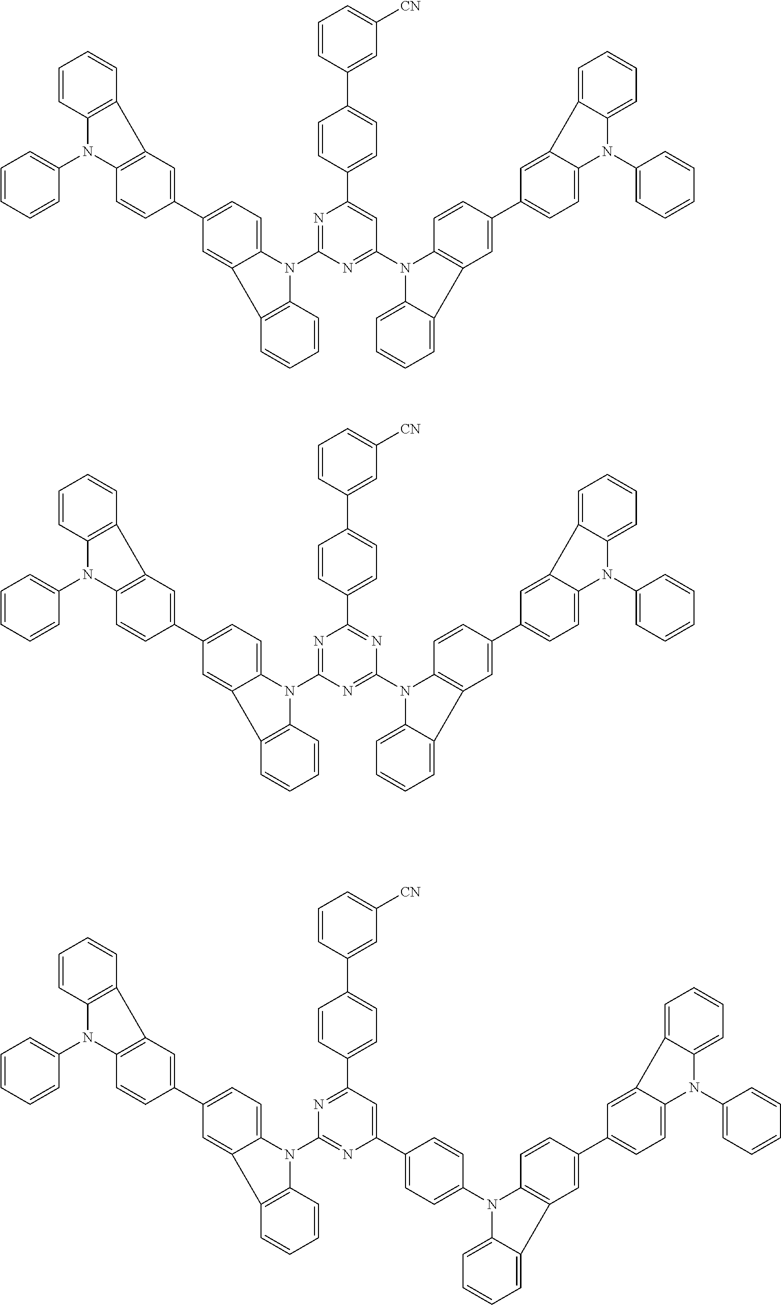 [SMILES: N#Cc1cccc(-c2ccc(-c3cc(-n4c5ccccc5c5cc(-c6ccc7c(c6)c6ccccc6n7-c6ccccc6)ccc54)nc(-n4c5ccccc5c5cc(-c6ccc7c(c6)c6ccccc6n7-c6ccccc6)ccc54)n3)cc2)c1 ; N#Cc1cccc(-c2ccc(-c3nc(-n4c5ccccc5c5cc(-c6ccc7c(c6)c6ccccc6n7-c6ccccc6)ccc54)nc(-n4c5ccccc5c5cc(-c6ccc7c(c6)c6ccccc6n7-c6ccccc6)ccc54)n3)cc2)c1 ; N#Cc1cccc(-c2ccc(-c3cc(-c4ccc(-n5c6ccccc6c6cc(-c7ccc8c(c7)c7ccccc7n8-c7ccccc7)ccc65)cc4)nc(-n4c5ccccc5c5cc(-c6ccc7c(c6)c6ccccc6n7-c6ccccc6)ccc54)n3)cc2)c1]

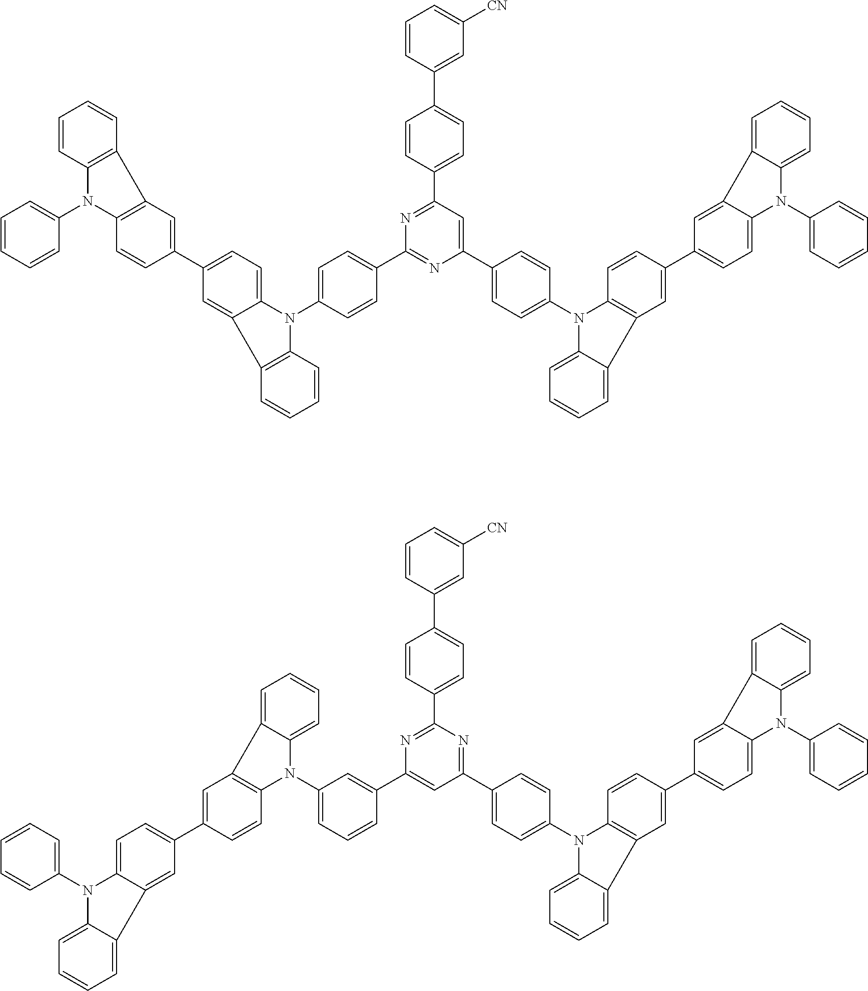 [SMILES: N#Cc1cccc(-c2ccc(-c3cc(-c4ccc(-n5c6ccccc6c6cc(-c7ccc8c(c7)c7ccccc7n8-c7ccccc7)ccc65)cc4)nc(-c4ccc(-n5c6ccccc6c6cc(-c7ccc8c(c7)c7ccccc7n8-c7ccccc7)ccc65)cc4)n3)cc2)c1 ; N#Cc1cccc(-c2ccc(-c3nc(-c4ccc(-n5c6ccccc6c6cc(-c7ccc8c(c7)c7ccccc7n8-c7ccccc7)ccc65)cc4)cc(-c4cccc(-n5c6ccccc6c6cc(-c7ccc8c(c7)c7ccccc7n8-c7ccccc7)ccc65)c4)n3)cc2)c1]

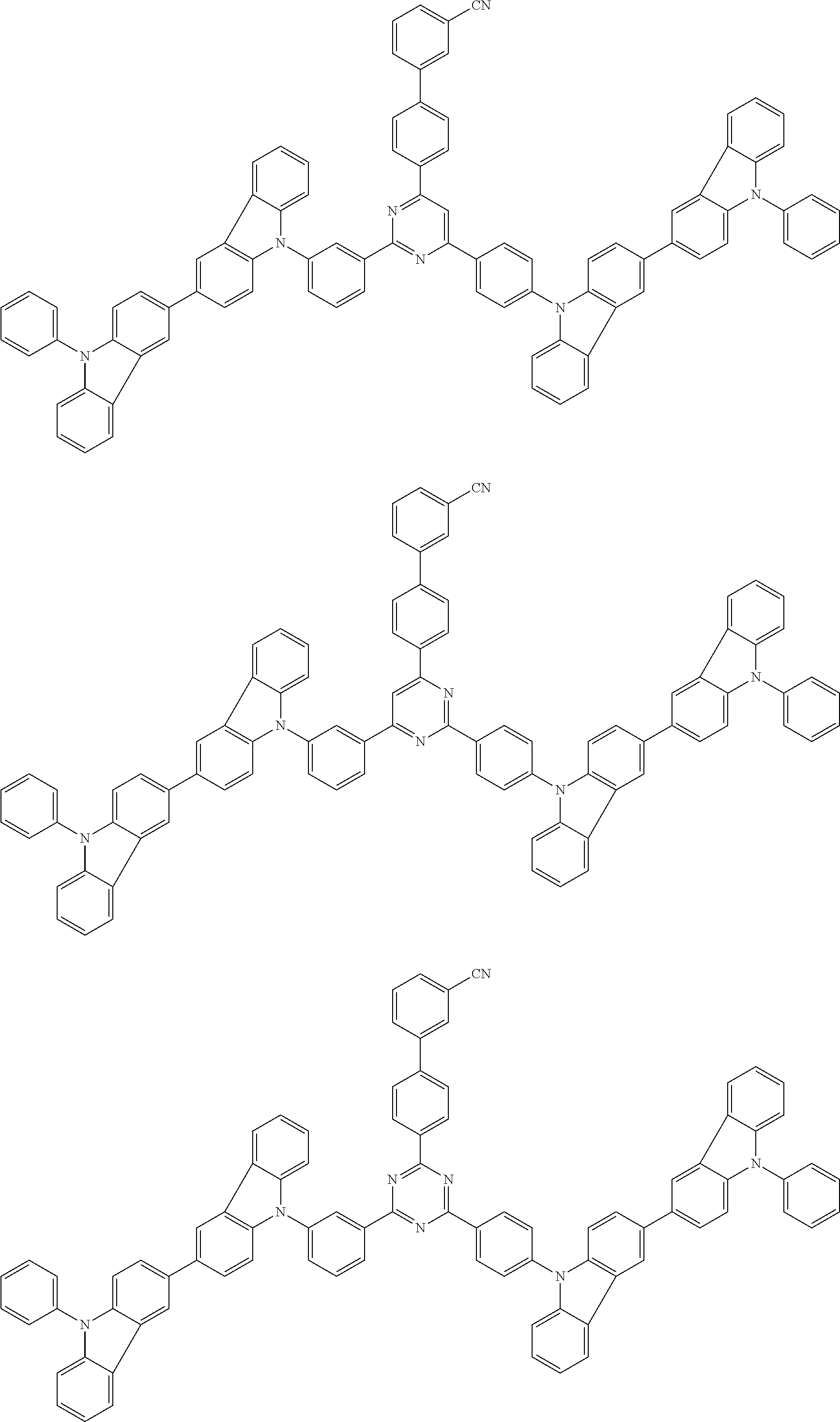

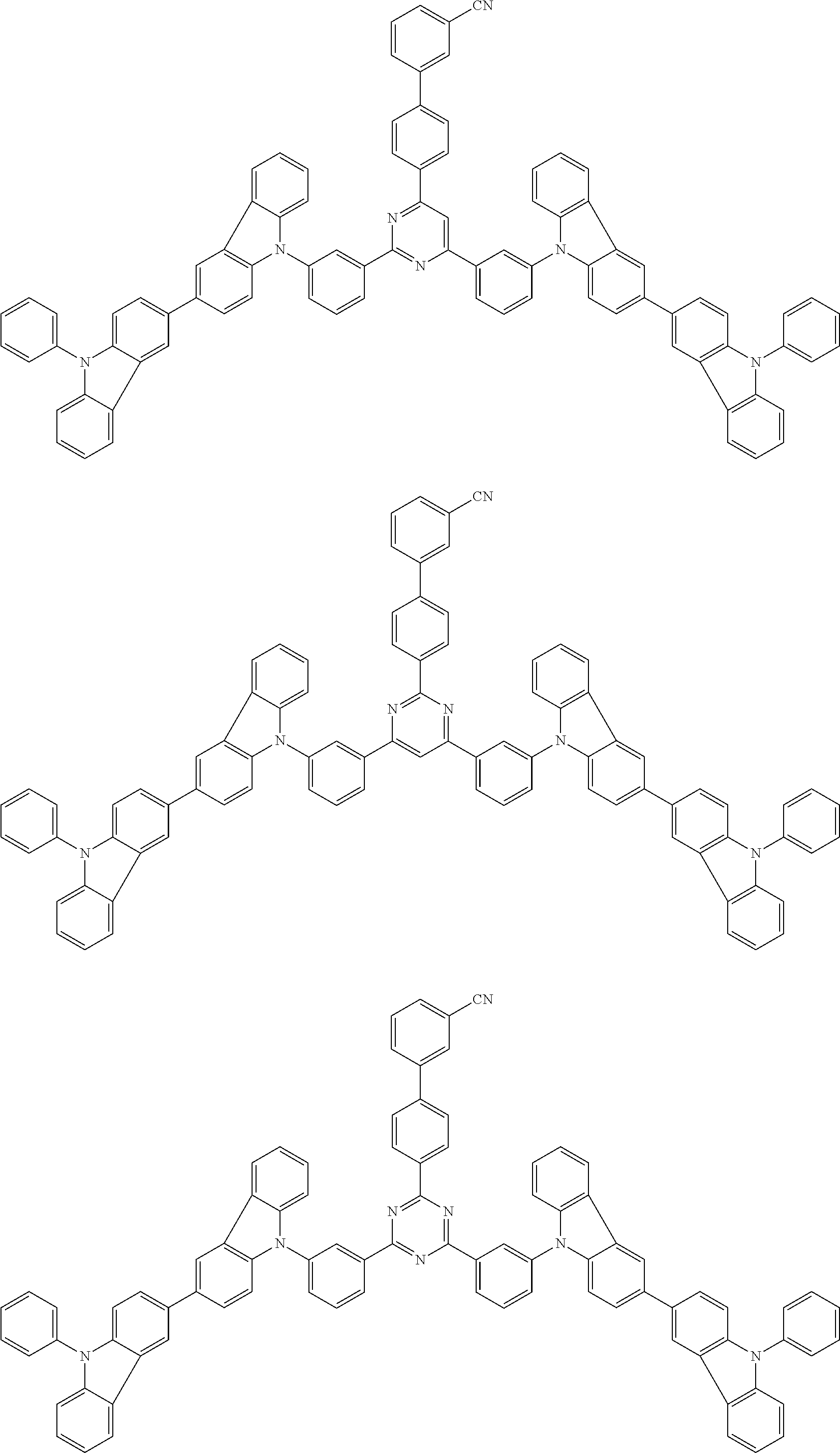

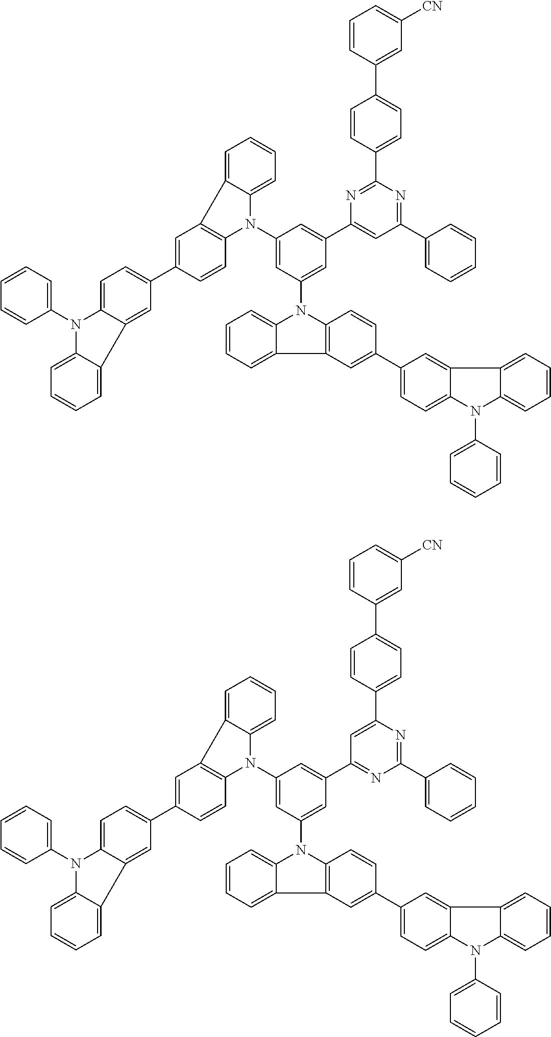 [SMILES: N#Cc1cccc(-c2ccc(-c3nc(-c4ccccc4)cc(-c4cc(-n5c6ccccc6c6cc(-c7ccc8c(c7)c7ccccc7n8-c7ccccc7)ccc65)cc(-n5c6ccccc6c6cc(-c7ccc8c(c7)c7ccccc7n8-c7ccccc7)ccc65)c4)n3)cc2)c1 ; N#Cc1cccc(-c2ccc(-c3cc(-c4cc(-n5c6ccccc6c6cc(-c7ccc8c(c7)c7ccccc7n8-c7ccccc7)ccc65)cc(-n5c6ccccc6c6cc(-c7ccc8c(c7)c7ccccc7n8-c7ccccc7)ccc65)c4)nc(-c4ccccc4)n3)cc2)c1]

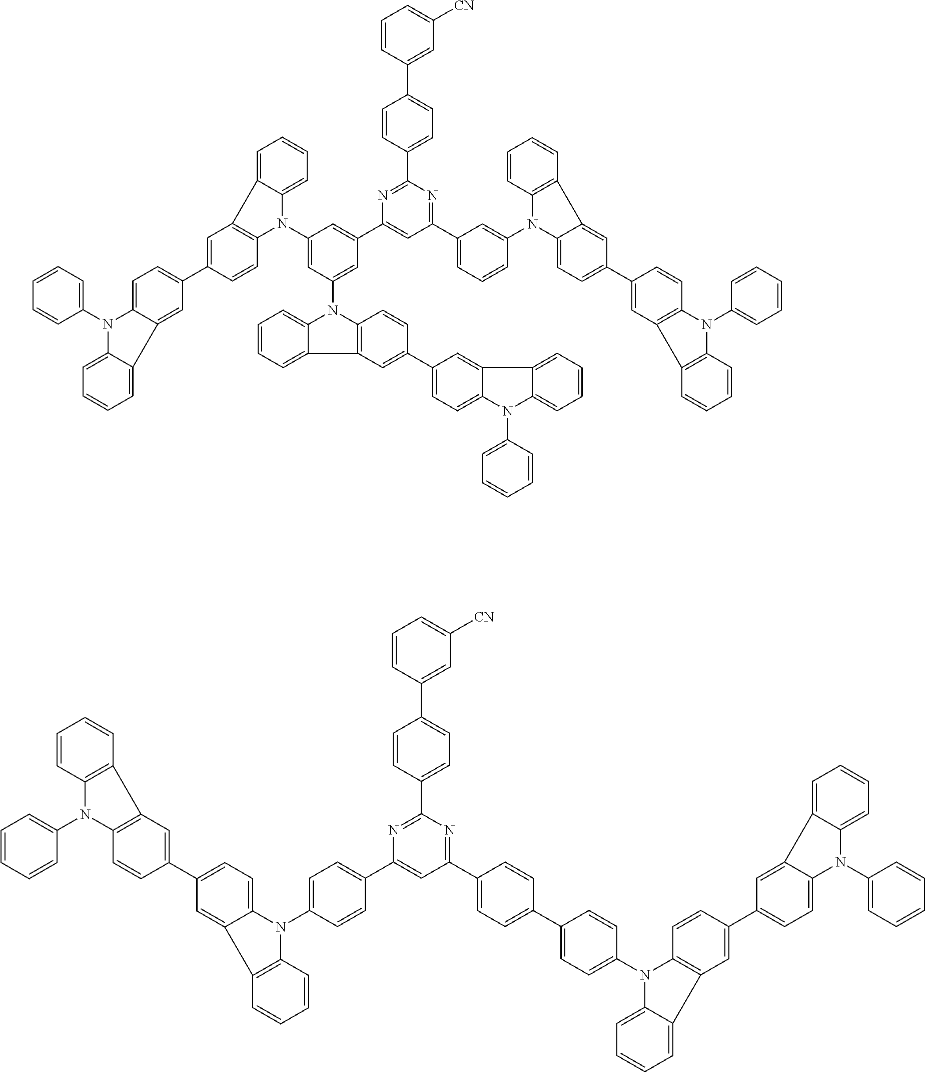 [SMILES: N#Cc1cccc(-c2ccc(-c3nc(-c4cccc(-n5c6ccccc6c6cc(-c7ccc8c(c7)c7ccccc7n8-c7ccccc7)ccc65)c4)cc(-c4cc(-n5c6ccccc6c6cc(-c7ccc8c(c7)c7ccccc7n8-c7ccccc7)ccc65)cc(-n5c6ccccc6c6cc(-c7ccc8c(c7)c7ccccc7n8-c7ccccc7)ccc65)c4)n3)cc2)c1 ; N#Cc1cccc(-c2ccc(-c3nc(-c4ccc(-c5ccc(-n6c7ccccc7c7cc(-c8ccc9c(c8)c8ccccc8n9-c8ccccc8)ccc76)cc5)cc4)cc(-c4ccc(-n5c6ccccc6c6cc(-c7ccc8c(c7)c7ccccc7n8-c7ccccc7)ccc65)cc4)n3)cc2)c1]

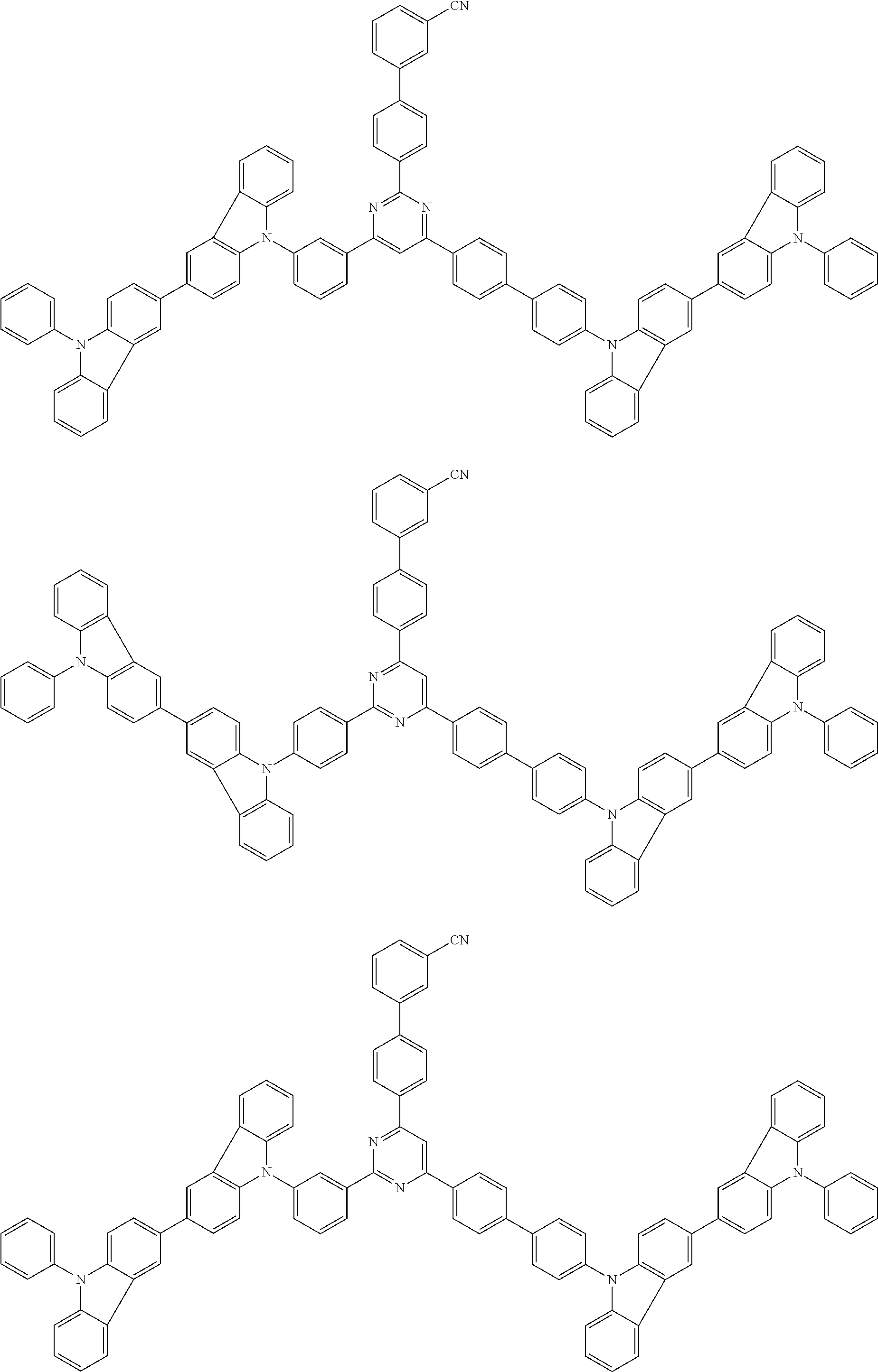

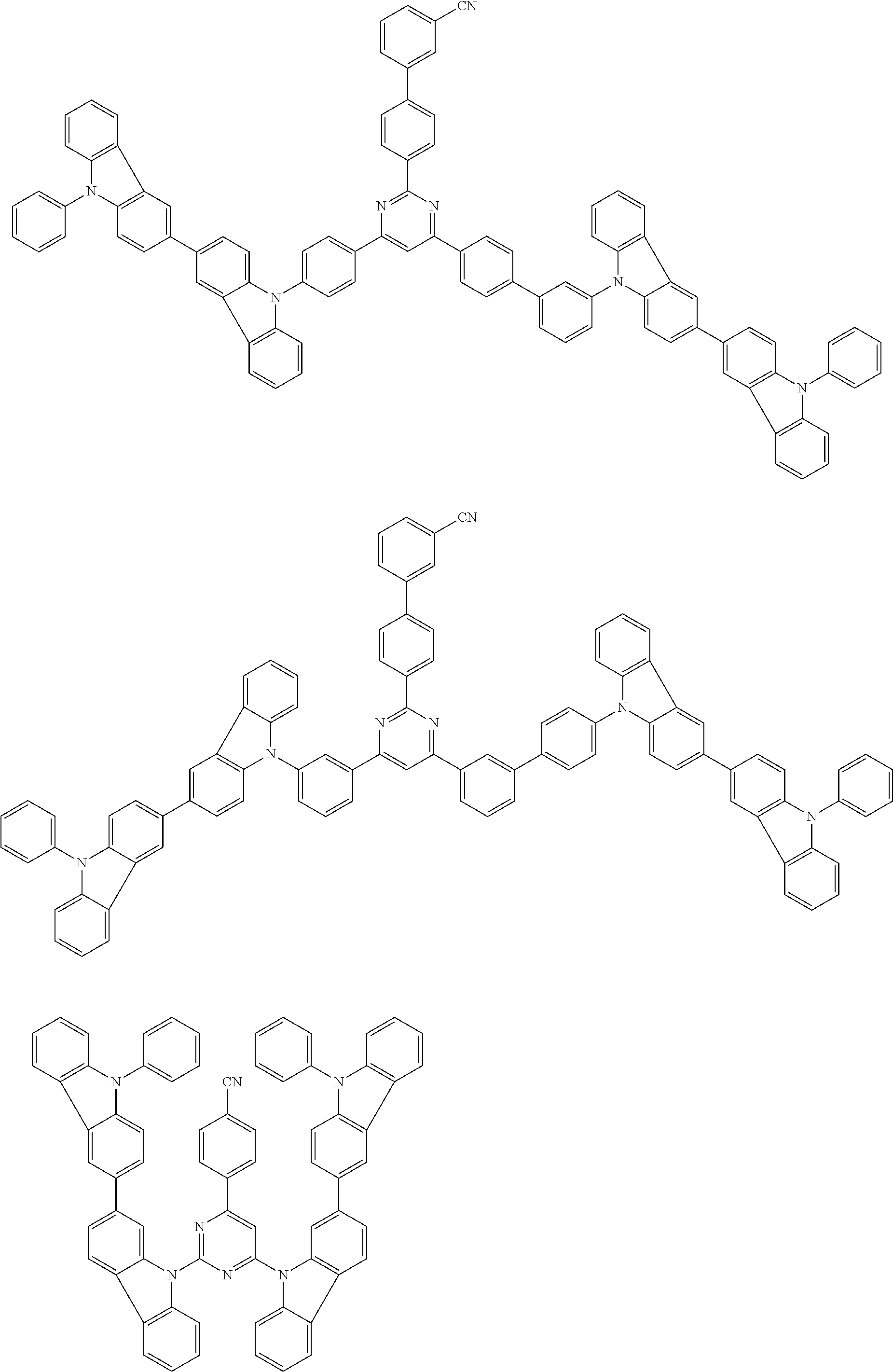 [SMILES: N#Cc1cccc(-c2ccc(-c3nc(-c4ccc(-c5cccc(-n6c7ccccc7c7cc(-c8ccc9c(c8)c8ccccc8n9-c8ccccc8)ccc76)c5)cc4)cc(-c4ccc(-n5c6ccccc6c6cc(-c7ccc8c(c7)c7ccccc7n8-c7ccccc7)ccc65)cc4)n3)cc2)c1 ; N#Cc1cccc(-c2ccc(-c3nc(-c4cccc(-c5ccc(-n6c7ccccc7c7cc(-c8ccc9c(c8)c8ccccc8n9-c8ccccc8)ccc76)cc5)c4)cc(-c4cccc(-n5c6ccccc6c6cc(-c7ccc8c(c7)c7ccccc7n8-c7ccccc7)ccc65)c4)n3)cc2)c1 ; N#Cc1ccc(-c2cc(-n3c4ccccc4c4ccc(-c5ccc6c(c5)c5ccccc5n6-c5ccccc5)cc43)nc(-n3c4ccccc4c4ccc(-c5ccc6c(c5)c5ccccc5n6-c5ccccc5)cc43)n2)cc1]

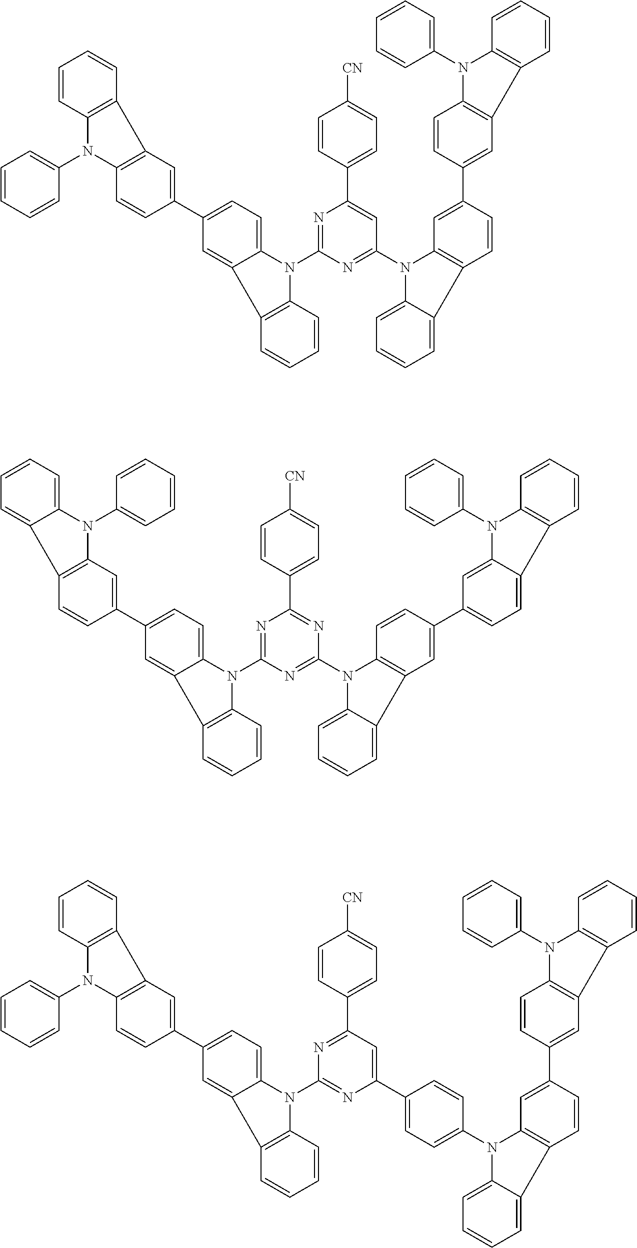 [SMILES: N#Cc1ccc(-c2cc(-n3c4ccccc4c4ccc(-c5ccc6c(c5)c5ccccc5n6-c5ccccc5)cc43)nc(-n3c4ccccc4c4cc(-c5ccc6c(c5)c5ccccc5n6-c5ccccc5)ccc43)n2)cc1 ; N#Cc1ccc(-c2nc(-n3c4ccccc4c4cc(-c5ccc6c7ccccc7n(-c7ccccc7)c6c5)ccc43)nc(-n3c4ccccc4c4cc(-c5ccc6c7ccccc7n(-c7ccccc7)c6c5)ccc43)n2)cc1 ; N#Cc1ccc(-c2cc(-c3ccc(-n4c5ccccc5c5ccc(-c6ccc7c(c6)c6ccccc6n7-c6ccccc6)cc54)cc3)nc(-n3c4ccccc4c4cc(-c5ccc6c(c5)c5ccccc5n6-c5ccccc5)ccc43)n2)cc1]

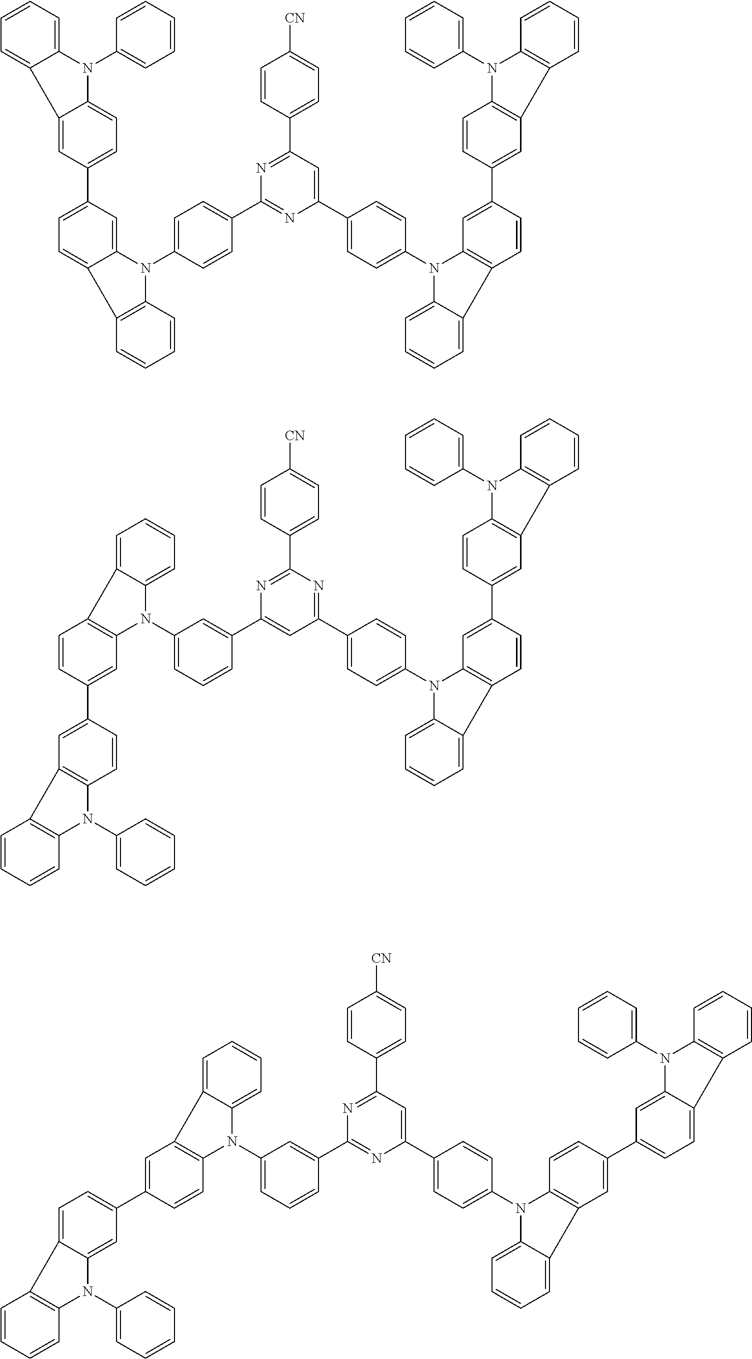 [SMILES: N#Cc1ccc(-c2cc(-c3ccc(-n4c5ccccc5c5ccc(-c6ccc7c(c6)c6ccccc6n7-c6ccccc6)cc54)cc3)nc(-c3ccc(-n4c5ccccc5c5ccc(-c6ccc7c(c6)c6ccccc6n7-c6ccccc6)cc54)cc3)n2)cc1 ; N#Cc1ccc(-c2nc(-c3ccc(-n4c5ccccc5c5ccc(-c6ccc7c(c6)c6ccccc6n7-c6ccccc6)cc54)cc3)cc(-c3cccc(-n4c5ccccc5c5ccc(-c6ccc7c(c6)c6ccccc6n7-c6ccccc6)cc54)c3)n2)cc1 ; N#Cc1ccc(-c2cc(-c3ccc(-n4c5ccccc5c5cc(-c6ccc7c8ccccc8n(-c8ccccc8)c7c6)ccc54)cc3)nc(-c3cccc(-n4c5ccccc5c5cc(-c6ccc7c8ccccc8n(-c8ccccc8)c7c6)ccc54)c3)n2)cc1]

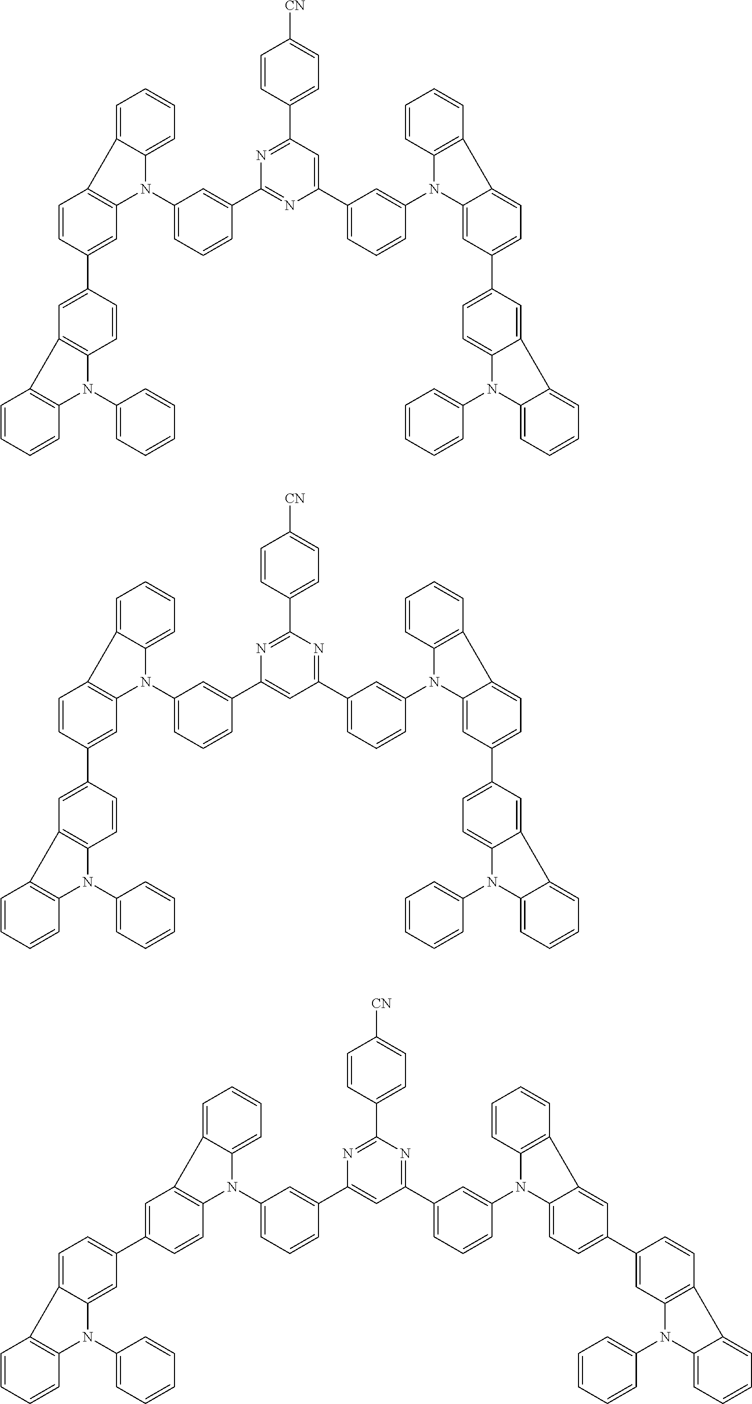 [SMILES: N#Cc1ccc(-c2cc(-c3cccc(-n4c5ccccc5c5ccc(-c6ccc7c(c6)c6ccccc6n7-c6ccccc6)cc54)c3)nc(-c3cccc(-n4c5ccccc5c5ccc(-c6ccc7c(c6)c6ccccc6n7-c6ccccc6)cc54)c3)n2)cc1 ; N#Cc1ccc(-c2nc(-c3cccc(-n4c5ccccc5c5ccc(-c6ccc7c(c6)c6ccccc6n7-c6ccccc6)cc54)c3)cc(-c3cccc(-n4c5ccccc5c5ccc(-c6ccc7c(c6)c6ccccc6n7-c6ccccc6)cc54)c3)n2)cc1 ; N#Cc1ccc(-c2nc(-c3cccc(-n4c5ccccc5c5cc(-c6ccc7c8ccccc8n(-c8ccccc8)c7c6)ccc54)c3)cc(-c3cccc(-n4c5ccccc5c5cc(-c6ccc7c8ccccc8n(-c8ccccc8)c7c6)ccc54)c3)n2)cc1]

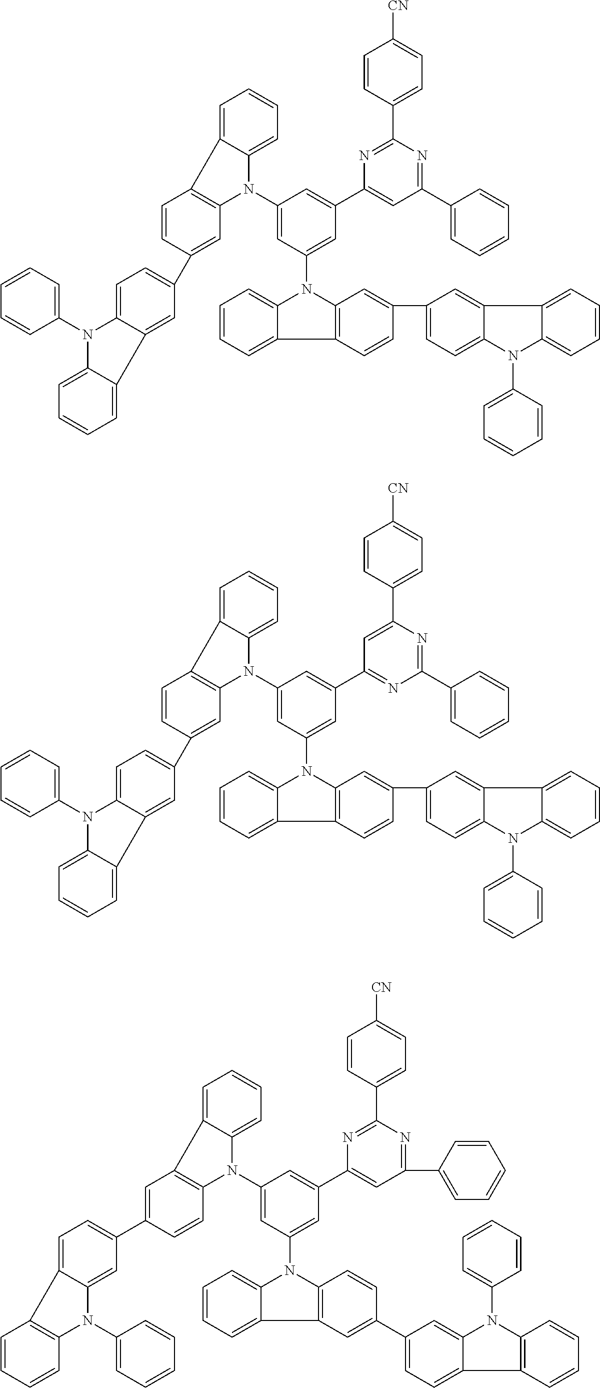 [SMILES: N#Cc1ccc(-c2nc(-c3ccccc3)cc(-c3cc(-n4c5ccccc5c5ccc(-c6ccc7c(c6)c6ccccc6n7-c6ccccc6)cc54)cc(-n4c5ccccc5c5ccc(-c6ccc7c(c6)c6ccccc6n7-c6ccccc6)cc54)c3)n2)cc1 ; N#Cc1ccc(-c2cc(-c3cc(-n4c5ccccc5c5ccc(-c6ccc7c(c6)c6ccccc6n7-c6ccccc6)cc54)cc(-n4c5ccccc5c5ccc(-c6ccc7c(c6)c6ccccc6n7-c6ccccc6)cc54)c3)nc(-c3ccccc3)n2)cc1 ; N#Cc1ccc(-c2nc(-c3ccccc3)cc(-c3cc(-n4c5ccccc5c5cc(-c6ccc7c8ccccc8n(-c8ccccc8)c7c6)ccc54)cc(-n4c5ccccc5c5cc(-c6ccc7c8ccccc8n(-c8ccccc8)c7c6)ccc54)c3)n2)cc1]

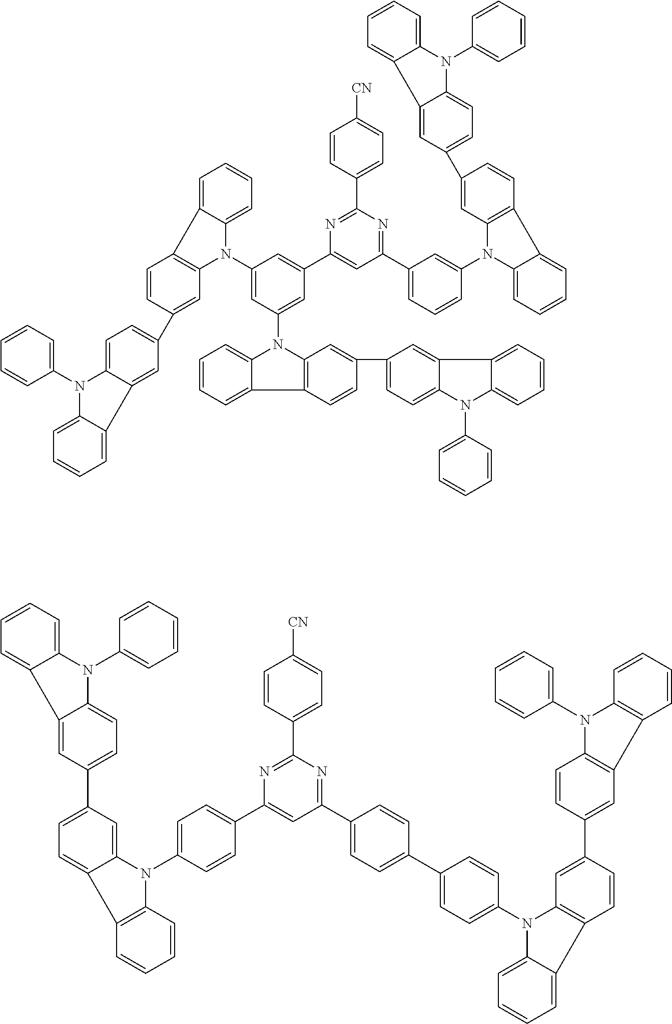 [SMILES: N#Cc1ccc(-c2nc(-c3cccc(-n4c5ccccc5c5ccc(-c6ccc7c(c6)c6ccccc6n7-c6ccccc6)cc54)c3)cc(-c3cc(-n4c5ccccc5c5ccc(-c6ccc7c(c6)c6ccccc6n7-c6ccccc6)cc54)cc(-n4c5ccccc5c5ccc(-c6ccc7c(c6)c6ccccc6n7-c6ccccc6)cc54)c3)n2)cc1 ; N#Cc1ccc(-c2nc(-c3ccc(-c4ccc(-n5c6ccccc6c6ccc(-c7ccc8c(c7)c7ccccc7n8-c7ccccc7)cc65)cc4)cc3)cc(-c3ccc(-n4c5ccccc5c5ccc(-c6ccc7c(c6)c6ccccc6n7-c6ccccc6)cc54)cc3)n2)cc1]

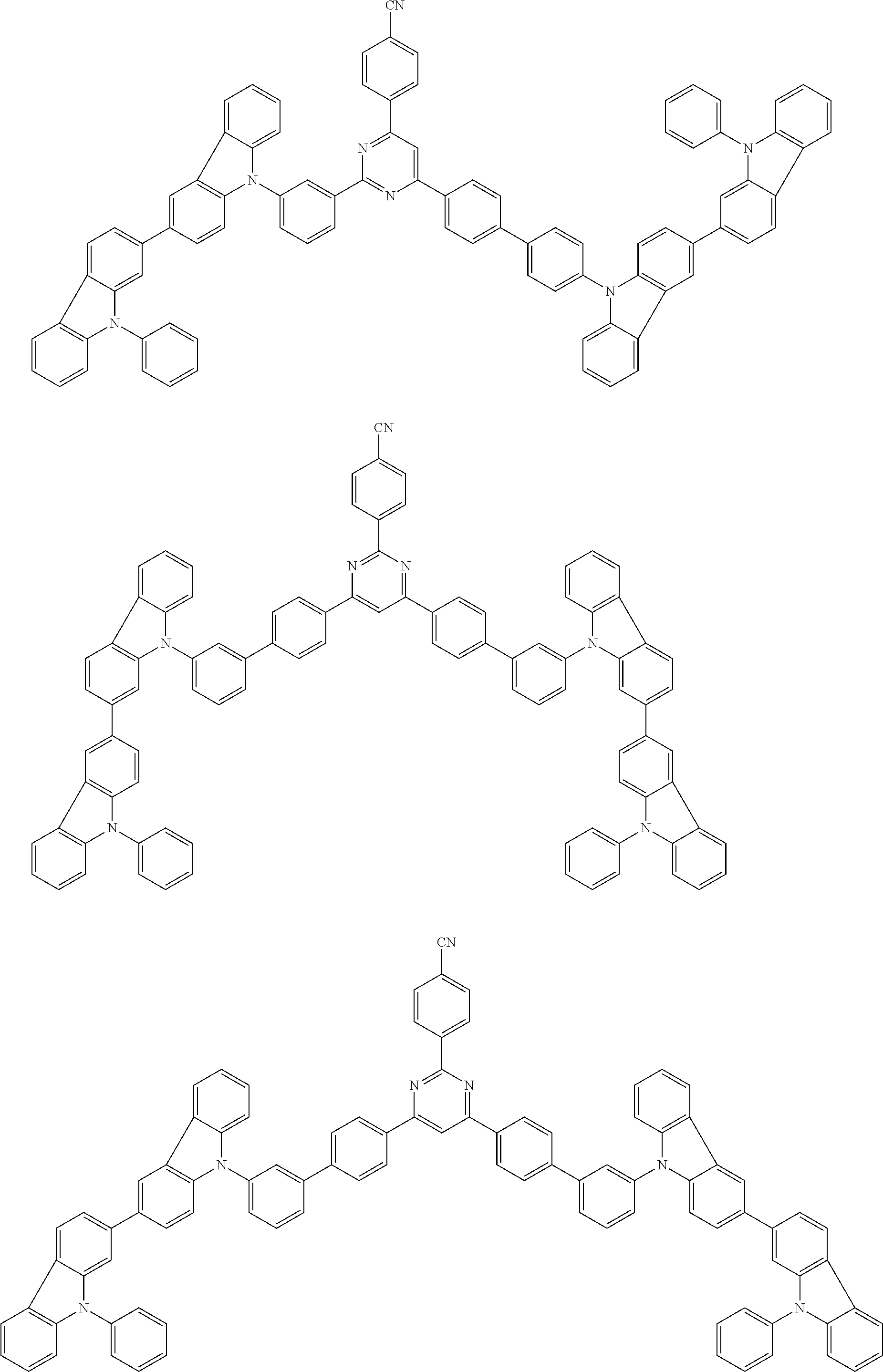

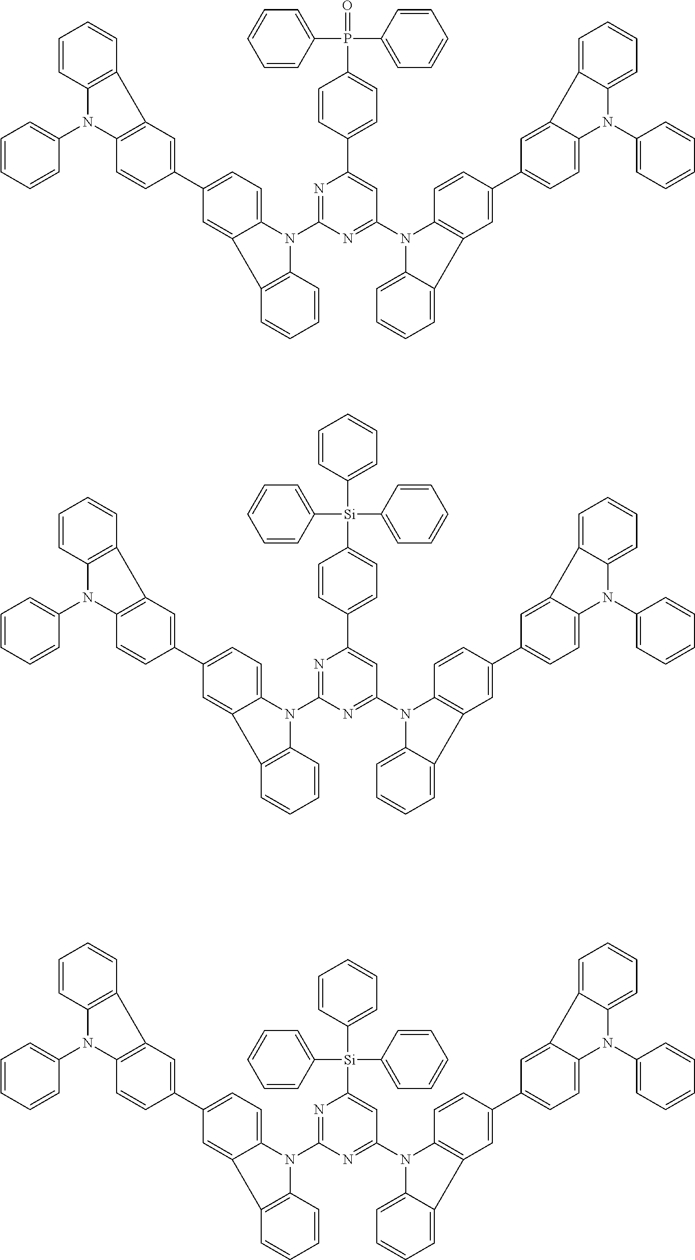 [SMILES: O=P(c1ccccc1)(c1ccccc1)c1ccc(-c2cc(-n3c4ccccc4c4cc(-c5ccc6c(c5)c5ccccc5n6-c5ccccc5)ccc43)nc(-n3c4ccccc4c4cc(-c5ccc6c(c5)c5ccccc5n6-c5ccccc5)ccc43)n2)cc1 ; c1ccc(-n2c3ccccc3c3cc(-c4ccc5c(c4)c4ccccc4n5-c4cc(-c5ccc([Si](c6ccccc6)(c6ccccc6)c6ccccc6)cc5)nc(-n5c6ccccc6c6cc(-c7ccc8c(c7)c7ccccc7n8-c7ccccc7)ccc65)n4)ccc32)cc1 ; c1ccc(-n2c3ccccc3c3cc(-c4ccc5c(c4)c4ccccc4n5-c4cc([Si](c5ccccc5)(c5ccccc5)c5ccccc5)nc(-n5c6ccccc6c6cc(-c7ccc8c(c7)c7ccccc7n8-c7ccccc7)ccc65)n4)ccc32)cc1]

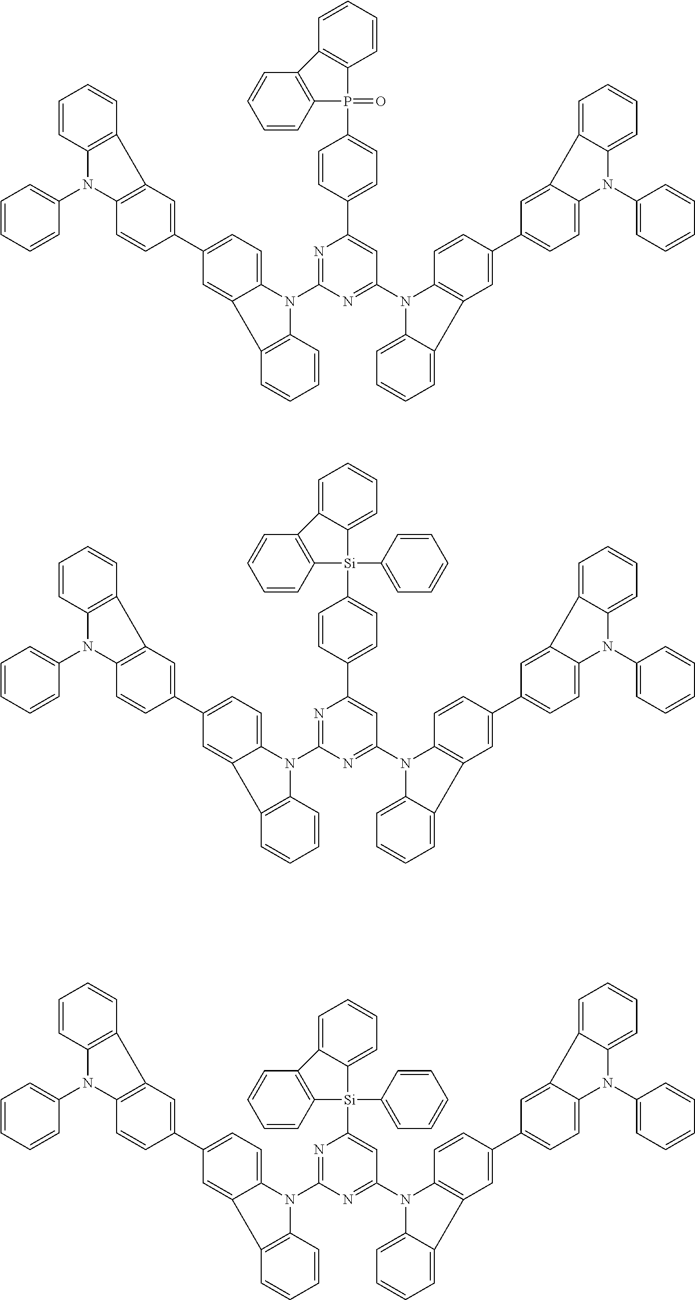 [SMILES: O=P1(c2ccc(-c3cc(-n4c5ccccc5c5cc(-c6ccc7c(c6)c6ccccc6n7-c6ccccc6)ccc54)nc(-n4c5ccccc5c5cc(-c6ccc7c(c6)c6ccccc6n7-c6ccccc6)ccc54)n3)cc2)c2ccccc2-c2ccccc21 ; c1ccc(-n2c3ccccc3c3cc(-c4ccc5c(c4)c4ccccc4n5-c4cc(-c5ccc([Si]6(c7ccccc7)c7ccccc7-c7ccccc76)cc5)nc(-n5c6ccccc6c6cc(-c7ccc8c(c7)c7ccccc7n8-c7ccccc7)ccc65)n4)ccc32)cc1 ; c1ccc(-n2c3ccccc3c3cc(-c4ccc5c(c4)c4ccccc4n5-c4cc([Si]5(c6ccccc6)c6ccccc6-c6ccccc65)nc(-n5c6ccccc6c6cc(-c7ccc8c(c7)c7ccccc7n8-c7ccccc7)ccc65)n4)ccc32)cc1]

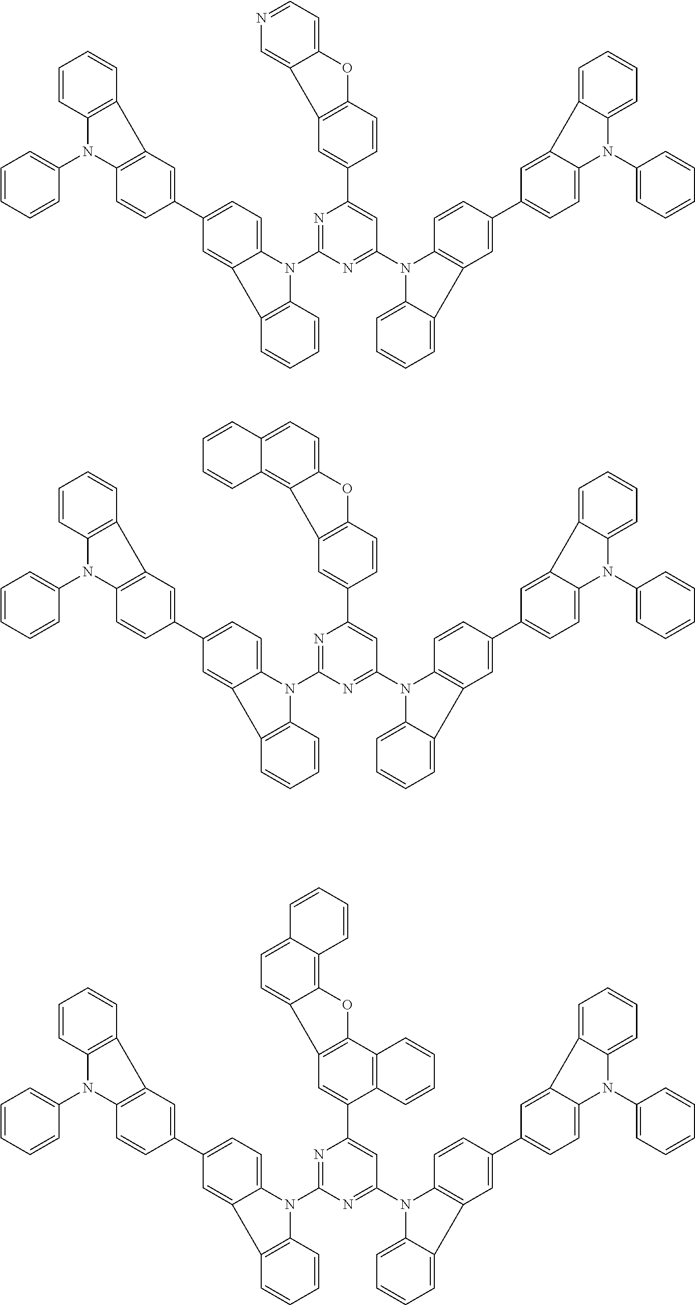 [SMILES: c1ccc(-n2c3ccccc3c3cc(-c4ccc5c(c4)c4ccccc4n5-c4cc(-c5ccc6oc7ccncc7c6c5)nc(-n5c6ccccc6c6cc(-c7ccc8c(c7)c7ccccc7n8-c7ccccc7)ccc65)n4)ccc32)cc1 ; c1ccc(-n2c3ccccc3c3cc(-c4ccc5c(c4)c4ccccc4n5-c4cc(-c5ccc6oc7ccc8ccccc8c7c6c5)nc(-n5c6ccccc6c6cc(-c7ccc8c(c7)c7ccccc7n8-c7ccccc7)ccc65)n4)ccc32)cc1 ; c1ccc(-n2c3ccccc3c3cc(-c4ccc5c(c4)c4ccccc4n5-c4cc(-c5cc6c7ccc8ccccc8c7oc6c6ccccc56)nc(-n5c6ccccc6c6cc(-c7ccc8c(c7)c7ccccc7n8-c7ccccc7)ccc65)n4)ccc32)cc1]

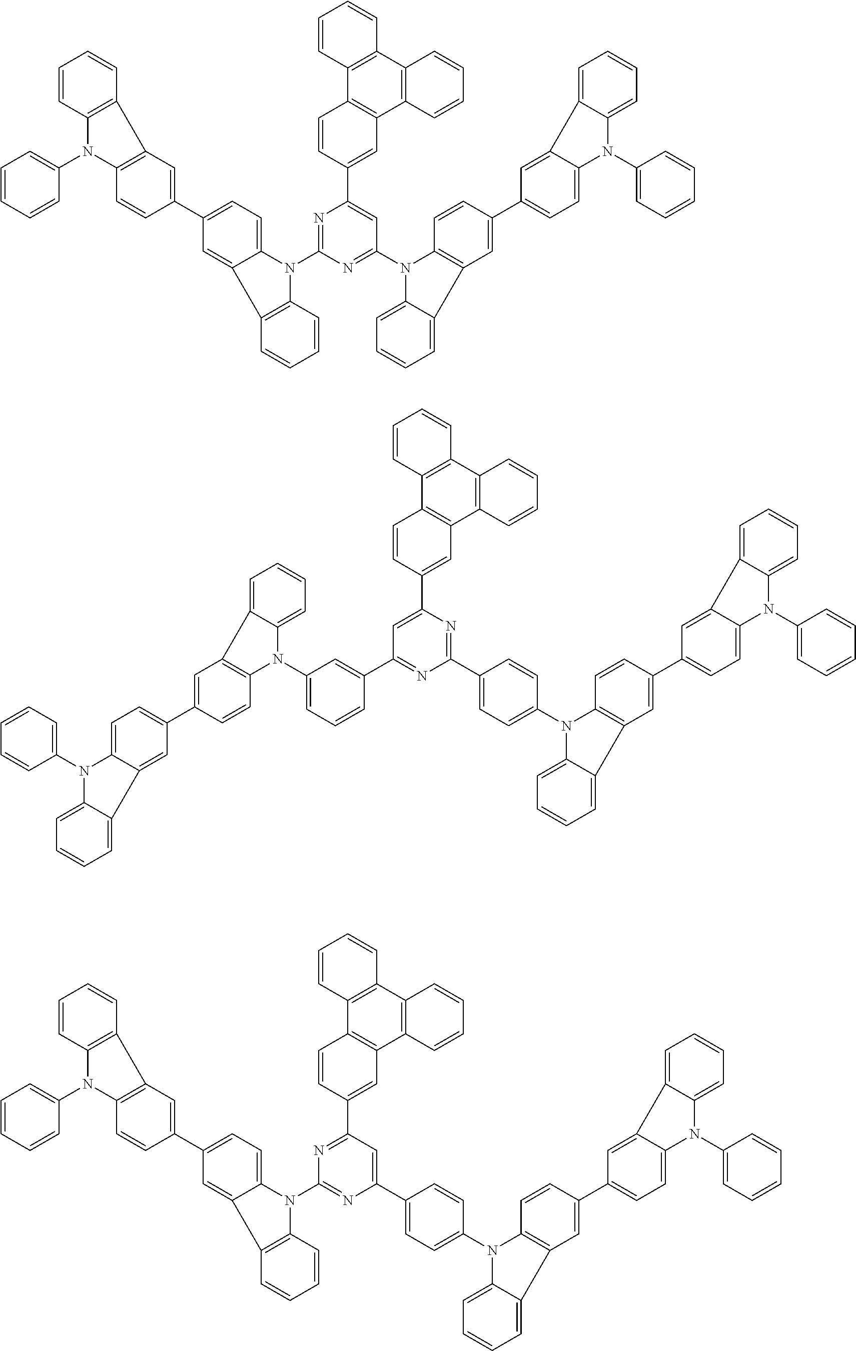

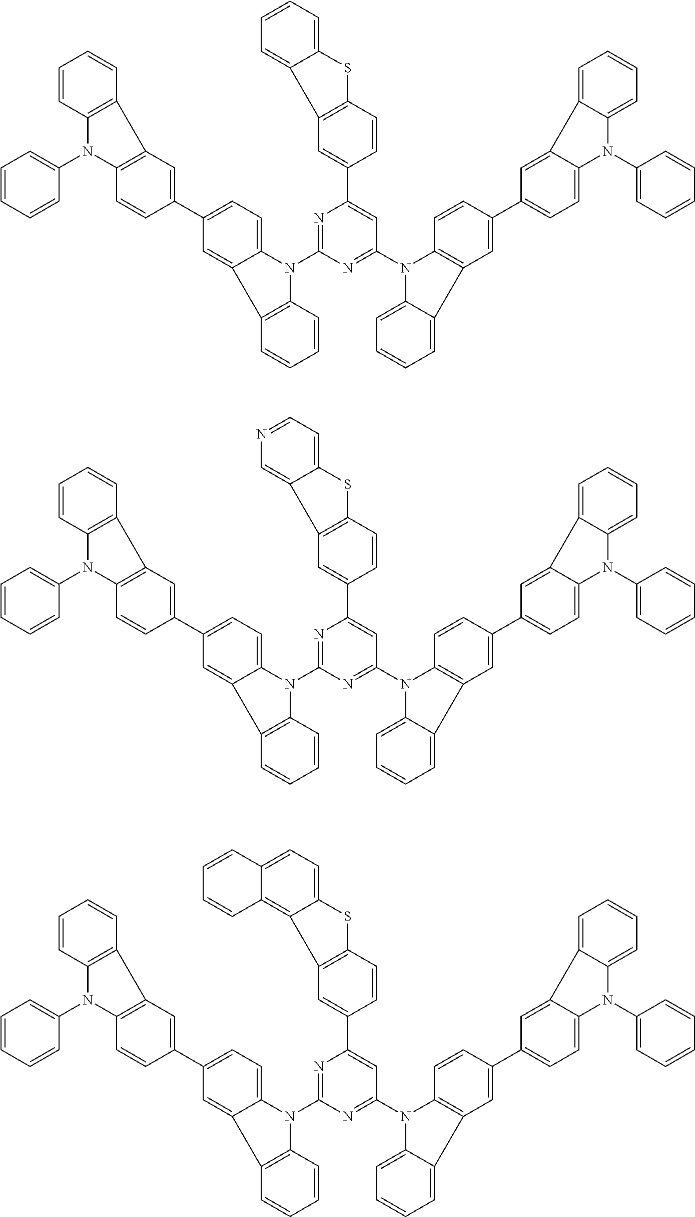 [SMILES: c1ccc(-n2c3ccccc3c3cc(-c4ccc5c(c4)c4ccccc4n5-c4cc(-c5ccc6sc7ccccc7c6c5)nc(-n5c6ccccc6c6cc(-c7ccc8c(c7)c7ccccc7n8-c7ccccc7)ccc65)n4)ccc32)cc1 ; c1ccc(-n2c3ccccc3c3cc(-c4ccc5c(c4)c4ccccc4n5-c4cc(-c5ccc6sc7ccncc7c6c5)nc(-n5c6ccccc6c6cc(-c7ccc8c(c7)c7ccccc7n8-c7ccccc7)ccc65)n4)ccc32)cc1 ; c1ccc(-n2c3ccccc3c3cc(-c4ccc5c(c4)c4ccccc4n5-c4cc(-c5ccc6sc7ccc8ccccc8c7c6c5)nc(-n5c6ccccc6c6cc(-c7ccc8c(c7)c7ccccc7n8-c7ccccc7)ccc65)n4)ccc32)cc1]

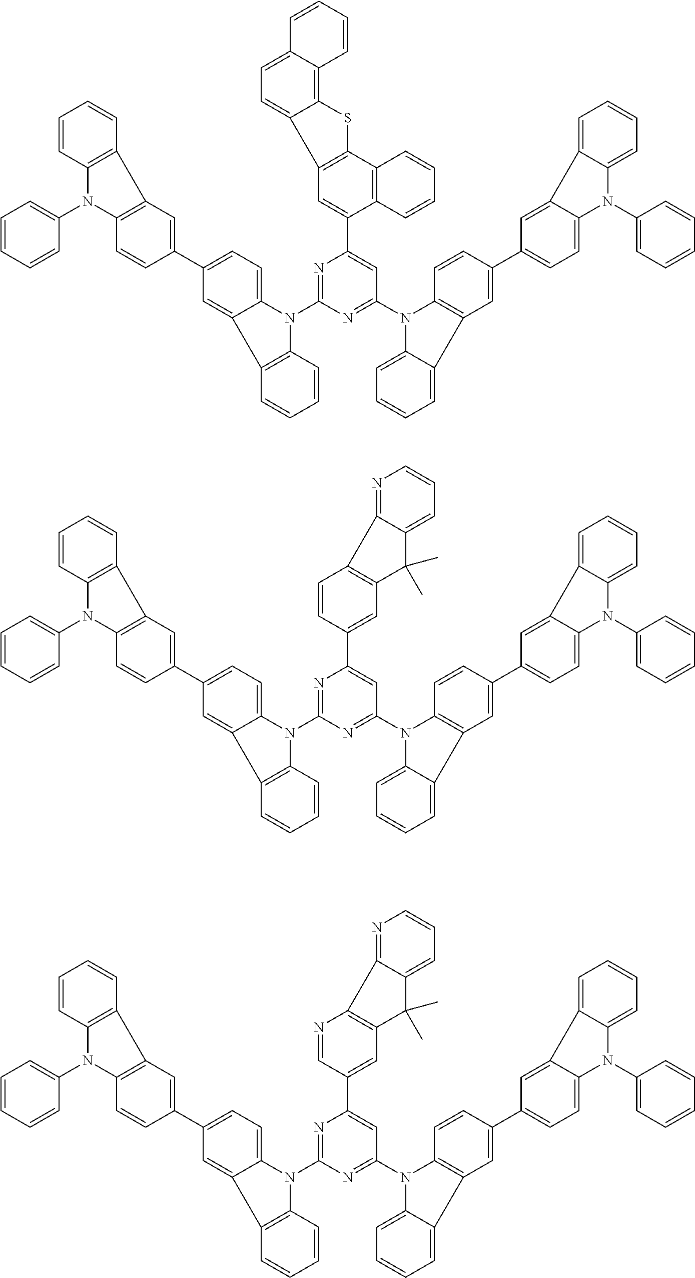 [SMILES: c1ccc(-n2c3ccccc3c3cc(-c4ccc5c(c4)c4ccccc4n5-c4cc(-c5cc6c7ccc8ccccc8c7sc6c6ccccc56)nc(-n5c6ccccc6c6cc(-c7ccc8c(c7)c7ccccc7n8-c7ccccc7)ccc65)n4)ccc32)cc1 ; CC1(C)c2cc(-c3cc(-n4c5ccccc5c5cc(-c6ccc7c(c6)c6ccccc6n7-c6ccccc6)ccc54)nc(-n4c5ccccc5c5cc(-c6ccc7c(c6)c6ccccc6n7-c6ccccc6)ccc54)n3)ccc2-c2ncccc21 ; CC1(C)c2cccnc2-c2ncc(-c3cc(-n4c5ccccc5c5cc(-c6ccc7c(c6)c6ccccc6n7-c6ccccc6)ccc54)nc(-n4c5ccccc5c5cc(-c6ccc7c(c6)c6ccccc6n7-c6ccccc6)ccc54)n3)cc21]

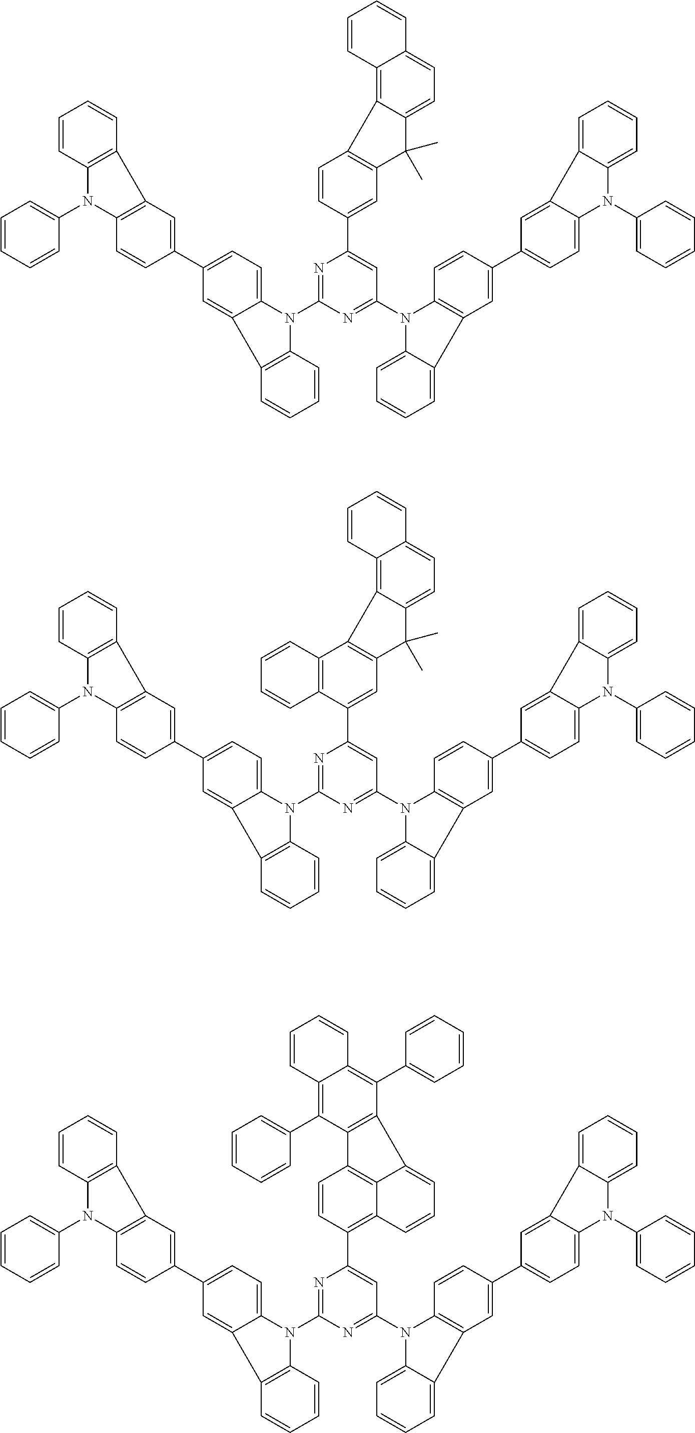 [SMILES: CC1(C)c2cc(-c3cc(-n4c5ccccc5c5cc(-c6ccc7c(c6)c6ccccc6n7-c6ccccc6)ccc54)nc(-n4c5ccccc5c5cc(-c6ccc7c(c6)c6ccccc6n7-c6ccccc6)ccc54)n3)ccc2-c2c1ccc1ccccc21 ; CC1(C)c2ccc3ccccc3c2-c2c1cc(-c1cc(-n3c4ccccc4c4cc(-c5ccc6c(c5)c5ccccc5n6-c5ccccc5)ccc43)nc(-n3c4ccccc4c4cc(-c5ccc6c(c5)c5ccccc5n6-c5ccccc5)ccc43)n1)c1ccccc21 ; c1ccc(-c2c3c(c(-c4ccccc4)c4ccccc24)-c2ccc(-c4cc(-n5c6ccccc6c6cc(-c7ccc8c(c7)c7ccccc7n8-c7ccccc7)ccc65)nc(-n5c6ccccc6c6cc(-c7ccc8c(c7)c7ccccc7n8-c7ccccc7)ccc65)n4)c4cccc-3c24)cc1]

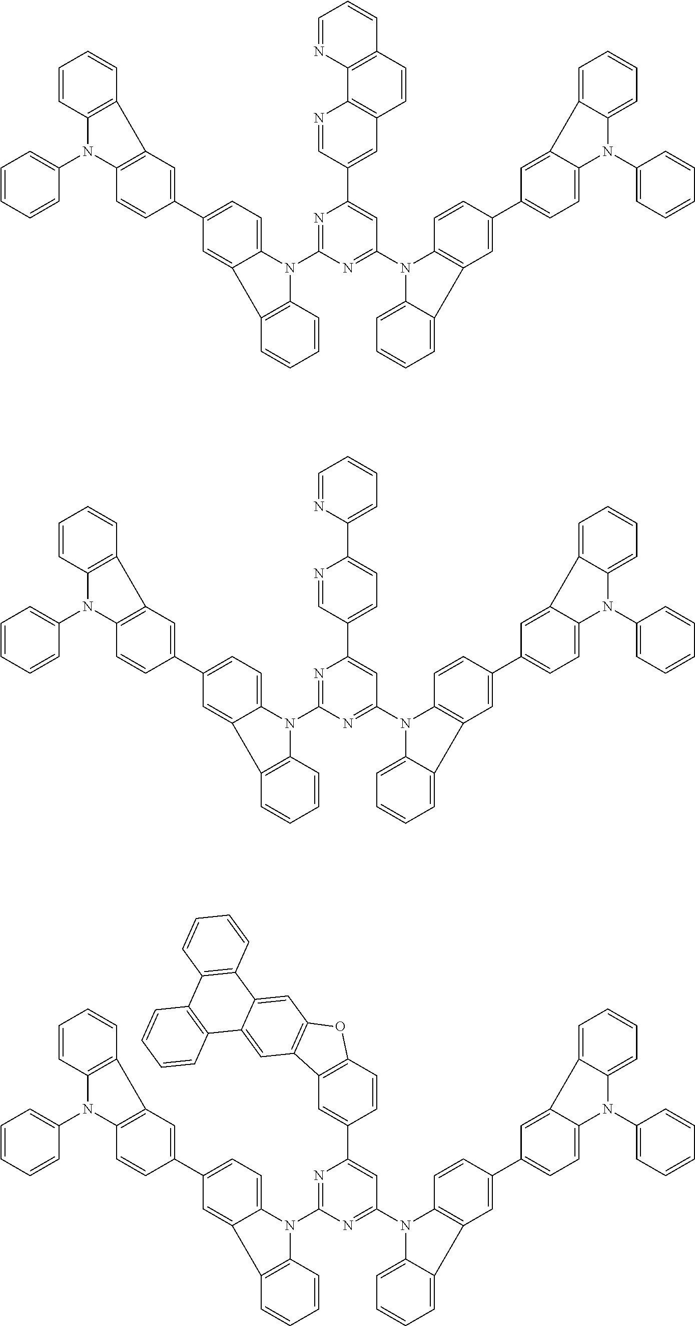 [SMILES: c1ccc(-n2c3ccccc3c3cc(-c4ccc5c(c4)c4ccccc4n5-c4cc(-c5cnc6c(ccc7cccnc76)c5)nc(-n5c6ccccc6c6cc(-c7ccc8c(c7)c7ccccc7n8-c7ccccc7)ccc65)n4)ccc32)cc1 ; c1ccc(-n2c3ccccc3c3cc(-c4ccc5c(c4)c4ccccc4n5-c4cc(-c5ccc(-c6ccccn6)nc5)nc(-n5c6ccccc6c6cc(-c7ccc8c(c7)c7ccccc7n8-c7ccccc7)ccc65)n4)ccc32)cc1 ; c1ccc(-n2c3ccccc3c3cc(-c4ccc5c(c4)c4ccccc4n5-c4cc(-c5ccc6oc7cc8c9ccccc9c9ccccc9c8cc7c6c5)nc(-n5c6ccccc6c6cc(-c7ccc8c(c7)c7ccccc7n8-c7ccccc7)ccc65)n4)ccc32)cc1]

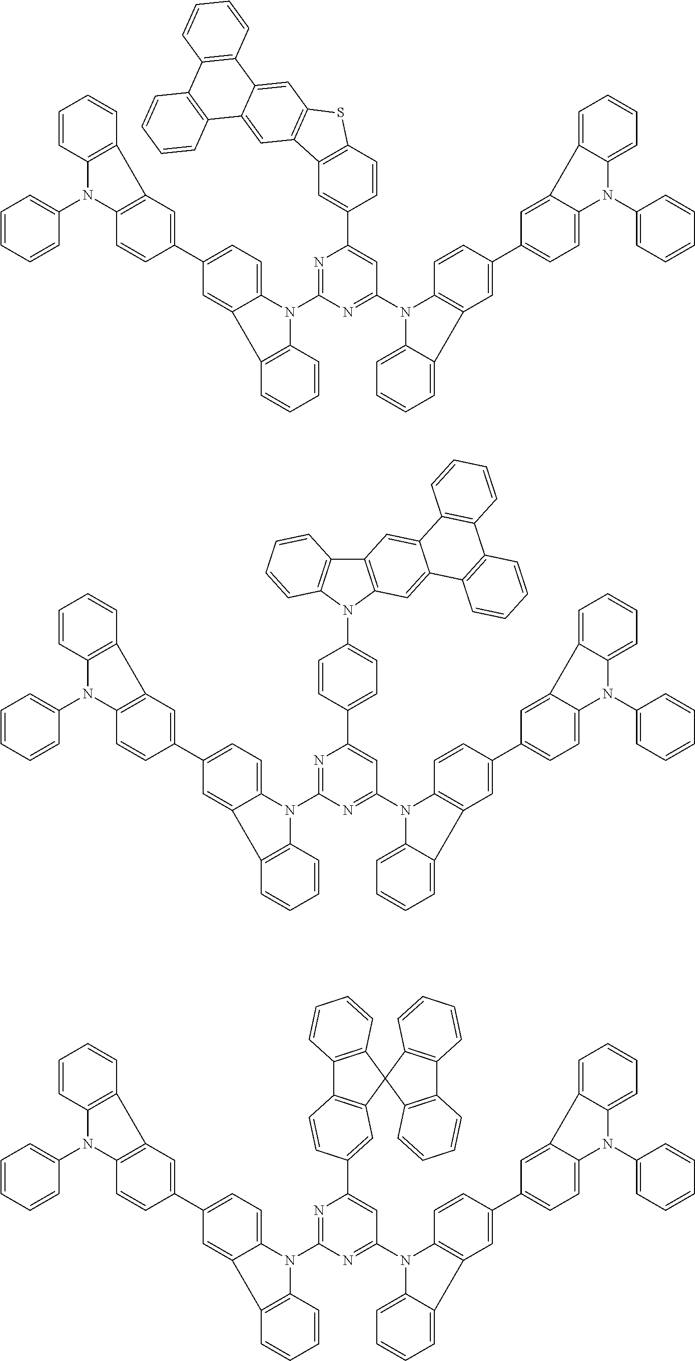 [SMILES: c1ccc(-n2c3ccccc3c3cc(-c4ccc5c(c4)c4ccccc4n5-c4cc(-c5ccc6sc7cc8c9ccccc9c9ccccc9c8cc7c6c5)nc(-n5c6ccccc6c6cc(-c7ccc8c(c7)c7ccccc7n8-c7ccccc7)ccc65)n4)ccc32)cc1 ; c1ccc(-n2c3ccccc3c3cc(-c4ccc5c(c4)c4ccccc4n5-c4cc(-c5ccc(-n6c7ccccc7c7cc8c9ccccc9c9ccccc9c8cc76)cc5)nc(-n5c6ccccc6c6cc(-c7ccc8c(c7)c7ccccc7n8-c7ccccc7)ccc65)n4)ccc32)cc1 ; c1ccc(-n2c3ccccc3c3cc(-c4ccc5c(c4)c4ccccc4n5-c4cc(-c5ccc6c(c5)C5(c7ccccc7-c7ccccc75)c5ccccc5-6)nc(-n5c6ccccc6c6cc(-c7ccc8c(c7)c7ccccc7n8-c7ccccc7)ccc65)n4)ccc32)cc1]

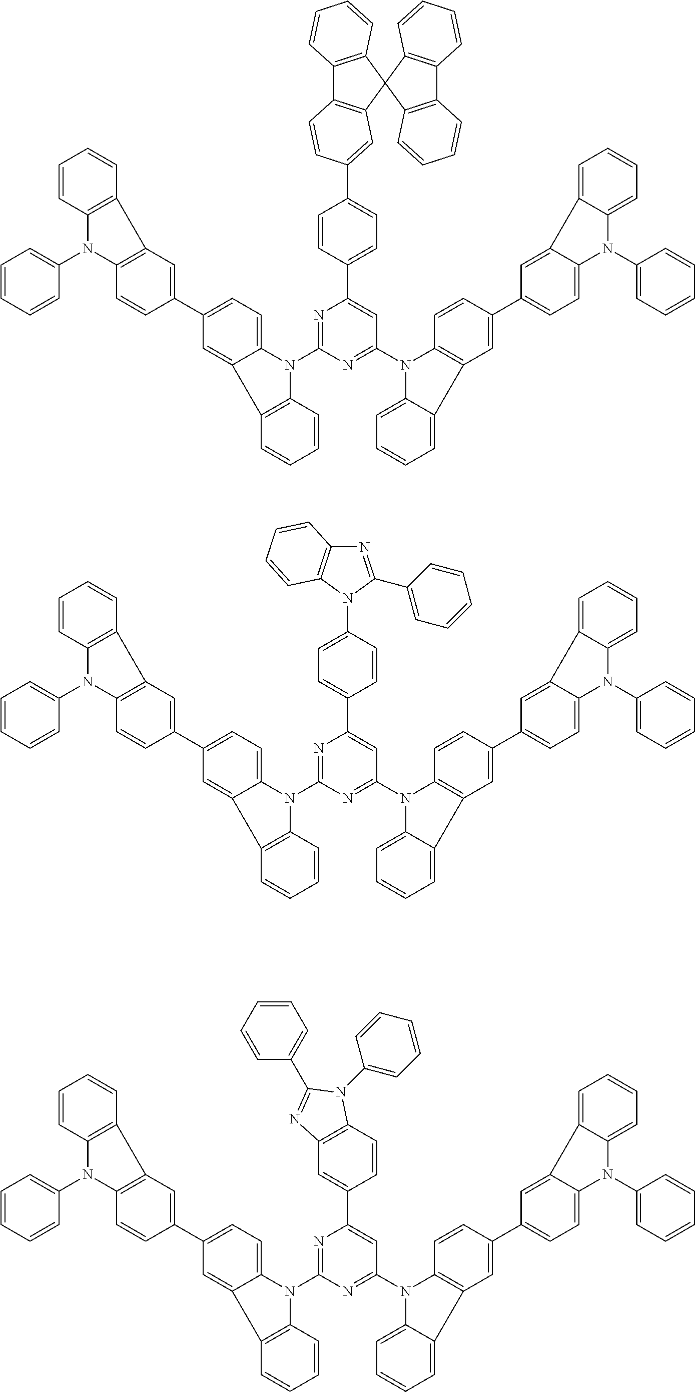 [SMILES: c1ccc(-n2c3ccccc3c3cc(-c4ccc5c(c4)c4ccccc4n5-c4cc(-c5ccc(-c6ccc7c(c6)C6(c8ccccc8-c8ccccc86)c6ccccc6-7)cc5)nc(-n5c6ccccc6c6cc(-c7ccc8c(c7)c7ccccc7n8-c7ccccc7)ccc65)n4)ccc32)cc1 ; c1ccc(-c2nc3ccccc3n2-c2ccc(-c3cc(-n4c5ccccc5c5cc(-c6ccc7c(c6)c6ccccc6n7-c6ccccc6)ccc54)nc(-n4c5ccccc5c5cc(-c6ccc7c(c6)c6ccccc6n7-c6ccccc6)ccc54)n3)cc2)cc1 ; c1ccc(-c2nc3cc(-c4cc(-n5c6ccccc6c6cc(-c7ccc8c(c7)c7ccccc7n8-c7ccccc7)ccc65)nc(-n5c6ccccc6c6cc(-c7ccc8c(c7)c7ccccc7n8-c7ccccc7)ccc65)n4)ccc3n2-c2ccccc2)cc1]

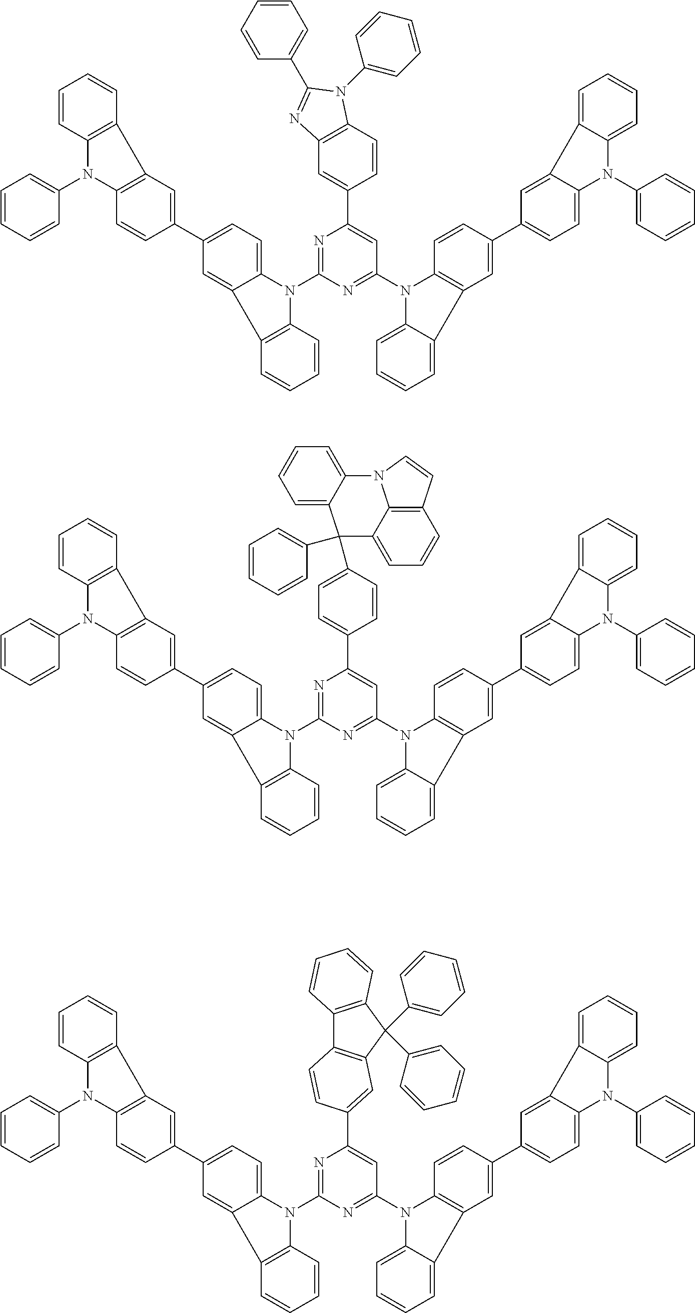 [SMILES: c1ccc(-c2nc3cc(-c4cc(-n5c6ccccc6c6cc(-c7ccc8c(c7)c7ccccc7n8-c7ccccc7)ccc65)nc(-n5c6ccccc6c6cc(-c7ccc8c(c7)c7ccccc7n8-c7ccccc7)ccc65)n4)ccc3n2-c2ccccc2)cc1 ; c1ccc(-n2c3ccccc3c3cc(-c4ccc5c(c4)c4ccccc4n5-c4cc(-c5ccc(C6(c7ccccc7)c7ccccc7-n7ccc8cccc6c87)cc5)nc(-n5c6ccccc6c6cc(-c7ccc8c(c7)c7ccccc7n8-c7ccccc7)ccc65)n4)ccc32)cc1 ; c1ccc(-n2c3ccccc3c3cc(-c4ccc5c(c4)c4ccccc4n5-c4cc(-c5ccc6c(c5)C(c5ccccc5)(c5ccccc5)c5ccccc5-6)nc(-n5c6ccccc6c6cc(-c7ccc8c(c7)c7ccccc7n8-c7ccccc7)ccc65)n4)ccc32)cc1]

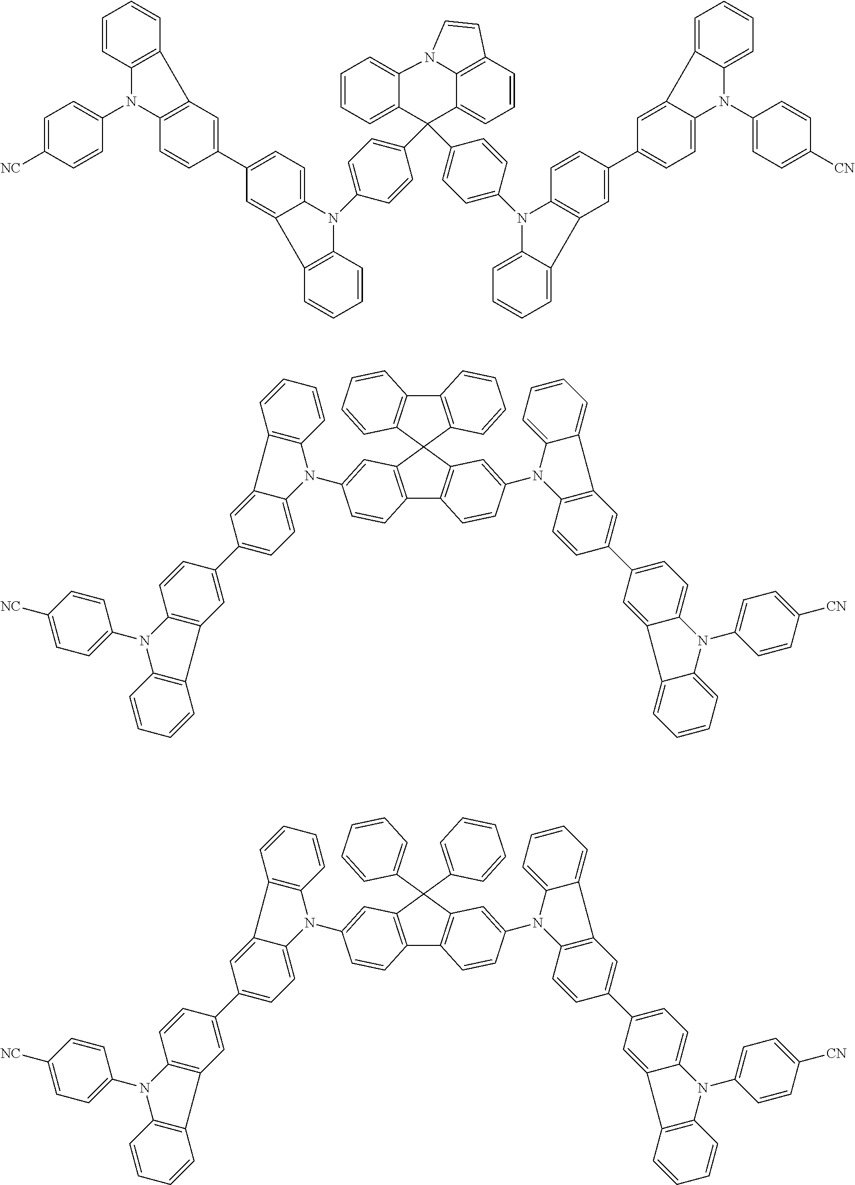 [SMILES: N#Cc1ccc(-n2c3ccccc3c3cc(-c4ccc5c(c4)c4ccccc4n5-c4ccc(C5(c6ccc(-n7c8ccccc8c8cc(-c9ccc%10c(c9)c9ccccc9n%10-c9ccc(C#N)cc9)ccc87)cc6)c6ccccc6-n6ccc7cccc5c76)cc4)ccc32)cc1 ; N#Cc1ccc(-n2c3ccccc3c3cc(-c4ccc5c(c4)c4ccccc4n5-c4ccc5c(c4)C4(c6ccccc6-c6ccccc64)c4cc(-n6c7ccccc7c7cc(-c8ccc9c(c8)c8ccccc8n9-c8ccc(C#N)cc8)ccc76)ccc4-5)ccc32)cc1 ; N#Cc1ccc(-n2c3ccccc3c3cc(-c4ccc5c(c4)c4ccccc4n5-c4ccc5c(c4)C(c4ccccc4)(c4ccccc4)c4cc(-n6c7ccccc7c7cc(-c8ccc9c(c8)c8ccccc8n9-c8ccc(C#N)cc8)ccc76)ccc4-5)ccc32)cc1]

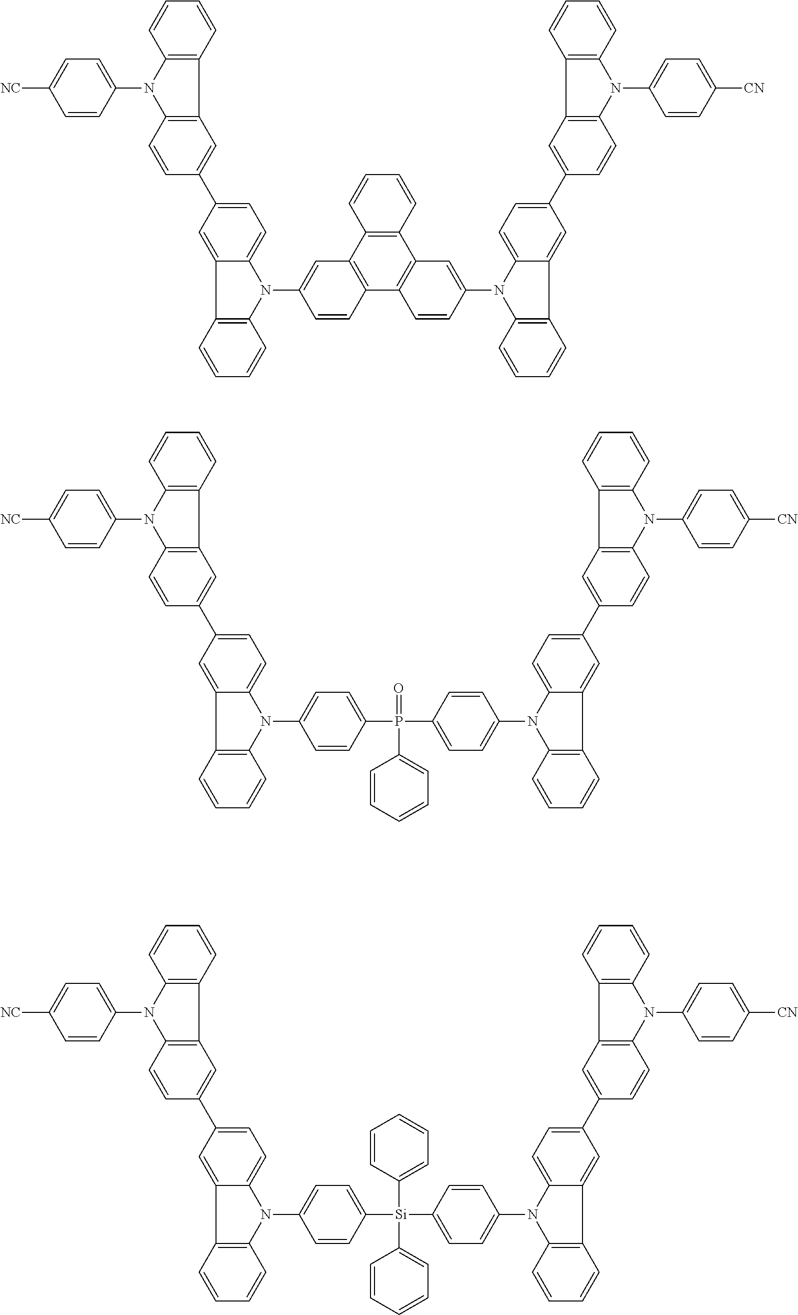 [SMILES: N#Cc1ccc(-n2c3ccccc3c3cc(-c4ccc5c(c4)c4ccccc4n5-c4ccc5c6ccc(-n7c8ccccc8c8cc(-c9ccc%10c(c9)c9ccccc9n%10-c9ccc(C#N)cc9)ccc87)cc6c6ccccc6c5c4)ccc32)cc1 ; N#Cc1ccc(-n2c3ccccc3c3cc(-c4ccc5c(c4)c4ccccc4n5-c4ccc(P(=O)(c5ccccc5)c5ccc(-n6c7ccccc7c7cc(-c8ccc9c(c8)c8ccccc8n9-c8ccc(C#N)cc8)ccc76)cc5)cc4)ccc32)cc1 ; N#Cc1ccc(-n2c3ccccc3c3cc(-c4ccc5c(c4)c4ccccc4n5-c4ccc([Si](c5ccccc5)(c5ccccc5)c5ccc(-n6c7ccccc7c7cc(-c8ccc9c(c8)c8ccccc8n9-c8ccc(C#N)cc8)ccc76)cc5)cc4)ccc32)cc1]

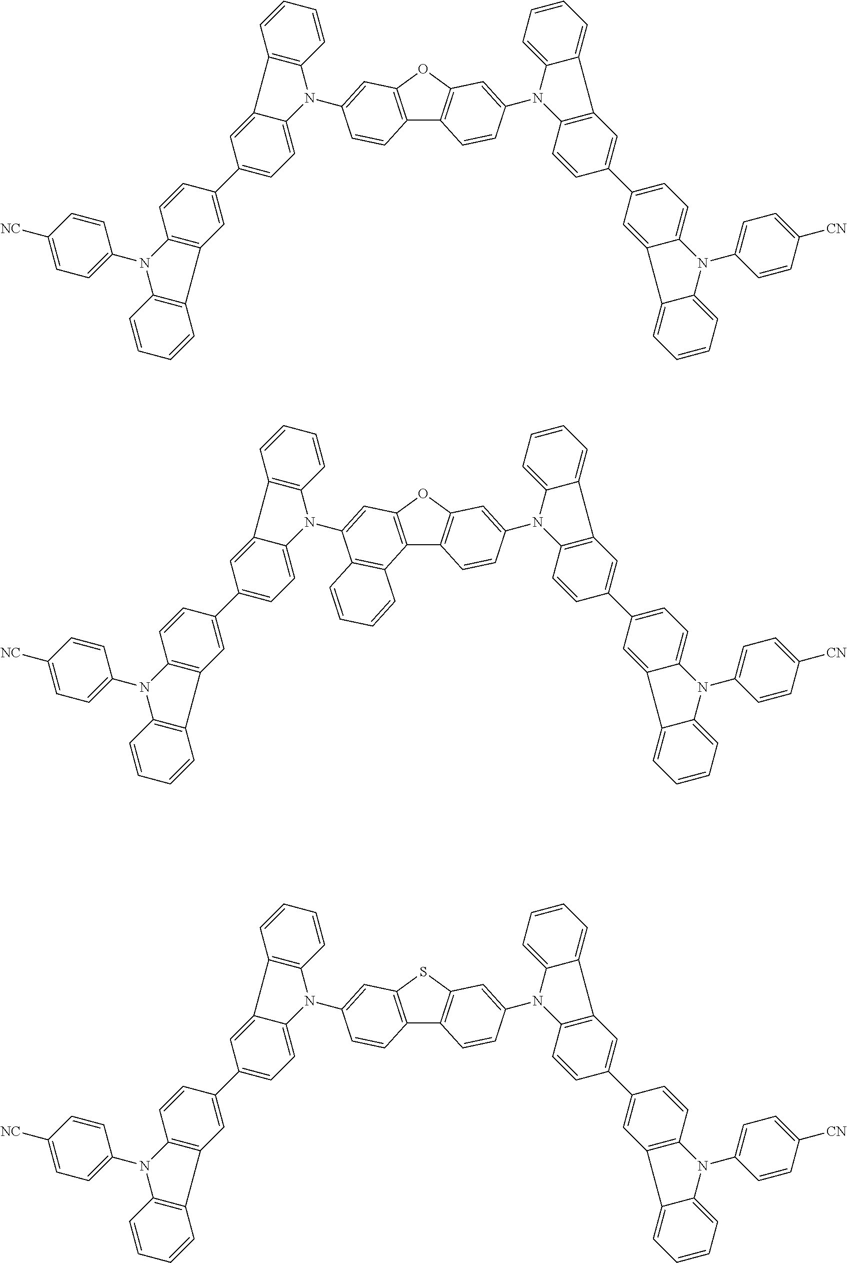 [SMILES: N#Cc1ccc(-n2c3ccccc3c3cc(-c4ccc5c(c4)c4ccccc4n5-c4ccc5c(c4)oc4cc(-n6c7ccccc7c7cc(-c8ccc9c(c8)c8ccccc8n9-c8ccc(C#N)cc8)ccc76)ccc45)ccc32)cc1 ; N#Cc1ccc(-n2c3ccccc3c3cc(-c4ccc5c(c4)c4ccccc4n5-c4ccc5c(c4)oc4cc(-n6c7ccccc7c7cc(-c8ccc9c(c8)c8ccccc8n9-c8ccc(C#N)cc8)ccc76)c6ccccc6c45)ccc32)cc1 ; N#Cc1ccc(-n2c3ccccc3c3cc(-c4ccc5c(c4)c4ccccc4n5-c4ccc5c(c4)sc4cc(-n6c7ccccc7c7cc(-c8ccc9c(c8)c8ccccc8n9-c8ccc(C#N)cc8)ccc76)ccc45)ccc32)cc1]

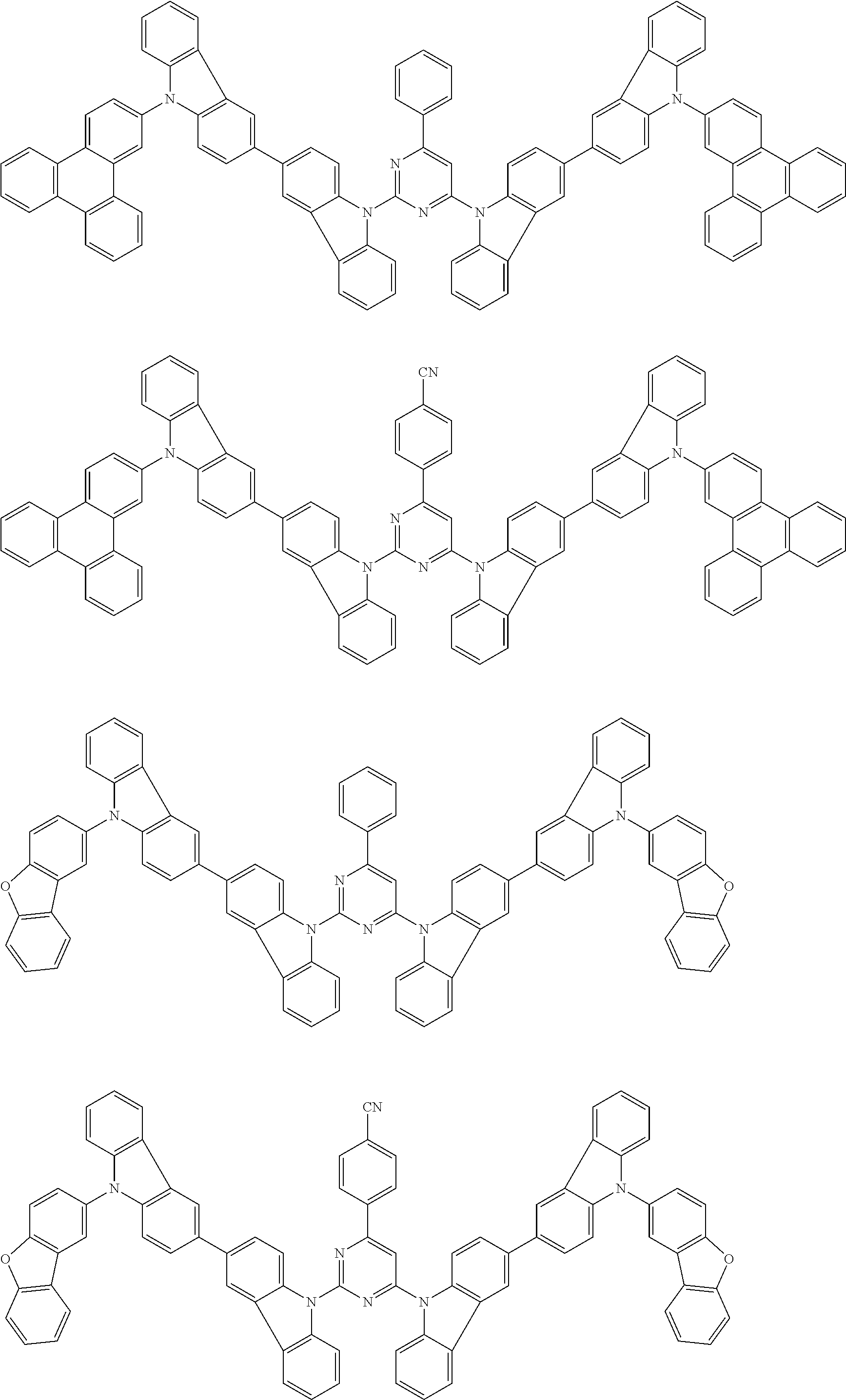

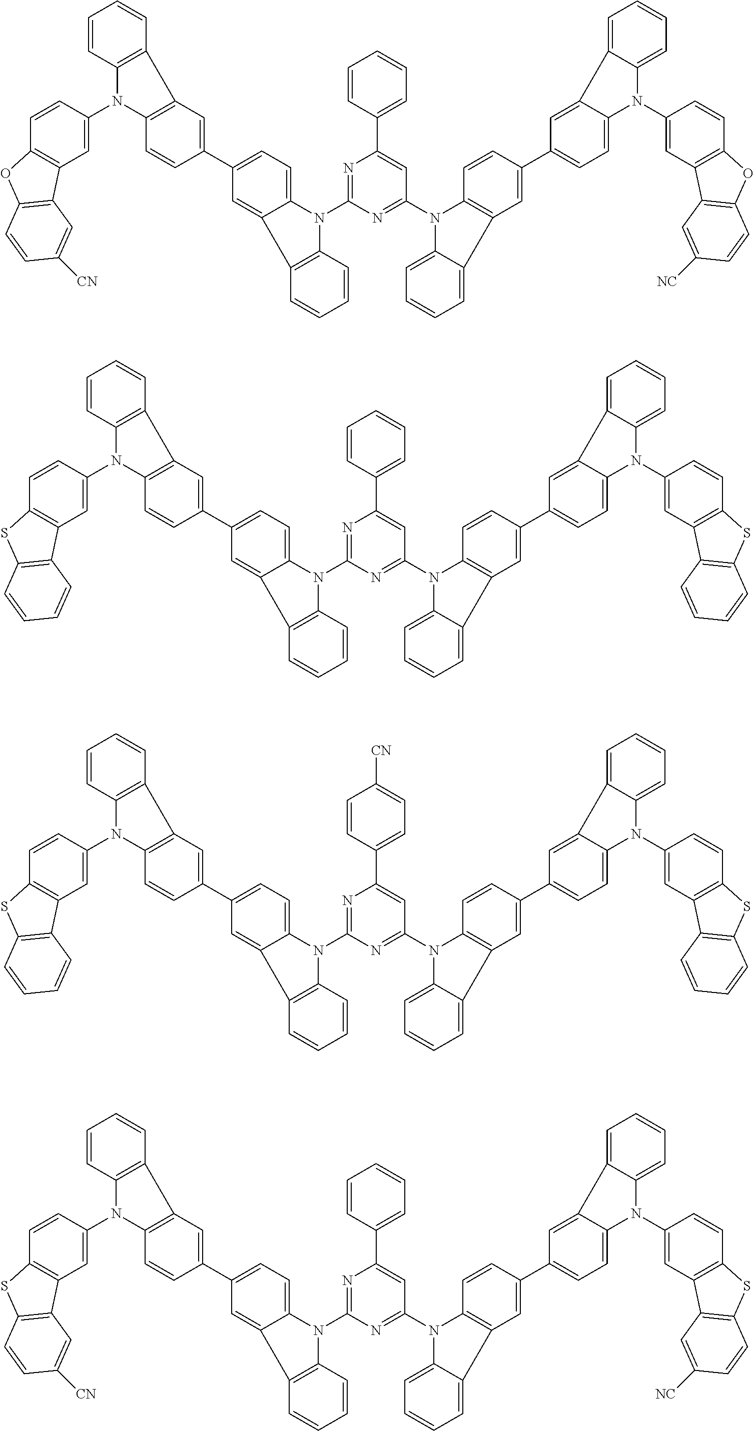

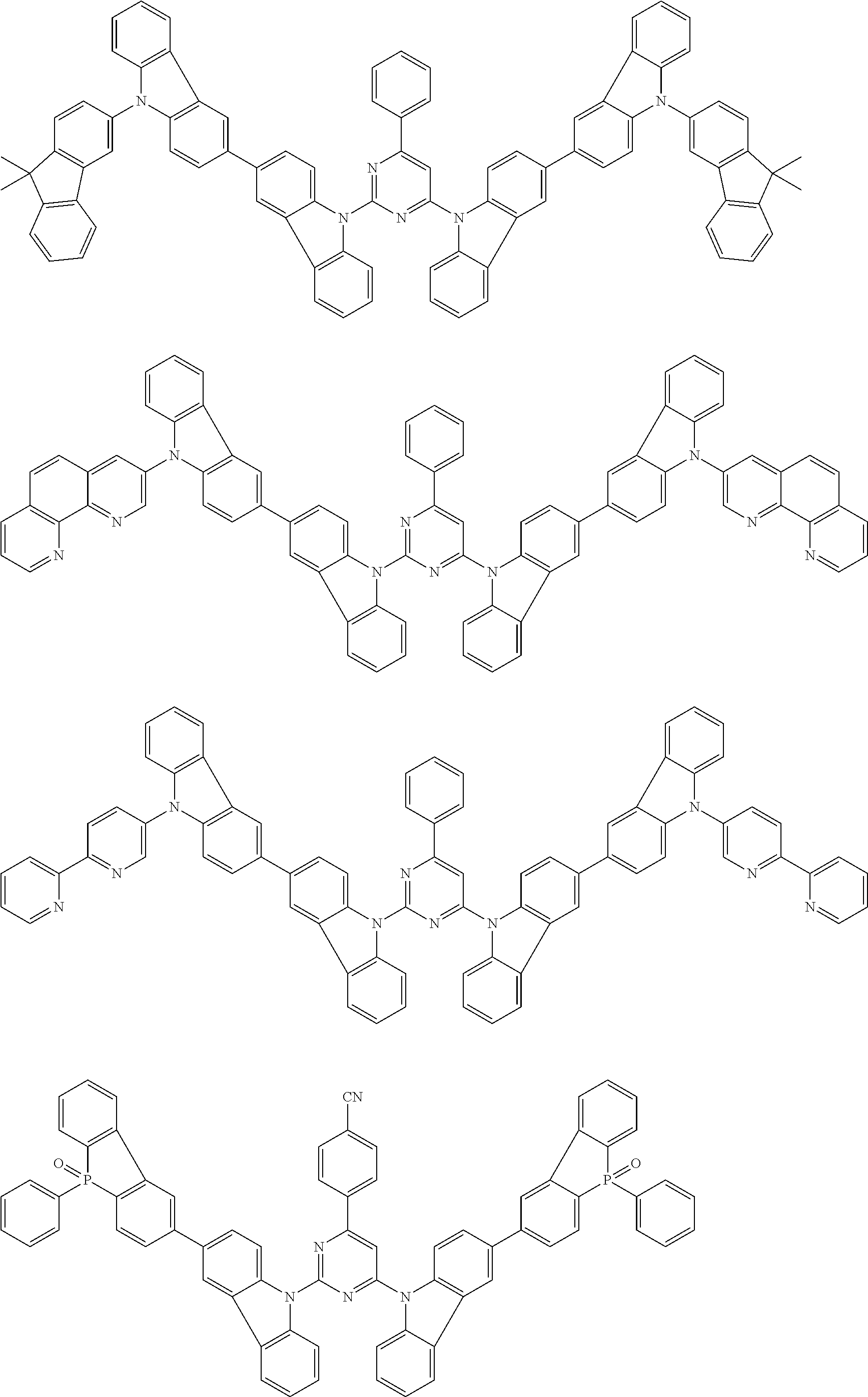

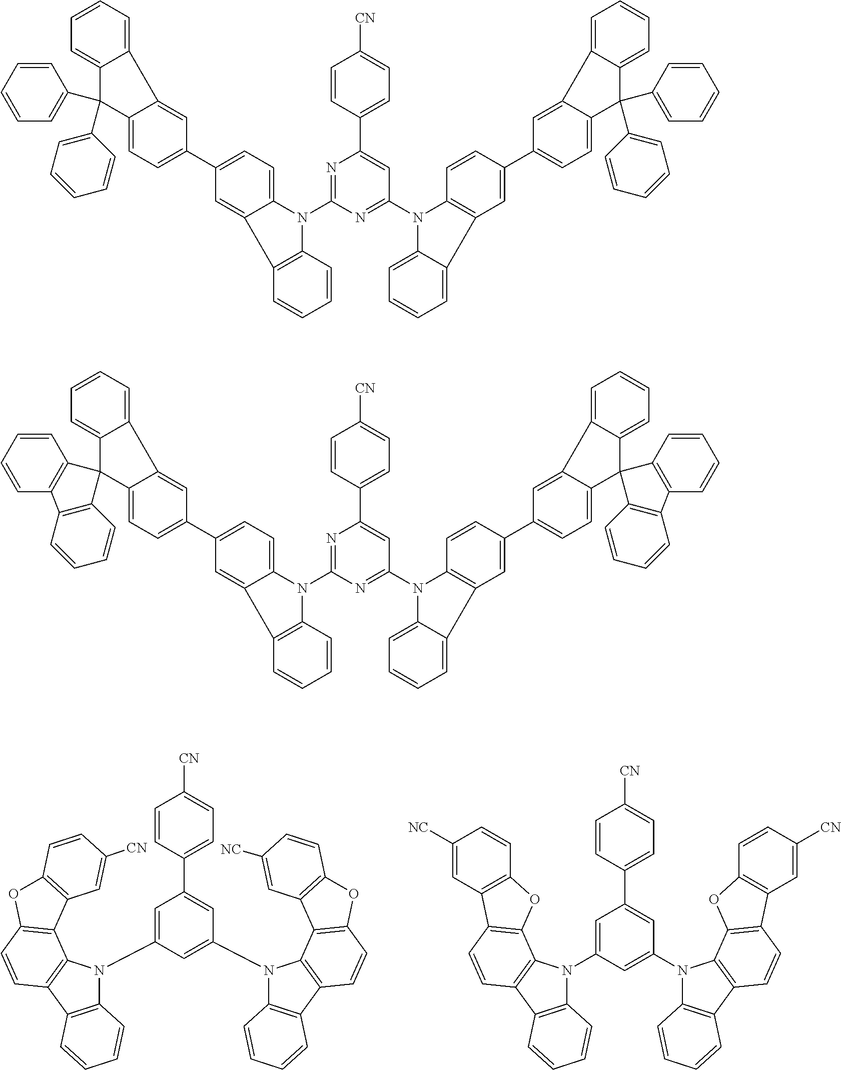

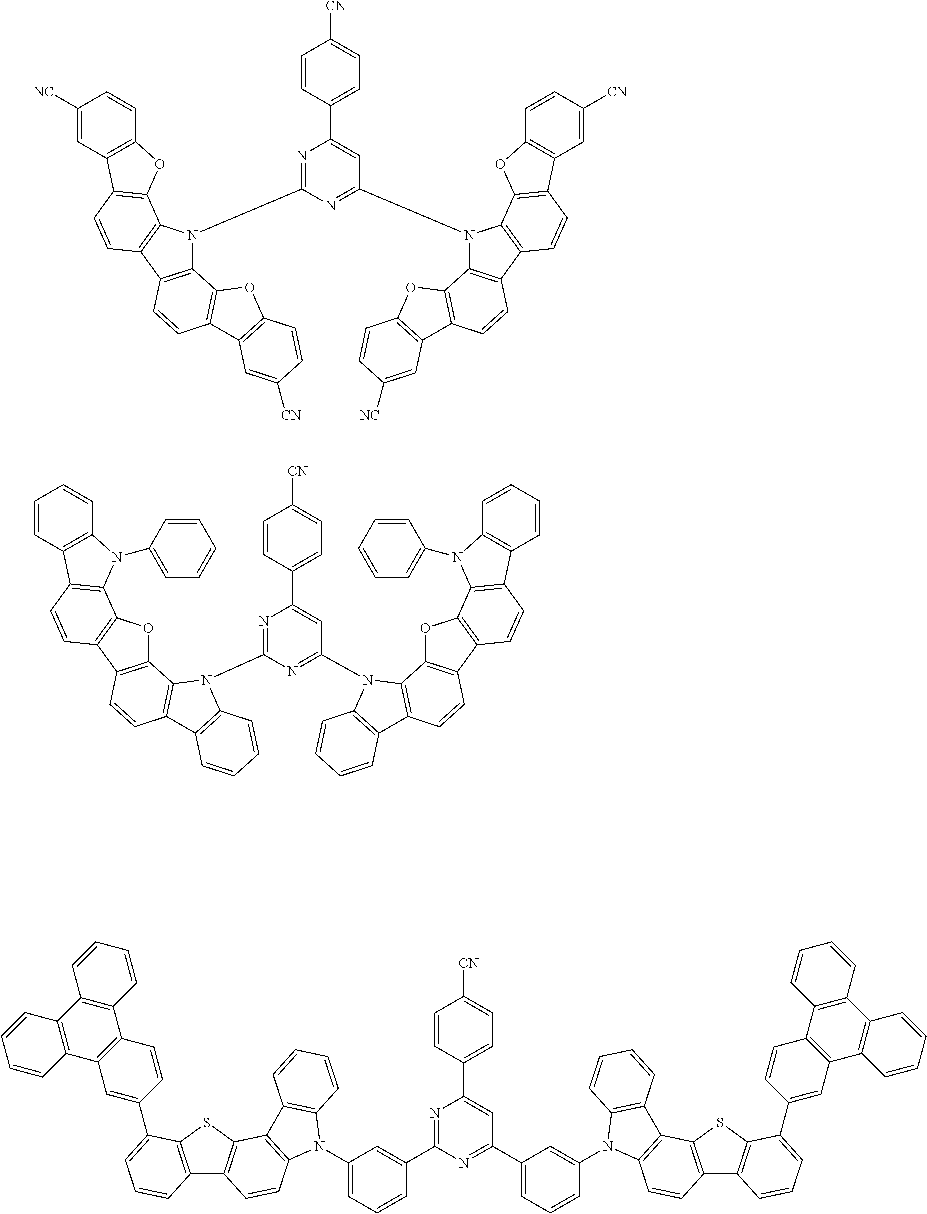 [SMILES: N#Cc1ccc(-c2cc(-n3c4c(ccc5c6cc(C#N)ccc6oc54)c4ccc5c6cc(C#N)ccc6oc5c43)nc(-n3c4c(ccc5c6cc(C#N)ccc6oc54)c4ccc5c6cc(C#N)ccc6oc5c43)n2)cc1 ; N#Cc1ccc(-c2cc(-n3c4ccccc4c4ccc5c6ccc7c8ccccc8n(-c8ccccc8)c7c6oc5c43)nc(-n3c4ccccc4c4ccc5c6ccc7c8ccccc8n(-c8ccccc8)c7c6oc5c43)n2)cc1 ; N#Cc1ccc(-c2cc(-c3cccc(-n4c5ccccc5c5c6sc7c(-c8ccc9c%10ccccc%10c%10ccccc%10c9c8)cccc7c6ccc54)c3)nc(-c3cccc(-n4c5ccccc5c5c6sc7c(-c8ccc9c%10ccccc%10c%10ccccc%10c9c8)cccc7c6ccc54)c3)n2)cc1]

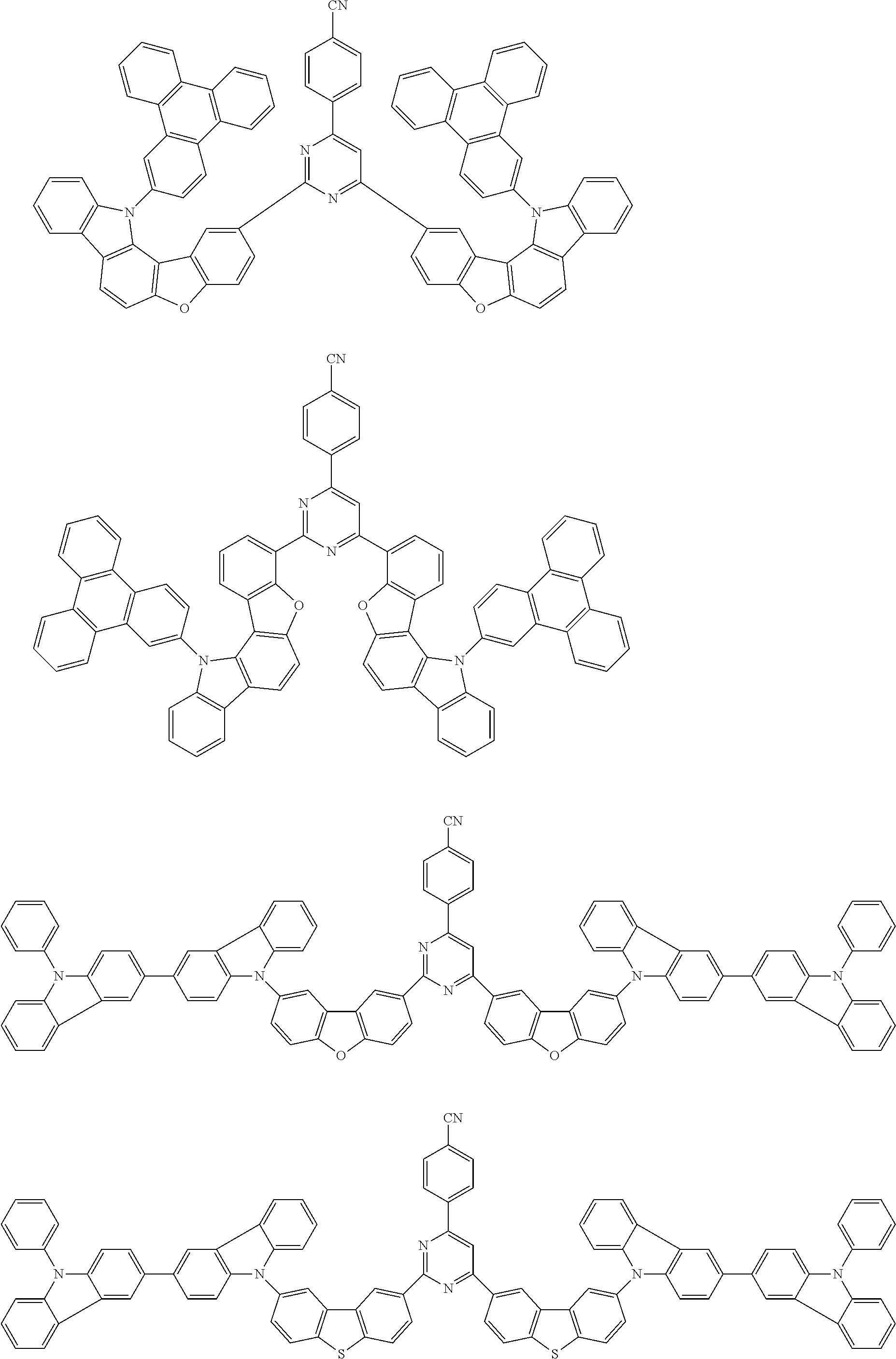

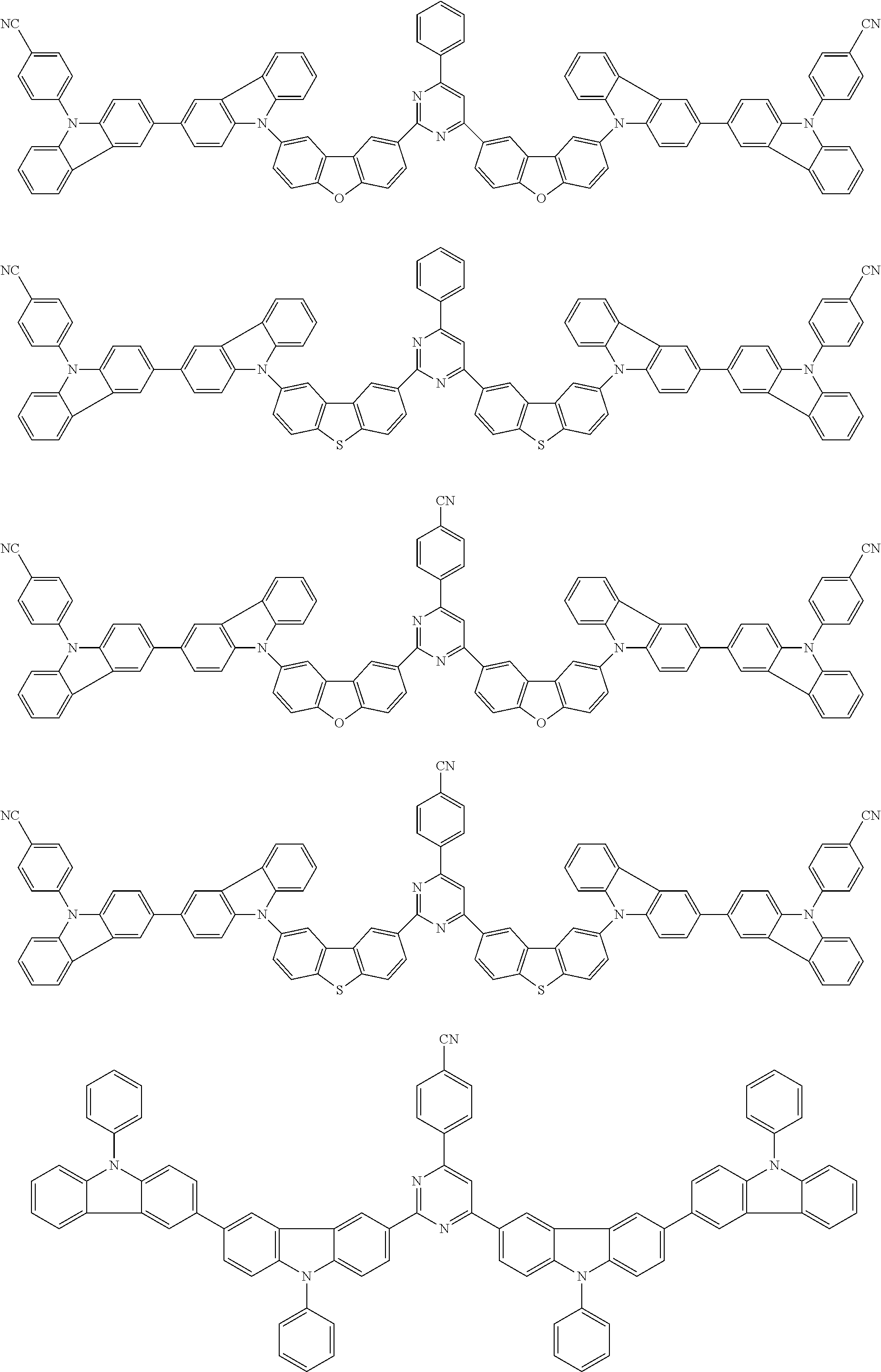

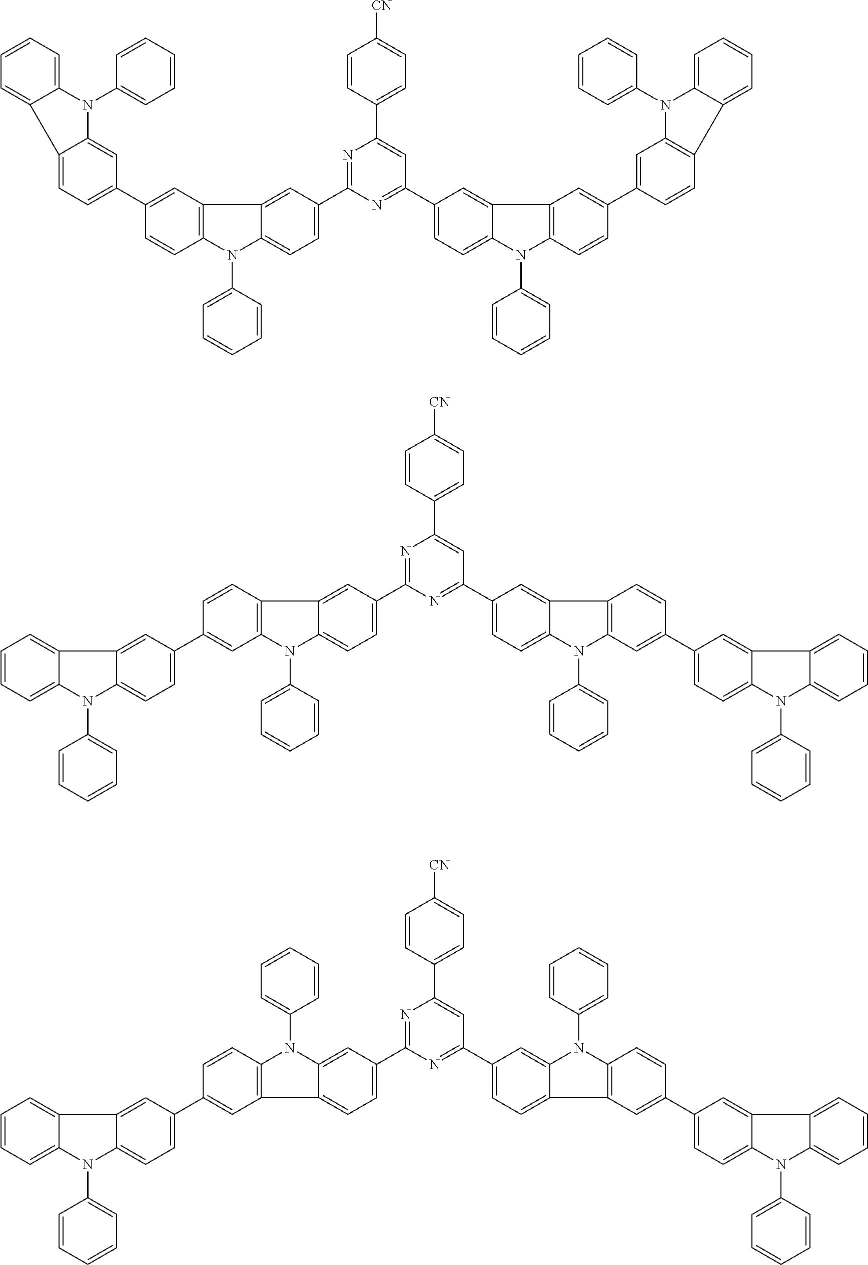 [SMILES: N#Cc1ccc(-c2cc(-c3ccc4c(c3)c3cc(-c5ccc6c7ccccc7n(-c7ccccc7)c6c5)ccc3n4-c3ccccc3)nc(-c3ccc4c(c3)c3cc(-c5ccc6c7ccccc7n(-c7ccccc7)c6c5)ccc3n4-c3ccccc3)n2)cc1 ; N#Cc1ccc(-c2cc(-c3ccc4c(c3)c3ccc(-c5ccc6c(c5)c5ccccc5n6-c5ccccc5)cc3n4-c3ccccc3)nc(-c3ccc4c(c3)c3ccc(-c5ccc6c(c5)c5ccccc5n6-c5ccccc5)cc3n4-c3ccccc3)n2)cc1 ; N#Cc1ccc(-c2cc(-c3ccc4c5cc(-c6ccc7c(c6)c6ccccc6n7-c6ccccc6)ccc5n(-c5ccccc5)c4c3)nc(-c3ccc4c5cc(-c6ccc7c(c6)c6ccccc6n7-c6ccccc6)ccc5n(-c5ccccc5)c4c3)n2)cc1]

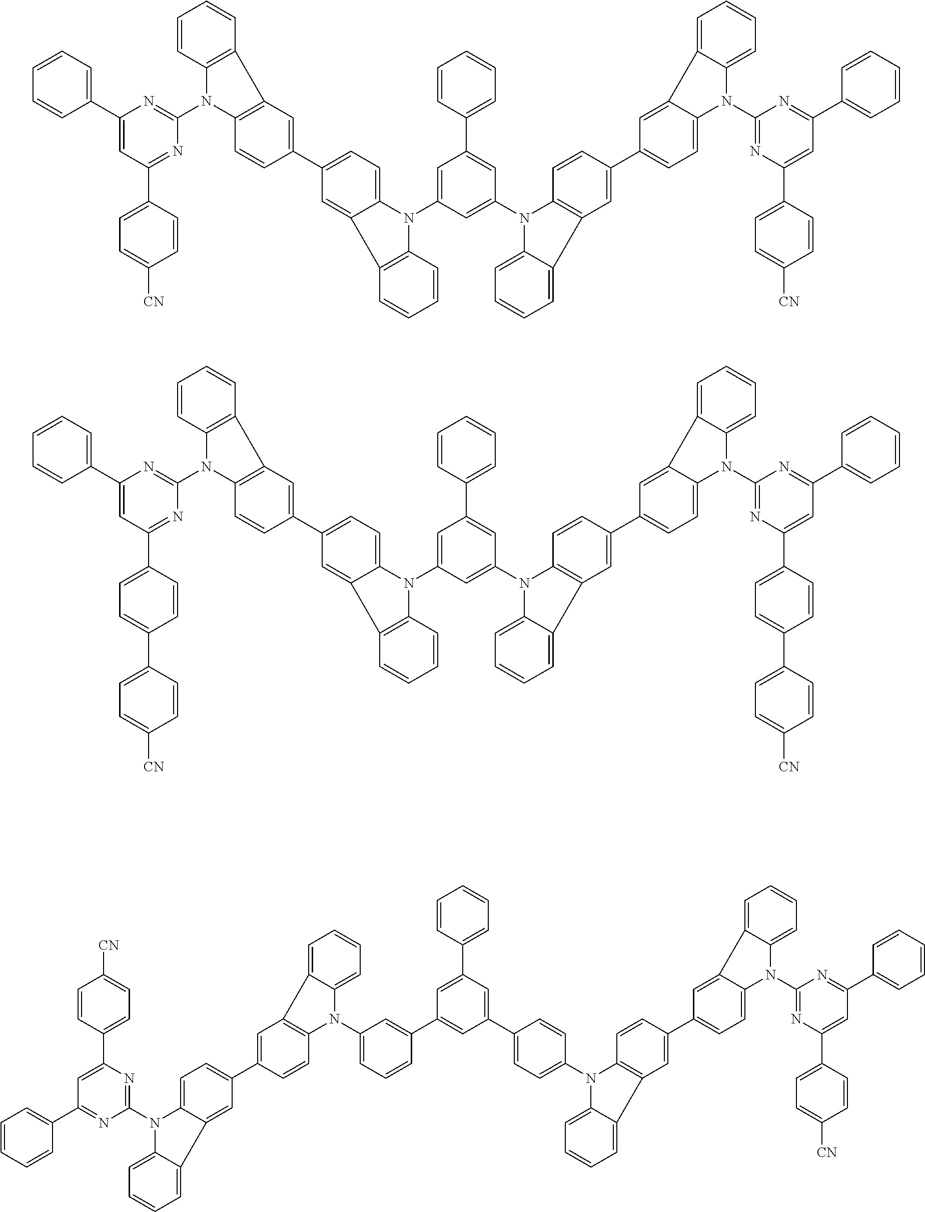

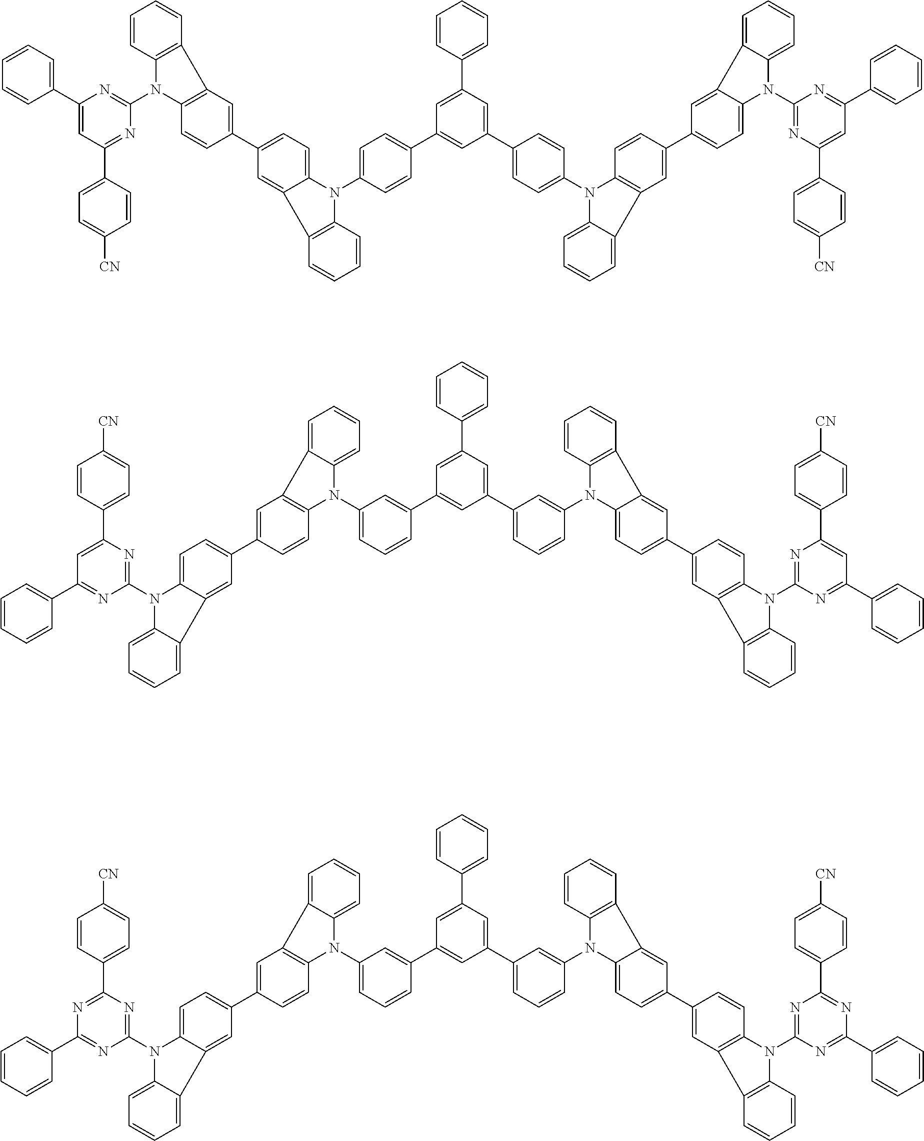

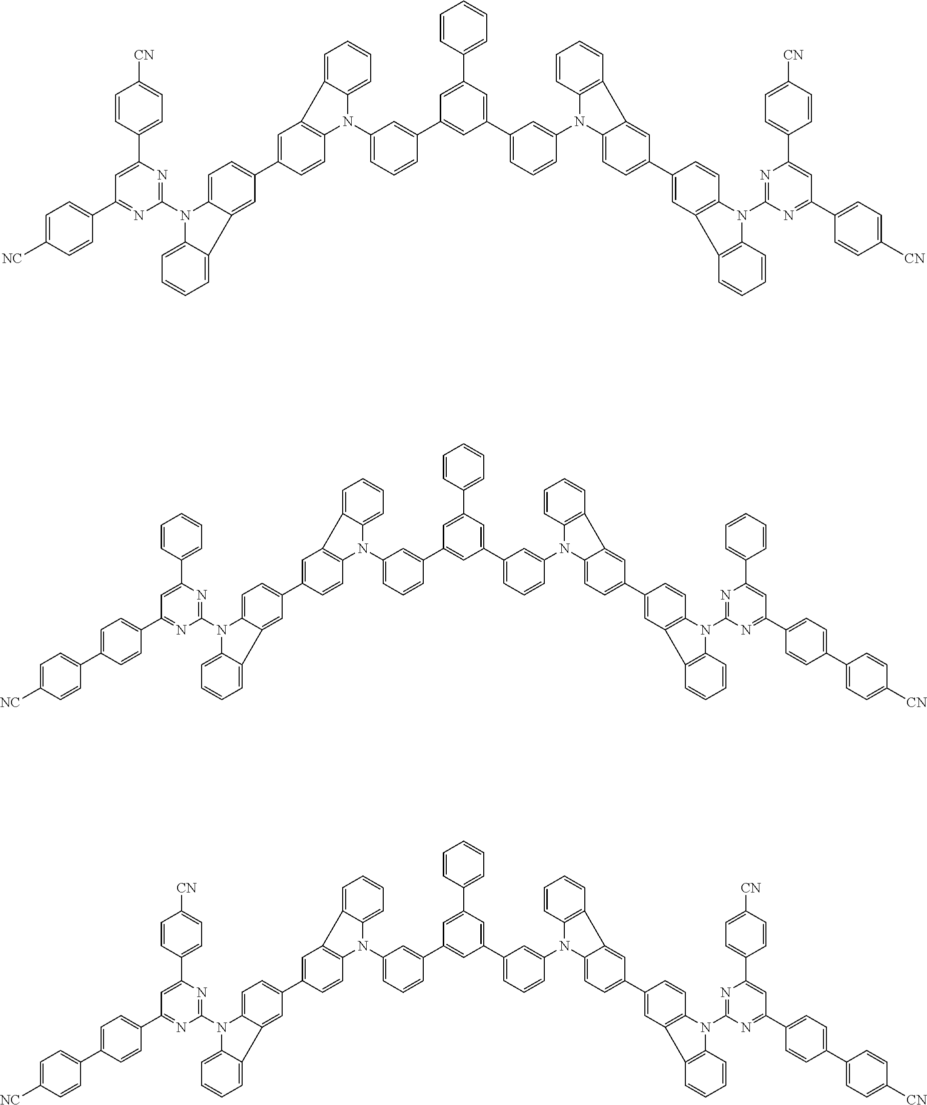

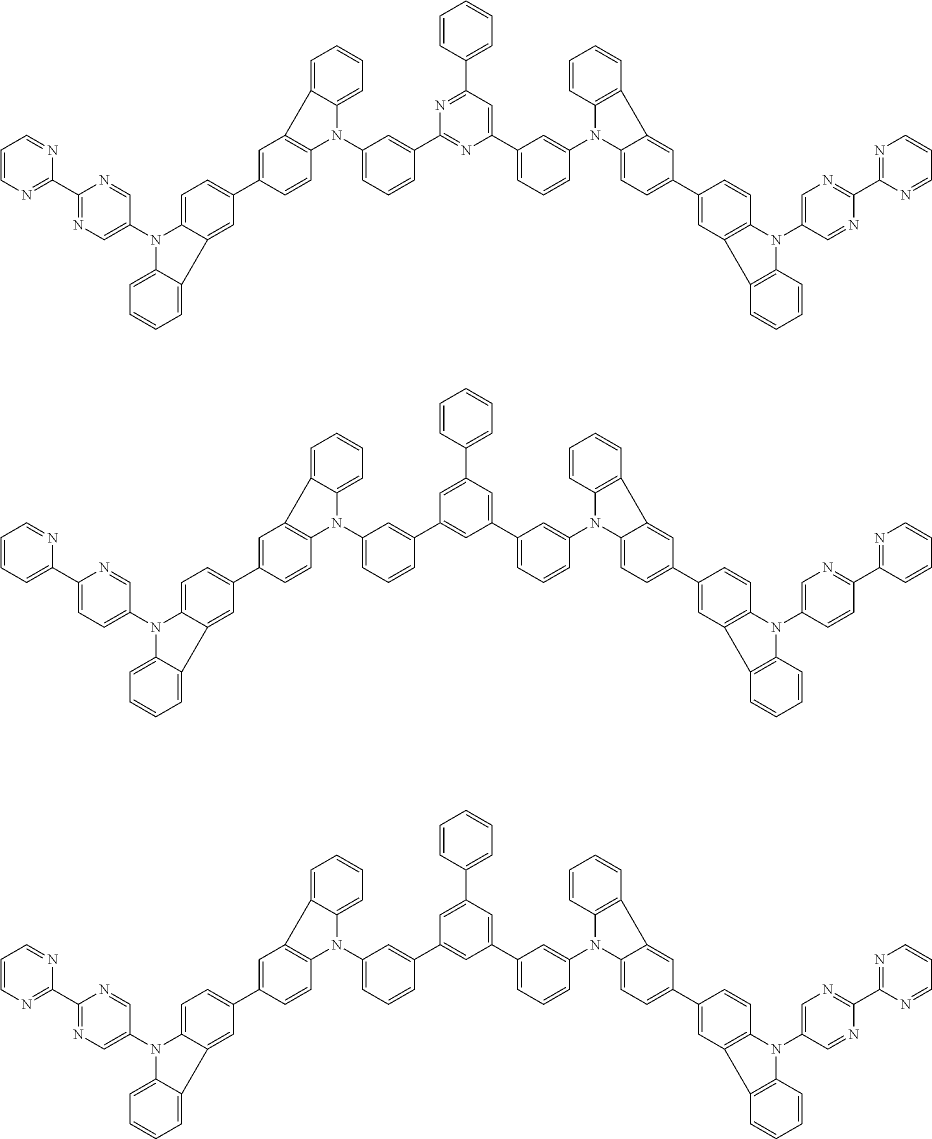

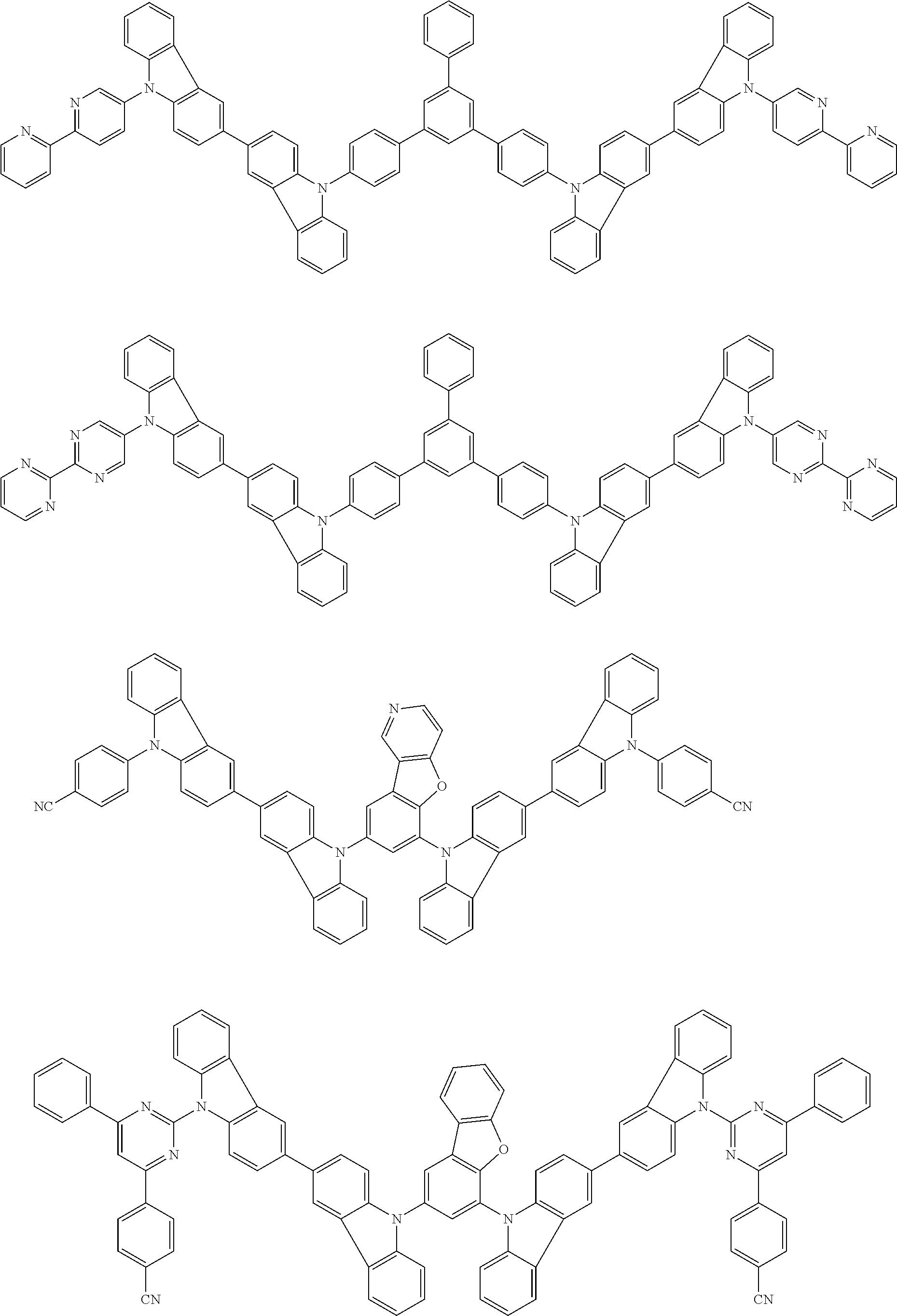

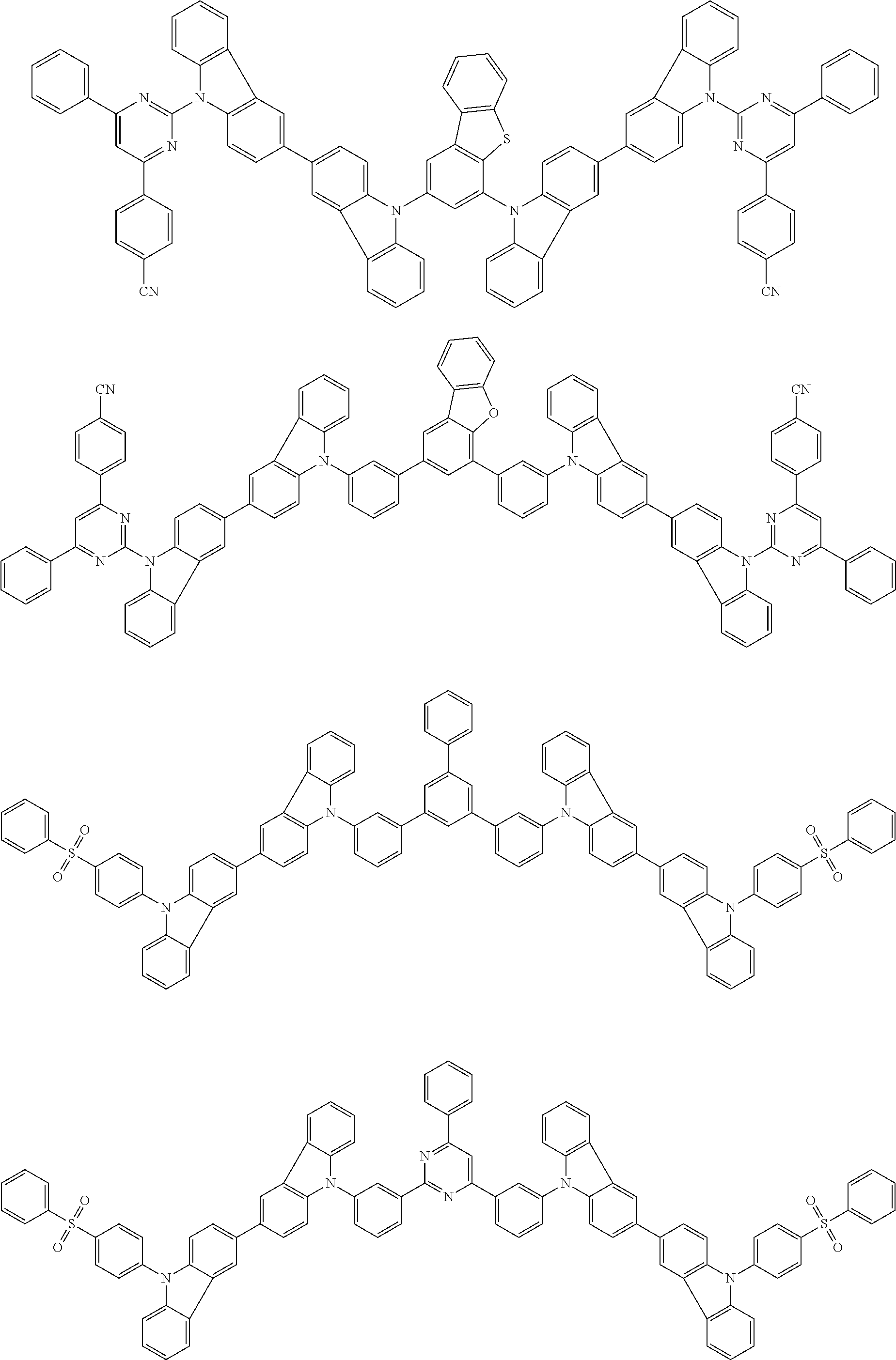

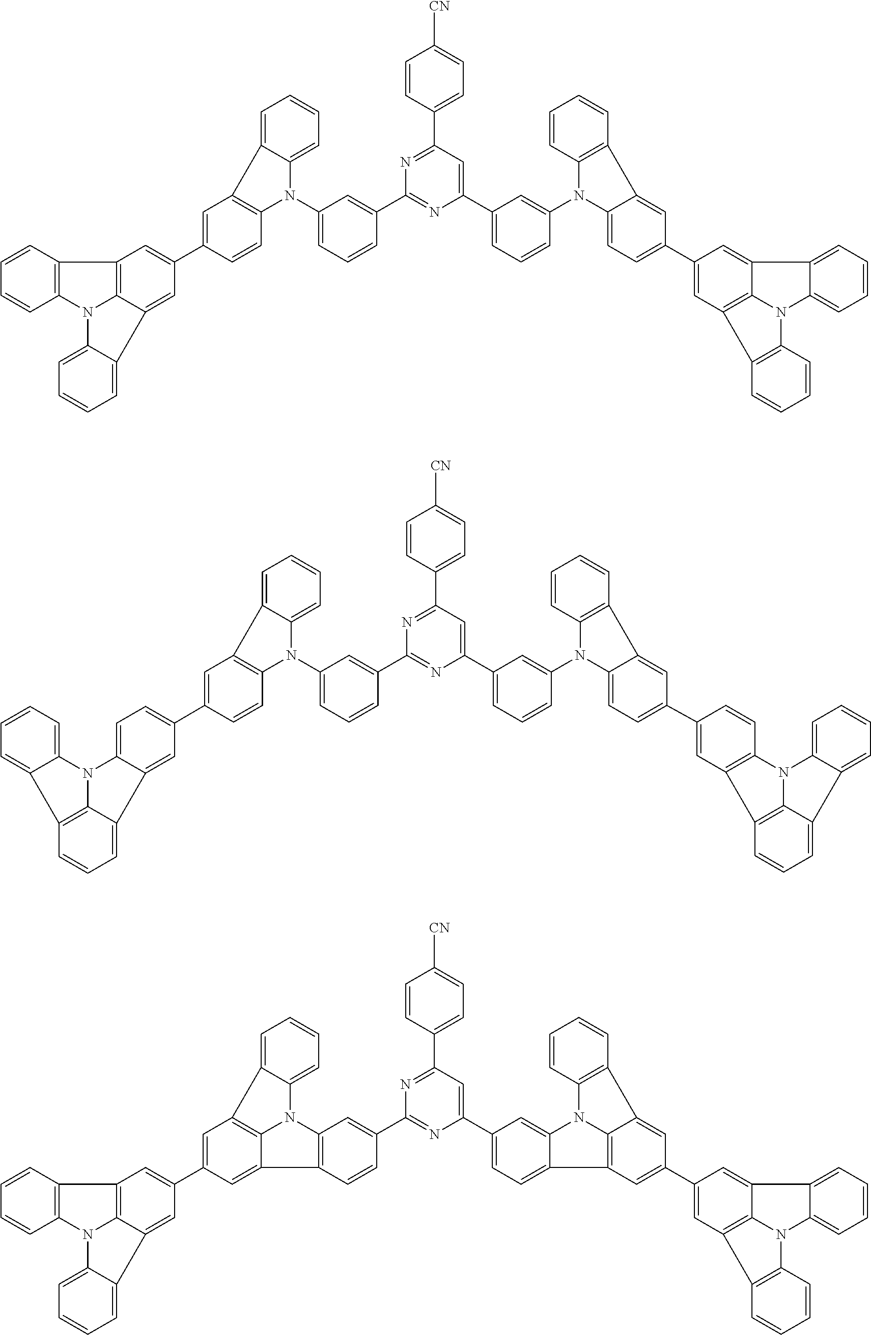 [SMILES: N#Cc1ccc(-c2cc(-c3cccc(-n4c5ccccc5c5cc(-c6cc7c8ccccc8n8c9ccccc9c(c6)c78)ccc54)c3)nc(-c3cccc(-n4c5ccccc5c5cc(-c6cc7c8ccccc8n8c9ccccc9c(c6)c78)ccc54)c3)n2)cc1 ; N#Cc1ccc(-c2cc(-c3cccc(-n4c5ccccc5c5cc(-c6ccc7c(c6)c6cccc8c9ccccc9n7c86)ccc54)c3)nc(-c3cccc(-n4c5ccccc5c5cc(-c6ccc7c(c6)c6cccc8c9ccccc9n7c86)ccc54)c3)n2)cc1 ; N#Cc1ccc(-c2cc(-c3ccc4c5cc(-c6cc7c8ccccc8n8c9ccccc9c(c6)c78)cc6c7ccccc7n(c4c3)c65)nc(-c3ccc4c5cc(-c6cc7c8ccccc8n8c9ccccc9c(c6)c78)cc6c7ccccc7n(c4c3)c65)n2)cc1]

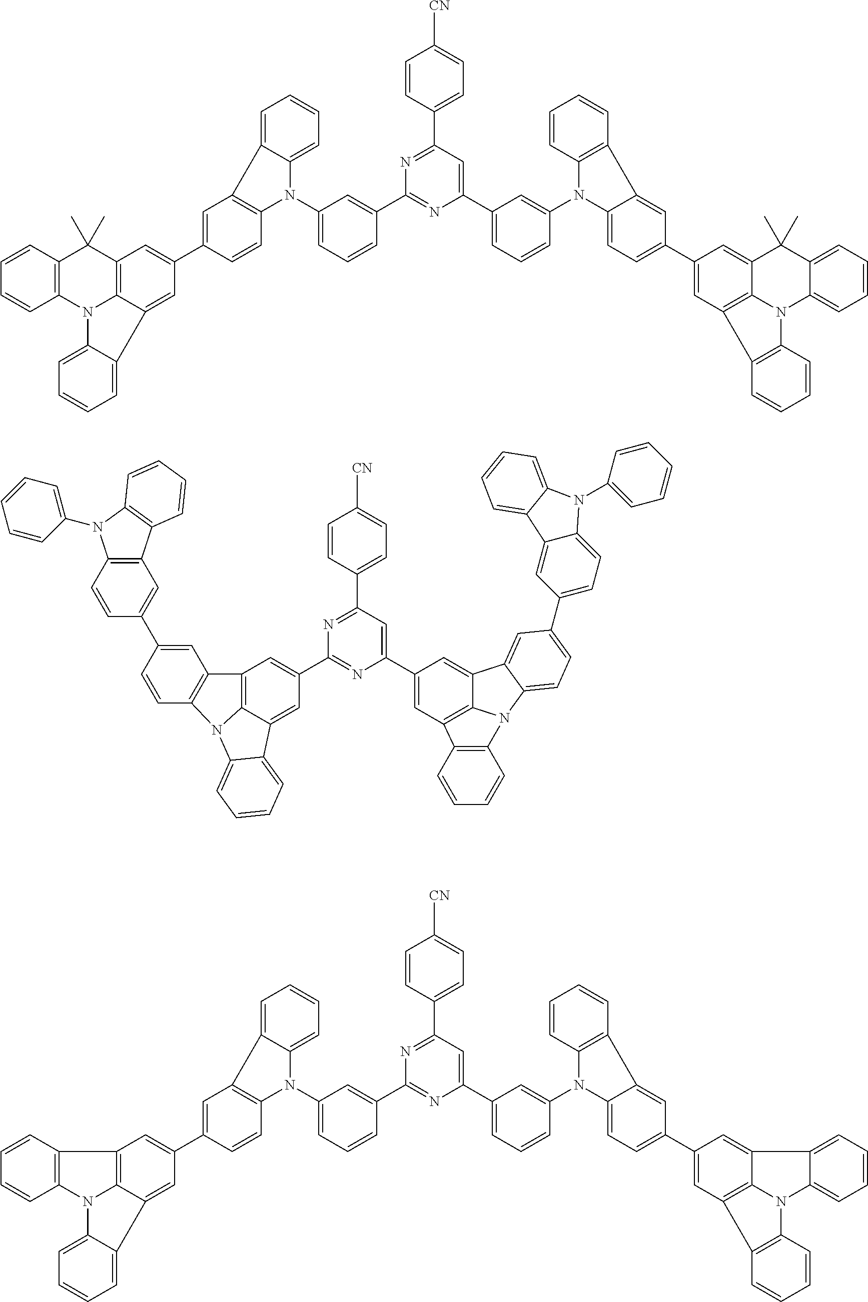 [SMILES: CC1(C)c2ccccc2-n2c3ccccc3c3cc(-c4ccc5c(c4)c4ccccc4n5-c4cccc(-c5cc(-c6ccc(C#N)cc6)nc(-c6cccc(-n7c8ccccc8c8cc(-c9cc%10c%11c(c9)c9ccccc9n%11-c9ccccc9C%10(C)C)ccc87)c6)n5)c4)cc1c32 ; N#Cc1ccc(-c2cc(-c3cc4c5ccccc5n5c6ccc(-c7ccc8c(c7)c7ccccc7n8-c7ccccc7)cc6c(c3)c45)nc(-c3cc4c5ccccc5n5c6ccc(-c7ccc8c(c7)c7ccccc7n8-c7ccccc7)cc6c(c3)c45)n2)cc1 ; N#Cc1ccc(-c2cc(-c3cccc(-n4c5ccccc5c5cc(-c6cc7c8ccccc8n8c9ccccc9c(c6)c78)ccc54)c3)nc(-c3cccc(-n4c5ccccc5c5cc(-c6cc7c8ccccc8n8c9ccccc9c(c6)c78)ccc54)c3)n2)cc1]

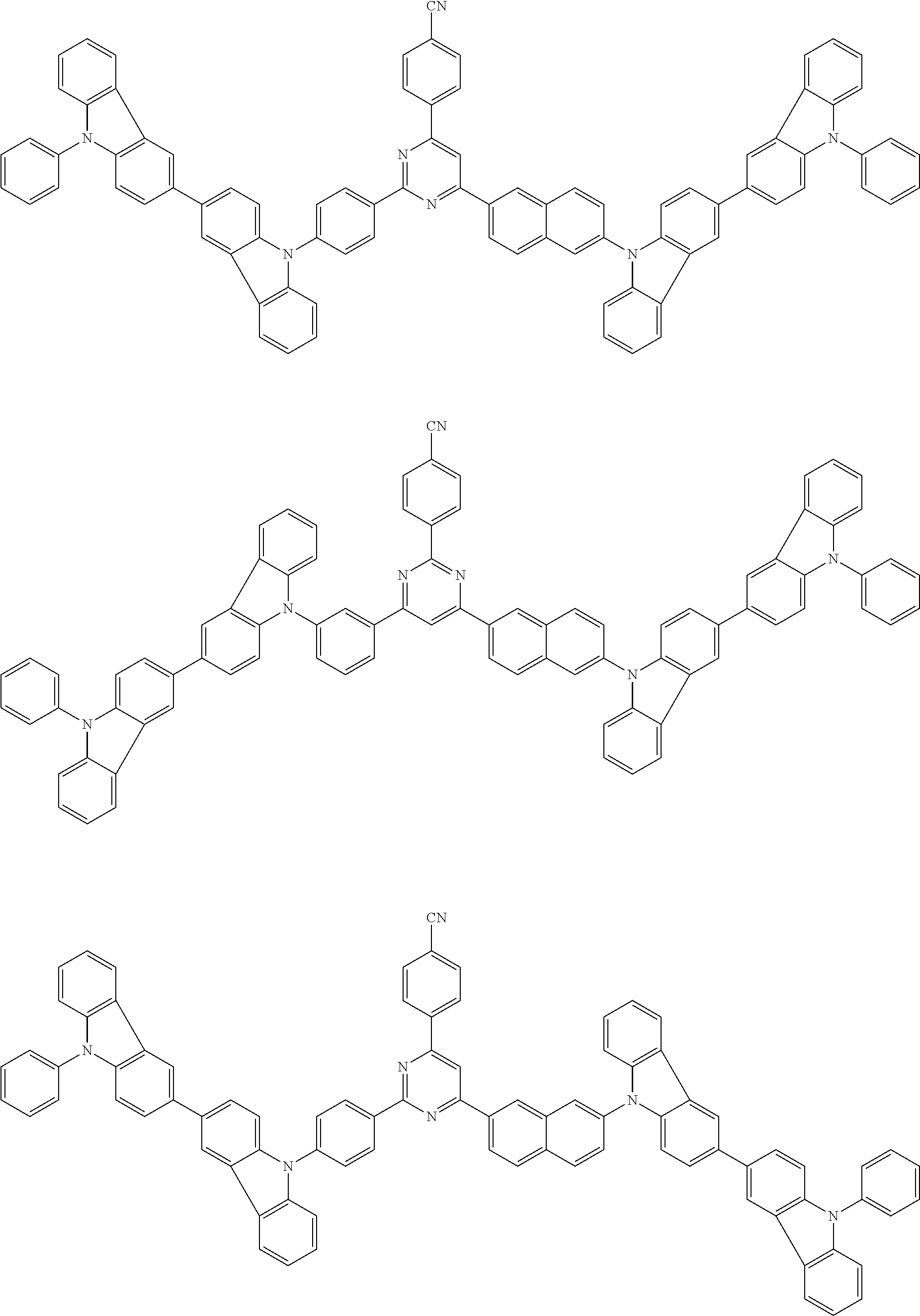 [SMILES: N#Cc1ccc(-c2cc(-c3ccc4cc(-n5c6ccccc6c6cc(-c7ccc8c(c7)c7ccccc7n8-c7ccccc7)ccc65)ccc4c3)nc(-c3ccc(-n4c5ccccc5c5cc(-c6ccc7c(c6)c6ccccc6n7-c6ccccc6)ccc54)cc3)n2)cc1 ; N#Cc1ccc(-c2nc(-c3cccc(-n4c5ccccc5c5cc(-c6ccc7c(c6)c6ccccc6n7-c6ccccc6)ccc54)c3)cc(-c3ccc4cc(-n5c6ccccc6c6cc(-c7ccc8c(c7)c7ccccc7n8-c7ccccc7)ccc65)ccc4c3)n2)cc1 ; N#Cc1ccc(-c2cc(-c3ccc4ccc(-n5c6ccccc6c6cc(-c7ccc8c(c7)c7ccccc7n8-c7ccccc7)ccc65)cc4c3)nc(-c3ccc(-n4c5ccccc5c5cc(-c6ccc7c(c6)c6ccccc6n7-c6ccccc6)ccc54)cc3)n2)cc1]

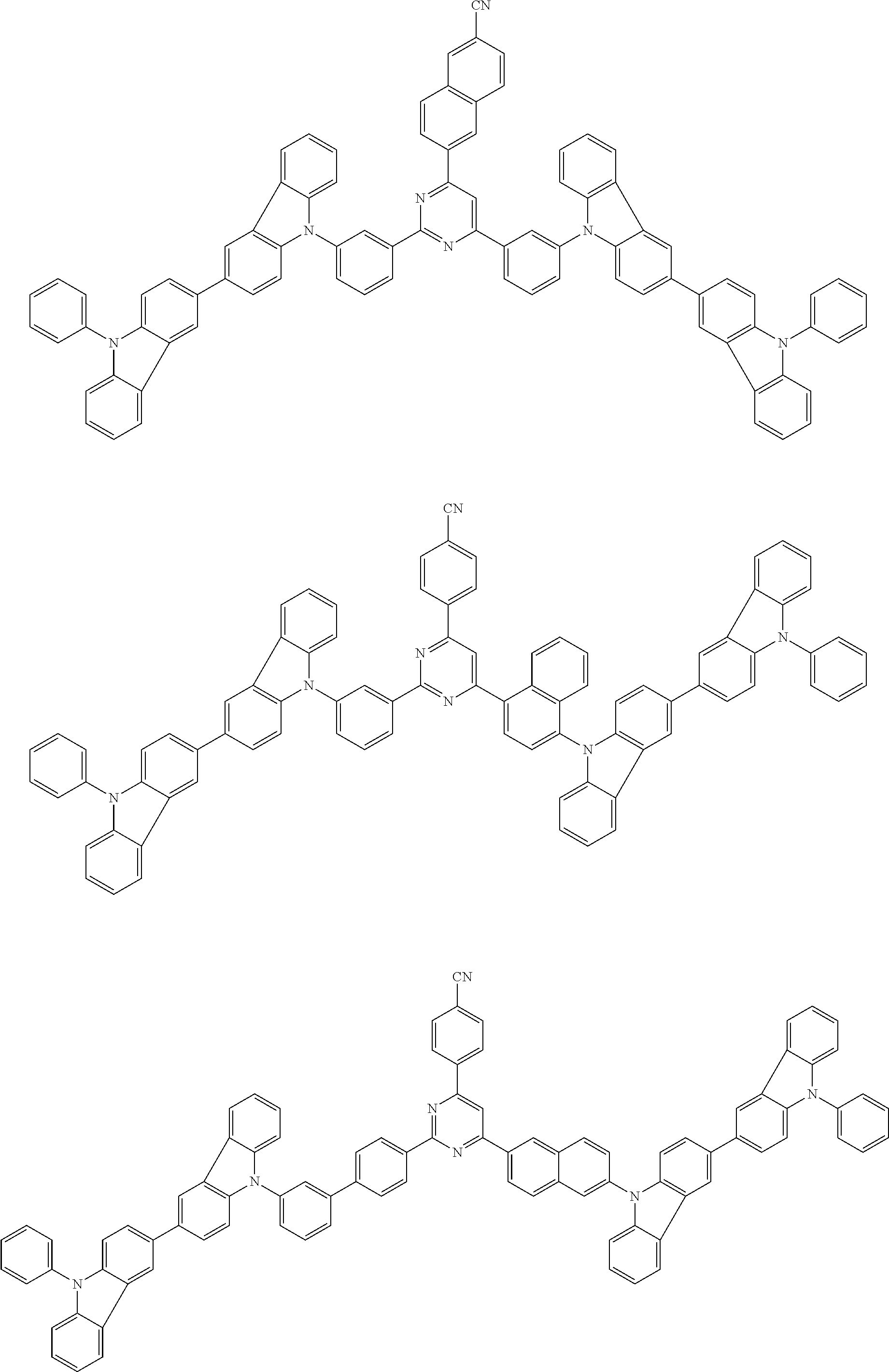 [SMILES: N#Cc1ccc2cc(-c3cc(-c4cccc(-n5c6ccccc6c6cc(-c7ccc8c(c7)c7ccccc7n8-c7ccccc7)ccc65)c4)nc(-c4cccc(-n5c6ccccc6c6cc(-c7ccc8c(c7)c7ccccc7n8-c7ccccc7)ccc65)c4)n3)ccc2c1 ; N#Cc1ccc(-c2cc(-c3ccc(-n4c5ccccc5c5cc(-c6ccc7c(c6)c6ccccc6n7-c6ccccc6)ccc54)c4ccccc34)nc(-c3cccc(-n4c5ccccc5c5cc(-c6ccc7c(c6)c6ccccc6n7-c6ccccc6)ccc54)c3)n2)cc1 ; N#Cc1ccc(-c2cc(-c3ccc4cc(-n5c6ccccc6c6cc(-c7ccc8c(c7)c7ccccc7n8-c7ccccc7)ccc65)ccc4c3)nc(-c3ccc(-c4cccc(-n5c6ccccc6c6cc(-c7ccc8c(c7)c7ccccc7n8-c7ccccc7)ccc65)c4)cc3)n2)cc1]

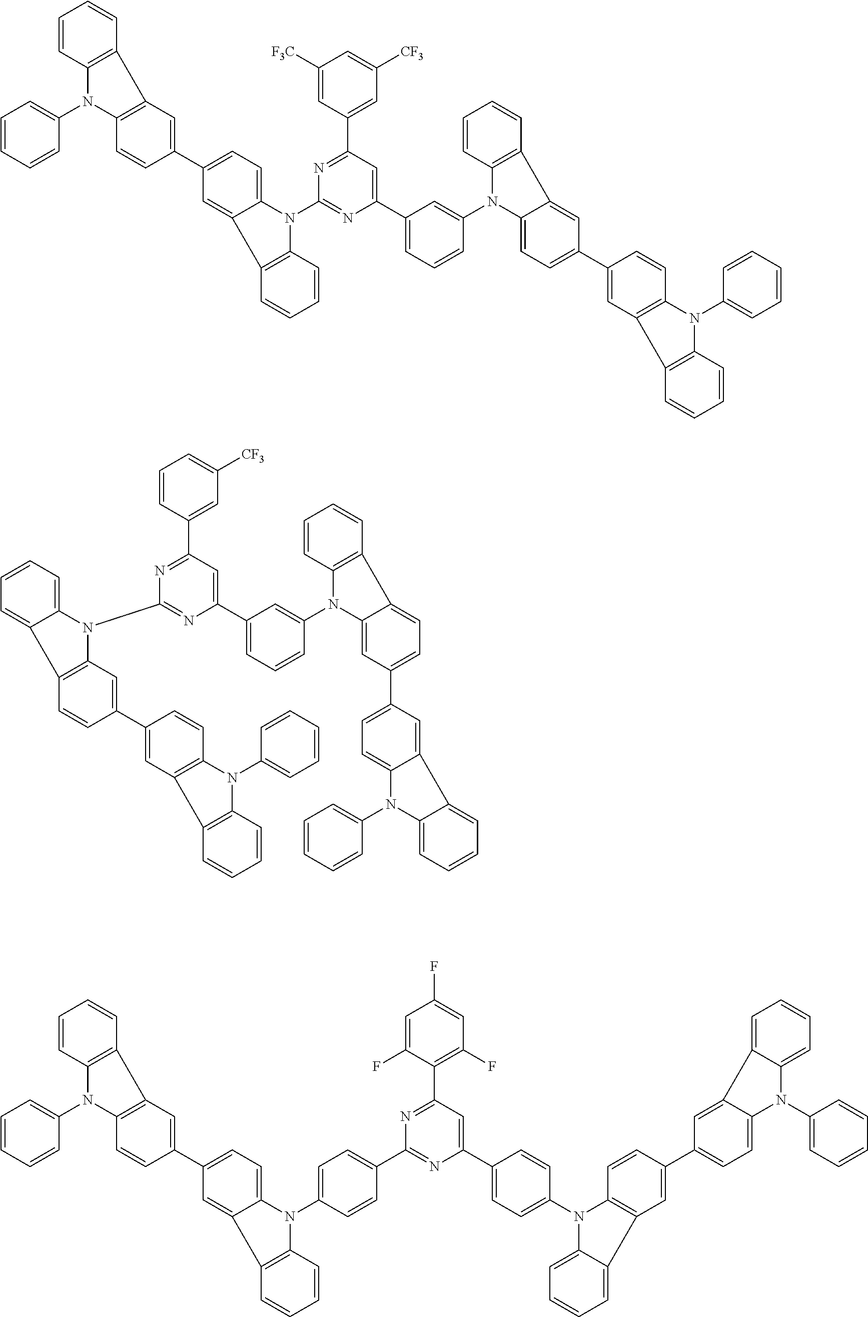 [SMILES: FC(F)(F)c1cc(-c2cc(-c3cccc(-n4c5ccccc5c5cc(-c6ccc7c(c6)c6ccccc6n7-c6ccccc6)ccc54)c3)nc(-n3c4ccccc4c4cc(-c5ccc6c(c5)c5ccccc5n6-c5ccccc5)ccc43)n2)cc(C(F)(F)F)c1 ; FC(F)(F)c1cccc(-c2cc(-c3cccc(-n4c5ccccc5c5ccc(-c6ccc7c(c6)c6ccccc6n7-c6ccccc6)cc54)c3)nc(-n3c4ccccc4c4ccc(-c5ccc6c(c5)c5ccccc5n6-c5ccccc5)cc43)n2)c1 ; Fc1cc(F)c(-c2cc(-c3ccc(-n4c5ccccc5c5cc(-c6ccc7c(c6)c6ccccc6n7-c6ccccc6)ccc54)cc3)nc(-c3ccc(-n4c5ccccc5c5cc(-c6ccc7c(c6)c6ccccc6n7-c6ccccc6)ccc54)cc3)n2)c(F)c1]

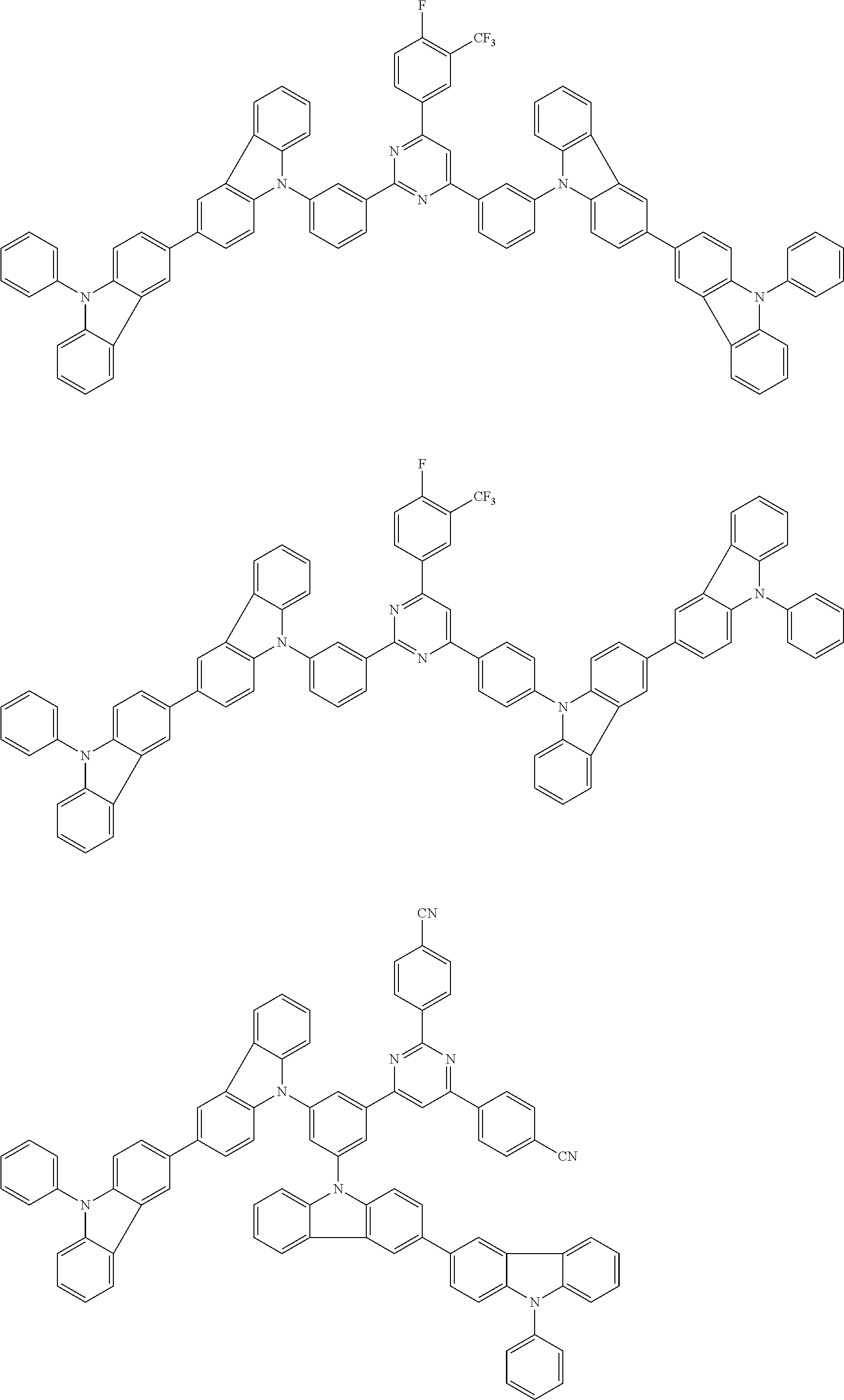 [SMILES: Fc1ccc(-c2cc(-c3cccc(-n4c5ccccc5c5cc(-c6ccc7c(c6)c6ccccc6n7-c6ccccc6)ccc54)c3)nc(-c3cccc(-n4c5ccccc5c5cc(-c6ccc7c(c6)c6ccccc6n7-c6ccccc6)ccc54)c3)n2)cc1C(F)(F)F ; Fc1ccc(-c2cc(-c3ccc(-n4c5ccccc5c5cc(-c6ccc7c(c6)c6ccccc6n7-c6ccccc6)ccc54)cc3)nc(-c3cccc(-n4c5ccccc5c5cc(-c6ccc7c(c6)c6ccccc6n7-c6ccccc6)ccc54)c3)n2)cc1C(F)(F)F ; N#Cc1ccc(-c2cc(-c3cc(-n4c5ccccc5c5cc(-c6ccc7c(c6)c6ccccc6n7-c6ccccc6)ccc54)cc(-n4c5ccccc5c5cc(-c6ccc7c(c6)c6ccccc6n7-c6ccccc6)ccc54)c3)nc(-c3ccc(C#N)cc3)n2)cc1]

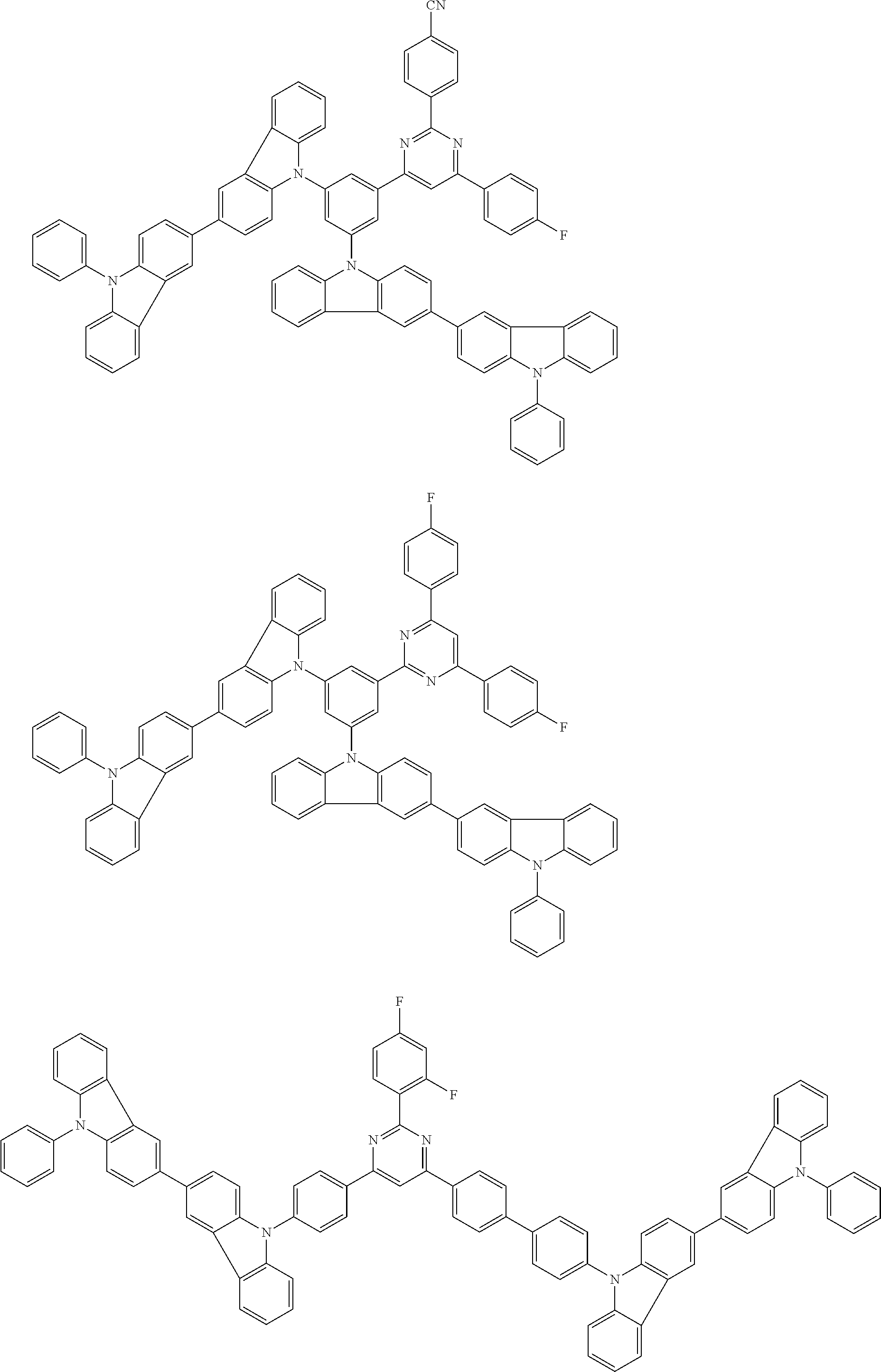 [SMILES: N#Cc1ccc(-c2nc(-c3ccc(F)cc3)cc(-c3cc(-n4c5ccccc5c5cc(-c6ccc7c(c6)c6ccccc6n7-c6ccccc6)ccc54)cc(-n4c5ccccc5c5cc(-c6ccc7c(c6)c6ccccc6n7-c6ccccc6)ccc54)c3)n2)cc1 ; Fc1ccc(-c2cc(-c3ccc(F)cc3)nc(-c3cc(-n4c5ccccc5c5cc(-c6ccc7c(c6)c6ccccc6n7-c6ccccc6)ccc54)cc(-n4c5ccccc5c5cc(-c6ccc7c(c6)c6ccccc6n7-c6ccccc6)ccc54)c3)n2)cc1 ; Fc1ccc(-c2nc(-c3ccc(-c4ccc(-n5c6ccccc6c6cc(-c7ccc8c(c7)c7ccccc7n8-c7ccccc7)ccc65)cc4)cc3)cc(-c3ccc(-n4c5ccccc5c5cc(-c6ccc7c(c6)c6ccccc6n7-c6ccccc6)ccc54)cc3)n2)c(F)c1]

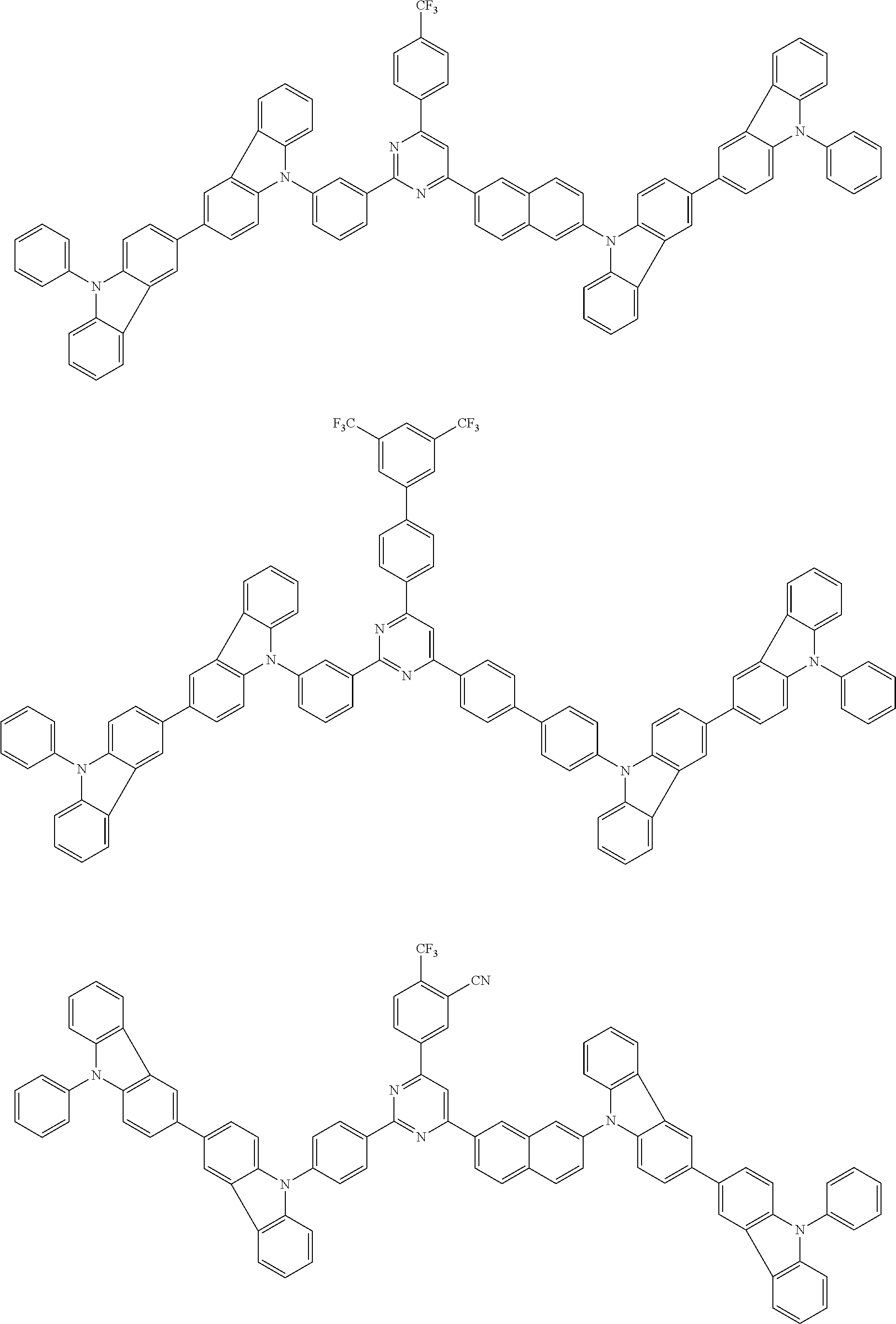

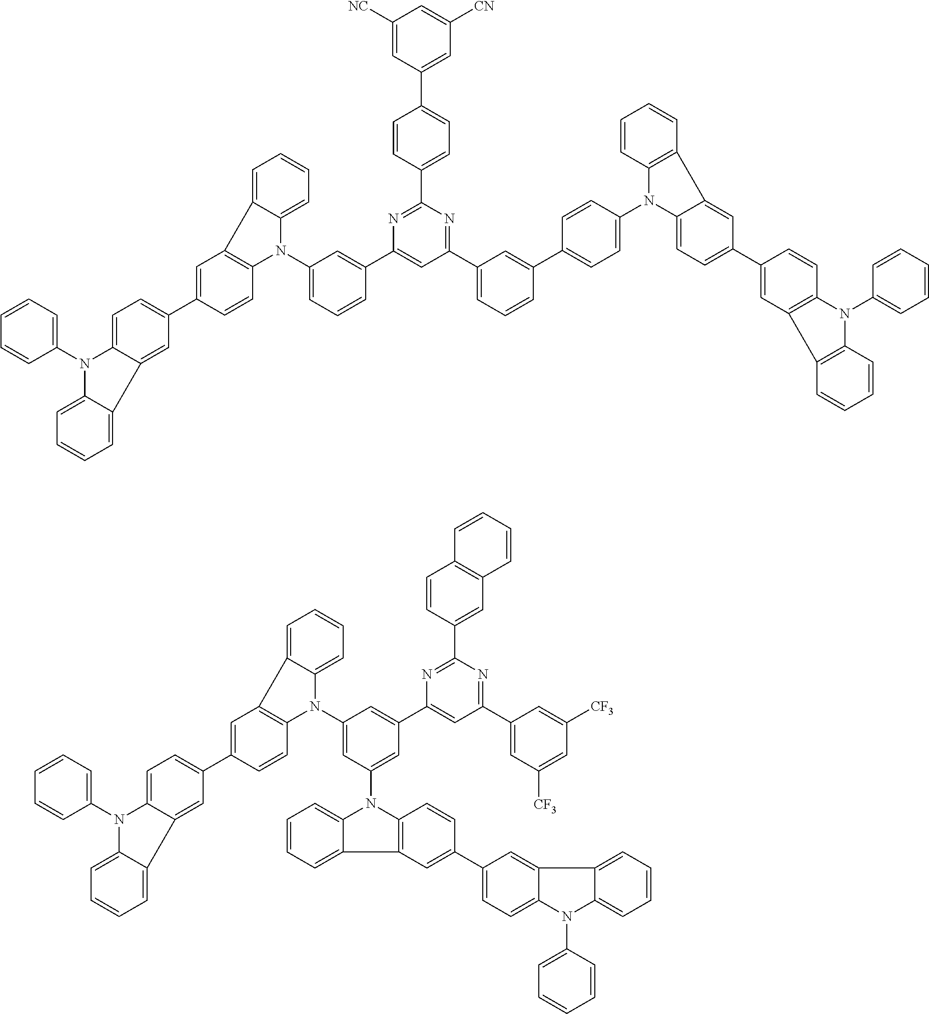 [SMILES: N#Cc1cc(C#N)cc(-c2ccc(-c3nc(-c4cccc(-c5ccc(-n6c7ccccc7c7cc(-c8ccc9c(c8)c8ccccc8n9-c8ccccc8)ccc76)cc5)c4)cc(-c4cccc(-n5c6ccccc6c6cc(-c7ccc8c(c7)c7ccccc7n8-c7ccccc7)ccc65)c4)n3)cc2)c1 ; FC(F)(F)c1cc(-c2cc(-c3cc(-n4c5ccccc5c5cc(-c6ccc7c(c6)c6ccccc6n7-c6ccccc6)ccc54)cc(-n4c5ccccc5c5cc(-c6ccc7c(c6)c6ccccc6n7-c6ccccc6)ccc54)c3)nc(-c3ccc4ccccc4c3)n2)cc(C(F)(F)F)c1]

### Material for Organic Electroluminescence Device, Solution of Material for Organic Electroluminescence Device and Organic Electroluminescence Device

The material for an organic EL device of the invention comprises the aromatic heterocyclic derivative described above.

The solution of a material for an organic EL device of the invention comprises the aromatic heterocyclic derivative dissolved in a solvent.

The organic EL device of the invention comprises a cathode, an anode, and one or more organic thin film layers which are disposed between the cathode and the anode and comprise a light emitting layer, wherein at least one layer of the one or more organic thin film layers comprises the aromatic heterocyclic derivative of the invention.

The aromatic heterocyclic derivative of the invention is used in at least one layer of the organic thin film layers of an organic EL device. Particularly, when using the aromatic heterocyclic derivative of the invention in a light emitting layer as a host material, in an electron transporting layer, or in a hole transporting layer, it is expected that the emission efficiency is increased and the lifetime is prolonged.

### First Embodiment

Examples of the structure of a multi-layered type organic EL device are shown below:

(1) anode/hole transporting layer (hole injecting layer)/light emitting layer/cathode;
(2) anode/light emitting layer/electron transporting layer (electron injecting layer)/cathode;
(3) anode/hole transporting layer (hole injecting layer)/light emitting layer/electron transporting layer (electron injecting layer)/cathode; and
(4) anode/hole transporting layer (hole injecting layer)/light emitting layer/hole blocking layer/electron transporting layer (electron injecting layer)/cathode.

The light emitting layer preferably comprises the aromatic heterocyclic derivative of the invention as a host material. In another preferred embodiment, the light emitting layer comprises a host material and a phosphorescent material, and the host material is the aromatic heterocyclic derivative of the invention and the lowest excited triplet energy is 1.6 to 3.2 eV, preferably 2.2 to 3.2 eV, and more preferably 2.5 to 3.2 eV. The “triplet energy” used herein is the energy difference between the lowest excited triplet state and the ground state.

The aromatic heterocyclic derivative of the invention also serves as a host material which is combinedly used with a phosphorescent material or an electron transporting material which is combinedly used with a phosphorescent material.

Since the phosphorescent quantum yield is high and the external quantum yield of emission device is further improved, the phosphorescent material is preferably a compound comprising iridium (Ir), osmium (Os), ruthenium (Ru) or platinum (Pt), more preferably a metal complex, such as an iridium complex, an osmium complex, a ruthenium complex and a platinum complex, still more preferably an iridium complex and a platinum complex, and most preferably an ortho-metallated complex of a metal atom selected from iridium, osmium (Os) and platinum (Pt). Examples of the metal complex, such as an iridium complex, an osmium complex, a ruthenium complex and a platinum complex are shown below.

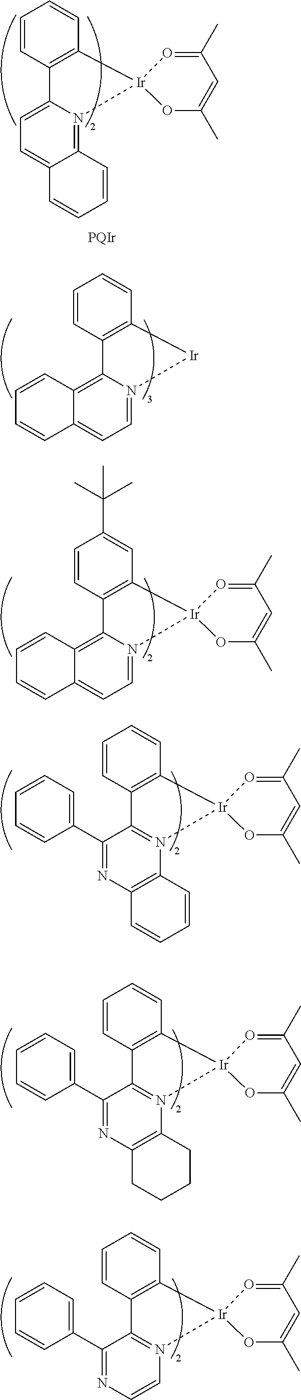

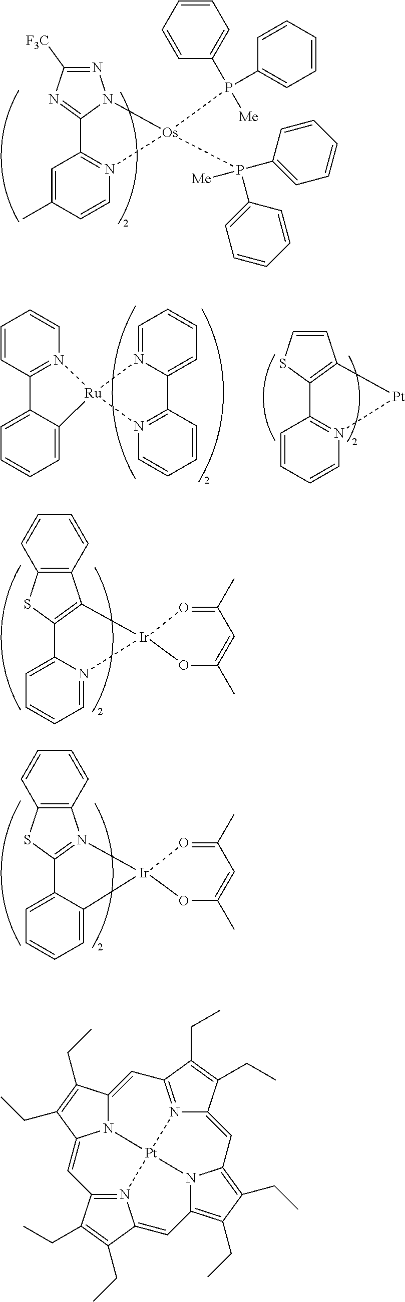

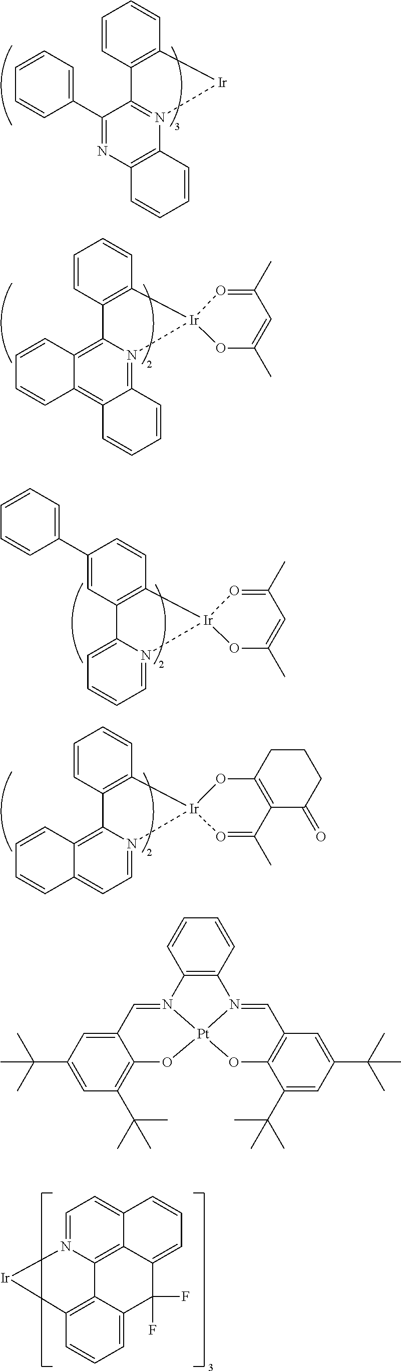

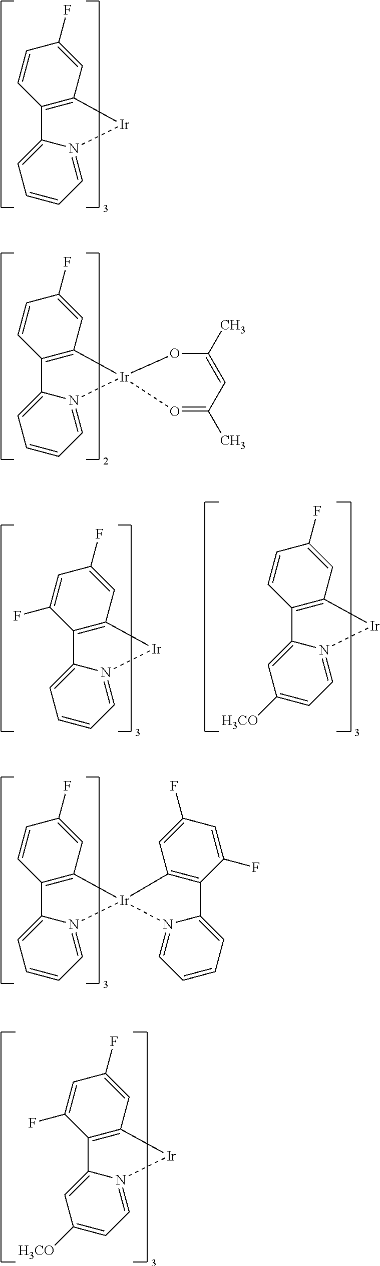

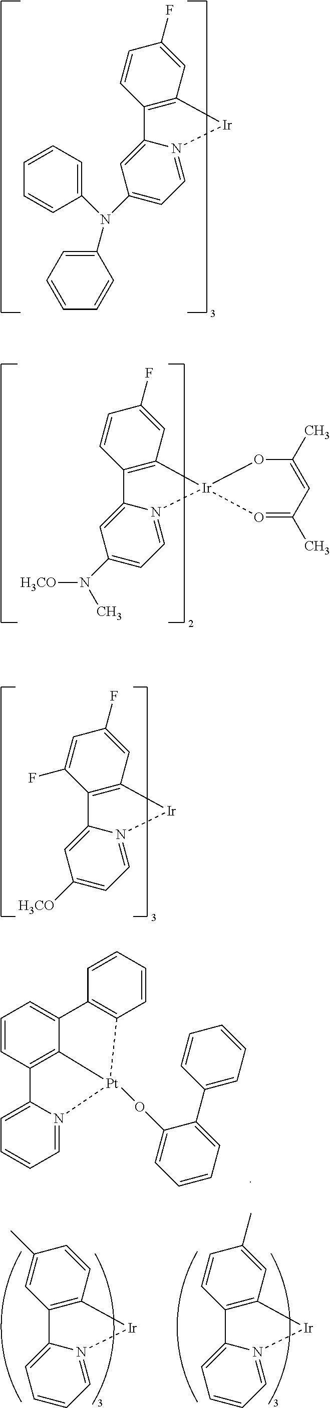

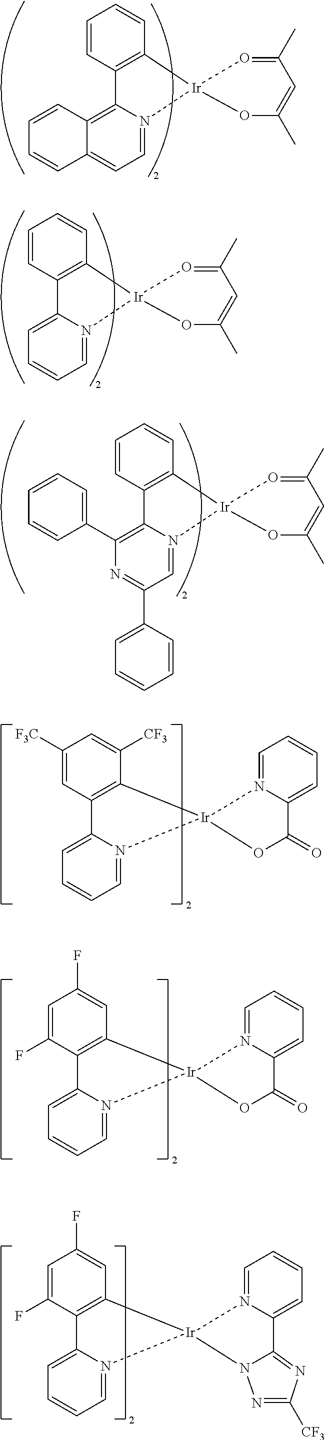

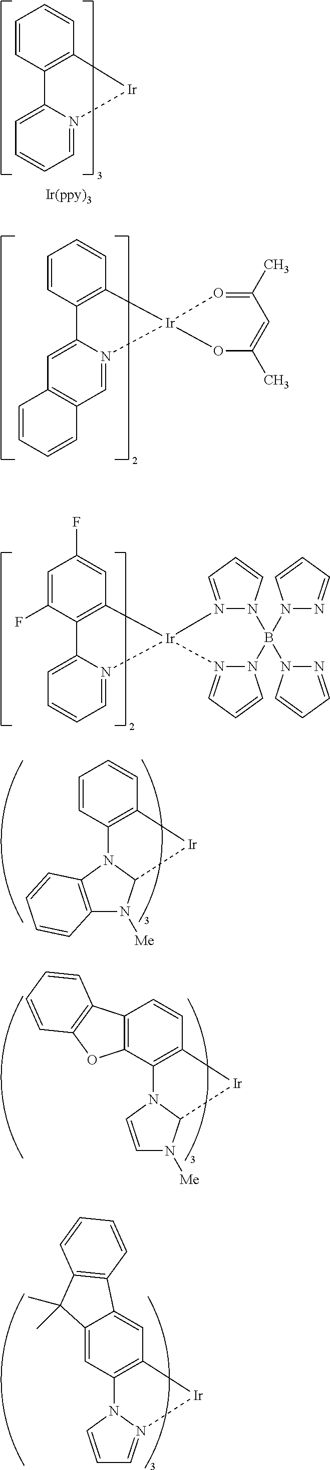

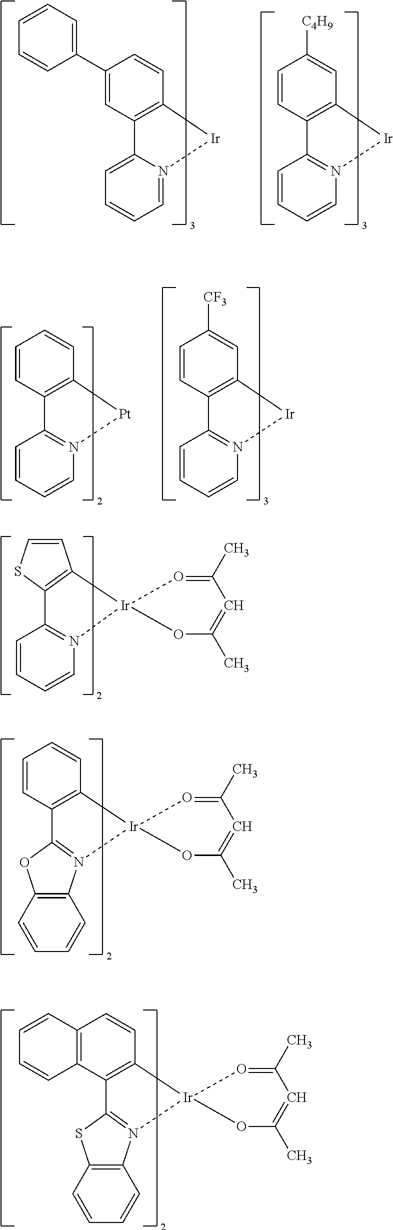

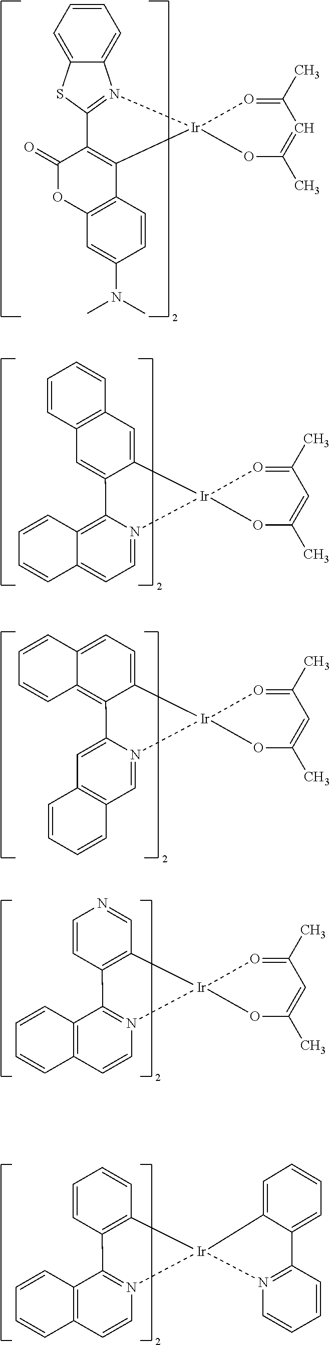

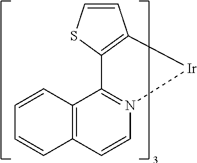

In another preferred embodiment, the light emitting layer comprises a host material, a phosphorescent material and further a metal complex emitting light with a peak wavelength of 450 nm or more and 750 nm or less.

The organic EL device of the present invention preferably comprises an reducing dopant in an interfacial region between the cathode and the organic thin film layer, for example, an electron injecting layer and a light emitting layer. The reducing dopant may be at least one selected from an alkali metal, an alkali metal complex, an alkali metal compound, an alkaline earth metal, an alkaline earth metal complex, an alkaline earth metal compound, a rare earth metal, a rare earth metal complex, and a rare earth metal compound.

Examples of the alkali metal include those having a work function of 2.9 eV or less, preferably Na (work function: 2.36 eV), K (work function: 2.28 eV), Rb (work function: 2.16 eV), and Cs (work function: 1.95 eV). with K, Rb, and Cs being more preferred, Rb and Cs being still more preferred, and Cs being most preferred.

Examples of the alkaline earth metal include those having a work function of 2.9 eV or less, preferably Ca (work function: 2.9 eV), Sr (work function: 2.0 to 2.5 eV), and Ba (work function: 2.52 eV).

Examples of the rare earth metal include those having a work function of 2.9 eV or less, preferably Sc, Y, Ce, Tb, and Yb.

Preferred metals of the above are those having a high reducing ability and capable of improving the luminance and prolonging the lifetime of an organic EL device by the addition to an electron injecting region in a relatively small amount.

Examples of the alkali metal compound include alkali oxide, such as Li₂O, Cs₂O, K₂O, and alkali halide, such as LiF, NaF, CsF, and KF, with LiF, Li₂O, and NaF being preferred.

Examples of the alkaline earth metal compound include BaO, SrO, CaO, and mixture thereof, such as BaₘSr₁₋ₘO (0<m<1) and BaₘCa₁₋ₘO (0<m<1), with BaO, SrO, and CaO being preferred.

Examples of the rare earth metal compound include YbF₃, ScF₃, ScO₃, Y₂O₃, Ce₂O₃, GdF₃, and TbF₃, with YbF₃, ScF₃, and TbF₃ being preferred.

Examples of the alkali metal complex, alkaline earth metal complex, and rare earth metal complex are not particularly limited as long as containing at least one metal ion selected from alkali metal ions, alkaline earth metal ions, and rare earth metal ions, respectively. The ligand is preferably, but not limited to, quinolinol, benzoquinolinol, acridinol, phenanthridinol, hydroxyphenyloxazole, hydroxyphenylthiazole, hydroxydiaryloxadiazole, hydroxydiarylthiadiazole, hydroxyphenylpyridine, hydroxyphenylbenzimidazole, hydroxybenzotriazole, hydroxyfulborane, bipyridyl, phenanthroline, phthalocyanine, porphyrin, cyclopentadiene, β-diketones, azomethines, and derivatives thereof.

The reducing dopant is added to the interfacial region preferably into a layered form or an island form. The reducing dopant is added preferably by co-depositing with an organic material, such as a light emitting material and an electron injecting material, to form the interfacial region by a resistance heating deposition method, thereby dispersing the reducing dopant into the organic material. The dispersion concentration expressed by the molar ratio of the organic material and the reducing dopant is 100:1 to 1:100 and preferably 5:1 to 1:5

When the reducing dopant is formed into a layered form, an interfacial organic layer is formed from a light emitting material or an electron injecting material in a layered form, and then, the reducing dopant alone is deposited by a resistance heating deposition method into a layer having a thickness of preferably 0.1 to 15 nm.

When the reducing dopant is formed into an island form, an interfacial organic layer is formed from a light emitting material or an electron injecting material in an island form, and then, the reducing dopant alone is deposited by a resistance heating deposition method into a form of island having a thickness of preferably 0.05 to 1 nm.

When the organic EL device of the invention comprises an electron injecting layer between the light emitting layer and the cathode, the electron transporting material for forming the electron injecting layer is preferably an aromatic heterocyclic compound having one or more heteroatoms in its molecule and particularly preferably a nitrogen-containing ring derivative.

The nitrogen-containing ring derivative is preferably, for example, a metal chelate complex of a nitrogen-containing ring represented by formula (A):

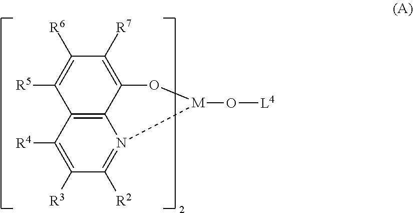

wherein each of R² to R⁷ independently represents a hydrogen atom, a halogen atom, an amino group, a hydrocarbon group having 1 to 40 carbon atoms, an alkoxy group, an aryloxy group, an alkoxycarbonyl group, or a heterocyclic group, each being optionally substituted;

M is aluminum (Al), gallium (Ga), or indium (In), with In being preferred; and

L⁴ is a group represented by formula (A′) or (A″):

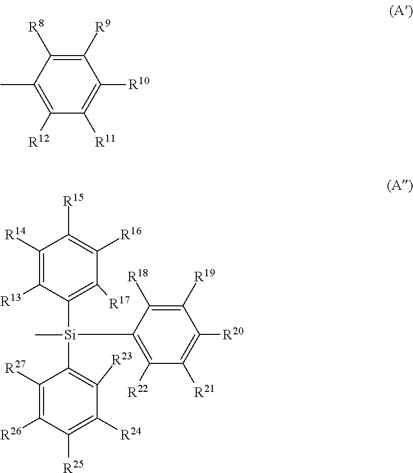

wherein each R⁸ to R¹² independently represents a hydrogen atom or a substituted or unsubstituted hydrocarbon group having 1 to 40 carbon atoms, and the adjacent two groups may form a ring structure; and each of R¹³ to R²⁷ independently represents a hydrogen atom or a substituted or unsubstituted hydrocarbon group having 1 to 40 carbon atoms, and the adjacent two groups may form a ring structure.

The nitrogen-containing ring derivative may include a nitrogen-containing compound which is not a metal complex, for example, a compound having a 5- or 6-membered ring which has a skeleton represented by formula (a) or having a structure represented by formula (b):

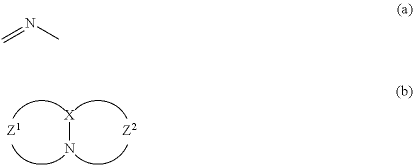

in formula (b), X is a carbon atom or a nitrogen atom and each of Z₁ and Z₂ independently represents a group of atoms for completing the nitrogen-containing heterocyclic ring.

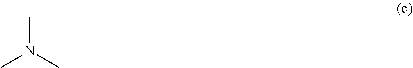

The nitrogen-containing ring derivative is preferably an organic compound which has a nitrogen-containing aromatic polycyclic ring comprising a 5-membered ring or a 6-membered ring. If two or more nitrogen atoms are included, the nitrogen-containing aromatic polycyclic compound preferably comprises a skeleton of a combination of (a) and (b) or a combination of (a) and (c).

The nitrogen-containing group of the nitrogen-containing heterocyclic derivative is selected, for example, from those shown below:

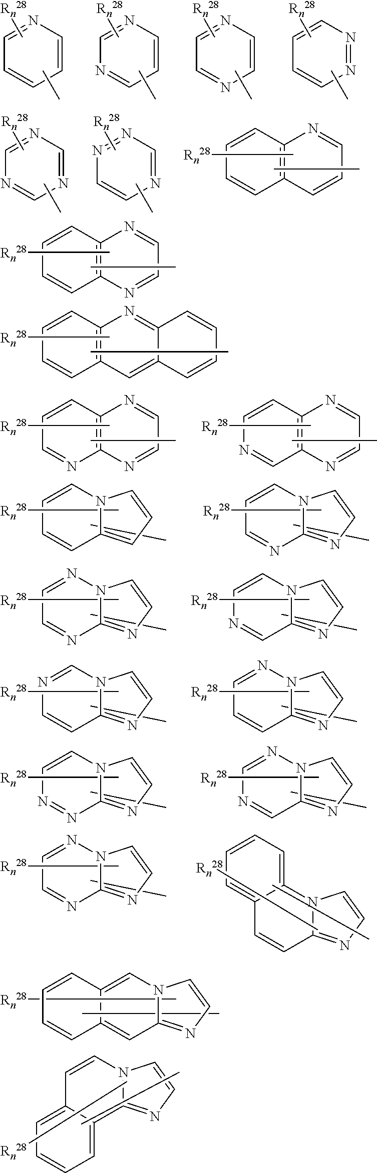

wherein R²⁸ is an aryl group having 6 to 40 carbon atoms, a heteroaryl group having 3 to 40 carbon atoms, an alkyl group having 1 to 20 carbon atoms, or an alkoxy group having 1 to 20 carbon atoms; and n is an integer of 0 to 5. When n is an integer of 2 or more, groups R²⁸ may be the same or different.

Another preferred compound is a nitrogen-containing heterocyclic derivative represented by the following formula:

HAr^{a}-L⁶-Ar^{b}—Ar^{c}

wherein HAr^{a} is a nitrogen-containing heterocyclic group having 3 to 40 carbon atoms which may be substituted; L⁶ is a single bond, an arylene group having 6 to 40 carbon atoms which may be substituted, or a heteroarylene group having 3 to 40 carbon atoms which may be substituted; Ar^{b} is a divalent aromatic hydrocarbon group having 6 to 40 carbon atoms which may be substituted; and Ar^{c} is an aryl group having 6 to 40 carbon atoms which may be substituted or a heteroaryl group having 3 to 40 carbon atoms which may be substituted.

HAr^{a} is selected, for example, from the following groups:

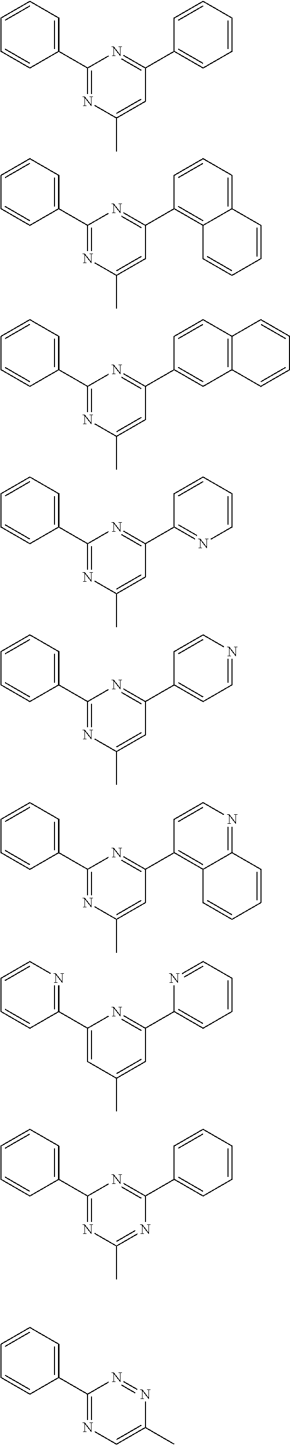

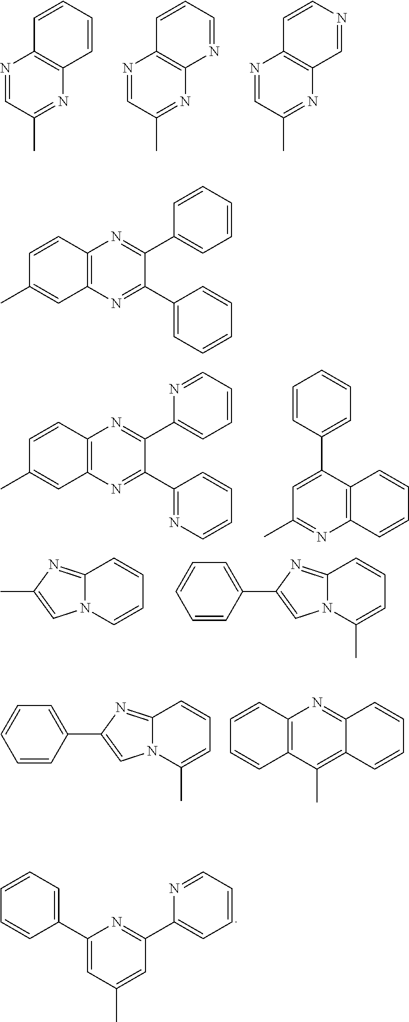

L⁶ is selected, for example, from the following groups:

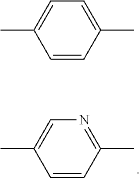

Ar^{c} is selected, for example, from the following groups:

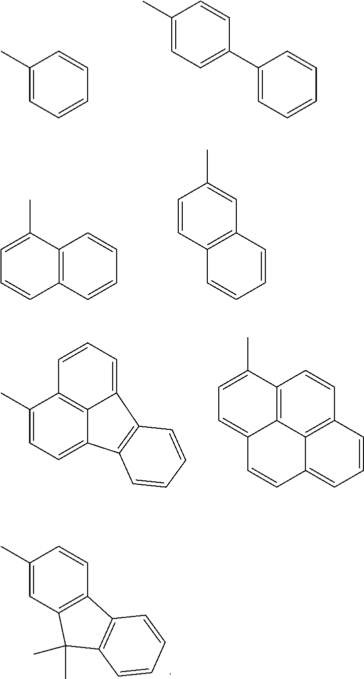

Ar^{b} is selected, for example, from the following arylanthranyl groups:

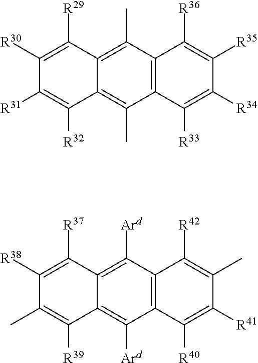

wherein each of R²⁹ to R⁴² independently represents a hydrogen atom, a halogen atom, an alkyl group having 1 to 20 carbon atoms, an alkoxy group having 1 to 20 carbon atoms, an aryloxy group having 6 to 40 carbon atoms, an aryl group having 6 to 40 carbon atoms which may be substituted, or a heteroaryl group having 3 to 40 carbon atoms which may be substituted; and Ar^{d} represents an aryl group having 6 to 40 carbon atoms which may be substituted or a heteroaryl group having 3 to 40 carbon atoms which may be substituted.

A nitrogen-containing heterocyclic derivative including Ar^{b} wherein R²⁹ to R³⁶ are all hydrogen atoms is preferred.

In addition, the following compound (see JP 9-3448A) is preferably used:

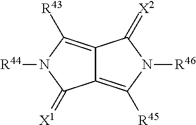

wherein each of R⁴³ to R⁴⁶ independently represents a hydrogen atom, a substituted or unsubstituted aliphatic group, a substituted or unsubstituted alicyclic group, a substituted or unsubstituted aromatic carbocyclic group, or a substituted or unsubstituted heterocyclic group; and each of X¹ and X² independently represents an oxygen atom, a sulfur atom, or a dicyanomethylene group.

In addition, the following compound (see JP 2000-173774A) is preferably used:

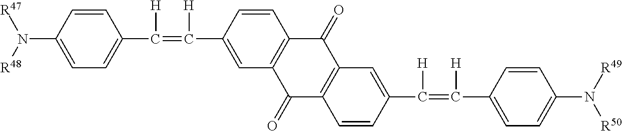

wherein R⁴⁷, R⁴⁸, R⁴⁹, and R⁵⁰ may be the same or different and each represents the following aryl group:

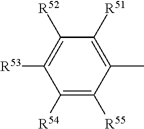

wherein R⁵¹, R⁵², R⁵³, R⁵⁴, and R⁵⁵ may be the same or different and each represents a hydrogen atom and at least one of them may be a saturated or unsaturated alkoxyl group, an alkyl group, an amino group, or an alkylamino group.

Further, a polymer constituting the nitrogen-containing heterocyclic group or the nitrogen-containing heterocyclic derivative is also usable.

The electron transporting layer preferably comprises a nitrogen-containing heterocyclic derivative, particularly a nitrogen-containing 5-membered ring derivative. Examples of the nitrogen-containing 5-membered ring include an imidazole ring, a triazole ring, a tetrazole ring, an oxadiazole ring, a thiadiazole ring, an oxatriazole ring, and a thiatriazole ring. Examples of the nitrogen-containing 5-membered ring derivative include a benzimidazole ring, a benzotriazole ring, a pyridinoimidazole ring, a pyrimidinoimidazole ring, and a pyridazinoimidazole ring.

The electron transporting layer preferably comprises at least one of the nitrogen-containing heterocyclic derivatives represented by formulae (201) to (203):

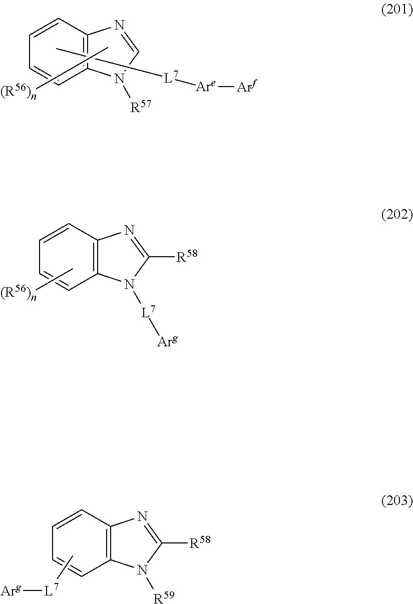

wherein:

R⁵⁶ represents a hydrogen atom, an aryl group having 6 to 60 carbon atoms which may be substituted, a pyridyl group which may be substituted, a quinolyl group which may be substituted, an alkyl group having 1 to 20 carbon atoms which may be substituted, or an alkoxy group having 1 to 20 carbon atoms which may be substituted;

n represents an integer of 0 to 4;

R⁵⁷ represents an aryl group having 6 to 60 carbon atoms which may be substituted, a pyridyl group which may be substituted, a quinolyl group which may be substituted, an alkyl group having 1 to 20 carbon atoms which may be substituted, or an alkoxy group having 1 to 20 carbon atoms which may be substituted;

each of R⁵⁸ and R⁵⁹ independently represents a hydrogen atom, an aryl group having 6 to 60 carbon atoms which may be substituted, a pyridyl group which may be substituted, a quinolyl group which may be substituted, an alkyl group having 1 to 20 carbon atoms which may be substituted, or an alkoxy group having 1 to 20 carbon atoms which may be substituted;

L⁷ represents a single bond, an arylene group having 6 to 60 carbon atoms which may be substituted, a pyridinylene group which may be substituted, a quinolinylene group which may be substituted, or a fluorenylene group which may be substituted;

Ar^{e} represents an arylene group having 6 to 60 carbon atoms which may be substituted, a pyridinylene group which may be substituted, or a quinolinylene group which may be substituted;

Ar^{f} represents a hydrogen atom, an aryl group having 6 to 60 carbon atoms which may be substituted, a pyridyl group which may be substituted, a quinolyl group which may be substituted, an alkyl group having 1 to 20 carbon atoms which may be substituted, or an alkoxy group having 1 to 20 carbon atoms which may be substituted; and

Ar^{g} represents an aryl group having 6 to 60 carbon atoms which may be substituted, a pyridyl group which may be substituted, a quinolyl group which may be substituted, an alkyl group having 1 to 20 carbon atoms which may be substituted, an alkoxy group having 1 to 20 carbon atoms which may be substituted, or a group represented by —Ar^{e}—Ar^{f}, wherein Ar^{e} and Ar^{f} are as defined above.

In addition to the aromatic heterocyclic derivative of the invention, the electron injecting layer and the electron transporting layer may comprise a compound which combinedly includes an electron deficient nitrogen-containing 5- or 6-membered ring skeleton and a skeleton selected from a substituted or unsubstituted indole skeleton, a substituted or unsubstituted carbazole skeleton, and a substituted or unsubstituted azacarbazole skeleton. Preferred examples of the electron deficient nitrogen-containing 5- or 6-membered ring skeleton include skeletons of pyridine, pyrimidine, pyrazine, triazine, triazole, oxadiazole, pyrazole, imidazole, quinoxaline, and pyrrole and molecular skeletons in which the above skeletons are fused together, for example, benzimidazole and imidazopyridine. The combinations between the skeletons of pyridine, pyrimidine, pyrazine, and triazine with the skeletons of carbazole, indole, azacarbazole, and quinoxaline are preferred. These skeletons may be substituted or unsubstituted.

The electron injecting layer and the electron transporting layer may be a single-layered structure comprising one or two of the materials mentioned above or a multi-layered structure in which layers may comprise the same material or different materials. The material for these layers preferably comprises a π-electron deficient nitrogen-containing heterocyclic group.

In addition to the nitrogen-containing ring derivative, an inorganic compound, such as an insulating material and a semiconductor, is preferably used in the electron injecting layer. The electron injecting layer comprising the insulating material or the semiconductor effectively prevents the leak of electric current to enhance the electron injecting ability.

The insulating material is preferably at least one metal compound selected from the group consisting of alkali metal chalcogenides, alkaline earth metal chalcogenides, alkali metal halides and alkaline earth metal halides. The alkali metal chalcogenide. mentioned above are preferred because the electron injecting ability of the electron injecting layer is further enhanced. Examples of preferred alkali metal chalcogenide include Li₂O, K₂O, Na₂S, Na₂Se and Na₂O, and examples of preferred alkaline earth metal chalcogenide include CaO, BaO, SrO, BeO, BaS and CaSe. Examples of preferred alkali metal halide include LiF, NaF, KF, LiCl, KCl and NaCl. Examples of the alkaline earth metal halide include fluorides, such as CaF₂, BaF₂, SrF₂, MgF₂ and BeF₂, and halides other than fluorides.

Examples of the semiconductor include oxides, nitrides or oxynitrides of at least one element selected from the group consisting of Ba, Ca, Sr, Yb, Al, Ga, In, Li, Na, Cd, Mg, Si, Ta, Sb and Zn. These semiconductors may be used alone or in combination of two or more. The inorganic compound included in the electron injecting layer preferably forms a microcrystalline or amorphous insulating thin film. If the electron injecting layer is formed from such an insulating thin film, the pixel defects, such as dark spots, can be decreased because a more uniform thin film is formed. Examples of such inorganic compound include the alkali metal chalcogenide, the alkaline earth metal chalcogenide, the alkali metal halide and the alkaline earth metal halide.

The reducing dopant mentioned above is preferably used in the electron injecting layer.

The thickness of the electron injecting layer or the electron transporting layer is preferably 1 to 100 nm, although not particularly limited thereto.

The hole injecting layer and the hole transporting layer (inclusive of a hole injecting/transporting layer) preferably comprise an aromatic amine compound, for example, an aromatic amine derivative represented by formula (I):

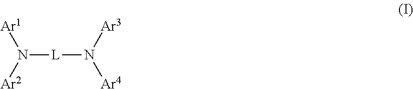

wherein:

each of Ar¹ to Ar⁴ represents a substituted or unsubstituted aryl group having 6 to 50 ring carbon atoms or a substituted or unsubstituted heteroaryl group having 5 to 50 ring atoms;

L represents a linking group, for example, a substituted or unsubstituted arylene group having 6 to 50 ring carbon atoms, a substituted or unsubstituted heteroarylene group having 5 to 50 ring atoms, or a divalent group obtained by bonding two or more arylene groups or heteroarylene groups via a single bond, an ether group, a thioether group, an alkylene group having 1 to 20 carbon atoms, an alkenylene group having 2 to 20 carbon atoms, or an amino group.

An aromatic amine represented by formula (II) is also suitable for forming the hole injecting layer and the hole transporting layer:

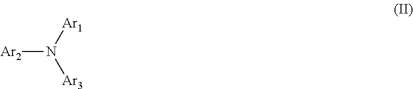

wherein Ar₁ to Ar₃ are the same as defined above with respect to Ar¹ to Ar⁴ of formula (I).

The aromatic heterocyclic derivative of the invention transports both holes and electrons, and therefore, usable in any of the hole injecting layer, the hole transporting layer, the electron injecting layer and the electron transporting layer.

The anode of the organic EL device injects holes to the hole transporting layer or the light emitting layer, and an anode having a work function of 4.5 eV or more is effective. Examples of the material for anode include indium tin oxide alloy (ITO), tin oxide (NESA), gold, silver, platinum, and copper. In view of facilitating the injection of electrons to the electron injecting layer or the light emitting layer, the cathode is preferably formed from a material having a small work function. Examples of the material for cathode include, but not limited to, indium, aluminum, magnesium, magnesium-indium alloy, magnesium-aluminum alloy, aluminum-lithium alloy, aluminum-scandium-lithium alloy, and magnesium-silver alloy.

The method for forming each layer of the organic EL device of the invention is not particularly limited, and the film-forming method, such as a known vapor deposition method and spin coating method are usable. The organic thin film layer comprising the aromatic heterocyclic derivative of the invention can be formed by forming a solution of the aromatic heterocyclic derivative in a solvent into a film by a known coating method, such as a dipping method, a spin-coating method, a casting method, a bar-coating method, and a roll-coating method.

The thickness of each organic thin film layer in the organic EL device is not particularly limited and preferably several nanometers to 1 μm because an excessively small thickness may cause defects such as pin holes and an excessively large thickness may require a high driving voltage.

The layer comprising the aromatic heterocyclic derivative of the invention, particularly the light emitting layer, is preferably formed by forming a solution containing the aromatic heterocyclic derivative and another material, such as a dopant, into a film.

Examples of the film-forming method include known coating methods, and preferably a spin coating method, a casting method, a microgravure coating method, a gravure coating method, a bar coating method, a roll coating method, a slit coating method, a wire bar coating method, a dip coating method, a spray coating method, a screen printing method, a flexographic printing method, an off-set printing method, an ink-jet printing method, and a nozzle printing method. When a pattern is formed, a screen printing method, a flexographic printing method, an off-set printing method, and an ink-jet printing method are preferred. The film formation by these methods can be made under the conditions well known by a skilled person.

After coating, the solvent is removed by heating (up to 250° C.) and drying under vacuum, and the irradiation of light and the high temperature heating exceeding 250° C. for polymerization reaction are not needed. Therefore, the deterioration of the device in its performance due to the irradiation of light and the high temperature heating exceeding 250° C. can be prevented.

The film-forming solution contains at least one aromatic heterocyclic derivative of the invention and may further contain another material, for example, a hole transporting material, an electron transporting material, a light emitting material, an acceptor material, a solvent, and an additive such, as a stabilizer.

The film-forming solution may contain an additive for controlling the viscosity and/or surface tension, for example, a thickener (high molecular weight compounds, poor solvents of the polymer of the invention, etc.), a viscosity depressant (low molecular weight compounds, etc.) and a surfactant. In addition, an antioxidant not adversely affecting the performance of the organic EL device, for example, a phenol antioxidant and a phosphine antioxidant, may be included so as to improve the storage stability.

The content of the aromatic heterocyclic derivative in the film-forming solution is preferably 0.1 to 15% by mass and more preferably 0.5 to 10% by mass based on the total of the film-forming solution.

Examples of the high molecular weight compound usable as the thickener include an insulating resin and a copolymer thereof, such as polystyrene, polycarbonate, polyarylate, polyester, polyamide, polyurethane, polysulfone, polymethyl methacrylate, polymethyl acrylate, and cellulose; a photoconductive resin, such as poly-N-vinylcarbazole and polysilane; and a conductive resin, such as polythiophene and polypyrrole.

Examples of the solvent for the film-forming solution include a chlorine-containing solvent such as chloroform, methylene chloride, 1,2-dichloroethane, 1,1,2-trichloroethane, chlorobenzene, and o-dichlorobenzene; an ether solvent such as tetrahydrofuran, dioxane, dioxolane, and anisole; an aromatic hydrocarbon solvent such as toluene and xylene; an aliphatic hydrocarbon solvent such as cyclohexane, methylcyclohexane, n-pentane, n-hexane, n-heptane, n-octane, n-nonane, and n-decane; a ketone solvent such as acetone, methyl ethyl ketone, cyclohexanone, benzophenone, and acetophenone; an ester solvent such as ethyl acetate, butyl acetate, ethyl cellosolve acetate, methyl benzoate, and phenyl acetate; a polyhydric alcohol and its derivatives such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, and 1,2-hexanediol; an alcoholic solvent such as methanol, ethanol, propanol, isopropanol, and cyclohexanol; a sulfoxide solvent such as dimethyl sulfoxide; and an amide solvent such as N-methyl-2-pyrrolidone and N,N-dimethylformamide. These solvents may be used alone or in combination of two or more.

Of the above solvents, in view of solubility, uniform film formation, viscosity, etc., preferred are the aromatic hydrocarbon solvent, the ether solvent, the aliphatic hydrocarbon solvent, the ester solvent and the ketone solvent, and more preferred are toluene, xylene, ethylbenzene, diethylbenzene, trimethylbenzene, n-propylbenzene, isopropylbenzene, n-butylbenzene, isobutylbenzene, 5-butylbenzene, n-hexylbenzene, cyclohexylbenzene, 1-methylnaphthalene, tetralin, 1,3-dioxane, 1,4-dioxane, 1,3-dioxolane, anisole, ethoxybenzene, cyclohexane, bicyclohexyl, cyclohexenylcyclohexanone, n-heptylcyclohexane, n-hexylcyclohexane, decalin, methyl benzoate, cyclohexanone, 2-propylcyclohexanone, 2-heptanone, 3-heptanone, 4-heptanone, 2-octanone, 2-nonanone, 2-decanone, dicyclohexyl ketone, acetophenone, and benzophenone.

### Second Embodiment

The organic EL device of this embodiment is a tandem device comprising at least two light emitting layers or at least two units each comprising a light emitting layer.

In such an organic EL device, for example, a charge generating layer (“CGL”) may be interposed between two units to provide an electron transporting zone to each unit.

The structure of the tandem device are shown below:

(11) anode/hole injecting/transporting layer/phosphorescent light emitting layer/charge generating layer/fluorescent light emitting layer/electron injecting/transporting layer/cathode; and
(12) anode/hole injecting/transporting layer/fluorescent light emitting layer/electron injecting/transporting layer/charge generating layer/phosphorescent light emitting layer/cathode.

In these organic EL devices, the aromatic heterocyclic derivative of the invention and the phosphorescent material described in the first embodiment can be used in the phosphorescent light emitting layer. With such a phosphorescent light emitting layer, the emission efficiency of the organic EL device and the device lifetime are further improved. The anode, the hole injecting/transporting layer, the electron injecting/transporting layer, and the cathode can be formed by using the materials described in the first embodiment. Each of the fluorescent light emitting layer and the charge generating layer can be formed by using a known material.

### Third Embodiment

The organic EL device of this embodiment comprises two or more light emitting layers and a charge blocking layer between any of two light emitting layers. The preferred structures for the organic EL device of this embodiment are described in JP 4134280B, US 2007/0273270A1, and WO 2008/023623A1.

For example, in a structure in which an anode, a first light emitting layer, a charge blocking layer, a second light emitting layer, and a cathode are laminated in this order, an electron transporting zone including a charge blocking layer is further disposed between the second light emitting layer and the cathode to prevent the diffusion of triplet excitons. The charge blocking layer used herein is a layer which controls the carrier injection into a light emitting layer and controls the carrier balance between electrons and holes in the light emitting layer by utilizing the energy barrier in HOMO level or LUMO level with those of the adjacent light emitting layer.

Examples of the structure are shown below:

(21) anode/hole injecting/transporting layer/first light emitting layer/charge blocking layer/second light emitting layer/electron injecting/transporting layer/cathode; and
(22) anode/hole injecting/transporting layer/first light emitting layer/charge blocking layer/second light emitting layer/third light emitting layer/electron injecting/transporting layer/cathode.

The aromatic heterocyclic derivative of the invention and the phosphorescent material described in the first embodiment are usable in at least one of the first light emitting layer, the second light emitting layer, and the third light emitting layer, thereby further improving the emission efficiency of the organic EL device and the device lifetime.

A white-emitting device can be obtained, for example, by allowing a first light emitting layer to emit red light, allowing a second light emitting layer to emit green light, and allowing a third light emitting layer to emit blue light. Such an organic EL device is useful as a flat light source for lighting and backlight.

The anode, the hole injecting/transporting layer, the electron injecting/transporting layer, and the cathode can be formed by using the materials described in the first embodiment. The charge blocking layer can be formed by using a known material.

### EXAMPLES

The present invention will be described below in more detail with reference to the examples. However, it should be noted that the scope of the present invention is not limited thereto.

### Example 1

### (1) Synthesis of Compound H-1

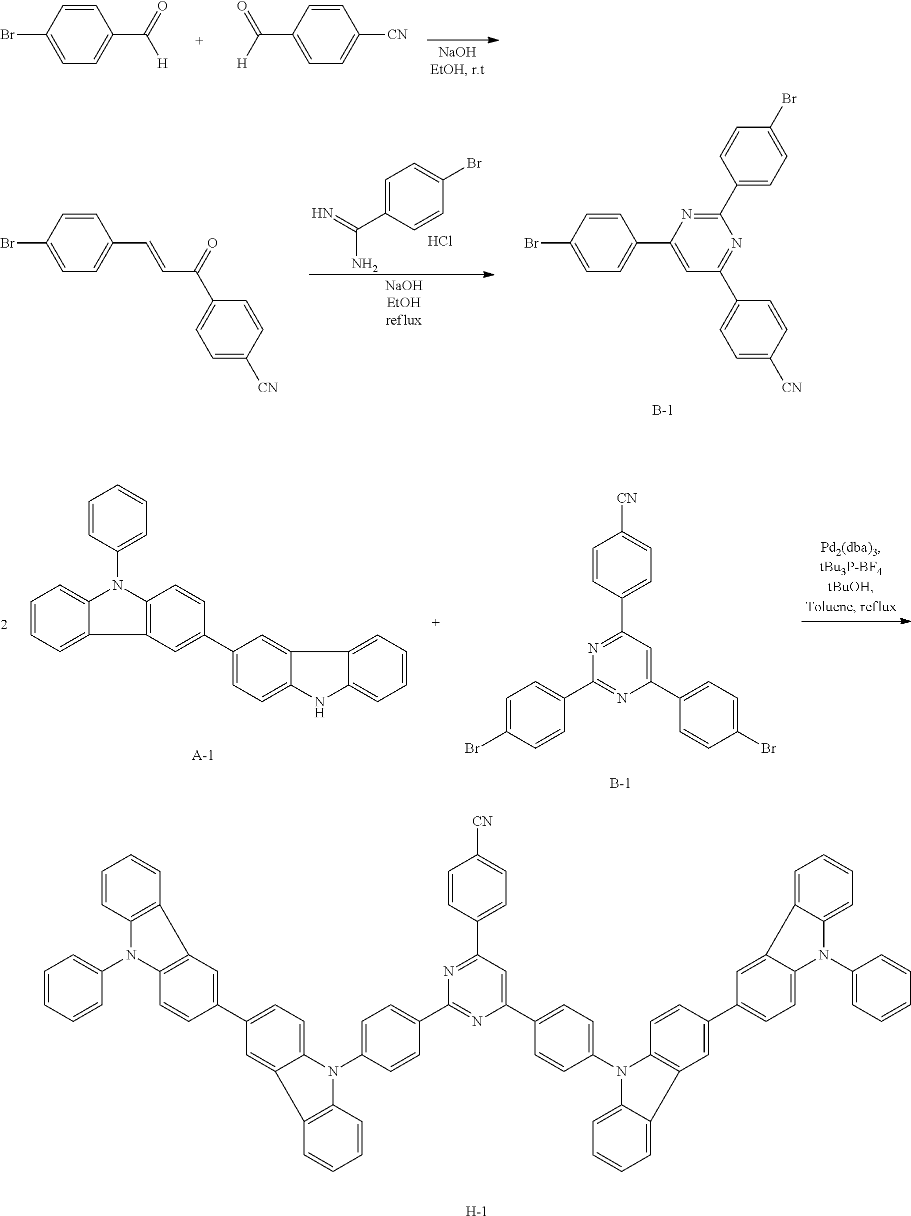

Into a solution of 4-bromobenzaldehyde (7.40 g, 40 mmol) and 4′-cyanoacetophenone (5.80 g, 40 mmol) in ethanol (80 mL), sodium hydroxide (0.16 g, 4 mmol) was added, and the resultant solution was stirred for 8 h at room temperature. After adding 4-bromobenzamidine hydrochloride (4.71 g, 20 mmol) and sodium hydroxide (1.60 g, 40 mmol), ethanol (40 mL) was further added. The reaction was allowed to proceed for 8 h while refluxing under heating. The generated white powder was collected by filtration, washed with ethanol until the filtrate became colorless, further washed with water and then ethanol, and vacuum-dried to obtain a pyrimidine intermediate B-1 (9.33 g, yield: 95%).

In an argon atmosphere, a mixture obtained by successively mixing the bicarbazolyl intermediate A-1 (2.57 g, 6.3 mmol), the pyrimidine intermediate B-1 (1.47 g, 3.0 mmol), tris(dibenzylideneacetone)dipalladium (0.055 g, 0.06 mmol), tri-t-butylphosphonium tetrafluoroborate (0.070 g, 0.24 mmol), t-butoxysodium (0.87 g, 9.0 mmol), and dry toluene (60 mL) was refluxed for 12 h under heating.

After cooling the reaction solution to room temperature, the insolubles were removed by filtration, and the organic solvent was evaporated off under reduced pressure. The obtained residue was purified by silica gel column chromatography to obtain H-1 (2.82 g, yield: 82%).

The obtained compound was analyzed by HPLC (High Performance Liquid Chromatography), FD-MS (Field Desorption ionization-Mass Spectrometry), and ¹H-NMR. The results are shown below.

HPLC: 99.2% purity

FD-MS: calcd for C83H51N7=1145.42,

found m/z=1145 (M+, 100), 1146 (92)

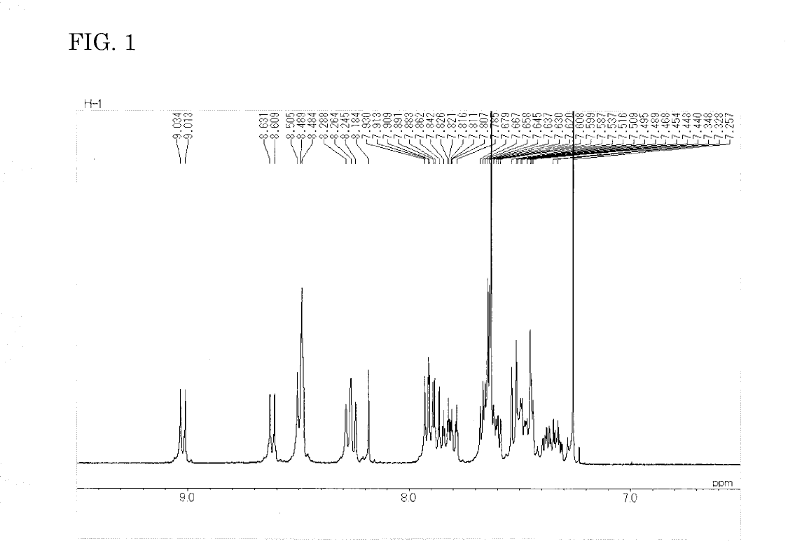

¹H-NMR (400 MHz, CDCl₃, TMS): FIG. 1

σ 7.3-7.7 (m, 26H), 7.75-7.95 (m, 10H), 8.18 (s, 1H), 8.26 (t, 4H), 8.45-8.55 (d+s, 6H), 8.62 (d, 2H), 9.02 (d, 2H)

### (2) Production of Organic EL Device

### Preparation of Base Substrate

PEDOT:PSS (Clevious AI4083 manufactured by H.C. Starck) was diluted by two times with isopropyl alcohol and spin-coated on ITO substrate for 60 s at a rotation speed of 4000 rpm. After spin-coating, the portion corresponding to the extraction electrode was wiped off with ultra-pure water. Then, the obtained product was baked in air for 30 min on a hot plate at 200° C.

### Preparation of Ink for Light Emitting Layer

A 2.5 wt % ink for a light emitting layer was prepared by ultrasonically dissolving 20 mg of the compound H-1 and 5 mg of the following complex in a desired amount of toluene.

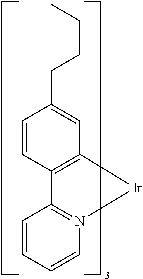

### Formation of Light Emitting Layer by Coating

The ink for a light emitting layer was spin-coated for 60 s at a rotation speed of 3000 rpm. After spin-coating, the portion corresponding to the extraction electrode was wiped off with toluene. Then, the obtained product was dried for 30 min under heating on a hot plate at 100° C. to produce a substrate laminated with a coating film. The film-forming operations were all conducted in a glove box under a nitrogen atmosphere.

### Vapor Deposition and Sealing

On the substrate laminated with a coating film, the following compound as an electron transporting material, lithium fluoride, and aluminum were vapor deposited into films, each having a thickness of 20 nm, 1 nm, and 80 nm, respectively. The device having the vapor-deposited films was sealed with a bored glass in a nitrogen atmosphere to produce a device for evaluation.

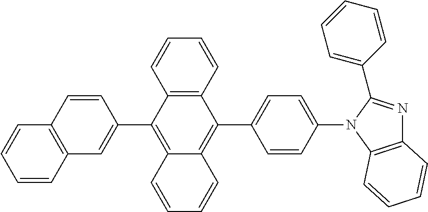 [SMILES: c1ccc(-c2nc3ccccc3n2-c2ccc(-c3c4ccccc4c(-c4ccc5ccccc5c4)c4ccccc34)cc2)cc1]

### (3) Evaluation of EL Performance

The device for evaluation was evaluated for its EL performance. The electroluminescence with an emission peak wavelength of 590 nm was observed.

The organic EL device was measured for the voltage (V) at a current density of 1 mA/cm², the efficiency (cd/A), and a lifetime until the luminance was reduced to 90% of the initial value (LT90, 5200 cd/m² of initial luminance) while allowing the device to emit light under a direct current drive. The measured results are shown in Table 1.

### Example 2

### (1) Synthesis of Compound H-2

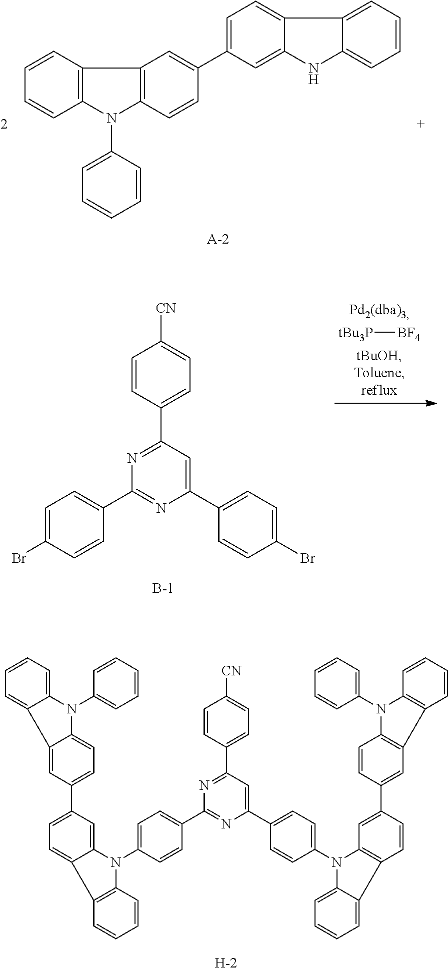

In an argon atmosphere, a mixture obtained by successively mixing the bicarbazolyl intermediate A-2 (2.57 g, 6.3 mmol), the pyrimidine intermediate B-1 (1.47 g, 3.0 mmol), tris(dibenzylideneacetone)dipalladium (0.055 g, 0.06 mmol), tri-t-butylphosphonium tetrafluoroborate (0.070 g, 0.24 mmol), t-butoxysodium (0.87 g, 9.0 mmol), and dry toluene (60 mL) was refluxed for 16 h under heating.

After cooling the reaction solution to room temperature, the insolubles were removed by filtration, and the organic solvent was evaporated off under reduced pressure. The obtained residue was purified by silica gel column chromatography to obtain H-2 (2.61 g, yield: 76%).

The obtained compound was analyzed by HPLC, FD-MS, and ¹H-NMR. The results are shown below.

HPLC: 98.6% purity

FD-MS: calcd for C83H51N7=1145.42,

found m/z=1145 (M+, 100), 1146 (92)

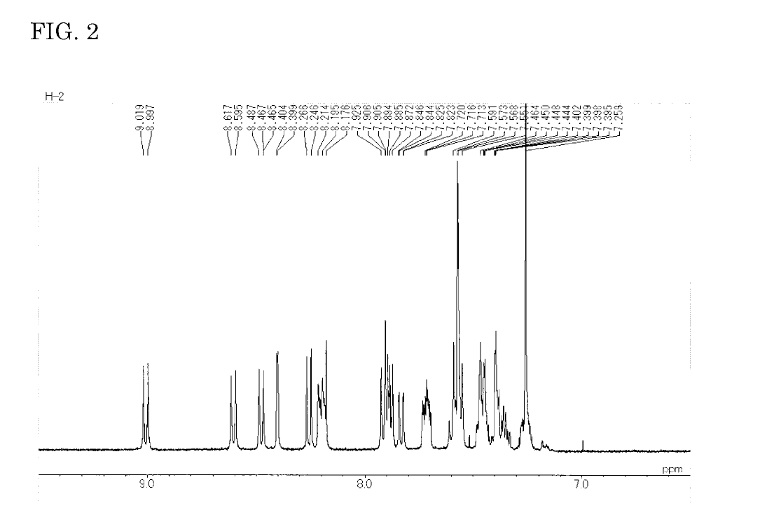

¹H-NMR (400 MHz, CDCl₃, TMS): FIG. 2

σ 7.3-7.6 (m, 24H), 7.65-7.75 (m, 4H), 7.84 (d, 2H), 7.85-7.95 (m, 6H), 8.15-8.25 (m, 5H), 8.26 (d, 2H), 8.40 (s, 2H), 8.48 (d, 2H), 8.61 (d, 2H), 9.01 (d, 2H)

### (2) Production of Organic EL Device

An organic EL device was produced in the same manner as in Example 1 except for using the compound H-2 in place of the compound H-1.

### (3) Evaluation of EL Performance

The evaluation was made in the same manner as in Example 1. The evaluation results are shown in Table 1.

### Example 3

### (1) Synthesis of Compound H-3

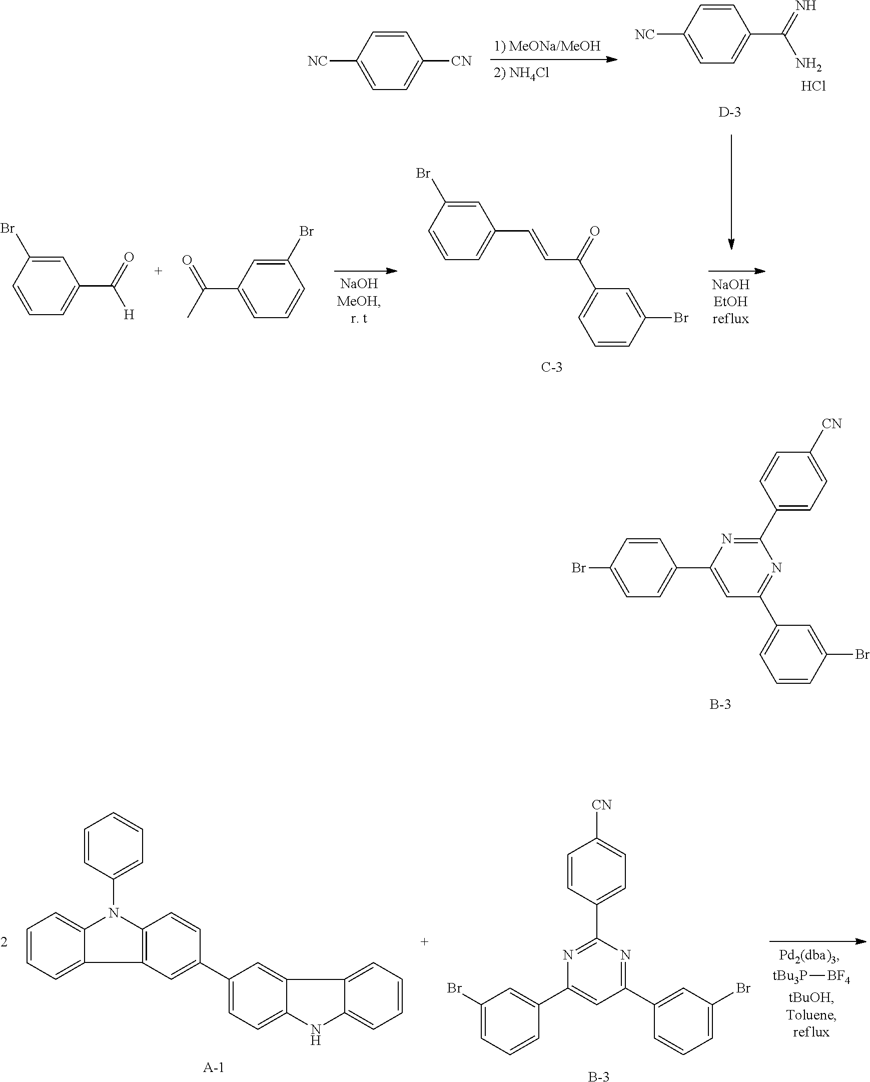

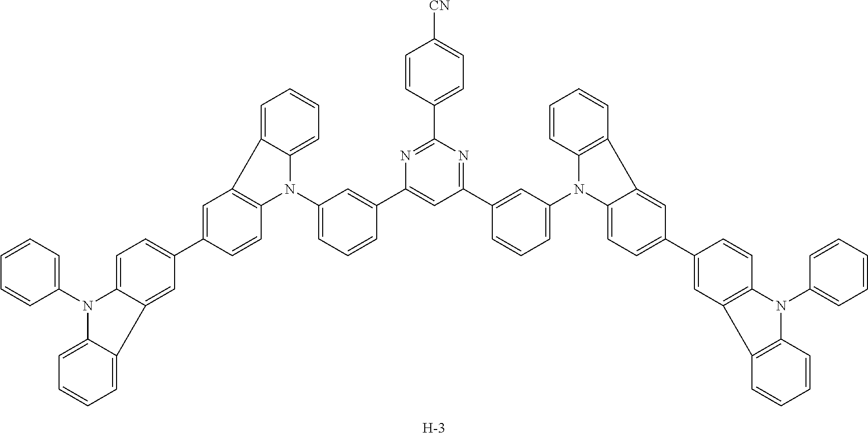

Into a solution of 3-bromobenzaldehyde (7.40 g, 40 mmol) and 3′-bromoacetophenone (7.96 g, 40 mmol) in methanol (80 mL), sodium hydroxide (0.16 g, 4 mmol) was added, and the resultant solution was stirred for 8 h at room temperature. The precipitated chalcone intermediate C3 was collected by filtration and dried. Into a solution of terephthalonitrile (2.56 g, 20 mmol) in 200 mL of dry methanol, 2 mL of a 1 N methanol solution of sodium methoxide was added, and the resultant solution was stirred for 2 h at room temperature. After adding ammonium chloride (1.18 g, 22 mmol), the solution was further stirred for 4 h at room temperature. The solvent was evaporated off under reduced pressure to obtain the benzamidine hydrochloride intermediate D-3, which was dissolved in ethanol (120 mL). After adding the chalcone intermediate C-3 synthesized above and sodium hydroxide (1.60 g, 40 mmol) to the obtained solution, the reaction was allowed to proceed for 8 h while refluxing under heating. The generated white powder was collected by filtration, washed with ethanol until the filtrate became colorless, further washed with water and then ethanol, and vacuum-dried to obtain the aimed pyrimidine intermediate B-3 (7.37 g, yield: 75%).

In an argon atmosphere, a mixture obtained by successively mixing the bicarbazolyl intermediate A-1 (2.57 g, 6.3 mmol), the pyrimidine intermediate B-3 (1.47 g, 3.0 mmol), tris(dibenzylideneacetone)dipalladium (0.055 g, 0.06 mmol), tri-t-butylphosphonium tetrafluoroborate (0.070 g, 0.24 mmol), t-butoxysodium (0.87 g, 9.0 mmol), and dry toluene (60 mL) was refluxed for 16 h under heating.

After cooling the reaction solution to room temperature, the insolubles were removed by filtration, and the organic solvent was evaporated off under reduced pressure. The obtained residue was purified by silica gel column chromatography to obtain H-3 (2.78 g, yield: 81%).

The obtained compound was analyzed by HPLC, FD-MS, and ¹H-NMR. The results are shown below.

HPLC: 98.7% purity

FD-MS: calcd for C83H51N7=1145.42,

found m/z=1145 (M+, 100), 1146 (92)

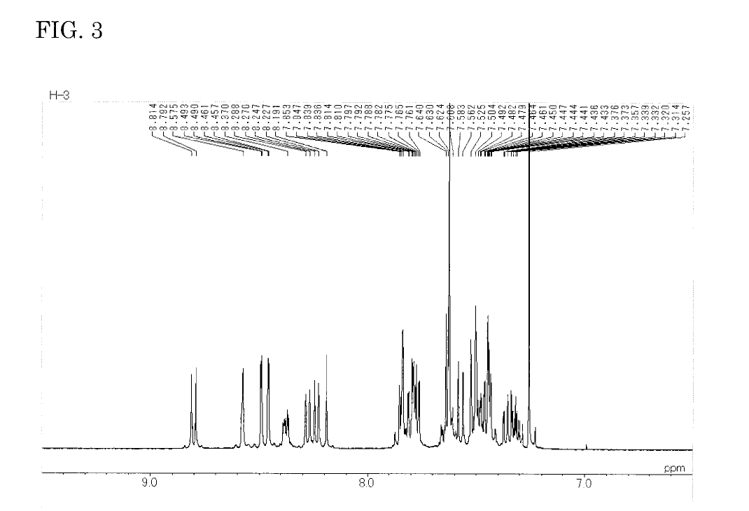

¹H-NMR (400 MHz, CDCl₃, TMS): FIG. 3

σ 7.3-7.7 (m, 26H), 7.75-7.9 (m, 10H), 8.19 (s, 1H), 8.24 (d, 2H), 8.28 (d, 2H), 8.35-8.4 (m, 2H), 8.48 (d, 4H), 8.58 (s, 2H), 8.80 (d, 2H)

### (2) Production of Organic EL Device

An organic EL device was produced in the same manner as in Example 1 except for using the compound H-3 in place of the compound H-1.

### (3) Evaluation of EL Performance

The evaluation was made in the same manner as in Example 1. The evaluation results are shown in Table 1.

### Example 4

### (1) Synthesis of Compound H-4

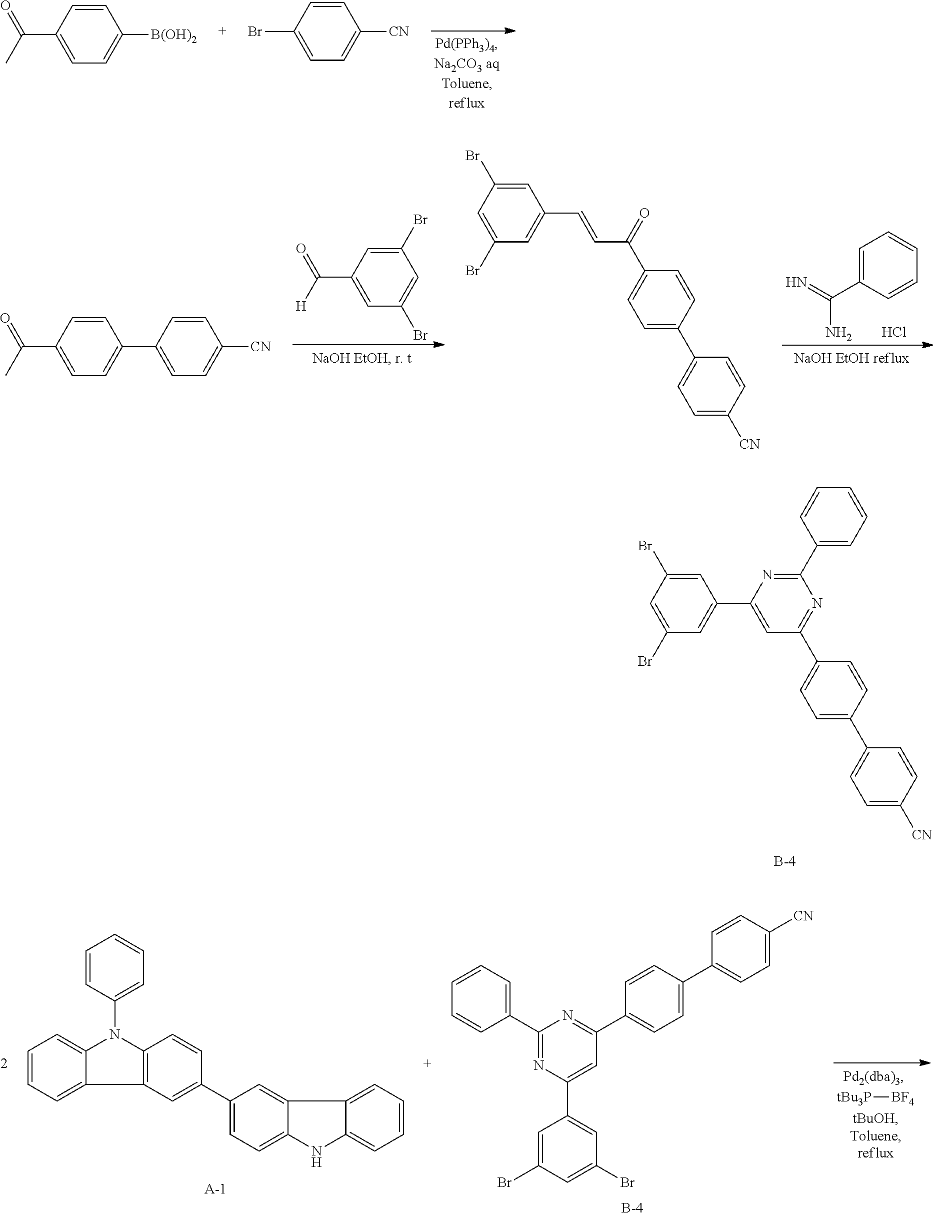

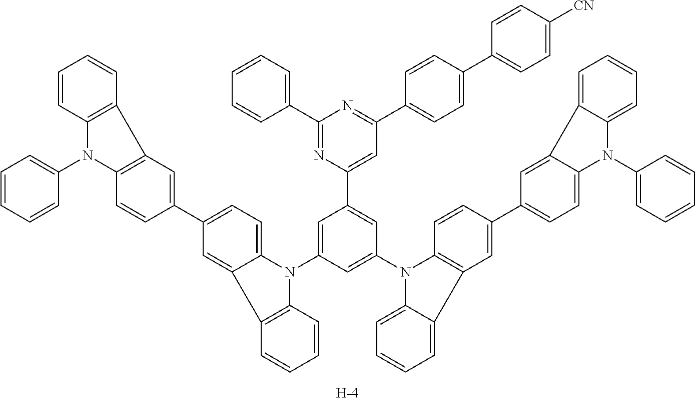

Into a solution of 4-acetyl-4′-cyanobiphenyl (8.85 g, 40 mmol) (synthesized by Suzuki coupling reaction of 4-acetylphenylboronic acid and 4-bromobenzonitrile) and 3,5-dibromobenzaldehyde (10.56 g, 40 mmol) in ethanol (80 mL), sodium hydroxide (0.16 g, 4 mmol) was added, and the resultant solution was stirred for 8 h at room temperature. After adding benzamidine hydrochloride (3.13 g, 20 mmol) and sodium hydroxide (1.60 g, 40 mmol), ethanol (40 mL) was further added. The reaction was allowed to proceed for 8 h while refluxing under heating. The generated white powder was collected by filtration, washed with ethanol until the filtrate became colorless, further washed with water and then ethanol, and vacuum-dried to obtain the pyrimidine intermediate B-4 (8.62 g, yield: 76%).

In an argon atmosphere, a mixture obtained by successively mixing the bicarbazolyl intermediate A-1 (2.57 g, 6.3 mmol), the pyrimidine intermediate B-4 (1.70 g, 3.0 mmol), tris(dibenzylideneacetone)dipalladium (0.055 g, 0.06 mmol), tri-t-butylphosphonium tetrafluoroborate (0.070 g, 0.24 mmol), t-butoxysodium (0.87 g, 9.0 mmol), and dry toluene (60 mL) was refluxed for 16 h under heating.

After cooling the reaction solution to room temperature, the insolubles were removed by filtration, and the organic solvent was evaporated off under reduced pressure. The obtained residue was purified by silica gel column chromatography to obtain H-4 (2.67 g, yield: 73%).

The obtained compound was analyzed by HPLC, FD-MS, and ¹H-NMR. The results are shown below.

HPLC: 98.4% purity

FD-MS: calcd for C89H55N7=1221.45,

found m/z=1221 (M+, 100), 1222 (97)

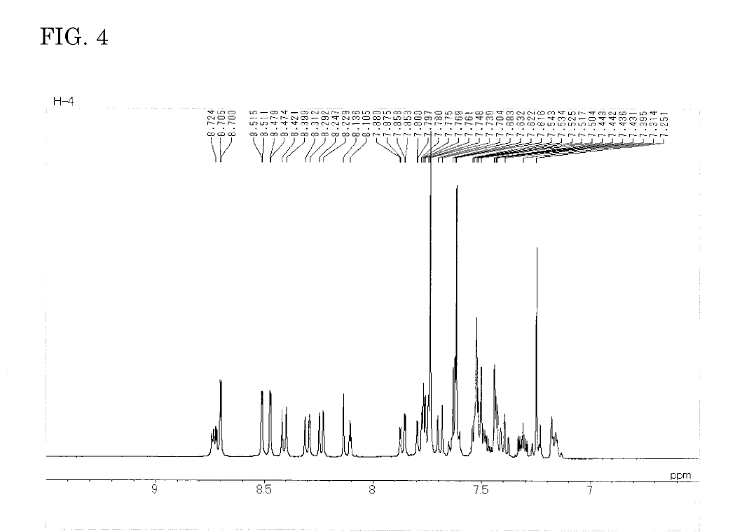

¹H-NMR (400 MHz, CDCl₃, TMS): FIG. 4

σ 7.3-7.8 (m, 37H), 7.87 (d, 2H), 8.11 (s, 1H), 8.14 (s, 1H), 8.24 (d, 2H), 8.30 (d, 2H), 8.41 (d, 2H), 8.46 (d, 4H), 8.70 (s, 2H), 8.7-8.75 (m, 2H)

### (2) Production of Organic EL Device

An organic EL device was produced in the same manner as in Example 1 except for using the compound H-4 in place of the compound H-1.

### (3) Evaluation of EL Performance

The evaluation was made in the same manner as in Example 1. The evaluation results are shown in Table 1.

### Example 5

### (1) Synthesis of Compound H-5

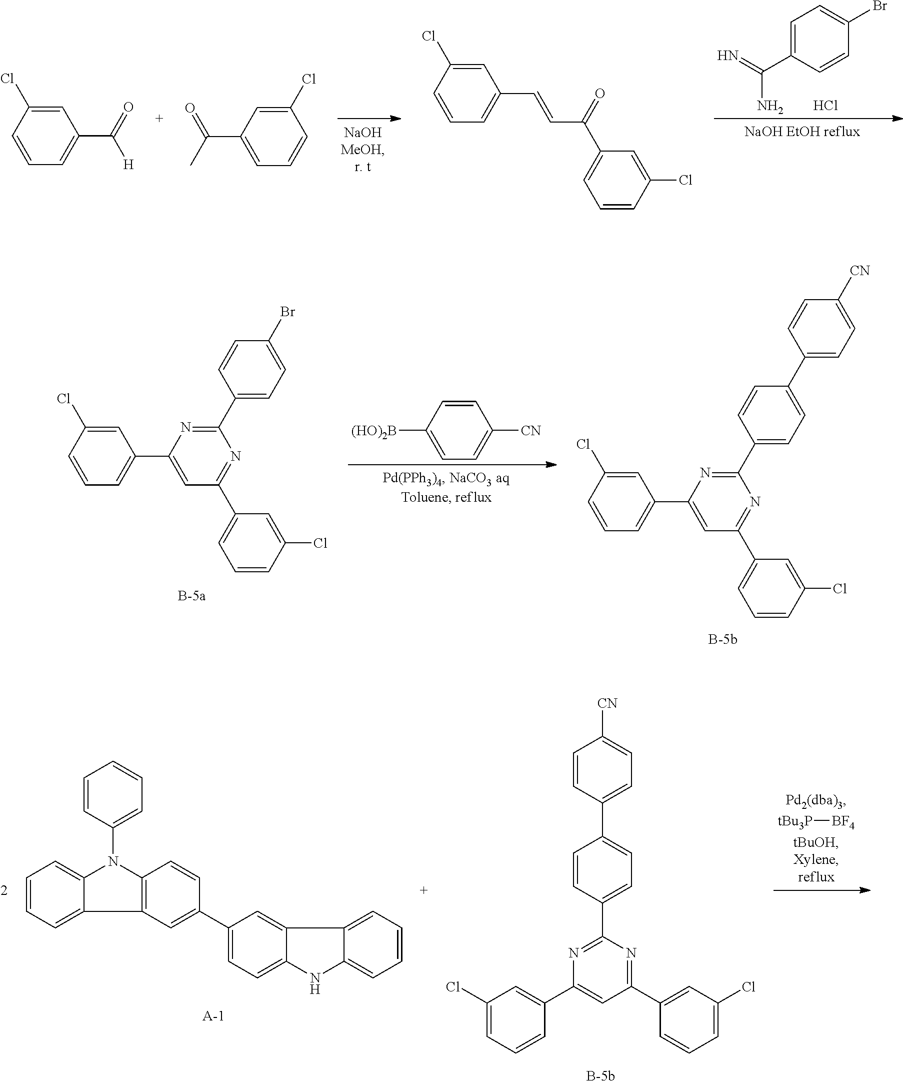

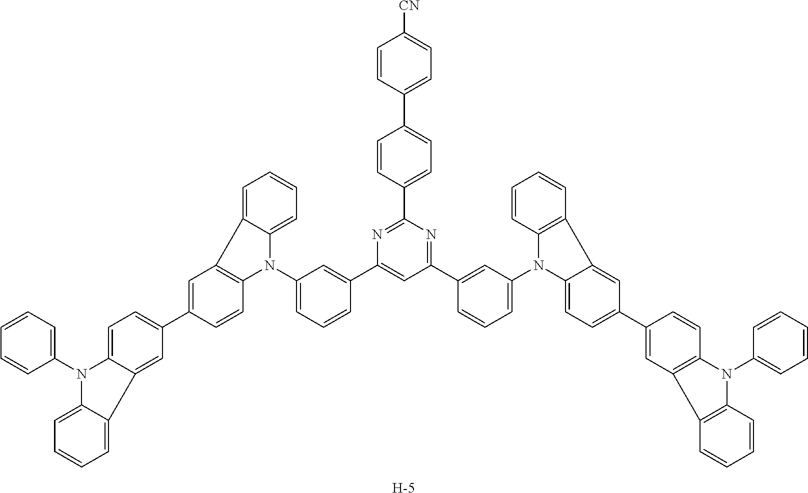

Into a solution of 3-chlorobenzaldehyde (5.62 g, 40 mmol) and 3′-chloroacetophenone (6.18 g, 40 mmol) in ethanol (80 mL), sodium hydroxide (0.16 g, 4 mmol) was added, and resultant solution was stirred for 8 h at room temperature. After adding 4-bromobenzamidine hydrochloride (4.71 g, 20 mmol) and sodium hydroxide (1.60 g, 40 mmol), ethanol (40 mL) was further added. The reaction was allowed to proceed for 8 h while refluxing under heating. The generated white powder was collected by filtration, washed with ethanol until the filtrate became colorless, further washed with water and then ethanol, and vacuum-dried to obtain the pyrimidine intermediate B-5a (3.65 g, 13.2 mmol, yield: 66%). After adding 4-cyanophenylboronic acid (2.20 g, 15 mmol), tetrakis(triphenylphosphine) palladium (346 mg, 0.3 mmol), toluene (45 mL), and a 2 M aqueous solution of sodium carbonate (22.5 mL, 45 mmol) to the obtained pyrimidine intermediate B-5a, the reaction was allowed to proceed for 8 h while refluxing under heating. After cooling the reaction solution to room temperature, the water layer was separated and removed, and the organic layer was dried over magnesium sulfate. Then, the insolubles were removed by filtration, and the organic solvent was evaporated off under reduced pressure. The obtained residue was purified by silica gel column chromatography to obtain the pyrimidine intermediate B-5b (5.18 g, yield: 82%).

In an argon atmosphere, a mixture obtained by successively mixing the bicarbazolyl intermediate A-1 (2.57 g, 6.3 mmol), the pyrimidine intermediate B-5b (1.50 g, 3.0 mmol), tris(dibenzylideneacetone)dipalladium (0.055 g, 0.06 mmol), tri-t-butylphosphonium tetrafluoroborate (0.070 g, 0.24 mmol), t-butoxysodium (0.87 g, 9.0 mmol), and dry xylene (60 mL) was refluxed for 16 h under heating.

After cooling the reaction solution to room temperature, the insolubles were removed by filtration, and the organic solvent was evaporated off under reduced pressure. The obtained residue was purified by silica gel column chromatography to obtain H-5 (2.82 g, yield: 77%).

The obtained compound was analyzed by HPLC, FD-MS, and ¹H-NMR. The results are shown below.

HPLC: 99.2% purity

FD-MS: calcd for C89H55N7=1221.45,

found m/z=1221 (M+, 100), 1222 (97)

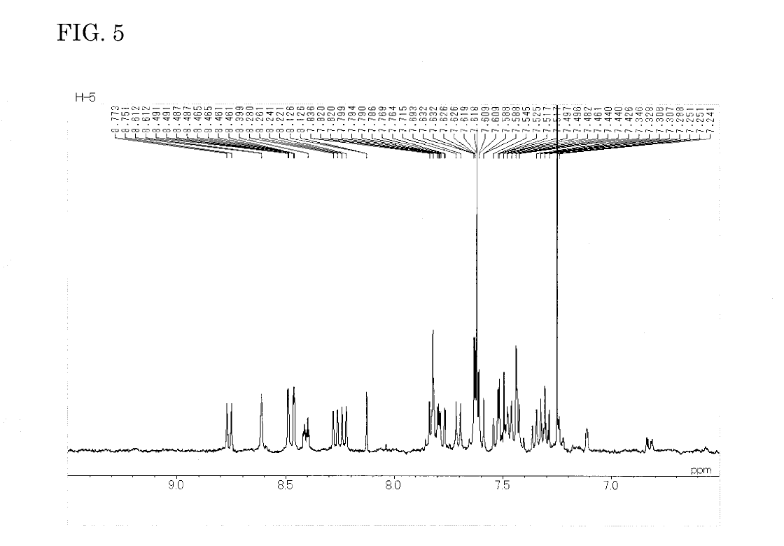

¹H-NMR (400 MHz, CDCl₃, TMS): FIG. 5

σ 7.3-7.65 (m, 30H), 7.74 (d, 2H), 7.75-7.85 (m, 8H), 8.13 (s, 1H), 8.23 (d, 2H), 8.27 (d, 2H), 8.4 (m, 2H), 8.48 (d, 4H), 8.61 (s, 2H), 8.76 (d, 2H)

### (2) Production of Organic EL Device

An organic EL device was produced in the same manner as in Example 1 except for using the compound H-5 in place of the compound H-1.

### (3) Evaluation of EL Performance

The evaluation was made in the same manner as in Example 1. The evaluation results are shown in Table 1.

### Example 6

### (1) Synthesis of Compound H-6

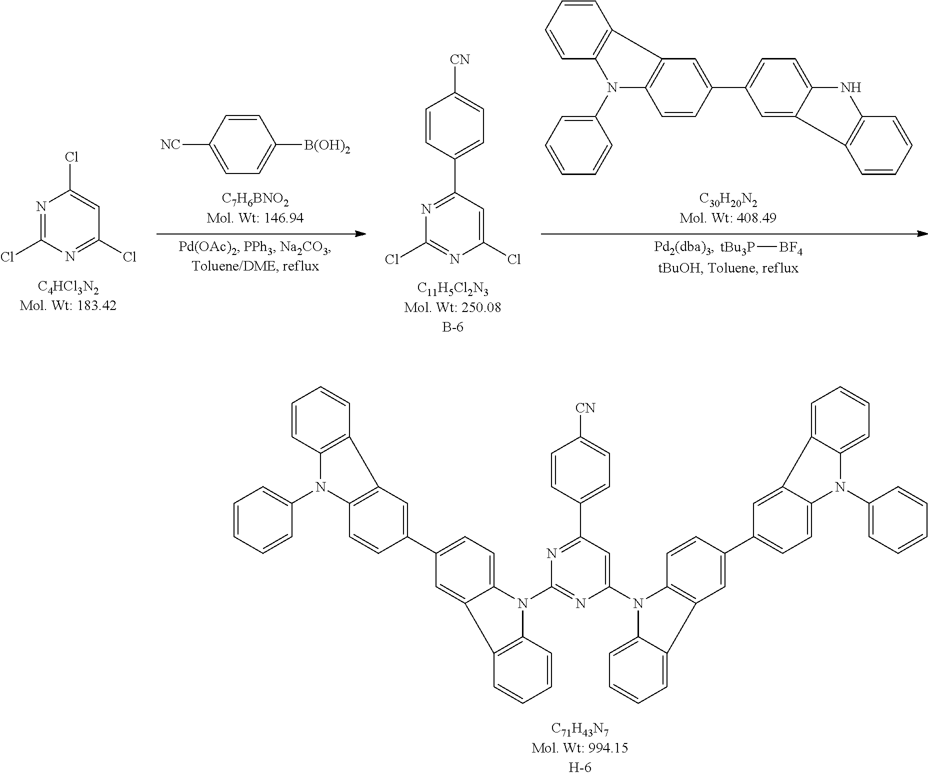

In an argon atmosphere, a mixture of trichloropyrimidine (2.29 g, 12.5 mmol), 4-cyanophenylboronic acid (1.91 g, 13 mmol), palladium acetate (70 mg, 0.32 mmol), toluene (10 mL), dimethoxy ether (30 ml), and a 2 M aqueous solution of sodium carbonate (19 mL, 37 mmol) was allowed to react for 8 h while refluxing under heating. After cooling the reaction solution to room temperature, the water layer was separated and removed, and the organic layer was dried over magnesium sulfate. Then, the insolubles were removed by filtration, and the organic solvent was evaporated off under reduced pressure. The obtained residue was purified by silica gel column chromatography to obtain the pyrimidine intermediate B-6 (2.5 g, yield: 80%).

In an argon atmosphere, a mixture obtained by successively mixing the bicarbazolyl intermediate A-1 (2.57 g, 6.3 mmol), the pyrimidine intermediate B-6 (0.75 g, 3.0 mmol), tris(dibenzylideneacetone)dipalladium (55 mg, 0.06 mmol), tri-t-butylphosphonium tetrafluoroborate (0.070 g, 0.24 mmol), t-butoxysodium (0.87 g, 9.0 mmol), and dry xylene (60 mL) was refluxed for 16 h under heating.

After cooling the reaction solution to room temperature, the insolubles were removed by filtration, and the organic solvent was evaporated off under reduced pressure The obtained residue was purified by silica gel column chromatography to obtain H-6 (2.24 g, yield: 75%).

The obtained compound was analyzed by HPLC and FD-MS. The results are shown below.

HPLC: 99.2% purity

FD-MS: calcd for C71H43N7=994.15,

found m/z=994 (M+, 100)

### (2) Production of Organic EL Device

An organic EL device was produced in the same manner as in Example 1 except for using the compound H-6 in place of the compound H-1.

### (3) Evaluation of EL Performance

The evaluation was made in the same manner as in Example 1. The evaluation results are shown in Table 1.

### Example 7

### (1) Synthesis of Compound H-7

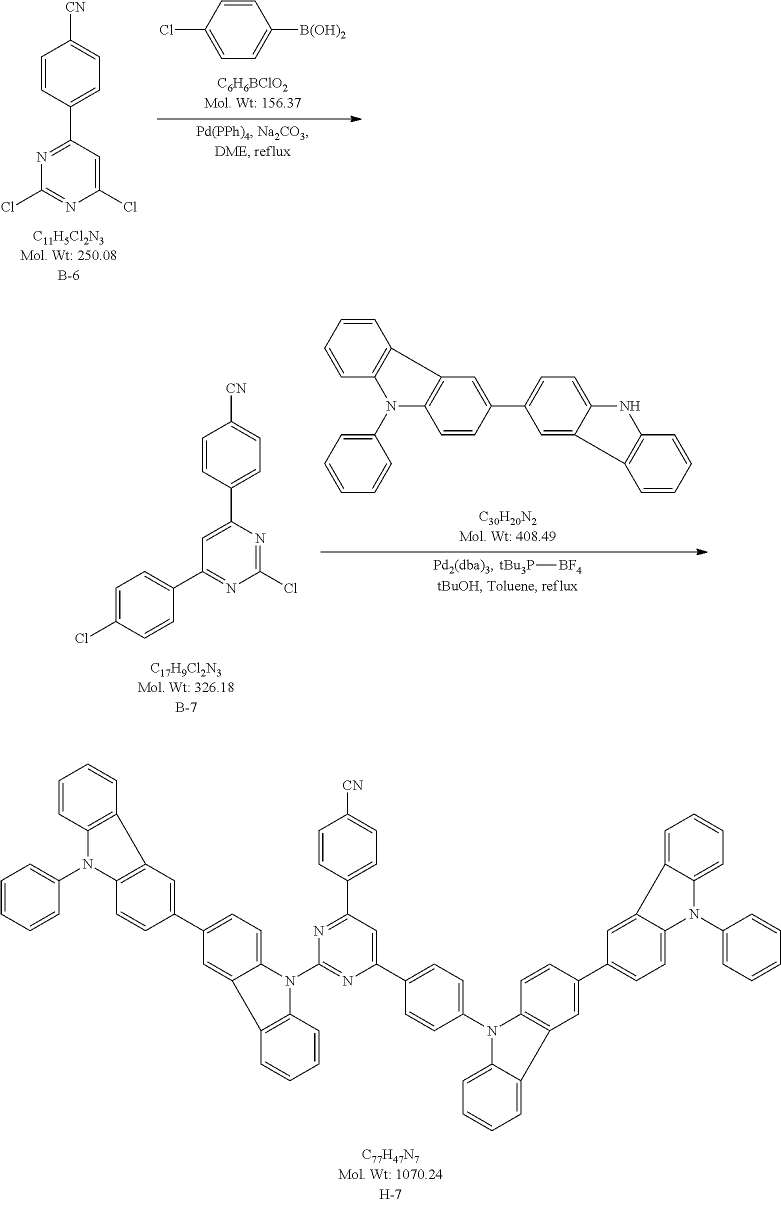

In an argon atmosphere, a mixture of B-6 (3.13 g, 12.5 mmol), 4-chlorophenylboronic acid (2.03 g, 13 mmol), tetrakis(triphenylphosphine) palladium (289 mg, 0.25 mmol), toluene (45 mL), and a 2 M aqueous solution of sodium carbonate (22.5 mL, 45 mmol) was allowed to react for 8 h while refluxing under heating. After cooling the reaction solution to room temperature, the water layer was separated and removed, and the organic layer was dried over magnesium sulfate. The insolubles were removed by filtration, and the organic solvent was evaporated off under reduced pressure. The obtained residue was purified by silica gel column chromatography to obtain the pyrimidine intermediate B-7 (3.22 g, yield: 79%).

In an argon atmosphere, a mixture obtained by successively mixing the bicarbazolyl intermediate A-1 (2.57 g, 6.3 mmol), the pyrimidine intermediate B-7 (0.98 g, 3.0 mmol), tris(dibenzylideneacetone)dipalladium (55 mg, 0.06 mmol), tri-t-butylphosphonium tetrafluoroborate (0.070 g, 0.24 mmol), t-butoxysodium (0.87 g, 9.0 mmol), and dry xylene (60 mL) was refluxed for 16 h under heating.

After cooling the reaction solution to room temperature, the insolubles were removed by filtration, and the organic solvent was evaporated off under reduced pressure. The obtained residue was purified by silica gel column chromatography to obtain H-7 (2.44 g, yield: 76%).

The obtained compound was analyzed by HPLC and FD-MS. The results are shown below.

HPLC: 99.3% purity

FD-MS: calcd for C77H47N7=1070.24,

found m/z=1070 (M+, 100)

### (2) Production of Organic EL Device

An organic EL device was produced in the same manner as in Example 1 except for using the compound H-7 in place of the compound H-1.

### (3) Evaluation of EL Performance

The evaluation was made in the same manner as in Example 1. The evaluation results are shown in Table 1.

### Example 8

### (1) Synthesis of Compound H-8

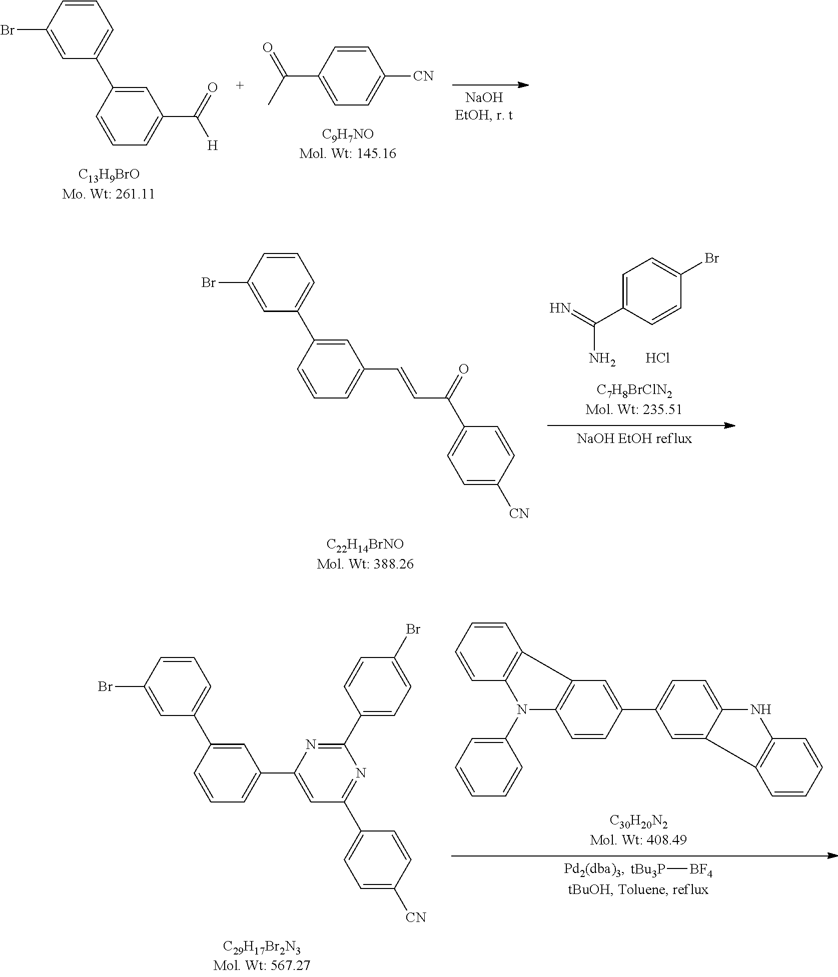

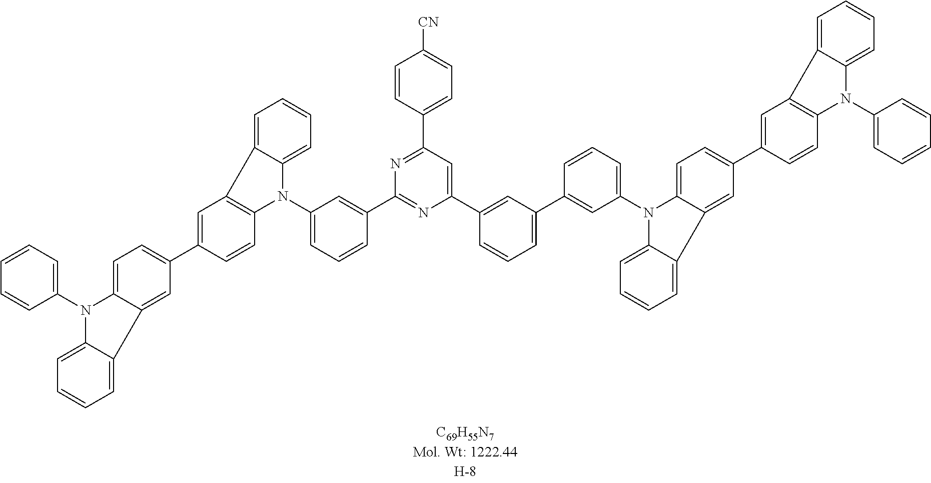 [SMILES: N#Cc1ccc(-c2cc(-c3cccc(-c4cccc(-n5c6ccccc6c6cc(-c7ccc8c(c7)c7ccccc7n8-c7ccccc7)ccc65)c4)c3)nc(-c3cccc(-n4c5ccccc5c5cc(-c6ccc7c(c6)c6ccccc6n7-c6ccccc6)ccc54)c3)n2)cc1]

Into a solution of 3′-bromo-[1,1′-biphenyl]-3-aldehyde (10.44 g, 40 mmol) and 3′-cyanoacetophenone (5.81 g, 40 mmol) in ethanol (80 mL), sodium hydroxide (0.16 g, 4 mmol) was added, and the resultant solution was stirred for 8 h at room temperature. After adding 4-bromobenzamidine hydrochloride (4.71 g, 20 mmol) and sodium hydroxide (1.60 g, 40 mmol), ethanol (40 mL) was further added. The reaction was allowed to proceed for 8 h while refluxing under heating. The generated white powder was collected by filtration, washed with ethanol until the filtrate became colorless, further washed with water and then ethanol, and vacuum-dried to obtain the pyrimidine intermediate B-8 (6.81 g, 12.0 mmol, yield: 60%).

In an argon atmosphere, a mixture obtained by successively mixing the bicarbazolyl intermediate A-1 (2.57 g, 6.3 mmol), the pyrimidine intermediate B-8 (1.70 g, 3.0 mmol), tris(dibenzylideneacetone)dipalladium (55 mg, 0.06 mmol), tri-t-butylphosphonium tetrafluoroborate (0.070 g, 0.24 mmol), t-butoxysodium (0.87 g, 9.0 mmol), and dry xylene (60 mL) was refluxed for 16 h under heating.

After cooling the reaction solution to room temperature, the insolubles were removed by filtration, and the organic solvent was evaporated off under reduced pressure. The obtained residue was purified by silica gel column chromatography to obtain H-8 (2.57 g, yield: 70%).

The obtained compound was analyzed by HPLC and FD-MS. The results are shown below.

HPLC: 99.3% purity

FD-MS: calcd for C89H55N7=1222.44,

found m/z=1222 (M+, 100)

### (2) Production of Organic EL Device

An organic EL device was produced in the same manner as in Example 1 except for using the compound H-8 in place of the compound H-1.

### (3) Evaluation of EL Performance

The evaluation was made in the same manner as in Example 1. The evaluation results are shown in Table 1.

### Example 9

### (1) Synthesis of Compound H-9

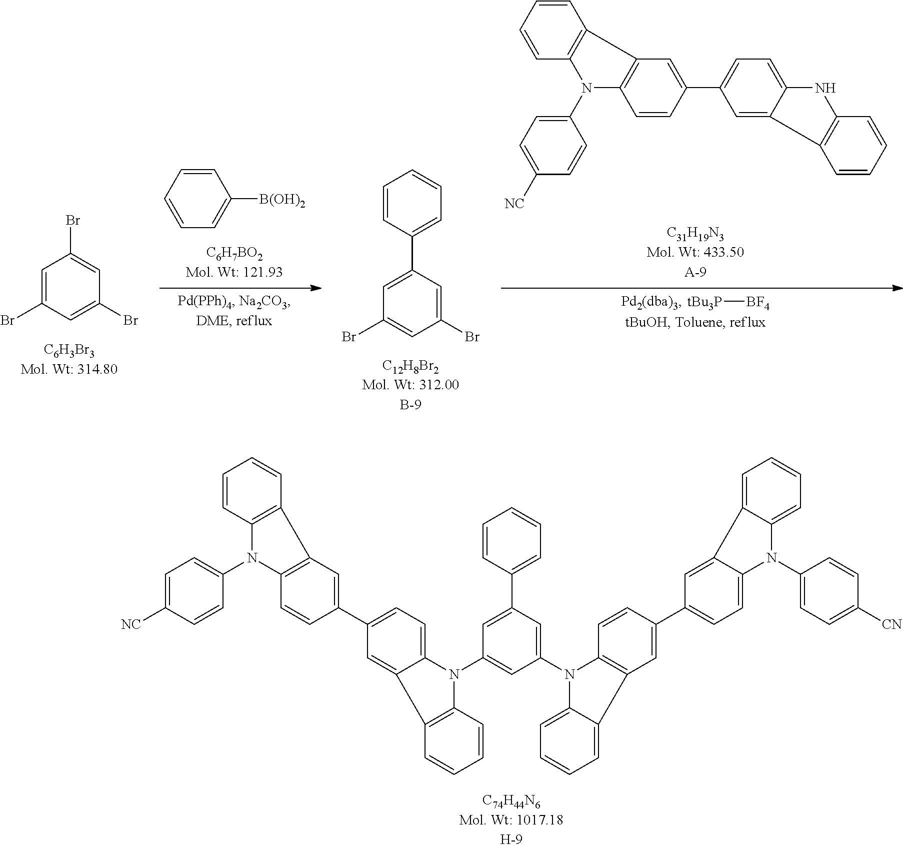

In an argon atmosphere, a mixture of 1,3,5-tribromobenzene (9.44 g, 30 mmol), phenylboronic acid (1.22 g, 10 mmol), tetrakis(triphenylphosphine) palladium (231 mg, 0.2 mmol), DME (50 mL), and a 2 M aqueous solution of sodium carbonate (10 mL, 20 mmol) was allowed to react for 8 h while refluxing under heating. After cooling the reaction solution to room temperature, the water layer was separated and removed, and the organic layer was dried over magnesium sulfate. Then, the insolubles were removed by filtration, and the organic solvent was evaporated off under reduced pressure. The obtained residue was purified by silica gel column chromatography to obtain the intermediate B-9 (2.03 g, yield: 65%).

In an argon atmosphere, a mixture obtained by successively mixing the bicarbazolyl intermediate A-9 (2.73 g, 6.3 mmol), the intermediate B-9 (0.94 g, 3.0 mmol), tris(dibenzylideneacetone)dipalladium (55 mg, 0.06 mmol), tri-t-butylphosphonium tetrafluoroborate (0.070 g, 0.24 mmol), t-butoxysodium (0.87 g, 9.0 mmol), and dry xylene (60 mL) was refluxed for 16 h under heating.

After cooling the reaction solution to room temperature, the insolubles were removed by filtration, and the organic solvent was evaporated off under reduced pressure. The obtained residue was purified by silica gel column chromatography to obtain H-9 (2.44 g, yield: 76%).

The obtained compound was analyzed by HPLC and FD-MS. The results are shown below.

HPLC: 99.3% purity

FD-MS: calcd for C74H44N6=1017.18,

found m/z=1017 (M+, 100)

### (2) Production of Organic EL Device

An organic EL device was produced in the same manner as in Example 1 except for using a 1:1 by weight mixture of the compound H-6 and the compound H-9 in place of the compound H-1.

### (3) Evaluation of EL Performance

The evaluation was made in the same manner as in Example 1. The evaluation results are shown in Table 1.

### Example 10

### (1) Synthesis of Compound H-10

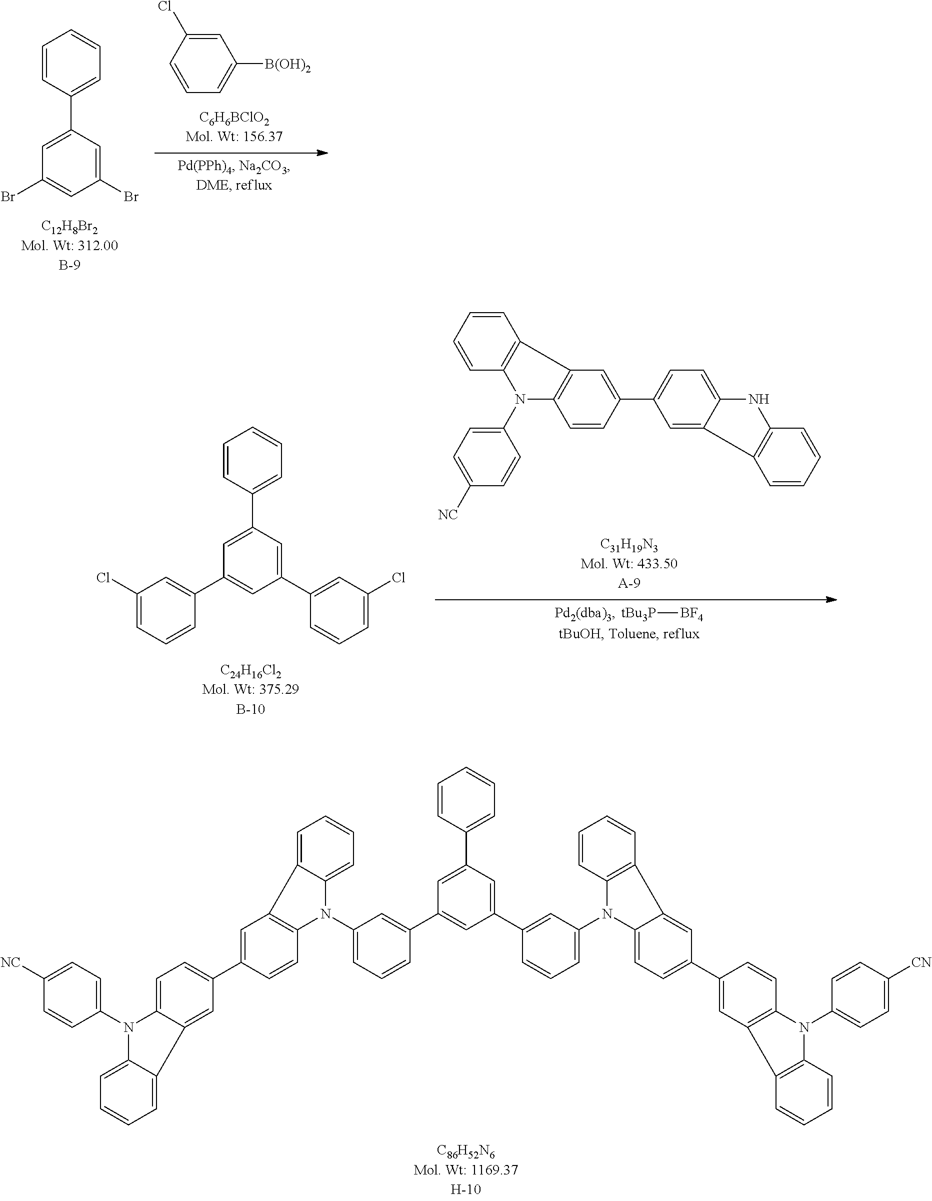

In an argon atmosphere, a mixture B-9 (3.12 g, 10 mmol), 3-chlorophenylboronic acid (3.44 g, 22 mmol), tetrakis(triphenylphosphine) palladium (508 mg, 0.44 mmol), DME (50 mL), and a 2 M aqueous solution of sodium carbonate (22 mL, 44 mmol) was allowed to react for 8 h while refluxing under heating. After cooling the reaction solution to room temperature, the water layer was separated and removed, and the organic layer was dried over magnesium sulfate. Then, the insolubles were removed by filtration, and the organic solvent was evaporated off under reduced pressure. The obtained residue was purified by silica gel column chromatography to obtain the intermediate B-10 (2.25 g, yield: 60%).

In an argon atmosphere, a mixture obtained by successively mixing the bicarbazolyl intermediate A-9 (2.73 g, 6.3 mmol), the intermediate B-10 (1.13 g, 3.0 mmol), tris(dibenzylideneacetone)dipalladium (55 mg, 0.06 mmol), tri-t-butylphosphonium tetrafluoroborate (0.070 g, 0.24 mmol), t-butoxysodium (0.87 g, 9.0 mmol), and dry xylene (60 mL) was refluxed for 16 h under heating.

After cooling the reaction solution to room temperature, the insolubles were removed by filtration, and the organic solvent was evaporated off under reduced pressure. The obtained residue was purified by silica gel column chromatography to obtain H-10 (2.60 g, yield: 74%).

The obtained compound was analyzed by HPLC and FD-MS. The results are shown below.

HPLC: 99.2% purity

FD-MS: calcd for C86H52N6=1169.37,

found m/z=1169 (M+, 100)

### (2) Production of Organic EL Device

An organic EL device was produced in the same manner as in Example 1 except for using a 1:1 by weight mixture of the compound H-3 and the compound H-10 in place of the compound H-1.

### (3) Evaluation of EL Performance

The evaluation was made in the same manner as in Example 1. The evaluation results are shown in Table 1.

### Example 11

### (1) Synthesis of Compound H-11

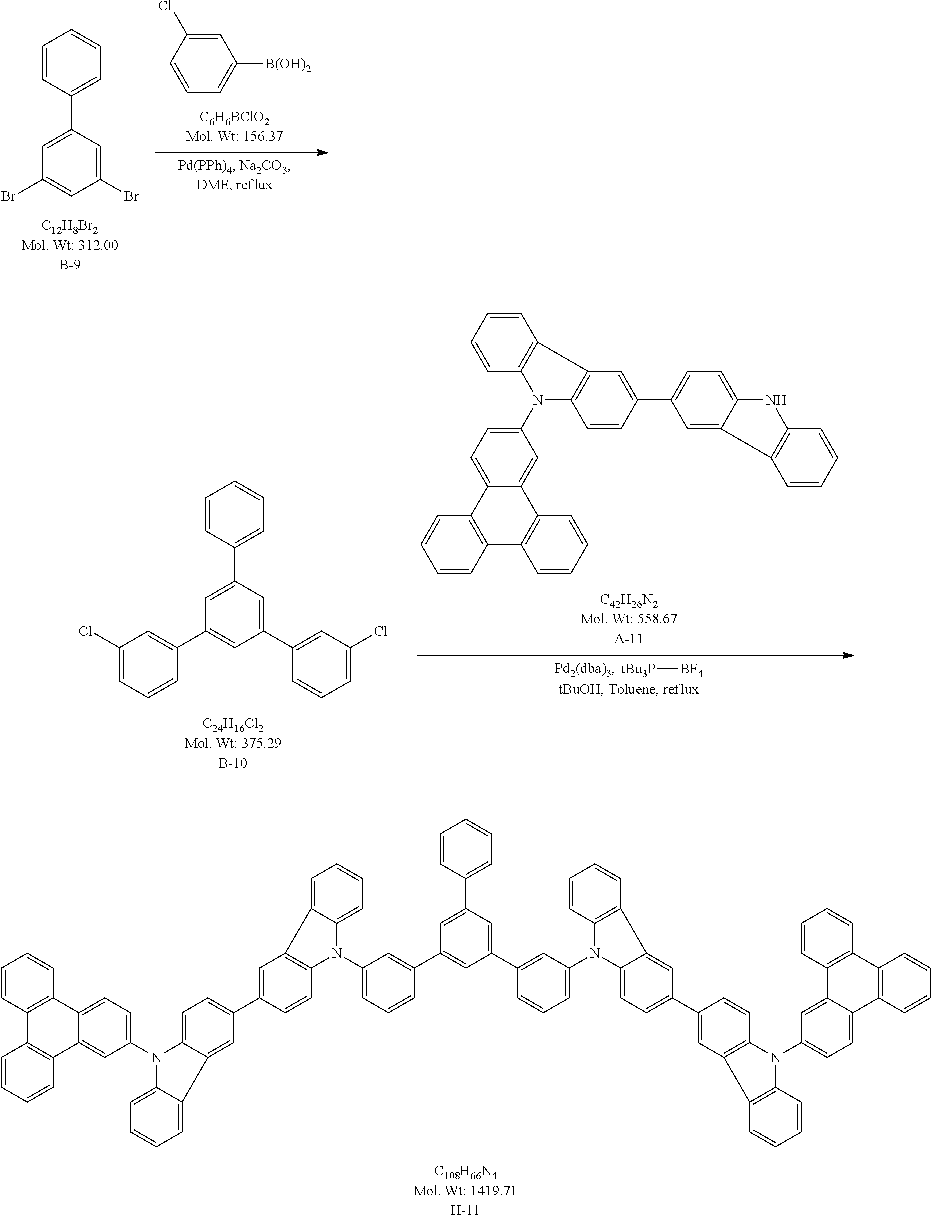

In an argon atmosphere, a mixture of the bicarbazolyl intermediate A-11 (3.52 g, 6.3 mmol), the intermediate B-10 (1.13 g, 3.0 mmol), tris(dibenzylideneacetone)dipalladium (55 mg, 0.06 mmol), tri-t-butylphosphonium tetrafluoroborate (0.070 g, 0.24 mmol), t-butoxysodium (0.87 g, 9.0 mmol), and dry xylene (60 mL) was refluxed for 16 h under heating.

After cooling the reaction solution to room temperature, the insolubles were removed by filtration, and the organic solvent was evaporated off under reduced pressure. The obtained residue was purified by silica gel column chromatography to obtain H-11 (2.98 g, yield: 70%).

The obtained compound was analyzed by HPLC and FD-MS. The results are shown below.

HPLC: 99.1% purity

FD-MS: calcd for C108H66N4=1419.71,

found m/z=1419 (M+, 100)

### (2) Production of Organic EL Device

An organic EL device was produced in the same manner as in Example 1 except for using a 1:1 by weight mixture of the compound H-3 and the compound H-11 in place of the compound H-1.

### (3) Evaluation of EL Performance

The evaluation was made in the same manner as in Example 1. The evaluation results are shown in Table 1.

### Example 12

### (1) Synthesis of Compound H-12

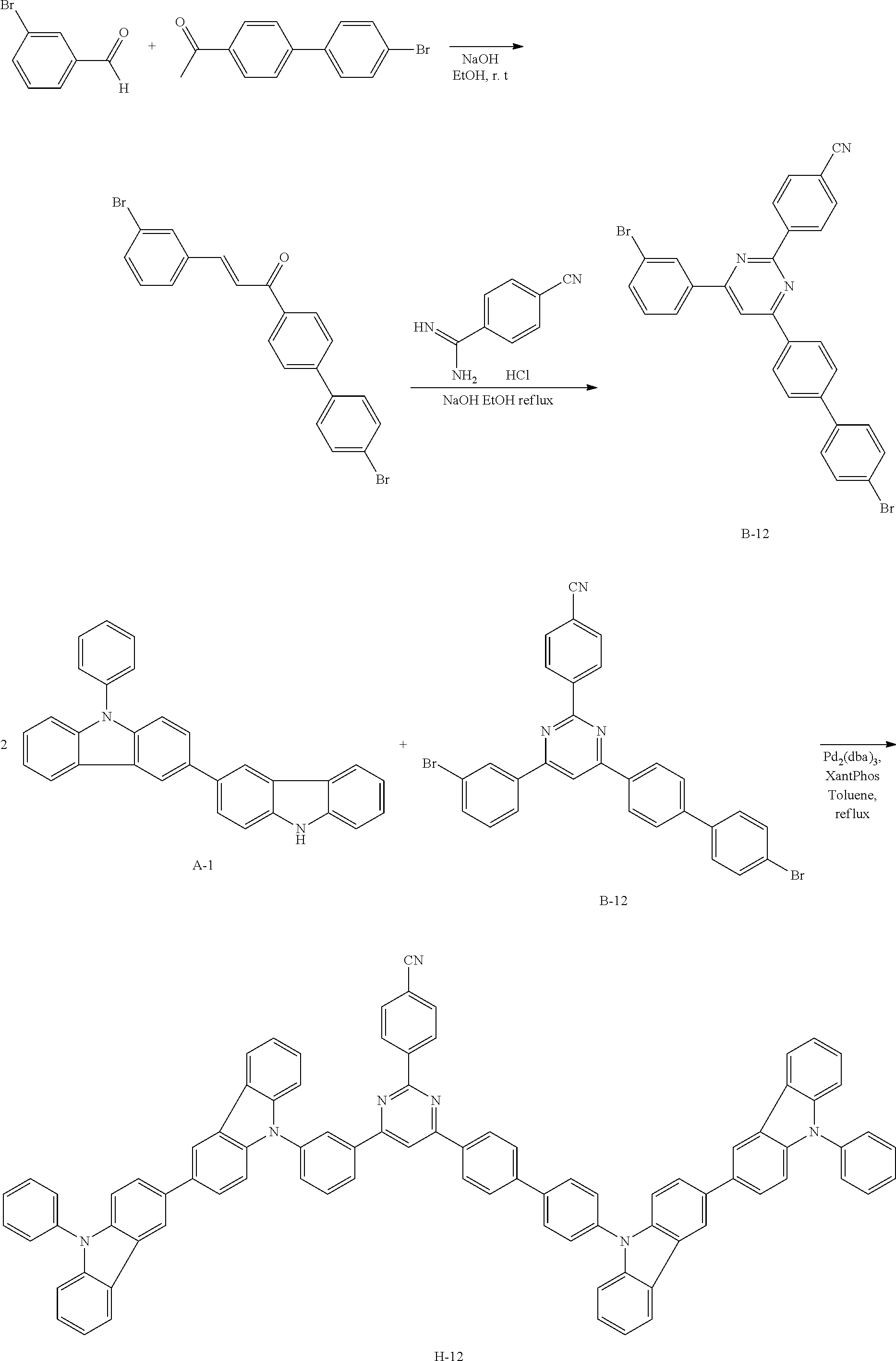

Into a solution of 3-bromobenzaldehyde (7.40 g, 40 mmol) and 4-acetyl-4′-bromobiphenyl (11.00 g, 40 mmol) in ethanol (80 mL), sodium hydroxide (0.16 g, 4 mmol) was added, and the resultant solution was stirred for 8 h at room temperature. After adding 4-cyanobenzamidine hydrochloride (3.63 g, 20 mmol) and sodium hydroxide (1.60 g, 40 mmol), ethanol (40 mL) was further added. The reaction was allowed to proceed for 8 h while refluxing under heating. The generated pale yellow powder was collected by filtration, washed with ethanol until the filtrate became colorless, further washed with water and then ethanol, and vacuum-dried to obtain the pyrimidine intermediate B-12 (8.85 g, yield: 78%).

In an argon atmosphere, a mixture obtained by successively mixing the bicarbazolyl intermediate A-1 (2.57 g, 6.3 mmol), the pyrimidine intermediate B12 (1.70 g, 3.0 mmol), tris(dibenzylideneacetone)dipalladium (0.055 g, 0.06 mmol), xantphos (4,5′-bis(diphenylphosphino)-9,9′-dimethylxanthene) (0.069 g, 0.12 mmol), t-butoxysodium (0.87 g, 9.0 mmol), and dry toluene (60 mL) was refluxed for 12 h under heating.

After cooling the reaction solution to room temperature, the insolubles were removed by filtration, and the organic solvent was evaporated off under reduced pressure. The obtained residue was purified by silica gel column chromatography to obtain H-12 (2.12 g, yield: 58%).

The obtained compound was analyzed by HPLC and FD-MS. The results are shown below.

HPLC: 99.1% purity

FD-MS: calcd for C89H55N7=1221.45

found m/z=1221 (M+, 100), 1222 (98)

### (2) Production of Organic EL Device

An organic EL device was produced in the same manner as in Example 1 except for using the compound H-12 in place of the compound H-1.

### (3) Evaluation of EL Performance

The evaluation was made in the same manner as in Example 1. The evaluation results are shown in Table 1.

### Example 13

### (1) Synthesis of Compound H-13

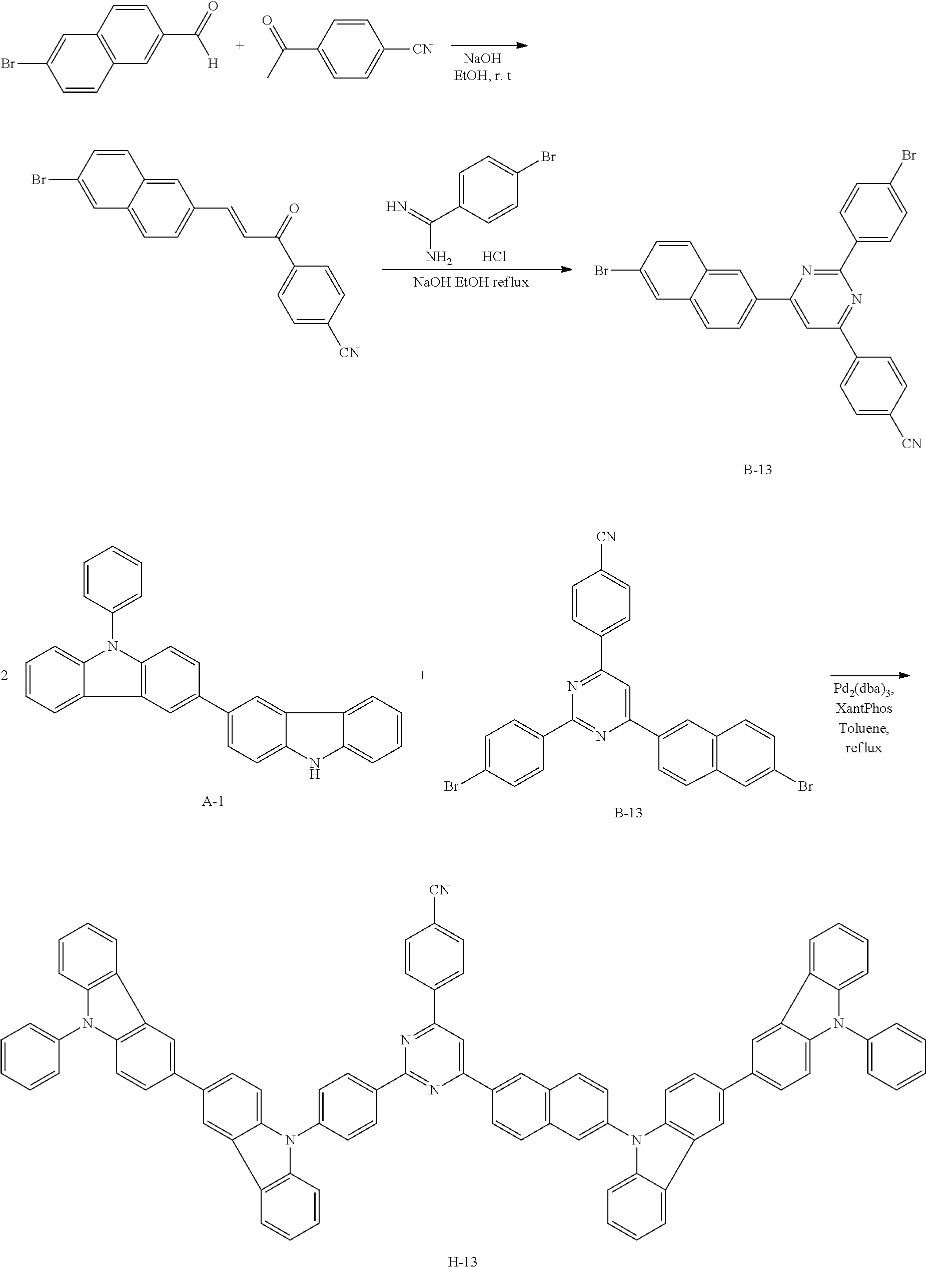

Into a solution of 6-bromo-2-naphthoaldehyde (9.40 g, 40 mmol) and 4′-cyanoacetophenone (5.80 g, 40 mmol) in ethanol (80 mL), sodium hydroxide (0.16 g, 4 mmol) was added, and the resultant solution was stirred for 8 h at room temperature. After adding 4-bromobenzamidine hydrochloride (4.71 g, 20 mmol) and sodium hydroxide (1.60 g, 40 mmol), ethanol (40 mL) was further added. The reaction was allowed to proceed for 8 h while refluxing under heating. The generated pale yellow powder was collected by filtration, washed with ethanol until the filtrate became colorless, further washed with water and then ethanol, and vacuum-dried to obtain the pyrimidine intermediate B-13 (7.79 g, yield: 72%).

In an argon atmosphere, a mixture obtained by successively mixing the bicarbazolyl intermediate A-1 (2.57 g, 6.3 mmol), the pyrimidine intermediate B-13 (1.62 g, 3.0 mmol), tris(dibenzylideneacetone)dipalladium (0.055 g, 0.06 mmol), xantphos (0.069 g, 0.12 mmol), t-butoxysodium (0.87 g, 9.0 mmol), and dry toluene (60 mL) was refluxed for 16 h under heating.

After cooling the reaction solution to room temperature, the insolubles were removed by filtration, and the organic solvent was evaporated off under reduced pressure. The obtained residue was purified by silica gel column chromatography to obtain H-13 (2.37 g, yield: 66%).

The obtained compound was analyzed by HPLC and FD-MS. The results are shown below.

HPLC: 98.7% purity

FD-MS: calcd for C87H53N7=1195.43

found m/z=1195 (M+, 100), 1196 (97)

### (2) Production of Organic EL Device

An organic EL device was produced in the same manner as in Example 1 except for using the compound H-13 in place of the compound H-1.

### (3) Evaluation of EL Performance

The evaluation was made in the same manner as in Example 1. The evaluation results are shown in Table 1.

### Example 14

### (1) Synthesis of Compound H-14

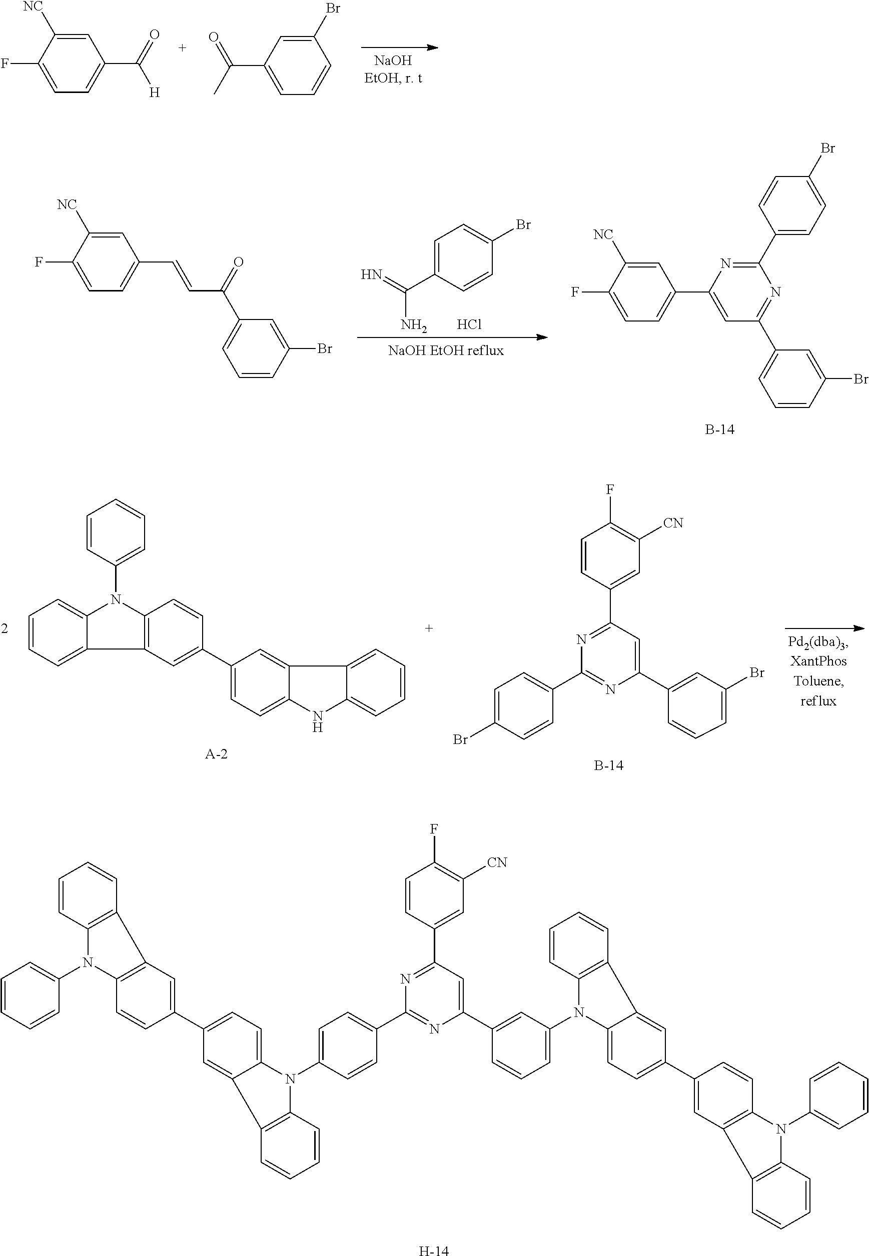

Into a solution of 3-cyano-4-fluorobenzaldehyde (5.96 g, 40 mmol) and 3′-bromoacetophenone (5.80 g, 40 mmol) in ethanol (80 mL), sodium hydroxide (0.16 g, 4 mmol) was added, and the resultant solution was stirred for 8 h at room temperature. After adding 4-bromobenzamidine hydrochloride (4.71 g, 20 mmol) and sodium hydroxide (1.60 g, 40 mmol), ethanol (40 mL) was further added. The reaction was allowed to proceed for 8 h while refluxing under heating. The generated white powder was collected by filtration, washed with ethanol until the filtrate became colorless, further washed with water and then ethanol, and vacuum-dried to obtain the pyrimidine intermediate B-14 (7.64 g, yield: 75%).

In an argon atmosphere, a mixture obtained by successively mixing the bicarbazolyl intermediate A-1 (2.57 g, 6.3 mmol), the pyrimidine intermediate B-14 (1.53 g, 3.0 mmol), tris(dibenzylideneacetone)dipalladium (0.055 g, 0.06 mmol), xantphos (0.069 g, 0.12 mmol), t-butoxysodium (0.87 g, 9.0 mmol), and dry toluene (60 mL) was refluxed for 16 h under heating.

After cooling the reaction solution to room temperature, the insolubles were removed by filtration, and the organic solvent was evaporated off under reduced pressure. The obtained residue was purified by silica gel column chromatography to obtain H-14 (2.37 g, yield: 66%).

The obtained compound was analyzed by HPLC and FD-MS. The results are shown below.

HPLC: 99.2% purity

FD-MS: calcd for C83H50FN7=1163.41

found m/z=1163 (M+, 100), 1164 (92)

### (2) Production of Organic EL Device

An organic EL device was produced in the same manner as in Example 1 except for using the compound H-14 in place of the compound H-1.

### (3) Evaluation of EL Performance

The evaluation was made in the same manner as in Example 1. The evaluation results are shown in Table 1.

### Example 15

### (1) Synthesis of Compound H-15

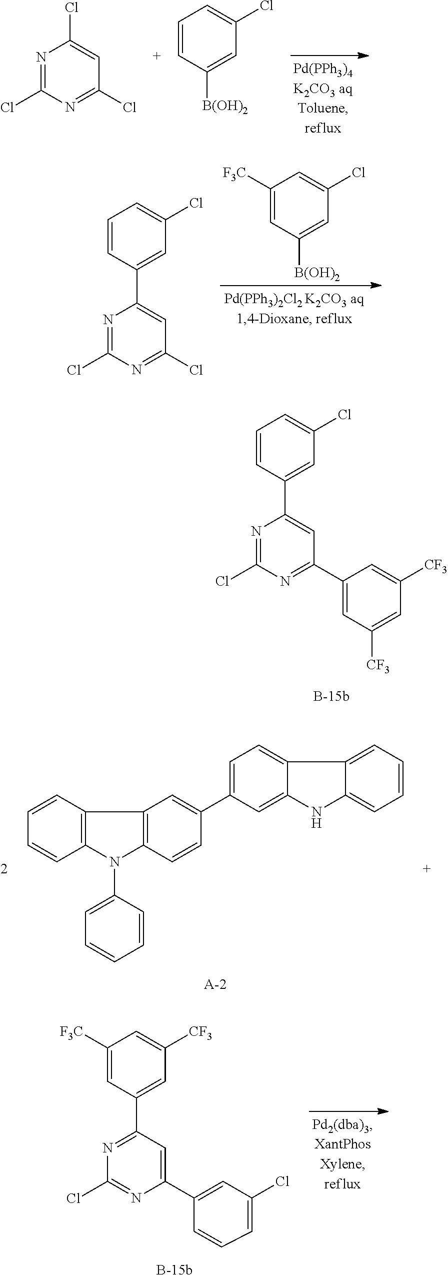

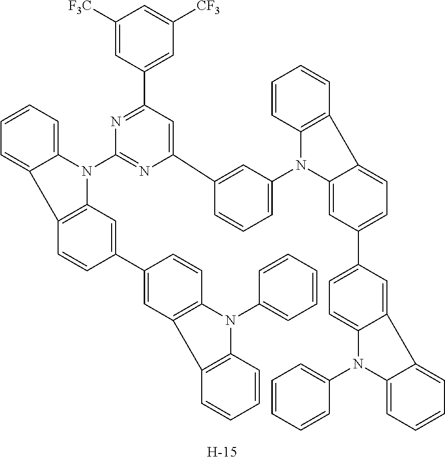

In a nitrogen atmosphere, a mixture of 2,4,6-trichloropyrimidine (5.50 g, 30 mmol), 3-chlorophenylboronic acid (4.69 g, 30 mmol), bistriphenylphosphine palladium dichloride (0.421 g, 0.6 mmol), potassium carbonate (8.29 g, 60 mmol), toluene (60 mL), and pure water (30 mL) was stirred for 7 h under refluxing. After cooling, the water layer was removed and the organic layer was washed twice with pure water and then the solvent was evaporated off. The obtained residue was purified by silica gel column chromatography to obtain the intermediate B-15a (4.01 g, yield: 51.4%). In a nitrogen atmosphere, a mixture of the intermediate B-15a (4.01 g, 15 mmol), 3,5-bis(trifluoromethyl)phenylboronic acid (3.98 g, 15 mmol), bistriphenylphosphine palladium dichloride (0.211 g, 0.3 mmol), potassium carbonate (4.15 g, 30 mmol), 1,4-dioxane (30 mL), and pure water (15 mL) was stirred for 4.5 h under refluxing. After cooling and adding 50 mL of toluene, the water layer was removed, the organic layer was washed twice with pure water, and then the solvent was evaporated off. The obtained residue was purified by silica gel column chromatography to obtain the intermediate B-15b (3.2 g, yield: 48.8%).

In a nitrogen atmosphere, a mixture obtained by successively mixing the bicarbazolyl intermediate A-2 (1.716 g, 4.2 mmol), the intermediate B-15b (0.874 g, 2 mmol), tris(dibenzylideneacetone)dipalladium (37 mg, 0.04 mmol), xantphos (23 mg, 0.08 mmol), t-butoxysodium (0.577 g, 6 mmol), and dry xylene (25 mL) was refluxed for 9 h under heating.

After cooling the reaction solution to room temperature, the insolubles were removed by filtration, and the organic solvent was evaporated off under reduced pressure. The obtained residue was purified by silica gel column chromatography to obtain H-15 (1.751 g, yield: 74.1%).

The obtained compound was analyzed by HPLC and FD-MS. The results are shown below.

HPLC: 98.7% purity

FD-MS: calcd for C78H46N6F6=1180.37

found m/z=1180 (M+, 100), 1181 (87)

### (2) Production of Organic EL Device

An organic EL device was produced in the same manner as in Example 1 except for using the compound H-15 in place of the compound H-1.

### (3) Evaluation of EL Performance

The evaluation was made in the same manner as in Example 1. The evaluation results are shown in Table 1.

### Example 16

### (1) Synthesis of Compound H-16

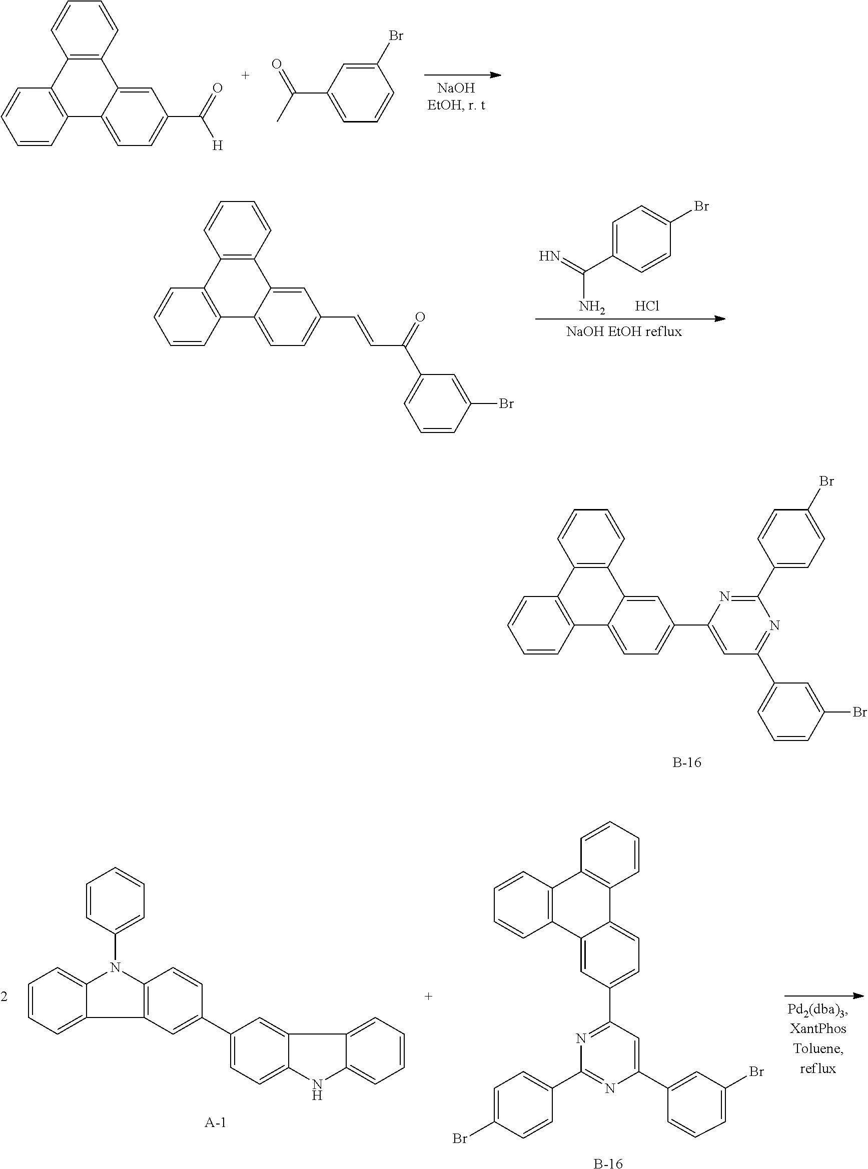

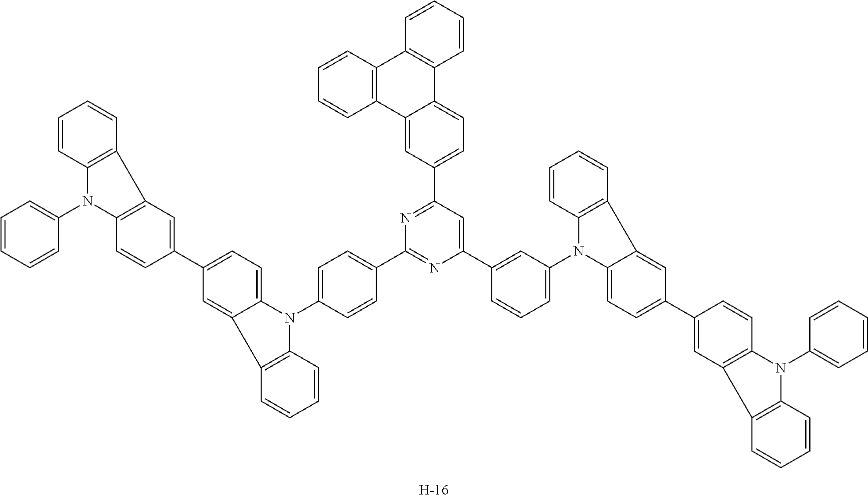

Into a solution of 2-formyltriphenylene (5.12 g, 20 mmol) and 3′-acetophenone (3.98 g, 20 mmol) in ethanol (40 mL), sodium hydroxide (0.08 g, 2 mmol) was added, and the resultant solution was stirred for 8 h at room temperature. After adding 4-bromobenzamidine hydrochloride (2.36 g, 10 mmol) and sodium hydroxide (0.80 g, 20 mmol), ethanol (40 mL) was further added. The reaction was allowed to proceed for 8 h while refluxing under heating. The generated white powder was collected by filtration, washed with ethanol until the filtrate became colorless, further washed with water and then ethanol, and vacuum-dried to obtain the pyrimidine intermediate B-16 (5.05 g, yield: 82%).

In an argon atmosphere, a mixture obtained by successively mixing the bicarbazolyl intermediate A-1 (2.57 g, 6.3 mmol), the pyrimidine intermediate B-16 (1.85 g, 3.0 mmol), tris(dibenzylideneacetone)dipalladium (0.055 g, 0.06 mmol), xantphos (0.069 g, 0.12 mmol), t-butoxysodium (0.87 g, 9.0 mmol), and dry toluene (60 mL) was refluxed for 16 h under heating.

After cooling the reaction solution to room temperature, the insolubles were removed by filtration, and the organic solvent was evaporated off under reduced pressure. The obtained residue was purified by silica gel column chromatography to obtain H-16 (2.98 g, yield: 78%).

The obtained compound was analyzed by HPLC and FD-MS. The results are shown below.

HPLC: 99.3% purity

FD-MS: calcd for C94H58N6=1270.47

found m/z=1270 (M+, 96), 1271 (100)

### (2) Production of Organic EL Device

An organic EL device was produced in the same manner as in Example 1 except for using the compound H-16 in place of the compound H-1.

### (3) Evaluation of EL Performance

The evaluation was made in the same manner as in Example 1. The evaluation results are shown in Table 1.

### Comparative Example 1

### (1) Production of Organic EL Device

An organic EL device was produced in the same manner as in Example 1 except for using a 1:3 by weight mixture of the compound h-1 and the compound h-2 in place of the compound H-1.

The structures of the compound h-1 and the compound h-2 are shown below. These compounds are disclosed in Patent Document 2.

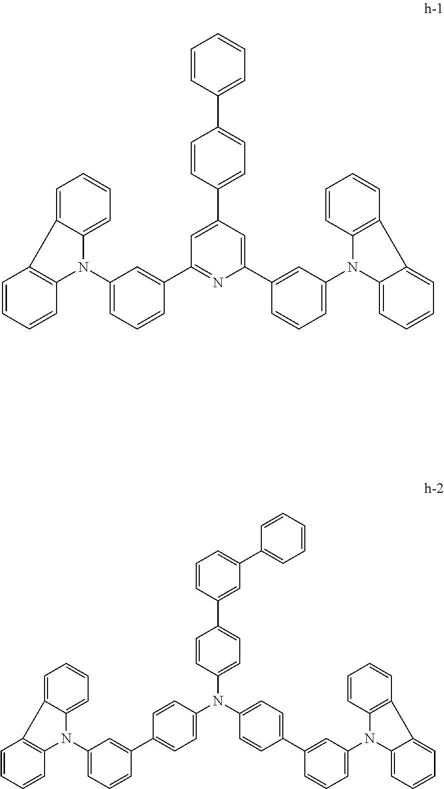 [SMILES: c1ccc(-c2ccc(-c3cc(-c4cccc(-n5c6ccccc6c6ccccc65)c4)nc(-c4cccc(-n5c6ccccc6c6ccccc65)c4)c3)cc2)cc1 ; c1ccc(-c2cccc(-c3ccc(N(c4ccc(-c5cccc(-n6c7ccccc7c7ccccc76)c5)cc4)c4ccc(-c5cccc(-n6c7ccccc7c7ccccc76)c5)cc4)cc3)c2)cc1]

### (2) Evaluation of EL Performance

The evaluation was made in the same manner as in Example 1. The evaluation results are shown in Table 1.

TABLE 1
Evaluation of device performance of organic EL device

| | | | Emission | Lifetime |
|---|---|---|---|---|
| | | Voltage (V) | efficiency (cd/A) | (h) |
| | Host | @10 mA/cm² | @10 mA/cm² | LT90 |
| Example 1 | H-1 | 5.0 | 49 | 235 |
| Example 2 | H-2 | 5.1 | 45 | 217 |
| Example 3 | H-3 | 4.9 | 48 | 243 |
| Example 4 | H-4 | 4.8 | 51 | 216 |
| Example 5 | H-5 | 4.7 | 49 | 213 |
| Example 6 | H-6 | 5.1 | 47 | 209 |
| Example 7 | H-7 | 5.0 | 48 | 223 |
| Example 8 | H-8 | 4.9 | 46 | 216 |
| Example 9 | H-6:H-9 | 4.7 | 53 | 248 |
| Example 10 | H-3:H-10 | 4.6 | 52 | 239 |
| Example 11 | H-3:H-11 | 4.7 | 49 | 237 |
| Example 12 | H-12 | 5.0 | 48 | 240 |
| Example 13 | H-13 | 4.9 | 47 | 222 |
| Example 14 | H-14 | 5.0 | 41 | 267 |
| Example 15 | H-15 | 4.2 | 43 | 209 |
| Example 16 | H-16 | 5.2 | 42 | 265 |
| Comparative | h-1:h-2 | 6.5 | 24.5 | 58 |
| Example 1 | | | | |

By using the material of the invention, an organic electroluminescence device exhibiting a higher efficiency and a longer lifetime and capable of driving at a lower voltage is obtained as compared with using a conventional material.

### INDUSTRIAL APPLICABILITY

The aromatic heterocyclic derivative of the invention is useful as the material for an organic electroluminescence device.

In addition, since the aromatic heterocyclic derivative of the invention is soluble and suitable for use in a coating process, it is useful for use as a solution for an organic electroluminescence device.

1. An aromatic heterocyclic derivative represented by formula (1):
AL¹-B)ₘ 1)
wherein:
A represents a substituted or unsubstituted aromatic hydrocarbon ring group, a substituted or unsubstituted aromatic heterocyclic group, a residue of a ring assembly which comprises at least two substituted or unsubstituted aromatic hydrocarbon rings, a residue of a ring assembly which comprises at least two substituted or unsubstituted aromatic heterocyclic rings, or a residue of a ring assembly which comprises at least one substituted or unsubstituted aromatic hydrocarbon ring and at least one substituted or unsubstituted aromatic heterocyclic ring;
L¹ represents a single bond, a substituted or unsubstituted aromatic hydrocarbon ring group, or a substituted or unsubstituted aromatic heterocyclic group;
B represents a residue of a structure represented by formula (2-b); and
m represents an integer of 2 or more, groups L¹ may be the same or different, and groups B may be the same or different,
provided that a group represented by formula (3) is bonded to at least one of A, L¹ and B; 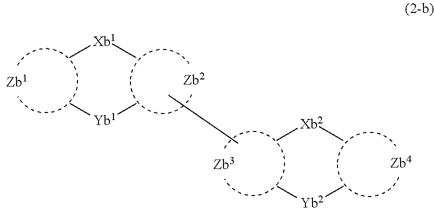
wherein:
one of Xb¹ and Yb¹ represents a single bond, —CR₂—, —NR—, —O—, —S—, —SiR₂—, a group represented by formula (i), or a group represented by formula (ii), and the other represents —NR—, —O—, —S—, —SiR₂—, a group represented by formula (i), or a group represented by formula (ii);
one of Xb² and Yb² represents a single bond, —CR₂—, —NR—, —O—, —S—, —SiR₂—, a group represented by formula (i), or a group represented by formula (ii), and the other represents —NR—, —O—, —S—, —SiR₂—, a group represented by formula (i), or a group represented by formula (ii); 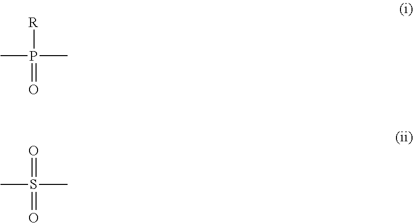
R represent a hydrogen atom, a substituted or unsubstituted alkyl group, a substituted or unsubstituted cycloalkyl group, a substituted or unsubstituted aromatic hydrocarbon ring group, or a substituted or unsubstituted aromatic heterocyclic group;
each of Zb¹, Zb², Zb³, and Zb⁴ independently represents a substituted or unsubstituted aliphatic hydrocarbon ring group, a substituted or unsubstituted aliphatic heterocyclic group, a substituted or unsubstituted aromatic hydrocarbon ring group, or a substituted or unsubstituted aromatic heterocyclic group;
-L³-F (3)
wherein
L³ represents a single bond, a substituted or unsubstituted aromatic hydrocarbon ring group, or a substituted or unsubstituted aromatic heterocyclic group;
when the group represented by formula (3) is bonded to A, F represents a group selected from the group consisting of a cyano group, a fluorine atom, a haloalkyl group, a substituted or unsubstituted triphenylenyl group, a substituted or unsubstituted azafluorenyl group, a substituted or unsubstituted spirofluorenyl group, a substituted or unsubstituted dibenzothiophenyl group, a substituted or unsubstituted bipyridinyl group, a substituted or unsubstituted bipyrimidinyl group, a substituted or unsubstituted quinazolinyl group, a substituted or unsubstituted imidazolyl group, a substituted or unsubstituted benzimidazolyl group, a phosphorus-containing group, a silicon-containing group, and benzene-fused or aza-substituted analogues of the preceding groups; and
when the group represented by formula (3) is bonded to L¹ or B, F represents a group selected from the group consisting of a cyano group, a fluorine atom, a haloalkyl group, a substituted or unsubstituted triphenylenyl group, a substituted or unsubstituted fluorenyl group, a substituted or unsubstituted spirofluorenyl group, a substituted or unsubstituted dibenzothiophenyl group, a substituted or unsubstituted dibenzofuranyl group, a substituted or unsubstituted pyridinyl group, a substituted or unsubstituted pyrimidinyl group, a substituted or unsubstituted triazinyl group, a substituted or unsubstituted bipyridinyl group, a substituted or unsubstituted bipyrimidinyl group, a substituted or unsubstituted quinazolinyl group, a substituted or unsubstituted imidazolyl group, a substituted or unsubstituted benzimidazolyl group, a phosphorus-containing group, a silicon-containing group, and benzene-fused or aza-substituted analogues of the preceding groups.

2. The aromatic heterocyclic derivative according to claim 1, wherein the structure represented by formula (2-b) is represented by formula (2-b-1): 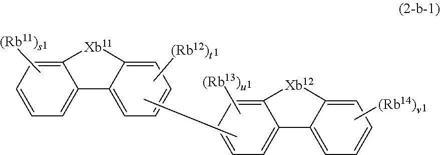
wherein:
each of Xb¹¹ and Xb¹² independently represents —NR—, —O—, —S—, —SiR₂—, the group represented by formula (i), or the group represented by formula (ii);
R is as defined above with respect to R in Xb¹, Xb², Yb¹, and Yb² of formula (2-b);
each of Rb¹¹, Rb¹², Rb¹³ and Rb¹⁴ independently represents a substituted or unsubstituted alkyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted aralkyl group having 7 to 24 carbon atoms, a substituted or unsubstituted silyl group, a substituted or unsubstituted aromatic hydrocarbon ring group having 6 to 24 ring carbon atoms, or a substituted or unsubstituted aromatic heterocyclic group having 2 to 24 ring carbon atoms;
s¹ represents an integer of 0 to 4, and when s¹ is 2 or more, groups Rb¹¹ may be the same or different;
t¹ represents an integer of 0 to 3, and when t¹ is 2 or more, groups Rb¹² may be the same or different;
u¹ represents an integer of 0 to 3, and when u¹ is 2 or more, groups Rb¹³ may be the same or different, and
v¹ represents an integer of 0 to 4, and when v¹ is 2 or more, groups Rb¹⁴ may be the same or different.

3. The aromatic heterocyclic derivative according to claim 2, wherein B in formula (1) is a group represented by formula (2-A) or a group represented by formula (2-B): 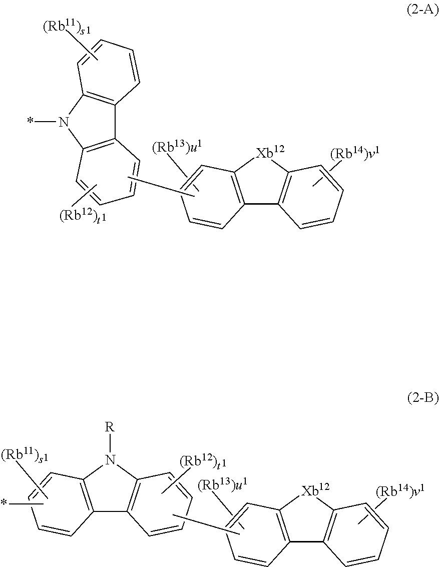
in formula (2-A):
Xb¹², Rb¹¹, Rb¹², Rb¹³, Rb¹⁴, s¹, t¹, u¹, and v¹ are as defined in formula (2-b-1);
* is bonded to L¹ of formula (1);
in formula (2-B):
s¹ is an integer of 0 to 3;
Xb¹², R, Rb¹¹, Rb¹², Rb¹³, Rb¹⁴, t¹, u¹, and v¹ are as defined in formula (2-b-1); and
* is bonded to L¹ of formula (1).

4. The aromatic heterocyclic derivative according to claim 1, wherein A of formula (1) is a residue of a ring assembly which comprises at least one substituted or unsubstituted aromatic hydrocarbon ring and at least one substituted or unsubstituted aromatic heterocyclic ring.

5. The aromatic heterocyclic derivative according to claim 4, wherein A of formula (1) is a residue of a ring assembly represented by formula (4-a) or a residue of a ring assembly represented by formula (4-b): 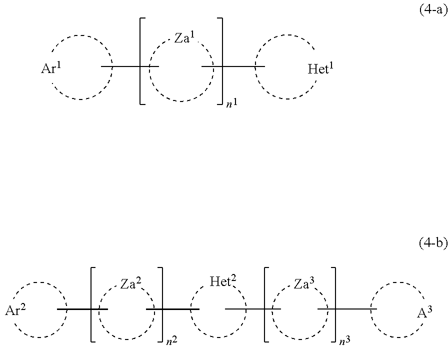
in formula (4-a):
Het¹ represents a substituted or unsubstituted aromatic heterocyclic group;
Ar¹ represents a substituted or unsubstituted aromatic hydrocarbon ring group;
Za¹ represents a substituted or unsubstituted aromatic hydrocarbon ring group or a substituted or unsubstituted aromatic heterocyclic group;
n¹ represents an integer of 0 to 2, and when n¹ is 2, groups Za¹ may be the same or different;
in formula (4-b):
Het² represents a substituted or unsubstituted aromatic heterocyclic group;
each of Ar² and Ar³ independently represents a substituted or unsubstituted aromatic hydrocarbon ring group;
each of Za² and Za³ independently represents a substituted or unsubstituted aromatic hydrocarbon ring group or a substituted or unsubstituted aromatic heterocyclic group;
n² represents an integer of 0 to 2, and when n² is 2, groups Za² may be the same or different; and
n³ represents an integer of 0 to 2, and when n³ is 2, groups Za³ may be the same or different.

6. The aromatic heterocyclic derivative according to claim 5,
wherein each of Het¹ in formula (4-a) and Het² in formula (4-b) is a substituted or unsubstituted nitrogen-containing aromatic heterocyclic group.

7. The aromatic heterocyclic derivative according to claim 1, wherein when the group represented by formula (3) is bonded to A, F is a group selected from the group consisting of a cyano group, a fluorine atom, a haloalkyl group, a substituted or unsubstituted triphenylenyl group, a substituted or unsubstituted azafluorenyl group, and a substituted or unsubstituted bipyridinyl group.

8. The aromatic heterocyclic derivative according to claim 7, wherein when the group represented by formula (3) is bonded to A, F is a group selected from the group consisting of a cyano group, a fluorine atom, and a haloalkyl group.

9. The aromatic heterocyclic derivative according to claim 1, wherein when the group represented by formula (3) is bonded to L¹ or B, F is a group selected from the group consisting of a cyano group, a fluorine atom, a haloalkyl group, a substituted or unsubstituted triphenylenyl group, a substituted or unsubstituted azafluorenyl group, a substituted or unsubstituted pyrimidinyl group, and a substituted or unsubstituted bipyridinyl group.

10. The aromatic heterocyclic derivative according to claim 9, wherein when the group represented by formula (3) is bonded to L¹ or B, F is a group selected from the group consisting of a cyano group, a fluorine atom, and a haloalkyl group.

11. A material for an organic electroluminescence device comprising the aromatic heterocyclic derivative according to claim 1.

12. A solution of a material for an organic electroluminescence device comprising a solvent and the aromatic heterocyclic derivative according to claim 1 which is dissolved in the solvent.

13. An organic electroluminescence device comprising a cathode, an anode, and one or more organic thin film layers which are disposed between the cathode and the anode and comprise a light emitting layer, wherein at least one layer of the one or more organic thin film layers comprises the aromatic heterocyclic derivative according to claim 1.

14. The organic electroluminescence device according to claim 13, wherein the light emitting layer comprises the aromatic heterocyclic derivative as a host.

15. The organic electroluminescence device according to claim 13, wherein the light emitting layer comprises a phosphorescent material.

16. The organic electroluminescence device according to claim 15, wherein the phosphorescent material is an ortho-metallated complex of a metal atom selected from the group consisting of iridium (Ir), osmium (Os), and platinum (Pt).

17. The organic electroluminescence device according to claim 13, wherein the organic electroluminescence device comprises an electron injecting layer between the cathode and the light emitting layer, and the electron injecting layer comprises a nitrogen-containing ring derivative.

18. The organic electroluminescence device according to claim 13, wherein the organic electroluminescence device comprises an electron transporting layer between the cathode and the light emitting layer, and the electron transporting layer comprises the aromatic heterocyclic.

19. The organic electroluminescence device according to claim 13, wherein the organic electroluminescence device comprises a hole transporting layer between the anode and the light emitting layer, and the hole transporting layer comprises the aromatic heterocyclic.

20. The organic electroluminescence device according to claim 13, wherein a reducing dopant is added to an interfacial region between the cathode and the organic thin film layer.
